(19) United States
(12) Patent Application Publication
(10) Pub. No.: US 2005/0176764 A1
(43) Pub. Date: Aug. 11, 2005

(54) Medicine for treating cancer

(75) Inventors: Chikage Mataki, Yokohama-shi Kanagawa (JP); Tatsuhiko Kodama, Tokyo (JP); Takeshi Doi, Tokyo (JP); Masahiro Tamura, Tokyo (JP); Toshiaki Oda, Tokyo (JP); Yukiyoshi Yamazaki, Tokyo (JP); Masahiro Nishikawa, Tokyo (JP); Shunji Takemura, Tokyo (JP); Masao Ohkuch, Saitama (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KOWA CO., LTD, Aichi (JP)

(21) Appl. No.: 10/510,759

(22) PCT Filed: Apr. 11, 2003
(86) PCT No.: PCT/JP03/04602

Related U.S. Application Data
(60) Provisional application No. 60/371,675, filed on Apr. 12, 2002.
(60) Provisional application No. 60/412,571, filed on Sep. 23, 2002.

(51) Int. Cl. A61K 31/4545; A61K 31/445

(52) U.S. Cl. 514/316; 514/317

(57) ABSTRACT

Abstract The present invention is directed to a method for treating cancer, a method for inhibiting histone deacetylase, and a method for facilitating gene therapy, comprising administering an effective amount of a cyclic amine compound represented by the following formula (1):(wherein R1, R2, and R3 each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W1 and W2, which are identical to or different from each other, represent N or CH; X represents O, NR4, CONR4, or NR4CO; R4 represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a hydrate thereof.208

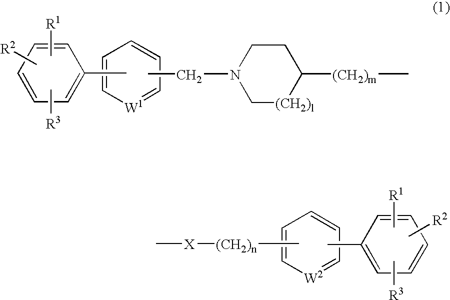

### TECHNICAL FIELD

The present invention relates to a medicine for treating cancer with reduced side effects.

### BACKGROUND ART

Trichostatin A (hereinafter referred to as “TSA”) was first isolated as an antifungal antibiotic from Streptomyces hygroscopicus by Tsuji and others in 1976 (J. Antibiot. (Tokyo), 1976 29(1): 1-6). Later, Yoshida and others reported that TSA is a potent inducer of differentiation in erythroleukemia cells (Cancer Res., 1987 47(14): 3688-91) and also acts as an inhibitor in G1 and G2 phases in the cell cycle (Exp. Cell. Res., 1988 177(1): 122-31), and also clarified that these actions are caused by inhibiting histone deacetylase (hereinafter referred to as “HDAC”) (J. Biol. Chem., 1990 265(28): 17174-9). It has been suggested that TSA inhibits HDAC by formation of a stable complex from the hydroxamic acid moiety in TSA structure and the amino acid in the active center of HDAC which are chelated via metallic zinc (Nature, 1999 401(6749): 188-93).

HDAC inhibition causes highly acetylated nuclear histones, which leads to expression of genes. Among the genes affected by inhibition of HDAC, quite a few are important ones having close relation with cancer. Therefore, a number of HDAC inhibitors have been studied for their potential use as an anticancer agents. Some actions of HDAC inhibitors include inhibition of proliferation, acceleration of differentiation, apoptosis induction, upraising of p21 expression, and upraising of MHC expression. Moreover, by virtue of gene expression promoting action of HDAC, they are expected to improve the efficacy of transferred genes in gene therapy (see, for example, “Ketsueki •Shuyo-ka,” 2001 42(5): 416-22; Gene & Medicine, 2002 6(1): 10-14; Japanese Application Laid-Open (kokai) No. 2000-256397).

Anticancer actions of HDAC inhibitors, particularly TSA, reported heretofore includes proliferation inhibition against cultured stomach cancer cells and oral cancer cell ant J. Cancer. 2000 88(6): 992-7); carcinostatic action against a rat breast cancer model (Clin. Cancer Res., 2001 7(4): 971-6); and proliferation inhibition and apoptosis induction for cultured liver cancer cells (J. Hepatol., 2002 36(2): 233-40).

Studies on HDAC inhibitors, which are expected to serve as anti-cancer drugs or to facilitate gene therapies, have focused on the synthesis of analogues of acetyl lysine, which acts as a substrate of HDAC. That is, a variety of HDAC inhibitors having a functional group which interacts with zinc (e.g., a hydroxamic acid group or an epoxy-ketone group) and those having a cap site consisting of an aromatic or cyclic peptide have been synthesized and studied. In addition, as a peptide not having an analogous structure of acetyl lysine as described above, FK228 and the like have been synthesized and studied as HDAC inhibitors (“Ketsueki •Shuyo-ka,” 2001 42(5): 416-22).

However, thus far HDAC inhibitors which are non-peptide compounds and are not analogues of acetyl lysine have virtually remained unknown.

Thus, the present invention provides a novel substance which inhibits HDAC and which is a non-peptide and is not an analogue of HDAC substrate; and a method for treating cancer using the substance with reduced side effects.

### DISCLOSURE OF THE INVENTION

Accordingly, by use of culture cell systems, the present inventors have searched for substances which affect HDAC, and quite unexpectedly have found that compounds represented by the following formula (1) exhibit excellent HDAC-inhibitory activity, gene therapy facilitating effect, and cancer cell proliferation-inhibiting action, and thus are useful medicines for treating cancer to complete the invention.

Accordingly, the present invention provides a medicine for treating cancer, comprising administering an effective amount of a cyclic amine compound represented by the following formula (1): 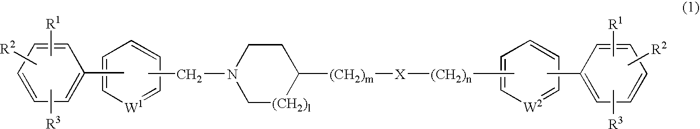
(wherein R¹, R², and R³ each independently represent a hydrogen atom, a halogen atom, a hydroxy group, an alkyl group, a halogen-substituted alkyl group, an alkoxy group, an alkylthio group, a carboxy group, an alkoxycarbonyl group, or an alkanoyl group; W¹ and W² each independently represent N or CH; X represents O, NR¹, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a hydrate thereof.

The present invention also provides a method for inhibiting HDAC, comprising administering an effective amount of the cyclic amine compound represented by the above formula (1), a salt thereof, or a hydrate thereof.

The present invention also provides a method for facilitating gene therapy, comprising administering an effective amount of a cyclic amine compound represented by the above formula (1), a salt thereof, or a hydrate thereof.

The present invention also provides a medicine for treating cancer and an HDAC inhibitor, comprising, as an active ingredient, a cyclic amine compound represented by the above formula (1), a salt thereof, or a hydrate thereof.

The present invention also provides use of a cyclic amine compound represented by the above formula (1), a salt thereof, or a hydrate thereof for producing a medicine for treating cancer and an HDAC inhibitor.

The present invention also provides a medicinal composition for treating cancer and an HDAC inhibiting composition, comprising a cyclic amine compound represented by the above formula (1), a salt thereof, or a hydrate thereof, and a pharmaceutically acceptable carrier.

### BRIEF DESCRIPTION OF THE DRAWINGS

FIG. 1 shows correlation in terms of various gene expression level.

FIG. 2 shows relative gene expression levels of several genes.

### BEST MODE FOR CARRYING OUT THE INVENTION

Examples of the halogen atom represented by R¹ to R³ in formula (1) include a fluorine atom, a chlorine atom, a bromine atom, and an iodine atom.

Examples of the alkyl group represented by R¹ to R⁴ include linear, branched, or cyclic C1-C8 alkyl groups. Examples of the linear or branched C1-C8 alkyl groups include a methyl group, an ethyl group, a propyl group, a butyl group, a pentyl group, a hexyl group, a heptyl group, and an octyl group. Examples of the cyclic C3-C8 alkyl groups include a cyclopropyl group, a cyclobutyl group, a cyclopentyl group, a cyclohexyl group, a cyclohexylmethyl group, and a cyclohexylethyl group. Of these, C1-C6 alkyl groups such as a methyl group, an ethyl group, a n-propyl group, an isopropyl group, and a n-butyl group are particularly preferred.

Examples of the halogen-substituted alkyl group represented by R¹ to R³ include C1-C8 alkyl groups substituted by one to three halogen atoms. Of these, C1-C6 alkyl groups substituted by one to three halogen atoms such as a trifluoromethyl group and a 2,2,2-trifluoroethyl group are particularly preferred.

Examples of the alkoxy group include linear, branched, or cyclic C1-C8 alkoxy groups. Examples of the linear or branched C1-C8 alkoxy groups include a methoxy group, an ethoxy group, a n-propoxy group, an iso-propoxy group, a n-butoxy group, an iso-butoxy group, a sec-butoxy group, a tert-butoxy group, a pentyloxy group, and a hexyloxy group. Examples of the C3-C8 cycloalkyloxy groups include a cyclopropyloxy group, a cyclobutyloxy group, a cyclopentyloxy group, a cyclohexyloxy group, a cyclohexylmethyloxy group, and a cyclohexylethyloxy group. Of these, a C1-C6 alkoxy group such as a methoxy group, an ethoxy group, a n-propoxy group, an isopropoxy group, or a n-butoxy group is particularly preferred.

Examples of the alkylthio group include C1-C8 alkylthio groups, and C1-C6 alkylthio groups such as a methylthio group, an ethylthio group, a n-propylthio group, and an isopropylthio group are preferred.

Examples of the alkoxycarbonyl group include C1-C6 alkoxycarbonyl groups, and C1-C4 alkoxycarbonyl groups such as a methoxycarbonyl group, an ethoxycarbonyl group, and a tert-butoxycarbonyl group are preferred.

Examples of the alkanoyl group include C1-C6 alkanoyl groups, and C1-C4 alkanoyl group such as an acetyl group, a propionyl group, a butyryl group, and an iso-butyryl group are preferred.

Examples of the alkenyl group represented by R⁴ include C3-C8 alkenyl groups, and C3-C6 alkenyl groups such as a 2-propenyl group and a 3-butenyl group are preferred. Examples of the alkynyl group include C3-C8 alkynyl groups, and C3-C6 alkynyl groups such as a 2-propynyl group and a 3-butynyl group are preferred.

Examples of the aryl group represented by R⁴ include C6-C14 aryl groups, and, among others, a phenyl group, a naphthyl group, an anthryl group, an indenyl group, an indanyl group, and a 5,6,7,8-tetrahydronaphthyl group are preferred.

Examples of the heteroaryl group represented by R⁴ include heteroaryl groups containing a 5- or 6-membered ring having one to four nitrogen atoms, and among others, an imidazolyl group, a pyridyl group, and a pyrimidinyl group are preferred.

Examples of the aralkyl group represented by R⁴ include a (C6-C14)-aryl-(C1-C6)-alkyl group, and a phenyl-(C1-C6)-alkyl group or a naphthyl-(C1-C6)-alkyl group such as a benzyl group, a naphthylmethyl group, a phenylethyl group, or a phenylpropyl group is exemplified. Examples of the heteroaralkyl group represented by R⁴ include heteroaryl-(C1-C6)-alkyl groups containing a 5- or 6-membered ring having one to four nitrogen atoms such as an imidazolyl-(C1-C6)-alkyl group, a pyridyl-(C1-C6)-alkyl group, or a pyrimidinyl-(C1-C6)-alkyl group.

The aforementioned aryl groups, heteroaryl groups, aralkyl groups, or heteroaralkyl groups may be substituted by a substituent. Examples of the substituent include one to three groups or atoms selected from an alkyl group, an alkoxy group, a halogen-substituted alkoxy group, an alkylthio group, an alkylsulfinyl group, an alkylsulfonyl group, a halogen atom, a nitro group, an amino group, an acetylamino group, a trifluoromethyl group, and an alkylenedioxy group. Examples of the alkyl group, the alkoxy group, and the alkylthio group include those described in relation to the R¹ to R³. Examples of the alkyl group contained in the alkylsulfinyl group and the alkylsulfonyl group include a C1-C3-alkyl group, particularly a methyl group, an ethyl group, a n-propyl group, and an isopropyl group. Preferable examples of the halogen-substituted alkoxy group include a C1-C8 alkoxy group substituted by one to three halogen atoms, particularly a C1-C4 alkoxy group substituted by one to three halogen atoms such as a trifluoromethoxy group or a 2,2,2-trifluoroethoxy group. Examples of the alkylenedioxy group include a C1-C3 alkylenedioxy group such as a methylenedioxy group, an ethylenedioxy group, or a propylenedioxy group.

X is preferably NR⁴, and R⁴ is more preferably a C1-C8 alkyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl-(C1-C6)-alkyl group, or a substituted or unsubstituted heteroaryl-(C1-C6)-alkyl group containing a 5- or 6-membered ring having one to four nitrogen atoms.

Preferably, R¹, R², and R³ are bonded at the 3-, 4-, and 5-positions, respectively, of the phenyl group. In this case, more preferably, R¹ and R³ (i.e., the groups bonded at the 3- and 5-positions of the phenyl group) are an alkoxy group or a halogen atom, and R² (i.e., the group bonded at the 4-position of the phenyl group) is a hydrogen atom, a halogen atom, a hydroxy group, an alkyl group, a halogen-substituted alkyl group, an alkoxy group, an alkylthio group, a carboxy group, an alkoxycarbonyl group, or an alkanoyl group.
l is a number of 0 or 1, with 1 being preferred.

W¹ is preferably N. W² is preferably N.

Among the compounds represented by formula (1), preferred is a compound in which X is NR⁴, and R⁴ is a C1-C8 alkyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl-(C1-C6)-alkyl group, or a substituted or unsubstituted heteroaryl-(C1-C6)-alkyl group containing a 5- or 6-membered ring having one to four nitrogen atoms. More preferably, R4 is a phenyl group or a pyridyl group which may be substituted by one or two groups or atoms selected from a halogen atom, an alkyl group, an alkoxy group, an alkylthio group, a trifluoromethyl group, and an alkylenedioxy group, or a C1-C8 alkyl group.

No particular limitations are imposed on the acid-addition salts of the compound (1) of the present invention, so long as the salts are pharmaceutically acceptable. Examples of the salts include addition salts of mineral acids such as hydrochlorides, hydrobromides, hydriodides, sulfates, and phosphates; and addition salts of organic acids such as benzoates, methanesulfonates, ethanesulfonates, benzenesulfonates, p-toluenesulfonates, oxalates, malates, fumarates, tartarates, citrates, and acetates.

The compound (1) of the present invention may form a solvate represented by hydrate, and the present invention encompasses such solvates.

The compound (1) of the present invention can be produced through the following methods A through L.
Process A: Preparation of the Compound of the Formula (1) wherein l=1, m=0, n=1 and X═CONR⁴ 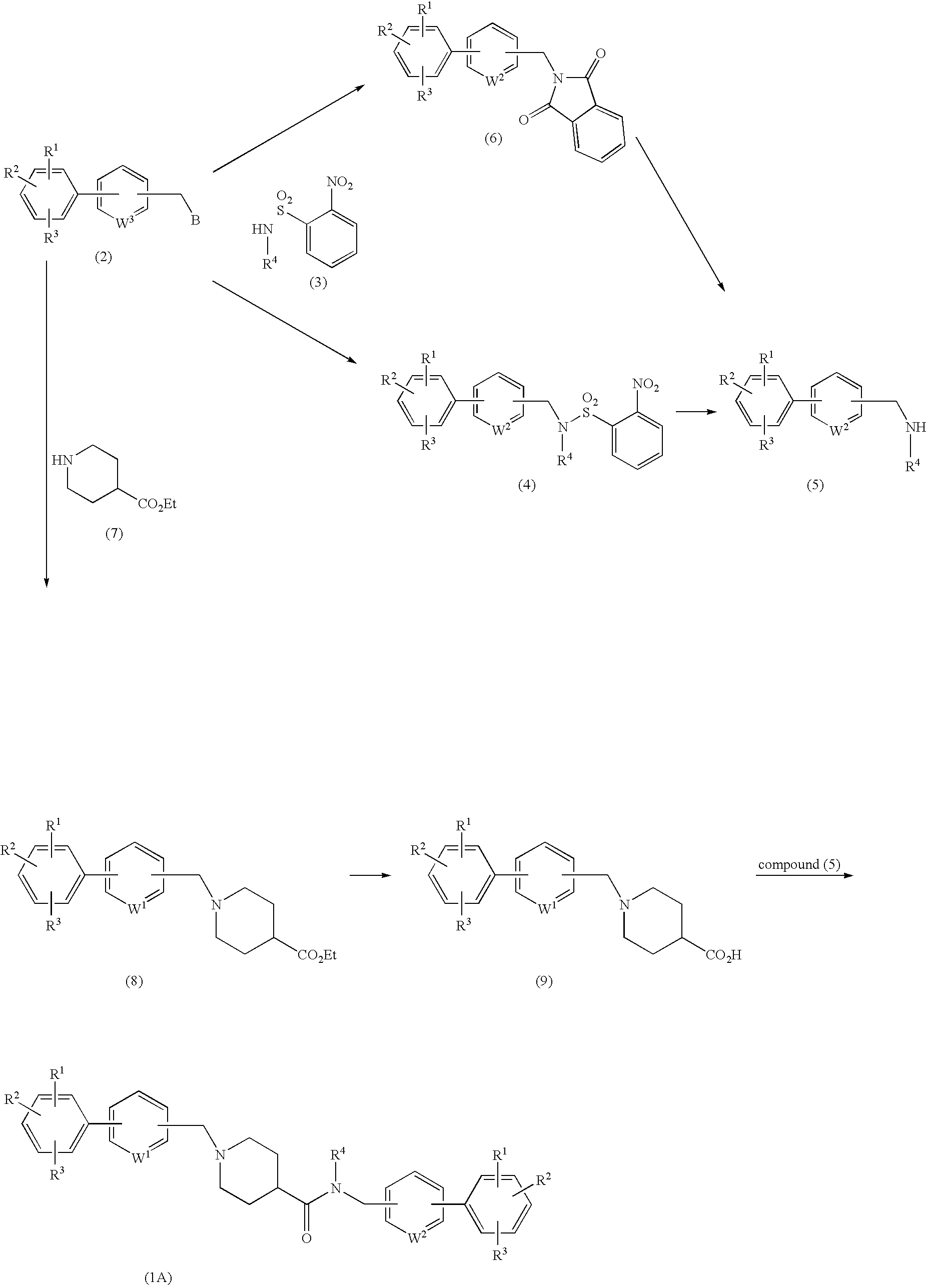
wherein, W¹, W², R¹, R², R³ and R⁴ are as defined above, W³ has the same meaning as W¹ or W², and B denotes a leaving group such as a halogen atom, or methanesulfonyloxy or p-toluenesulfonyloxy group.

Compound (2) and a N-(2-nitro)benzenesulfonylamine derivative (3) are reacted to give compound (4). The resulting compound (4) is treated with thiophenol in the presence of a base such as potassium carbonate to eliminate the 2-nitrobenzenesulfonyl group, thereby giving amine compound (5). Alternatively, when R⁴ is H, it is possible to react compound (2) with potassium phthalimide and then treat the resulting phthalimide derivative (6) with hydrazine to give the corresponding amine compound (5).

On the other hand, compound (2) is reacted with ethyl isonipecotate (7) in a solvent such as acetonitrile, N,N-dimethylformamide (DMF), dimethyl sulfoxide (DMSO), tetrahydrofuran (THF), dioxane, toluene, benzene, etc. in the presence of a base such as potassium carbonate or the like at a temperature between 0° C. and a reflux temperature for several hours to several days, preferably at room temperature overnight, to give compound (8). The compound (8) is subjected to a usual alkaline hydrolysis to give the corresponding carboxylic acid compound (9).

The carboxylic acid compound (9) is reacted with the amine compound (5) using a dehydration condensing agent such as 1-(3-dimethylaminopropyl)-3-ethylcarbodiimide hydrochloride (water-soluble carbodiimide), 2-(1H-benzotriazol -1-yl)-1,1,3,3-tetramethyluronium hexafluorophosphate (HBTU) or the like in a solvent such as chloroform, dichloroethane, THF, dioxane, acetonitrile, etc. at a temperature between 0° C. and a reflux temperature for several hours to several days, preferably at room temperature for 12 hours, to give an end product (1A).
Process B: Preparation of the Compound of the Formula (1) wherein l=1, m=0, n=1 and X═O 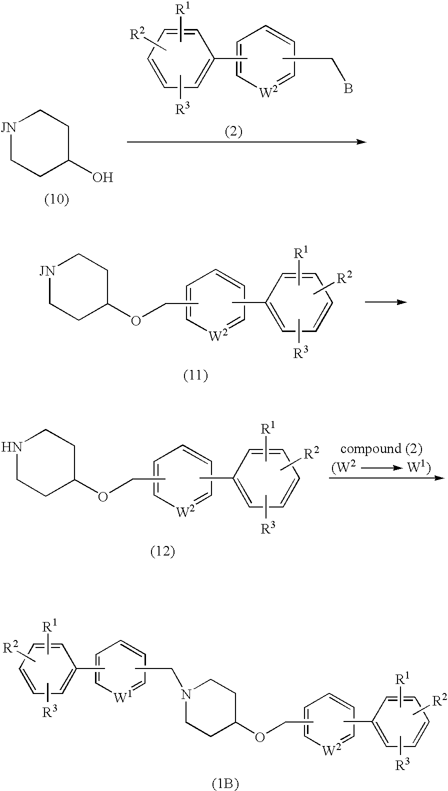
wherein, B, W¹, W², R¹, R² and R³ are as defined above, and J denotes a protecting group such as benzyloxycarbonyl, tert-butoxycarbonyl, acetyl, benzoyl or benzyl group. Incidentally, in the reaction schemes shown above and below, the expression “(W²→W¹)” following the term “compound (2)” means that W² in the formula representing compound (2) is changed to W¹.

4-hydroxypiperidine compound (10) with a protected amino group is reacted with compound (2) in the presence of sodium hydride and potassium iodide in a solvent such as DMF, DMSO, etc. at a temperature between 0° C. and a reflux temperature for several hours to several days, preferably at room temperature for 2 days, to give compound (11). The protecting group in the compound (11) is removed in a known manner. The resulting compound (12) is reacted with compound (2) in the presence of a base such as potassium carbonate in a solvent such as acetonitrile, DMF, DMSO, THF, dioxane, etc. at a temperature between 0° C. and a reflux temperature for several hours to several days, preferably at room temperature for 4 hours, to give an end product (1B).
Process C: Preparation of the Compound of the Formula (1) wherein l=1, m=0, n=0, X═NR⁴CO and R⁴═H or Me 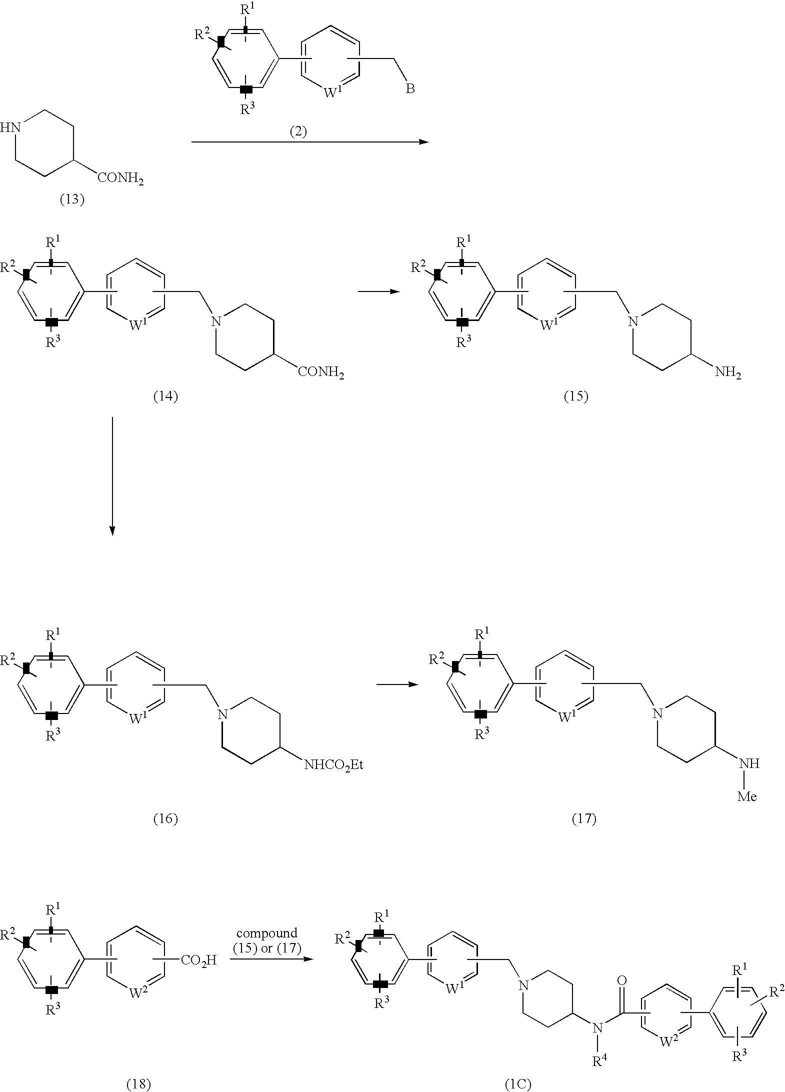
wherein, B, W¹, W², R¹, R² and R³ are as defined above, and R⁴ denotes a hydrogen atom or methyl group.

Isonipecotamide (13) is reacted with compound (2) in the presence of a base such as potassium carbonate, sodium carbonate or the like in a solvent such as acetonitrile, DMF, DMSO, THF, dioxane, etc. at a temperature between 0° C. and a reflux temperature for several hours to several days, preferably at room temperature for 4 hours, to give compound (14). The compound (14) is subjected to Hofmann rearrangement reaction to give amine compound (15).

On the other hand, by subjecting the compound (14) to Hofmann rearrangement reaction in ethanol, carbamate compound (16) is obtained. Then, by subjecting the compound (16) to a reduction reaction using lithium aluminum hydride, methylamine compound (17) is obtained.

By reacting carboxylic acid compound (18) with the amine compound (15) or methylamine compound (17) similarly to the condensation reaction in Process A, an end compound (1C) is obtained.
Process D: Preparation of the Compound of the Formula (1) wherein l=1, m=0, n=1 and X═NR⁴ 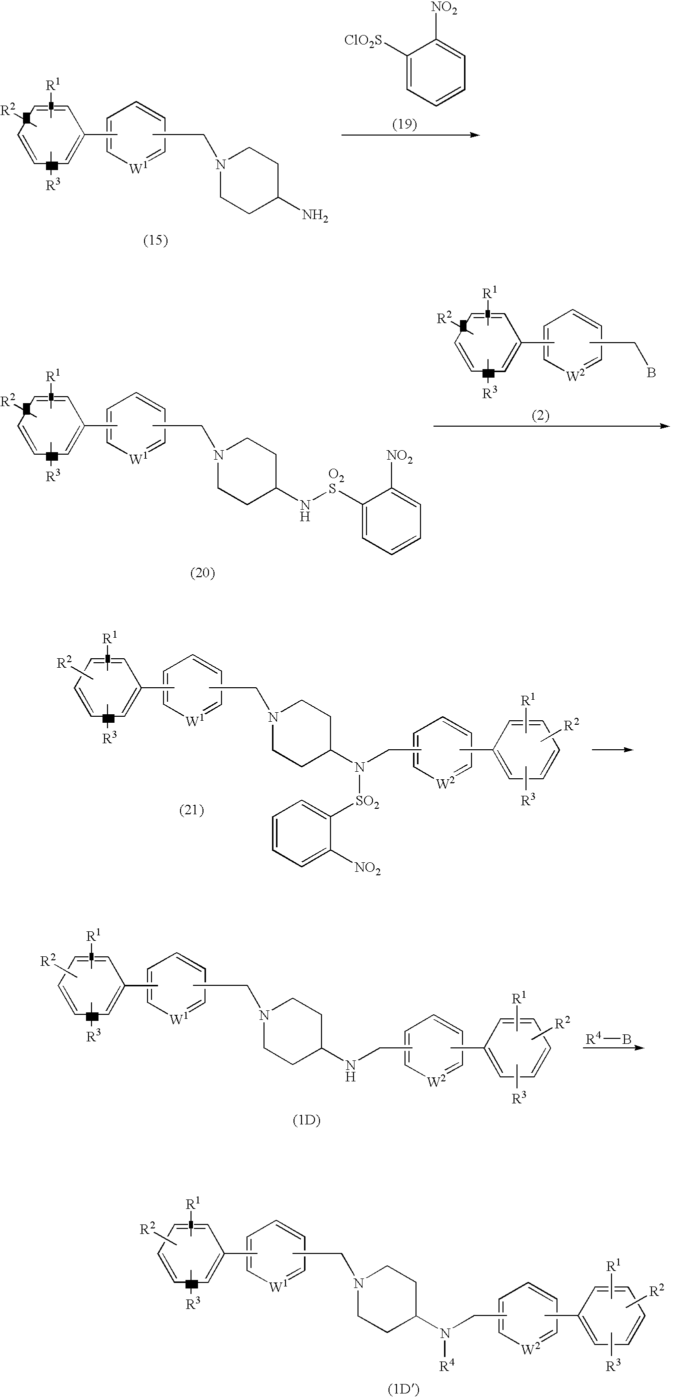 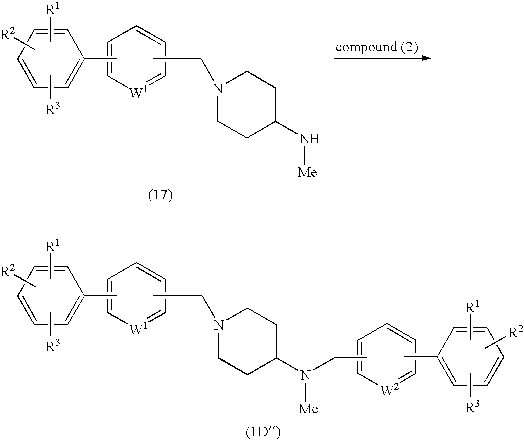
wherein, B, W¹, W², R¹, R² and R³ are as defined above, and R⁴ denotes an alkyl, alkenyl, alkynyl, aralkyl or heteroaralkyl group.

The amine compound (15) mentioned in the above is reacted with 2-nitrobenzenesulfonyl chloride (19) according to a known manner to give compound (20). The compound (20) is reacted with compound (2) in the presence of a base such as potassium carbonate in a solvent such as acetonitrile, DMF, DMSO, THF, dioxane or the like at a temperature between 0° C. and a reflux temperature for several hours to several days, preferably at room temperature for 4 hours, to give compound (21). The benzenesulfonyl group of the compound (21) is removed similarly to the procedure for the compound (4) in Process A to give an end compound (1D) (R⁴═H). The compound (1D) is reacted with R⁴-B in the presence of a base such as sodium carbonate, sodium bicarbonate, potassium carbonate, cesium carbonate or the like in a solvent such as acetonitrile, THF, dioxane, chloroform, dichloromethane, DMF, DMSO or the like at a temperature between 0° C. and a reflux temperature for several hours to several days, preferably at 80° C. for 12 hours, to give compound (1D′).

On the other hand, the methylamine compound (17) is reacted compound (2) in the presence of a base such as potassium carbonate in a solvent such as acetonitrile, DMF, DMSO, THF, dioxane or the like at a temperature between 0° C. and a reflux temperature for several hours to several days, preferably at room temperature for 4 hours, to give an end compound (1D″) (R⁴=Me).
Process E: Preparation of the Compound of the Formula (1) wherein l=1, m=0 or 1, n=1 and X═NR⁴, 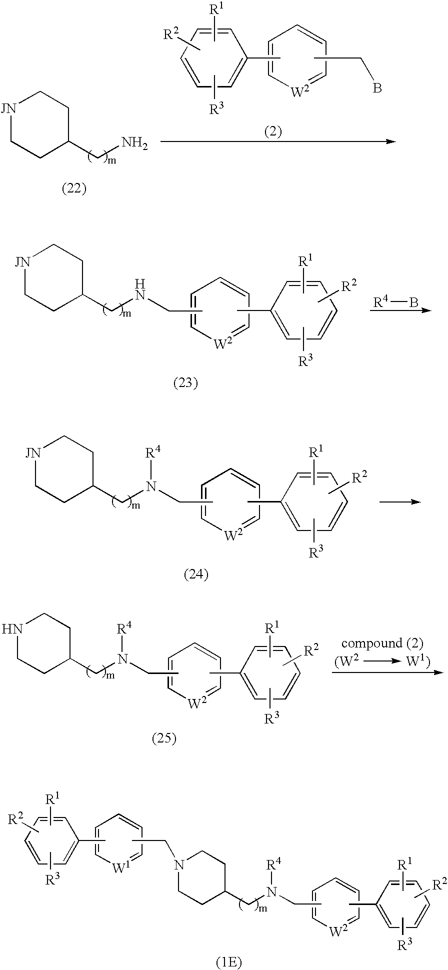
wherein, B, J, W¹, W², R¹, R² and R³ are as defined above, and R⁴ denotes an alkyl, alkenyl, alkynyl, aralkyl or heteroaralkyl group.

Aminopiperidine derivative (22) in which the amino group on the ring is protected is reacted with compound (2) in the presence of a base such as potassium carbonate in a solvent such as acetonitrile, DMF, DMSO, THF, dioxane or the like at a temperature between 0° C. and a reflux temperature for several hours to several days, preferably at room temperature for 4 hours, to give compound (23). The compound (23) is reacted with R⁴-B in the presence of a base such as sodium carbonate, sodium bicarbonate, potassium carbonate, cesium carbonate or the like in a solvent such as acetonitril, THF, dioxane, chloroform, dichloroethane, DMF, DMSO or the like at a temperature between 0° C. and a reflux temperature for several hours to several days, preferably at 80° C. for 12 hours, to give compound (24). After removal of the protecting group, the compound (25) is reacted compound (2) in the presence of a base such as potassium carbonate in a solvent such as acetonitrile, DMF, DMSO, THF, dioxane or the like at a temperature between 0° C. and a reflux temperature for several hours to several days, preferably at room temperature for 4 hours, to give compound (1E).
Process F: Preparation of the Compound of the Formula (1) wherein l=1, m=0, n=1 and X═NR⁴, 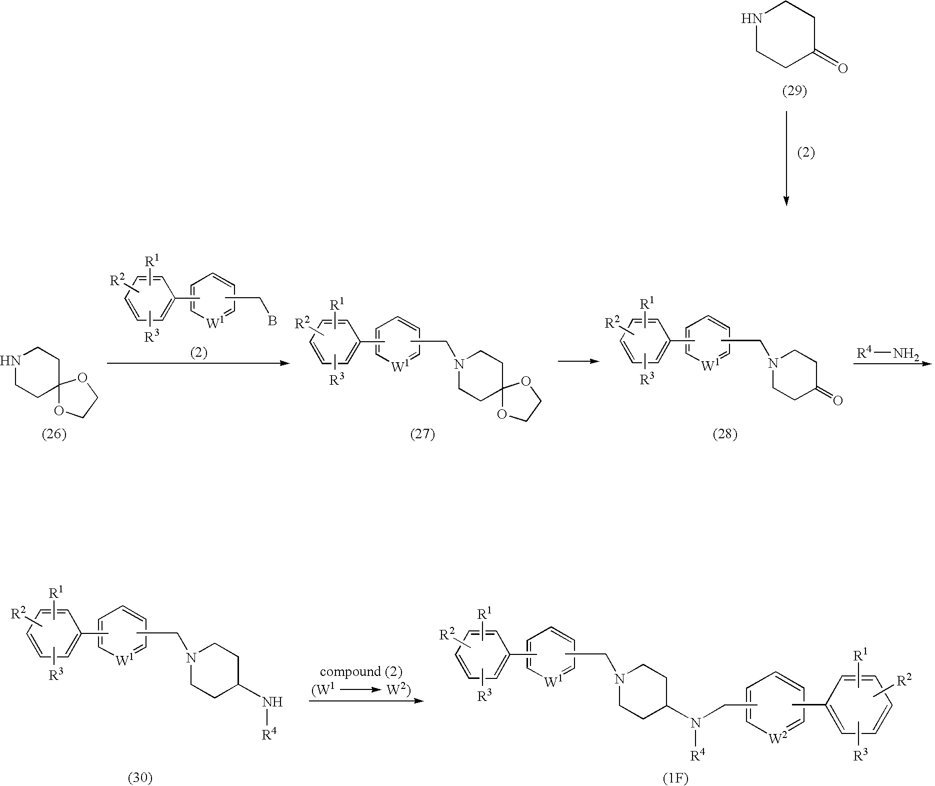
wherein, B, W¹, W², R¹, R² and R³ are as defined above, and R⁴ denotes an alkyl, alkenyl, alkynyl, aralkyl, heteroaralkyl, aryl or heteroaryl group.

4-piperidone ethylene ketal (26) is reacted with compound (2) in the presence of a base such as potassium carbonate in a solvent such as acetonitrile, DMF, DMSO, THF, dioxane, etc. at a temperature between 0° C. and a reflux temperature for several hours to several days, preferably at room temperature for 4 hours, to give compound (27), which in turn is deketalized by using an acid to give ketone compound (28).

On the other hand, 4-piperidone (29) is reacted compound (2) in the presence of a base such as potassium carbonate in a solvent such as acetonitrile, DMF, DMSO, THF, dioxane or the like at a temperature between 0° C. and a reflux temperature for several hours to several days, preferably at room temperature for 4 hours, to give compound (28). Using the compound (28), amine compound (30) can be prepared according to either of the following two synthesis processes:

Synthesis process 1: The compound (28) is reacted with an amine compound of the formula: R⁴—NH₂ in the presence of molecular sieves in toluene or benzene at a temperature between 0° C. and a reflux temperature for several hours to several days, preferably at reflux temperature for 12 hours, followed by reaction with a reducing agent such as sodium borohydride or sodium cyanoborohydride at a temperature between 0° C. and a reflux temperature for several minutes to several days, preferably at room temperature for 1 hour, to give the amine compound (30).

Synthesis process 2: The compound (28) is reacted with an amine compound of the formula: R⁴—NH₂ in the presence of a reducing agent such as sodium triacetoxy boron hydride in a solvent such as dichloromethane, 1,2-dichloroethane, methanol, ethanol, etc. at a temperature between 0° C. and a reflux temperature for several minutes to several days, preferably at room temperature for 4 hours, to give the amine compound (30).

The resulting compound (30) is reacted compound (2) in a solvent such as acetonitrile, DMF, DMSO, THF, dioxane, etc. at a temperature between 0° C. and a reflux temperature for several hours to several days, preferably at room temperature for 4 hours, to give an end product (IF).
Process G: Preparation of the Compound of the Formula (1) wherein l=1, m=0, n=1 and X═NR⁴ 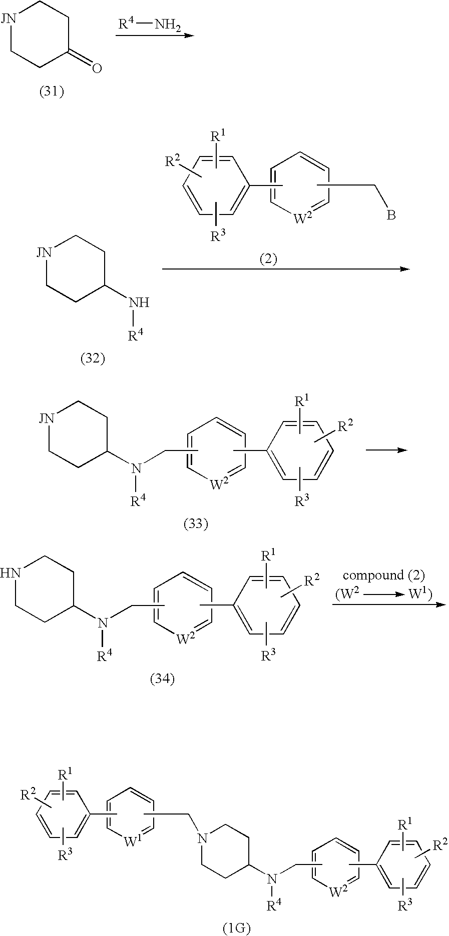
wherein, B, J, W¹, W², R¹, R² and R³ are as defined above, and R⁴ denotes an alkyl, alkenyl, alkynyl, aralkyl, heteroaralkyl, aryl or heteroaryl group.

4-piperidone derivative (31) in which the amino group on the ring is protected is reacted with an amine compound R⁴—NH₂ similarly to the procedure for preparation of compound (30) in Process F to give compound (32). The compound (32) is reacted with compound (2) in the presence of a base such as potassium carbonate in a solvent such as acetonitrile, DMF, DMSO, THF, dioxane, etc. at a temperature between 0° C. and a reflux temperature for several hours to several days, preferably at room temperature for 4 hours, to give compound (33). After removal of the protecting group from the compound (33), the resulting compound (34) is reacted with compound (2) in the presence of a base such as potassium carbonate in a solvent such as acetonitrile, DMF, DMSO, THF, dioxane, etc. at a temperature between 0° C. and a reflux temperature for several hours to several days, preferably at room temperature for 4 hours, to give an end product (1G).
Process H: Preparation of the Compound of the Formula (1) wherein l=0, m=0, n=1 and X═NH 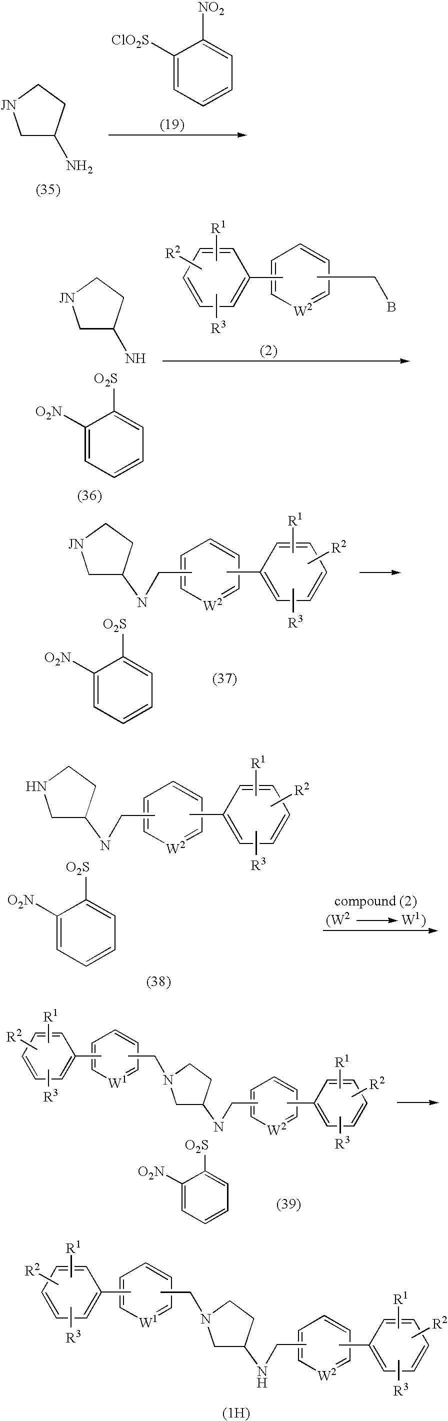
wherein, B, J, W¹, W², R¹, R² and R³ are as defied above.

3-aminopyrrolidine derivative (35) with a protected amino group on the ring is reacted with 2-nitrobenzenesulfonyl chloride (19) under usual conditions to give a benzenesulfonyl derivative (36). The derivative (36) is reacted with compound (2) in the presence of a base such as potassium carbonate in a solvent such as acetonitrile, DMF, DMSO, THF, dioxane, etc. at a temperature between 0° C. and a reflux temperature for several hours to several days, preferably at room temperature for 4 hours, to give compound (37). The protecting group of the amino group is removed from the compound (37) to give compound (38), which in turn is reacted with compound (2) in the presence of a base such as potassium carbonate in a solvent such as acetonitrile, DMF, DMSO, THF, dioxane, etc. at a temperature between 0° C. and a reflux temperature for several hours to several days, preferably at room temperature for 4 hours, to give compound (39). By subjecting the compound (39) to a reaction similar to that in the preparation of compound (5) in Process A, an end product (1H) is obtained.
Process I: Preparation of the Compound of the Formula (1) wherein l=0, m=0, n=1 and X═NR⁴ 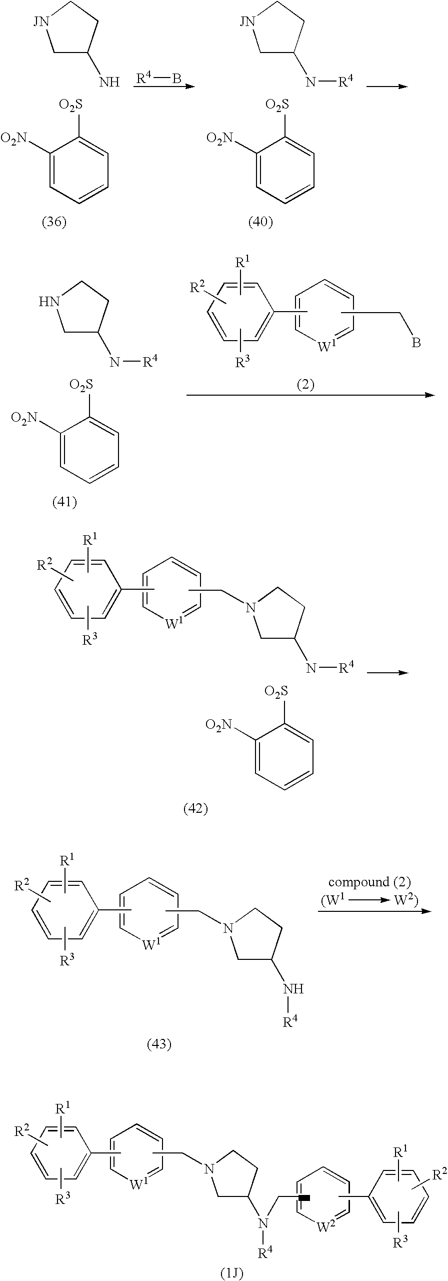
wherein, B, J, W¹, W², R¹, R² and R³ are as defined above, and R⁴ denotes an alkyl, alkenyl, alkynyl or aralkyl group.

Compound (36) is reacted with R⁴-B in the presence of a base such as sodium carbonate, potassium carbonate, etc. in a solvent such as acetonitrile, THF, dioxane, chloroform, dichloroethane, DMF, DMSO, etc. at a temperature between 0° C. and a reflux temperature for several hours to several days, preferably at 80° C. for 12 hours, to give compound (40). The amino-protecting group is removed from the compound (40), and the resulting compound (41) is reacted with compound (2) in the presence of a base such as potassium carbonate in a solvent such as acetonitrile, DMF, DMSO, THF, dioxane, etc. at a temperature between 0° C. and a reflux temperature for several hours to several days, preferably at room temperature for 4 hours, to give compound (42). By subjecting the compound (42) to a reaction similar to that in the preparation of compound (5) in Process A, compound (43) is obtained. The compound (43) is reacted with compound (2) in the presence of a base such as potassium carbonate in a solvent such as acetonitrile, DMF, DMSO, THF, dioxane, etc. at a temperature between 0° C. and a reflux temperature for several hours to several days, preferably at room temperature for 4 hours, to give an end product (1I).
Process J: Preparation of the Compound of the Formula (1) wherein R²═OH 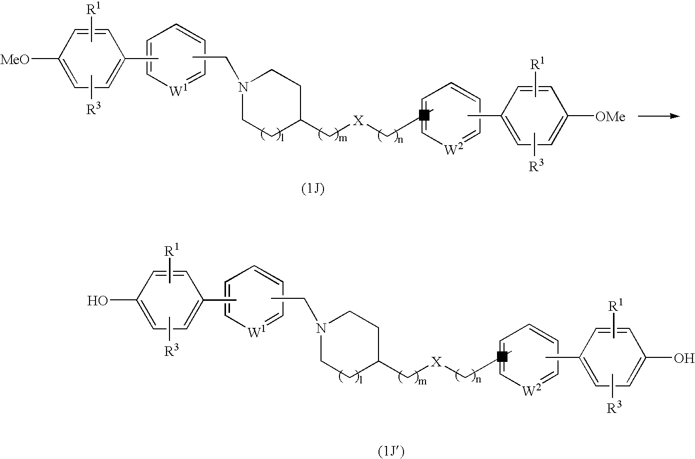
wherein, X, W¹, W², R¹, R³, l, m and n have the same meanings as initially defined.

By reacting methoxy compound (1J) with iodotrimethylsilane in a solvent such as toluene, benzene, chloroform, dichloromethane, etc. at a temperature between −25° C. and a reflux temperature for several minutes to several days, preferably at 0° C. for 2 hours, there can be obtained an end product (1J′).
Process K: Preparation of the Compound of the Formula (1) wherein l=1, m 0, n=0 and X═NR⁴CO 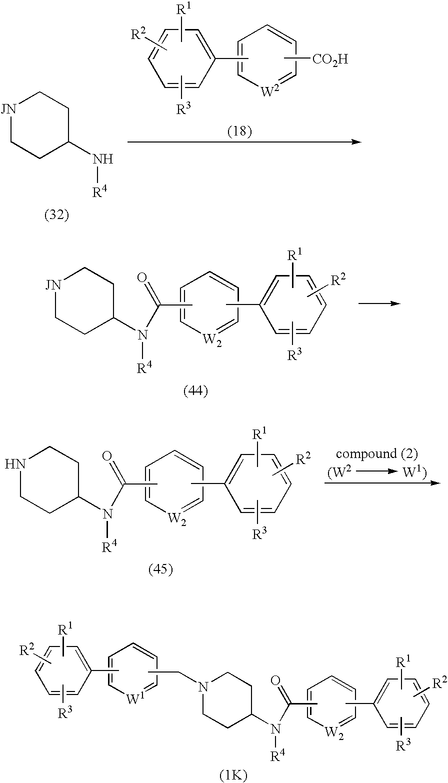
wherein, B, J, W¹, W², R¹, R² and R³ are as defined above, and R⁴ denotes an alkyl, alkenyl, alkynyl, aralkyl, heteroaralkyl, aryl or heteroaryl group.

Compound (32), which is described in the Process G, is reacted with compound (18) in the similar procedure as described in the preparation of compound (1A) in Process A to give gompound (44). After removal of the protecting group from the compound (44), the resulting compound (45) is reacted with compound (2) in the presence of a base such as potassium carbonate in a solvent such as acetonitrile, DMF, DMSO, THF, dioxane, etc. at a temperature between 0° C. and a reflux temperature for several hours to several days, preferably at room temperature for 4 hours, to give an end product (1 K).
Process L: Preparation of the Compound of the Formula (1) wherein l=1, m=0, n=1 and X=alkylsulfonylphenylamino Group 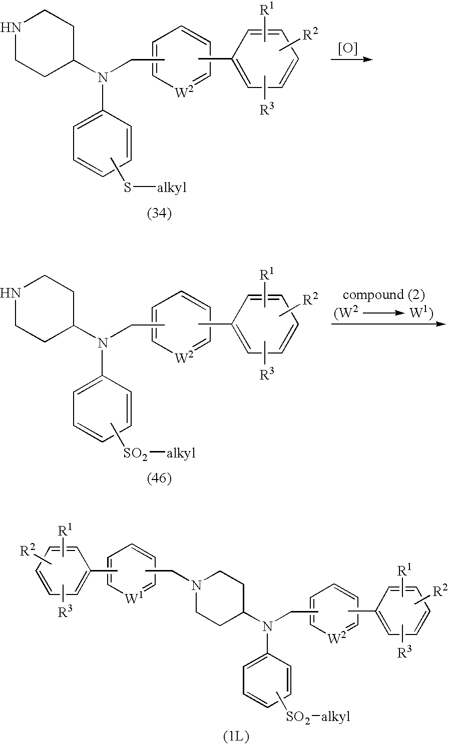
wherein, B, W¹, W², R¹, R² and R³ are as defined above.

Compound (34), which was prepared in the Process G (wherein X denotes alkylthiophenylamino group), is reacted with an oxdation agent such as 3-chloroperbenzoic acid, peracetic acid, hydrogen peroxide, etc. in the known manner to give an alkylsulfonyl derivative (46). Compound (46) is then reacted with compound (2) in the presence of a base such as potassium carbonate in a solvent such as acetonitrile, DMF, DMSO, THF, dioxane, etc. at a temperature between 0° C. and a reflux temperature for several hours to several days, preferably at 70° C. overnight, to give an end product (1L).

The compounds (1) according to the present invention are obtained by any of the above-described processes and may further be purified by using an ordinary purification means such as recrystallization or column chromatography as needed. As needed, the compounds may also be converted into the desired salts or solvates in a method known per se in the art. When the compounds (1) have an asymmetric carbon atom, the present invention includes any configurational isomers.

These compounds (1) according to the present invention possess the almost same profile of gene expression in human cells as TSA which has the HDAC inhibiting action, and exhibit potent growth inhibitory effect on cultured human cancer cells as shown in the test example.

The medicine for treating cancer according to the present invention comprises a compound (1), a salt thereof, or a solvate thereof as an active ingredient. The form of administration may be suitably selected as necessary for the therapeutic application intended without any particular limitation, including oral preparations, injections, suppositories, ointments, inhalants, eye drops, nose drops and plasters. A composition suitable for use in these administration forms can be prepared by blending a pharmaceutically acceptable carrier in accordance with the conventional preparation method publicly known by those skilled in the art.

When an oral solid preparation is formulated, an excipient, and optionally, a binder, disintegrator, lubricant, colorant, a taste corrigent, a smell corrigent and the like are added to compound (1) and the resulting composition can be formulated into tablets, coated tablets, granules, powders, capsules, etc. in accordance with methods known in the art.

As such additives described above, any additives may be used which are generally used in the pharmaceutical field. Examples include excipients such as lactose, sucrose, sodium chloride, glucose, starch, calcium carbonate, kaolin, microcrystalline cellulose and silicic acid; binders such as water, ethanol, propanol, simple syrup, glucose solution, starch solution, gelatin solution, carboxymethyl cellulose, hydroxypropyl cellulose, hydroxypropyl starch, methyl cellulose, ethyl cellulose, shellac, calcium phosphate and polyvinyl pyrrolidone; disintegrators such as dry starch, sodium alginate, agar powder, sodium hydrogencarbonate, calcium carbonate, sodium lauryl sulfate, monoglyceryl stearate and lactose; lubricants such as purified talc, stearic acid salts, borax and polyethylene glycol; and taste corrigents such as sucrose, orange peel, citric acid and tartaric acid.

When an oral liquid preparation is formulated, a taste corrigent, buffer, stabilizer, smell corrigent and/or the like are added to compound (1) and the resulting composition can be formulated into internal liquid preparations, syrup preparations, elixirs, etc. in accordance with methods known in the art. In this case, vanillin as the taste corrigent, may be used. As the buffer, sodium citrate may be mentioned. As examples of the stabilizer, tragacanth, gum arabic and gelatin may be mentioned.

When an injection is formulated, a pH adjustor, buffer, stabilizer, isotonicity agent, local anesthetic and the like may be added to compound (1) according to the present invention, and the resultant composition can be formulated into subcutaneous, intramuscular and intravenous injections in accordance with methods known in the art. Examples of the pH adjustor and buffer in this case include sodium citrate, sodium acetate and sodium phosphate. Examples of the stabilizer include sodium pyrosulfite, EDTA, thioglycolic acid and thiolactic acid. Examples of the local anesthetic include procaine hydrochloride and lidocaine hydrochloride. Examples of the isotonicity agent include sodium chloride and glucose.

When a suppository is formulated, a carrier preparation known in the art, for example, polyethylene glycol, lanoline, cacao butter, fatty acid triglyceride or the like, and optionally, a surfactant such as Tween (trade mark) and the like are added to the compound (1), and the resultant composition can be formulated into suppositories in accordance with methods known in the art.

When an ointment is formulated, a base material, stabilizer, wetting agent, preservative and the like, which are generally used, are blended with compound (1) as needed, and the resulting blend is mixed and formulated into ointments in accordance with known methods. Examples of the base material include liquid paraffin, white vaseline, bleached beeswax, octyldodecyl alcohol and paraffin. Examples of the preservative include methyl p-hydroxybenzoate, ethyl p-hydroxybenzoate and propyl p-hydroxybenzoate.

Besides the above preparations, inhalants, eye drops and nose drops may also be formulated in accordance with known methods.

The medicine for treating cancer of this invention is useful for treating various cancer and carcinoma Examples of such cancer and carcinoma include cancer or carcinoma of brain, nerve and oculus such as pituitary adenoma, acoustic neurilemoma, glioma, brain tumor; cancer and carcinoma of head and neck region such as oral cancer (i.e. tongue cancer, carcinoma of the mouth floor, carcinoma of gingiva, carcinoma of the buccal mucosa, etc.), pharyngeal cancer (i.e. nasopharyngeal cancer, oropharyngeal cancer, hypopharyngeal cancer), laryngeal cancer (i.e. glottic laryngeal cancer, etc.), maxillary cancer, thyroid cancer (i.e. papillary carcinoma, follicular carcinoma, medullary carcinoma, undifferentiated carcinoma, malignant lymphoma, etc.), sialoma (i.e. parotid abscess, cancer of submandibular gland, cancer of sublingual gland, etc.); cancer and carcinoma of breast such as thymoma, breast cancer, lung cancer, mesothelioma; cancer and carcinoma of digestive organ such as stomach cancer, esophageal cancer, colon cancer; cancer and carcinoma of liver, gallbladder and pancreas such as hepatocarcinoma, cholangiocarcinoma, pancreatic cancer, gallbladder cancer, pancreatic endocrine tumors; cancer and carcinoma of uropoietic organ such as penile carcinoma, testicular cancer, renal pelvic and ureter carcinoma, prostate cancer, renal cell carcinoma, bladder carcinoma; cancer and carcinoma of gynecologic such as vulvar cancer, uterine cancer, cervical cancer, corpus uteri carcinoma (endometrial carcinoma), uterine sarcoma, trophoblastic disease, vaginal cancer, mammary carcinoma, ovarian cancer, germ cell tumor of ovary; cancer and carcinoma of cutis such as melanoma, mycosis fungoides, skin cancer; cancer and carcinoma of bone and muscle such as malignant bone tumors (i.e. bone cancer, parosteal osteosarcoma, periosteal osteosarcoma, malignant fibrous histiocytoma, chordoma, diffuse endothelioma of bone, adamantinoma, chondrosarcoma, etc), soft part sarcoma (i.e. malignant fibrous histiocytoma, liposarcoma, synovial sarcoma, leiomyosarcoma, rhabdomyosarcoma, angiosarcoma, perithelioma, lymphagiosarcoma, neurosarcoma, malignant neuroepithelioma, soft part Ewing, extraskeletal chondrosarcoma, extraskeletal osteosarcoma, alveolar soft part sarcoma, epithelioid sarcoma, clear cell sarcoma, etc); cancer and carcinoma of blood and lymph such as malignant lymphoma, non-Hodgkin's lymphoma, Hodgkin's disease, myelodysplastic syndromes, multiple myeloma, acute myelogenous leukemia, acute lymphocytic leukemia, adult T-cell leukemia, chronic myelogenous leukemia, chronic lymphocytic leukemia, chronic myeloproliferative disorders; cancer and carcinoma of endocrine such as melanocytoma, pancreatic endocrine tumors, parathyroid cancer, adrenal tumor; cancer and carcinoma of childhood such as soft part sarcoma, cerebral tumor, retinoblastoma, Wilms' tumor, and other unidentified cancer.

The dose of the medicine for treating cancer according to the present invention varies according to the age, weight and condition of the patient to be treated, the administration method, the number of times of administration, and the like. It is however preferred that the medicine is generally orally or parenterally administered at once or in several portions in a dose of 1 to 1,000 mg per day in terms of compound (1), for an adult.

The present invention will hereinafter be described in more detail by Examples. However, the present invention is not limited to these examples.

### PREPARATION EXAMPLE 1

### Synthesis of Ethyl 2-(3,4,5-trimethoxyphenyl)isonicotinate

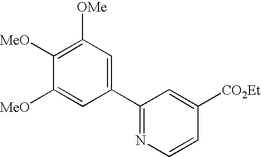

3,4,5-Trimethoxyphenylboronic acid (20.10 g) and ethyl 2-chloroisonicotinate (18.56 g) were suspended in a mixted solvent of toluene (200 mL) and THF (100 mL), and to the suspension 2 M sodium carbonate (200 mL) and tetrakis(triphenyl phosphine) palladium(0) (5.78 g) were added. The mixture was stirred at 90° C. overnight under an argon atmosphere. Ethyl acetate was added to the reaction mixture to separate an organic layer. The organic layer was washed with brine, dried over anhydrous sodium magnesium and concentrated under reduced pressure. The residue was purified by column chromatography on silica gel using hexane-ethyl acetate (5:1) to give the title compound.

Yield: 27.99 g (88%).

¹H-NMR (400 MHz, CDCl₃) δ: 1.45 (t, 3H, J=7.0 Hz), 3.92 (s, 3H), 3.99 (s, 6H), 4.46 (q, 2H, J=7.0 Hz), 7.30 (s, 2H), 7.76 (dd, 1H, J=5.1 Hz, 1.6 Hz), 8.24 (dd, 1H, J=1.6 Hz, 0.8 Hz), 8.81 (dd, 1H, J=5.1 Hz, 0.8 Hz).

### PREPARATION EXAMPLE 2

### Synthesis of 4-hydroxymethyl-2-(3,4,5-trimethoxyphenyl)pyridine

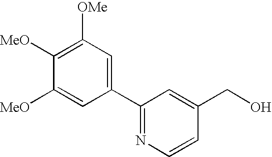

Ethyl 2-(3,4,5-trimethoxyphenyl)isonicotinate (24.57 g) was dissolved in dry THF (200 mL), and to the solution lithium aluminum hydride (2.94 g) was added at 0° C. under an argon atmosphere. The mixture was stirred at 0° C. for 1 hour as it is. A small amount of water and then sodium sulfate were added to the reaction mixture, and the reaction mixture was filtered through celite. The filtrate was evaporated, and the reultant crude crystals were recrystalized from ethyl acetate-hexane to give the title compound.

Yield: 17.53 g (820%).

¹H-NMR (400 MHz, CDCl₃) δ: 3.90 (s, 3H), 3.95 (s, 6H), 4.79 (s, 2H), 7.19 (d, 1II, J=5.1 Hz), 7.21 (s, 2H), 7.66 (s, 1H), 8.60 (d, 1H, J=5.1 Hz).

### PREPARATION EXAMPLE 3

### Synthesis of 4-chloromethyl-2-(3,4,5-trimethoxyphenyl)pyridine

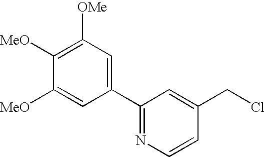

4-hydroxymethyl-2-(3,4,5-trimethoxyphenyl)pyridine (19.18 g) was dissolved in chloroform (100 mL), and to the solution thinly chloride (10.2 mL) was added at 0° C. After 30 minutes, the mixture was warmed to room temperature and stirred for 4 hours. The reaction mixture was washed with aqaueous saturated sodium hydrogendcarbonate and brine, dried over anhydrous sodium sulfate and evaporated. The residue was then recrystallized from ethyl acetate-hexane to give the title compound as pale yellow crystalline powder.

Yield: 18.24 g (89%).

¹H-NMR (400 MHz, CDCl₃) δ: 3.91 (s, 3H), 3.97 (s, 6H), 4.61 (s, 2H), 7.24 (s, 2H), 7.26 (d, 1H, J=5.1 Hz), 7.68 (s, 1H), 8.67 (d, 1H, J=5.1 Hz).

### PREPARATION EXAMPLE 4

### Synthesis of N-[[2-(3,4,5-trimethoxyphenyl)pyridin-4-yl]methyl]phthalimide

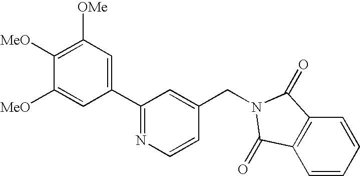

To a solution of 4-chloromethyl-2-(3,4,5-trimethoxyphenyl)pyridine (881 mg) in chloroform (10 mL) was added potassium phthalimide (556 mg). The mixture was stirred at room temperature overnight and water was added. After separating the organic layer, the aqueous layer was extracted with chloroform. Organic layers were combined, dried over anhydrous magnesium sulfate and evaporated to give the title compound as white powder.

Yield: 1.16 g (96%).

### PREPARATION EXAMPLE 5

### Synthesis of 4-aminomethyl-2-(3,4,5-trimethoxyphenyl)pyridine

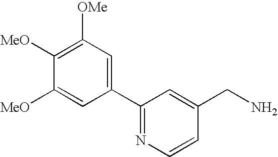

To a suspension of N-[[2-(3,4,5-trimethoxyphenyl)pyridin-4-yl]methyl] phthalimide (1.16 g) in ethanol (30 mL) was added hydrazine monohydrate (1 mL). The mixture was refluxed for 3 hours. After cooling, the precipitates were filtered off. The filtrate was evaporated and the residue was dissolved in chloroform. The solution was washed with saturated aqueous sodium hydrogen carbonate and brine, dried over anhydrous magnesium sulfate and evaporated to give the title compound as pale yellow oil.

Yield: 418 mg (53%).

### PREPARATION EXAMPLE 6

### Synthesis of Ethyl 1-[[2-(3,4,5-trimethoxyphenyl)pyridin yl]methyl]piperidine-4-carboxylate

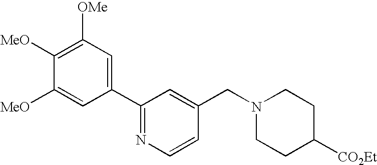

To a solution of ethyl piperidine-4-carboxylate (514 mg) and 4-chloromethyl-2-(3,4,5-trimethoxyphenyl)pyridine (969 mg) in acetonitrile (20 mL) was added potassium carbonate (452 mg). The mixture was stirred at room temperature for 4 hours and evaporated. The residual oil was subjected to a column of silica gel and eluted using hexane-ethyl acetate (2:1) and then chloroform-methanol (40:1). Fractions containing the product were collected and evaporated to give the title compound as white prisms.

Yield: 1.20 g (88%).

¹H-NMR (400 MHz, CDCl₃) δ: 1.25 (t, 3H, J=7.0 Hz), 1.72-1.93 (m, 4H), 2.10 (t, 2H, J=9.8 Hz), 2.27-2.35 (m, 1H), 2.86 (d, 2H, J=11.3 Hz), 3.55 (s, 2H), 3.91 (s, 3H), 3.98 (s, 6H), 4.14 (q, 2H, J=7.0 Hz), 7.21 (d, 1H, J=4.9 Hz), 7.24 (s, 2H), 7.63 (s, 1H), 8.59 (d, 1H, J=5.1 Hz).

### PREPARATION EXAMPLE 7

### Synthesis of 1-[[2-(3,4,5-trimethoxyphenyl)pyridinyl]methyl]piperidine-4-carboxylic Acid

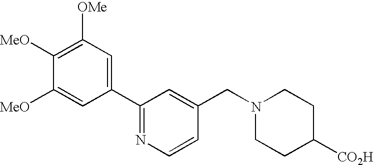

To a solution of ethyl 1-[[2-(3,4,5-trimethoxyphenyl)prydine-4-yl]methyl] piperidine-4-carboxylate (760 mg) in ethanol (10 mL) was added 1 M sodium hydroxide (10 mL). The mixture was stirred at room temperature for 4 hours and evaporated. The residue was dissolved in water (20 mL) and 5% aqueous potassium hydrogen sulfate was added dropwise until pH of the solution became 7. Precipitates were collected and the product was used for the next steps without further purification.

Yield: 779 mg (theoretical amount).

### EXAMPLE 1

### Synthesis of 1-[[2-(3,4,5-trimethoxyphenyl)pyridinyl]methyl]-4-[[2-(3,4,5-trimethoxyphenyl)pyridin-4-yl]methylaminocarbonyl]piperidine Maleate

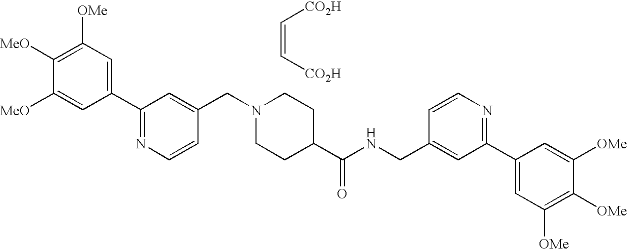

To a solution of 1-[[2-(3,4,5-trimethoxyphenyl)pyridin-4-yl]methyl] piperidine-4-carboxylic acid (97 mg) and 4-aminomethyl-2-(3,4,5-trimethoxyphenyl) pyridine (68 mg) in acetonitrile (5 mL) was added HBTU (95 mg). The mixture was stirred at room temperature for 12 hours and evaporated. The residual oil was dissolved in chloroform, washed with saturated aqueous sodium hydrogen carbonate and brine, dried over anhydrous magnesium sulfate and evaporated. Resulting residue was applied to a column of silica gel and eluted using chloroform-methanol (40:1) and then chloroform-methanol (20:1). Fractions containing the product were collected and evaporated. The free base of the product was then converted to a maleate by the usual method.

Yield: 93 mg (49%).

¹H-NMR (400 MHz, measured as a maleate, DMSO-d₆) δ: 1.87-2.01 (m, 4H), 2.48-2.56 (m, 1H), 2.78-2.86 (m, 2H), 3.26-3.31 (m, 2H), 3.78 (s, 3H), 3.79 (s, 3H), 3.87 (s, 6H), 3.90 (s, 6H), 4.15 (s, 2H), 4.39 (d, 2H, J=5.9 Hz), 6.16 (s, 2H), 7.16 (d, 1H, J=5.9 Hz), 7.35 (s, 2H), 7.39 (d, 1H, J=5.9 Hz), 7.39 (s, 2H), 7.73 (s, 1H), 7.95 (s, 1H), 8.15 (d, 1H, J=5.9 Hz), 8.54 (d, 1H, J=4.9 Hz), 8.68 (d, 1H, J=4.9 Hz).

### PREPARATION EXAMPLE 8

### Synthesis of 1-(benzyloxycarbonyl)-4-[[2-(3,4,5-trimethoxyphenyl)pyridin-4-yl]methyloxy]piperidine

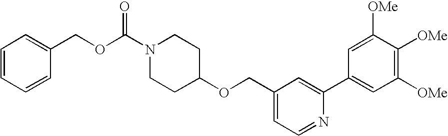

To a solution of 1-(benzyloxycarbonyl)-4-hydroxypiperidine (1.00 g) in DMF (20 mL) was added sodium hydride (55% dispersion in mineral oil, 222 mg). The mixture was stirred at room temperature for 1 hour and then, 4-chlolromethyl-2-(3,4,5-trimethoxyphenyl)pyridine (1.37 g) and potassium iodide (755 mg) was added. The mixture was stirred at 70° C. overnight, poured into water and extracted with chloroform. The organic layer was washed with brine, dried over anhydrous sodium sulfate and evaporated. The residual oil was applied to a column of silica gel and column chromatography was performed using chloroform-methanol (99:1) as an eluent giving the title compound.

Yield: 213 mg (10%).

¹H NMR (400 MHz, CDCl₃) δ: 1.63 (br, 2H), 1.89 (br, 2H), 3.20-3.35 (m, 2H), 3.57-3.68 (m, 1H), 3.84-3.92 (m, 5H), 3.94 (s, 6H), 4.62 (s, 2H), 5.11 (s, 2H), 7.21-7.35 (m, 8H), 7.61 (s, 1H), 8.61 (d, 1H, J=5.0 Hz).

### PREPARATION EXAMPLE 9

### Synthesis of 4-[[2-(3,4,5-trimethoxyphenyl)pyridin-4-yl]methyloxy]piperidine

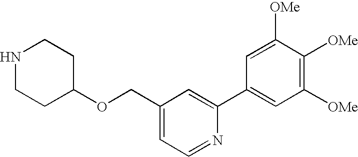

To a solution of 1-(benzyloxycarbonyl)-4-[[2-(3,4,5-trimethoxyphenyl) pyridin-4-yl]methyloxy]piperidine (213 mg) in methanol (10 mL) was added 40% aqueous potassium hydroxide (10 mL). The mixture was stirred at 100° C. for 3 hours and evaporated. Water was added to the residue and extracted with chloroform. The organic layer was washed with brine, dried over anhydrous sodium sulfate and evaporated. The residual oil was subjected to column chromatography of silica gel using chloroform-ammonia saturated methanol (20:1) to give the title compound.

Yield: 93 mg (60%).

¹H NMR (400 MHz, CDCl₃) δ: 1.55-1.68 (m, 2H), 2.01 (br, 2H), 2.67-2.72 (m, 2H), 3.13-3.18 (m, 2H), 3.50-3.60 (m, 1H), 3.91 (s, 3H), 3.97 (s, 6H), 4.64 (s, 2H), 7.22 (d, 1H, J=4.3 Hz), 7.24 (s, 2H), 7.64 (s, 1H), 8.63 (d, 1H, J=5.1 Hz).

### EXAMPLE 2

### Synthesis of 1-[[2-(3,4,5-trimethoxyphenyl)pyridinyl]methyl]-4-[[2-(3,4,5-trimethoxyphenyl)pyridin-4-yl]methyloxy]piperidine Trihydrochloride

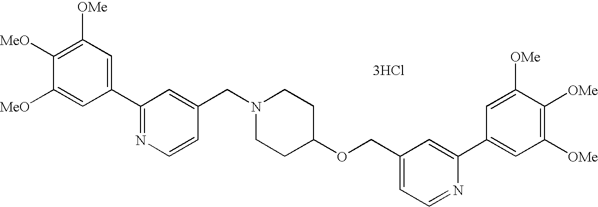

4-[[2-(3,4,5-trimethoxyphenyl)pyridin-4-yl]methyloxy]piperidine (70 mg), 4-chloromethyl-2-(3,4,5-trimethoxyphenyl)pyridine (22 mg), potassium carbonate (56 mg) and potassium iodide (40 mg) were suspended in acetonitrile (5 mL). The mixture was stirred at room temperature for 5 hr and evaporated. Chloroform and water were added to the residual oil and the organic layer was separated. Aqueous layer was then extracted with chloroform and the organic layers were combined, dried over anhydrous magnesium sulfate and evaporated. The residue was applied to a column of silica gel using chloroform-methanol (40:1) as an eluent. Fractions containing the product were collected and evaporated. The title compound was obtained by converting the free base to a trihydrochloride.

Yield: 42 mg (39%).

¹H NMR (400 MHz, measured as a free base, CDCl₃) δ: 1.53-2.42 (m, 6H), 2.80 (br, 2H), 3.57 (br, 3H), 3.88 (s, 6H), 3.94 (s, 61H), 3.95 (s, 6H), 4.60 (s, 2H), 7.18-7.24(m, 6H), 7.61 (s, 2H), 8.58-8.61 (m, 2H).

### PREPARATION EXAMPLE 10

### Synthesis of (3S)-1-(tert-butoxycarbonyl)-3-[(2-nitrobenzene)sulfonylamino] Pyrrolidine

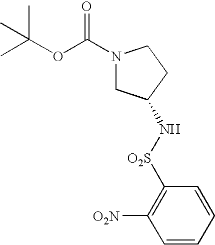

To an ice-cooled solution of (3S)-3-amino-1-(tert-butoxycarbonyl) pyrrolidine (404 mg) and triethylamine (220 mg) in THF (5 mL) was added 2-nitrobenzenesulfonyl chloride (481 mg). The mixture was stirred at room temperature for 30 minutes and evaporated. Ethyl acetate was added to the residue. The solution was washed with water and brine, dried over anhydrous sodium sulfate and evaporated. The residual oil was subjected to a column of silica gel and column chromatography was performed using chroloform-methanol (20:1) as an eluent. Fractions containing the product were collected and evaporated to give the title compound as pale yellow amorphous.

Yield: 597 mg (74%).

¹H-NMR (400 MHz, CDCl₃) δ: 1.44 (s, 9H), 1.80-2.12 (m, 2H), 3.14-3.44 (m, 4H), 4.02 (br, 1H), 5.48 (d, 1H, J=7.2 Hz), 7.77 (t, 2H, J=4.4 Hz), 7.87-7.90 (m, 1H), 8.17-8.19 (m, 1H).

### PREPARATION EXAMPLE 11

### Synthesis of (3S)-1-(tert-butoxycarbonyl)-3-[N-methyl-N-(2-nitrobenzene) sulfonylamino]pyrrolidine

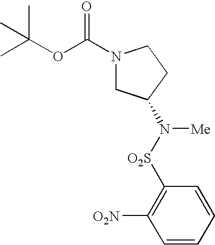

To a suspension of (3S)-1-(tert-butoxycarbonyl)-3-[(2-nitrobenzene) sulfonylamino]pyrrolidine (371 mg) and potassium carbonate (141 mg) in acetonitrile (10 mL) was added methyl iodide (141 mg). The mixture was stirred at 60° C. for 2 hours and evaporated. Ethyl acetate was added to the mixture. The solution was washed with saturated aqueous sodium hydrogen carbonate and brine, dried over anhydrous sodium sulfate and evaporated. The residue was applied to a column of silica gel using hexane-ethyl acetate (2:1) as an eluent. Fractions containing the product were collected and evaporated to give the title compound as yellow syrup.

Yield: 365 mg (95%).

¹H-NMR (400 MHz, CDCl₃) δ: 1.44 (s, 9H), 1.95 (br, 1H), 2.09 (br, 1H), 2.87 (s, 3H), 3.20-3.31 (m, 2H), 3.53 (br, 2H), 4.58 (br, 1H), 7.65 (br, 1H), 7.71 (br, 2H), 8.04 (br, 1H).

### PREPARATION EXAMPLE 12

### Synthesis of (3S)-3-[N-methyl-N-(2-nitrobenzene)sulfonylamino]pyrrolidine

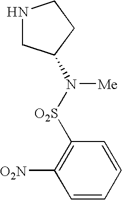

To an ice-cooled solution of (3S)-1-(tert-butoxycarbonyl)-3-[N-methyl-N-(2-nitrobenzenesulfonyl)amino]pyrrolidine (365 mg) in dichloromethane (25 mL) was added trifluoroacetic acid (1 mL). The mixture was stirred at room temperature for 3 hours and evaporated. The residue was dissolved in chloroform. The solution was washed with saturated aqueous sodium hydrogen carbonate and brine, dried over anhydrous sodium sulfate and evaporated to give the title compound as yellow syrup.

Yield: 135 mg (50%).

¹H-NMR (400 MHz, CDCl₃) δ: 1.69-1.74 (m, 1H), 1.87 (br, 1H), 1.95-2.02 (m, 1H), 2.80 (dd, 1H, J=11.7 Hz, 5.7 Hz), 2.84-2.91 (m, 4H), 2.96-3.05 (m, 1H), 3.10 (dd, 1H, J=11.7 Hz, 8.2 Hz), 4.48-4.56 (m, 1H), 7.61-7.63 (m, 1H), 7.66-7.73 (m, 2H), 8.01-8.04 (m, 1H).

### PREPARATION EXAMPLE 13

### Synthesis of (3S)-3-[N-methyl-N-(2-nitrobenzene)sulfonylamino]-1-[[2-(3,4,5-trimethoxyphenyl)pyridin-4-yl]methyl]pyrrolidine

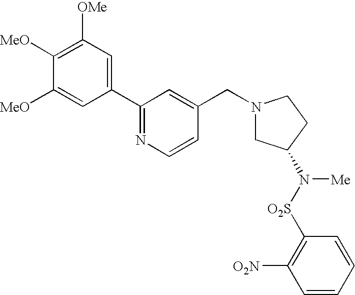

(3S)-3-[N-methyl-N-(2-nitrobenzene)sulfonylamino]pyrrolidine (135 mg) and 4-chloromethyl-2-(3,4,5-trimethoxyphenyl)pyridine (139 mg) were coupled in the same manner as described in Example 2 to give the title compound as yellow amorphous.

Yield: 247 mg (96%).

¹H-NMR (400 MHz, CDCl₃) δ: 1.80-1.87 (m, 1H), 2.15-2.30 (m, 2H), 2.52 (dd, 1H, J=10.5 Hz, 8.2 Hz), 2.71 (dd, 1H, J=10.5 Hz, 8.2 Hz), 2.90 (dt, 1H, J=8.8 Hz, 2.9 Hz), 2.96 (s, 3H), 3.53 (d, 1H, J=13.9 Hz), 3.68 (d, 1H, J=13.9 Hz), 3.90 (s, 3H), 3.96 (s, 6H), 4.61-4.68 (m, 1H), 7.16 (dd, 1H, J=4.9 Hz, 1.2 Hz), 7.21 (s, 2H), 7.58-7.60 (m, 2H), 7.64-7.69 (m, 2H), 7.99-8.02 (m, 1H), 8.58 (d, 1H, J=4.9 Hz,).

### PREPARATION EXAMPLE 14

### Synthesis of (3S)-3-methylamino-1-[[2-(3,4,5-trimethoxyphenyl)-pyridin-4-yl]methyl]pyrrolidine

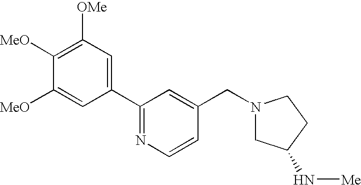

To a solution of (3S)-3-[N-methyl-N-(2-nitrobenzene)sulfonylamino]-1-[[2-(3,4,5-trimethoxyphenyl)pyridin-4-yl]methyl]pyrrolidine (242 mg) in acetonitrile (5 mL) was added potassium carbonate (94 mg) and thiophenol (75 mg). The mixture was stirred at 80° C. for 3 hours and evaporated. Ethyl acetate was added to the mixture, the solution was washed with saturated aqueous sodium hydrogen carbonate, water, and brine, dried over anhydrous sodium sulfate and evaporated. The residual oil was subjected to preparative TLC using chloroform-methanol (20:1) as a solvent system giving yellow syrup of the title compound.

Yield: 104 mg (64%).

¹H-NMR (400 MHz, CDCl₃) δ: 1.32 (br, 1H), 1.56-1.64 (m, 1H), 2.11-2.17 (m, 1H), 2.38 (s, 3H), 2.44 (dd, 1H, J=7.4 Hz, 4.5 Hz), 2.50-2.55 (m, 1H), 2.66-2.75 (m, 2H), 3.20-3.26 (m, 1H), 3.66 (s, 2H), 3.90 (s, 3H), 3.97 (s, 6H), 7.21 (d, 1H, J=4.1 Hz), 7.25 (s, 2H), 7.64 (s, 1H), 8.59 (d, 1H, J=4.9 Hz).

### EXAMPLE 3

### Synthesis of (3S)-3-[N-methyl-N-[[2-(3,4,5-trimethoxyphenyl)pyridin-4-yl]methyl]amino]-1-[[2-(3,4,5-trimethoxyphenyl)pyridin-4-yl]methyl]pyrrolidine Tertrahydrochloride

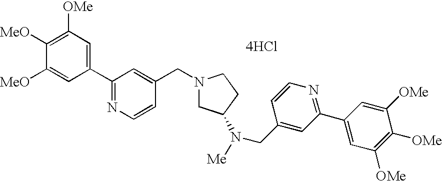

(3S)-3-methylamino-1-[[2-(3,4,5-trimethoxyphenyl)pyridin-4-yl]methyl] pyrrolidine (104 mg) and 4-chloromethyl-2-(3,4,5-trimethoxyphenyl)pyridine (85 mg) was condensed in the same manner as described in Example 2. Yellow syrup obtained was converted to a tetrahydrochloride by the usual method giving the title compound as yellow powder.

Yield: 151 mg (68%).

¹H-NMR (400 MHz, measured as a free base, CDCl₃) δ: 1.89-1.92 (m, 1H), 2.04-2.08 (m, 1H), 2.18 (s, 3H), 2.60-2.76 (m, 4H), 3.25-3.29 (m, 1H), 3.53 (d, 1H, J=14.3 Hz), 3.62 (d, 1H, J=14.3 Hz), 3.64 (d, 1H, J=13.9 Hz), 3.73 (d, 1H, J=13.9 Hz), 3.89 (s, 6H), 3.95 (s, 6H), 3.96 (s, 6H), 7.20-7.21 (m, 2H), 7.23 (s, 2H), 7.24 (s, 2H), 7.61 (s, 1H), 7.65 (s, 1H), 8.59 (d, 1H, J=5.7 Hz), 8.60 (d, 1H, J=5.3 Hz).

### PREPARATION EXAMPLE 15

### Synthesis of 1-[[2-(3,4,5-trimethoxyphenyl)pyridin-4-yl]methyl]piperidine-4-carboxamide

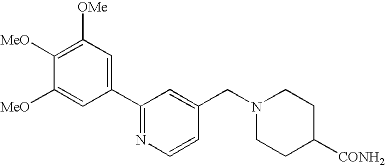

Piperidine-4-carboxamide (385 mg) and 4-chloromethyl-2-(3,4,5-trimethoxyphenyl)pyridine (881 mg) were condensed by the same method as described in Example 2 to give the title compound as white needles.

Yield: 1.01 g (87%).

¹H-NMR (400 MHz, CDCl₃) δ: 1.70-1.88 (m, 4H), 2.01-2.23 (m, 3H), 2.95 (d, 2H, J=11.0 Hz), 3.56 (s, 2H), 3.90 (s, 3H), 3.98 (s, 6H), 5.46 (d, 2H, J=16.3 Hz), 7.21 (d, 1H, J=5.0 Hz), 7.24 (s, 2H), 7.64 (s, 1H), 8.59 (d, 1H, J=5.0 Hz).

### PREPARATION EXAMPLE 16

### Synthesis of 4-amino-1-[[2-(3,4,5-trimethoxyphenyl)pyridin-4-yl]methyl]piperidine Trihydrochloride

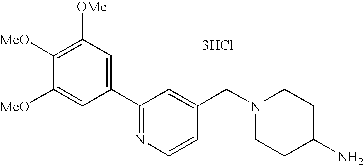

To a solution of 1-[[2-(3,4,5-trimethoxypheyl)pyridin-4-yl]methyl]piperidine-4-carboxamide (192 mg) in a mixed solvent of water (50 mL) and acetonitrile (50 mL) was added [bis(trifluoroacetoxy)iodo]benzene (323 mg). The mixture was stirred at room temperature overnight and evaporated. Saturated aqueous sodium hydrogen carbonate was added to the residue and extracted with chloroform. The organic layer was washed with brine, dried over anhydrous magnesium sulfate and evaporated. Yellow syrup obtained was then converted to trihydrochloride which gave yellow powder. The title compound was used for next step without further purification.

Yield: 201 mg (theoretical amount).

### PREPARATION EXAMPLE 17

### Synthesis of 2-(3,4,5-trimethoxyphenyl)isonicotinic Acid

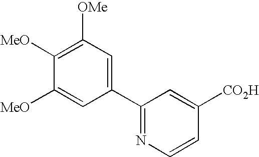

To a solution of ethyl 2-(3,4,5-trimethoxyphenyl)isonicotinate (3.17 g) in ethanol (40 mL) was added 10% potassium hydroxide (2.42 g). The mixture was stirred at room temperature for 5 hours and evaporated. Water was added to the residue and pH was adjusted to 7. White precipitates of the title compound were collected by filtration and the compound was used for next step without further purification.

Yield: 2.60 g (90%).

### EXAMPLE 4

### Synthiesis of 4-[2-(3,4,5-trimethoxyphenyl)pyridin-4-carbonylamino]-1-[[2-(3,4,5-trimethoxyphenyl)pyridin-4-yl]methyl]piperidine Maleate

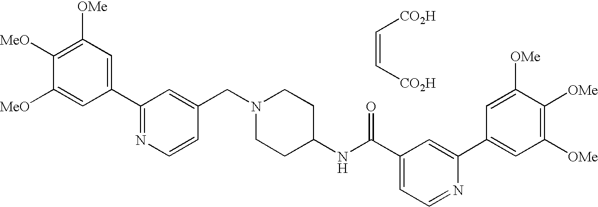

2-(3,4,5-trimethoxyphenyl)isonicotinic acid (72 mg) and 4-amino-1-[[2-(3,4,5-trimethoxyphenyl)pyridin-4-yl]methyl]piperidine (117 mg) were condensed in the same manner as described in Example 1. The title compound was obtained as a maleate.

Yield: 173 mg (93%).

¹H-NMR (400 MHz, measured as a maleate, DMSO-d₆) δ: 1.82-1.94 (m, 2H), 2.03-2.08 (m, 2H), 2.77-2.83 (m, 2H), 3.20-3.27 (m, 2H), 3.79 (s, 6H), 3.90 (s, 12H), 4.00 (br, 1H), 4.06 (s, 2H), 6.15 (s, 2H), 7.36-7.38 (m, 1H), 7.39 (s, 2H), 7.41 (s, 2H), 7.61-7.63 (m, 1H), 7.90 (s, 1H), 8.12 (s, 1H), 8.27-8.32 (m, 1H), 8.67 (d, 1H, J=4.9 Hz), 8.74 (d, 1H, J=5.1 Hz).

### PREPARATION EXAMPLE 18

### Synthesis of 4-[(2-nitrobenzene)sulfonylamino]-1-[[2-(3,4,5-trimethoxyphenyl) pyridin-4-yl]methyl]piperidine

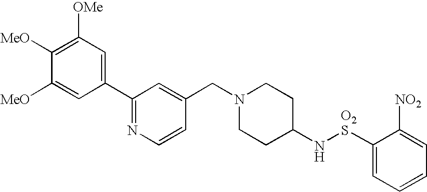

4-amino-1-[[2-(3,4,5-trimethoxyphenyl)pyridin-4-yl]methyl]piperidine (467 mg) and 2-nitrobenzenesulfonyl chloride (244 mg) were condensed in the same manner as described in Preparation Example 10 to give the title compound.

Yield: 494 mg (91%).

### PREPARATION EXAMPLE 19

### Synthesis of 4-[N-(2-nitrobenzene)sulfonyl-N-[[2-(3,4,5-trimethoxyphenyl)pyridin-4-yl]methyl]amino]-1-[[2-(3,4,5-trimethoxyphneyl)pyridin-4-yl]methyl]piperidine

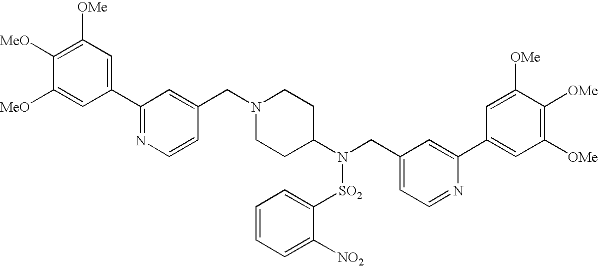

4-[(2-nitrobenzene)sulfonylamino]-1-[[2-(3,4,5-trimethoxyphenyl)pyridin-4-yl]methyl]piperidine (494 mg) and 4-chloromethyl-2-(3,4,5-trimethoxyphenyl)pyridine (267 mg) were condensed in the same manner as described in Example 2 to give the title compound.

Yield: 443 mg (61%).

### EXAMPLE 5

### Synthesis of 1-[[2-(3,4,5-trimethoxyphenyl)pyridin-4-yl]methyl]-4-[[2-(3,4,5-trimethoxyphenyl)pyridin-4-yl]methylamino]piperidine Difumalate

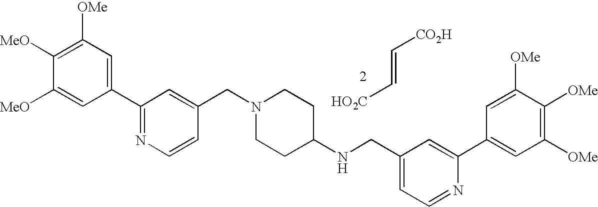

4-[N-(2-nitrobenzene)sulfonyl-N-[[2-(3,4,5-trimethoxyphenyl)pyridin-4-yl]methyl]amino]-1-[[2-(3,4,5-trimethoxyphneyl)pyridin-4-yl]methyl]piperidine (443 mg) was treated in the same manner as described in Preparation Example 14. The title compound was obtained after converting to a difumalate.

Yiled: 103 mg (24%).

¹H-NMR (400 MHz, measured as a free base, CDCl₃) δ: 1.44-1.53 (m, 2H), 1.87-1.91 (m, 2H), 2.15 (t, 2H, J=1.1 Hz), 2.57-2.64 (m, 1H), 2.82-2.85 (m, 2H), 3.59 (s, 2H), 3.78 (s, 6H), 3.89 (s, 12H), 3.90 (s, 2H), 6.63 (s, 4H), 7.24 (d, 1H, J=4.9 Hz), 7.29 (d, 1H, J=4.9 Hz), 7.35 (s, 2H), 7.37 (s, 2H), 7.76 (s, 1H), 7.85 (s, 1H), 8.53-8.56 (m, 2H).

### PREPARATION EXAMPLE 20

### Synthesis of 4-(ethoxycarbonylamino)-1-[[2-(3,4,5-trimethoxyphenyl)pyridin-4-yl]methyl]piperidine

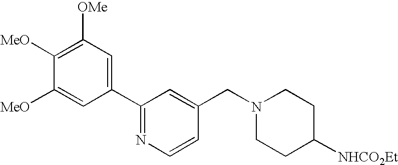

To a solution of 1-[[2-(3,4,5-trimethoxyphenyl)pyridin-4-yl]methyl] piperidine-4-carboxamide (528 mg) in a mixed solvent of ethanol (10 mL) and acetonitrile (10 mL) was added [bis(trifluoroacetoxy)iodo]benzene (884 mg). The mixture was stirred at room temperature overnight and evaporated. Saturated aqueous sodium hydrogen carbonate was added to the residue and extracted with chloroform. The organic layer was washed with brine, dried over anhydrous magnesium sulfate and evaporated. The residue was applied to a column of silica gel and purified using chloroform-methanol (20:1) as an eluent to give the title compound.

Yield: 566 mg (96%).

¹H-NMR (400 MHz, CDCl₃) δ: 1.21 (t, 3H, J=7.0 Hz), 1.40-1.51 (m, 2H), 1.92 (d, 2H, J=10.9 Hz), 2.15 (t, 2H, J=10.9 Hz), 2.78 (d, 2H, J=11.6 Hz), 3.52 (br, 3H), 3.87 (s, 3H), 3.94 (s, 6H), 4.07 (q, 2H, J=7.0 Hz), 4.56 (br, 1H), 7.17 (d, 1H, J=4.9 Hz), 7.21 (s, 2H), 7.59 (s, 1H), 8.56 (d, 1H, J=5.1 Hz).

### PREPARATION EXAMPLE 21

### Synthesis of 4-(methylamino)-1-[[2-(3,4,5-trimethoxyphenyl)pyridin-4-yl]methyl] piperidine

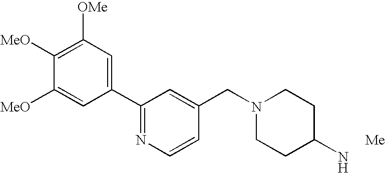

To a suspension of lithium aluminum hydride (100 mg) in dry THF (50 mL) was added a solution of 4-(ethoxycarbonylamino)-1-[[2-(3,4,5-trimethoxyphenyl) pyridin-4-yl]methyl]piperidine (566 mg) in dry THF (50 mL) under an argon atmosphere. The mixture was then refluxed overnight, then cooled down. Saturated aqueous ammonium chloride was added to the mixture and extracted with ethyl acetate. The organic layer was washed with brine, dried over anhydrous sodium sulfate and evaporated. The residue was subjected to silica gel column chromatography using chloroform-ammonia saturated methanol (9:1) to give the title compound as yellow oil.

Yiled: 379 mg (78%).

¹H-NMR (400 MHz, CDCl₃) δ: 1.36-1.46 (m, 2H), 1.89 (d, 2H, J=12.5 Hz), 2.10 (dt, 2H, J=11.5 Hz, 1.1 Hz), 2.35-2.43 (m, 1H), 2.43 (s, 3H), 2.86 (d, 2H, J=11.6 Hz), 3.56 (s, 2H), 3.90 (s, 3H), 3.97 (s, 6H), 7.21 (d, 1H, J=5.1 Hz), 7.24 (s, 2H), 7.64 (s, 1H), 8.59(d, 1H, J=4.9 Hz).

### PREPARATION EXAMPLE 22

### Synthesis of 1-[[2-(3,4,5-trimethoxyphenyl)pyridin-4-yl]methyl]-4-piperidone Ethylene Ketal

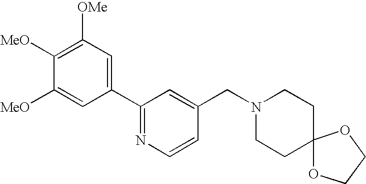

4-Piperidone ethylene ketal (12.0 g) and 4-chloromethyl-2-(3,4,5-trimethoxyphenyl)pyridine (12.3 g) was condensed in the same manner as described in Example 2 to give the title compound.

Yield: 19.0 g (theoretical amount).

¹H-NMR (400 MHz, CDCl₃) δ: 1.68 (t, 4H, J=5.6 Hz), 2.48 (br, 4H), 3.50 (s, 2H), 3.82 (s, 3H), 3.86 (s, 4H), 3.88 (s, 6H), 7.13 (d, 1H, J=4.9 Hz), 7.17 (s, 2H), 7.57 (s, 1H), 8.51 (d, 1H, J=4.9 Hz).

### PREPARATION EXAMPLE 23

### Synthesis of 1-[[2-(3,4,5-trimethoxyphenyl)pyridin-4-yl]methyl]-4-piperidone

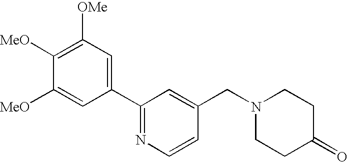

To a solution of 1-[[2-(3,4,5-trimethoxyphenyl)pyridin-4-yl]methyl]-4-piperidone ethylene ketal (19.0 g) in THF (200 mL) was added 1 M hydrochloric acid (200 mL). The mixture was stirred at 90° C. overnight, then neutralized with 2 M sodium hydroxide and extracted with ethyl acetate. The organic layer was washed with brine, dried over anhydrous sodium sulfate and evaporated. The residual oil was applied to a column of silica gel using chloroform-methanol (40:1) as an eluent. Fractions containing the product were collected and evaporated to give the title compound.

Yield: 15.0 g (75%).

¹H-NMR (400 MHz, CDCl₃) δ: 2.48 (t, 4H, J=6.1 Hz), 2.79 (t, 4H, J=6.0 Hz), 3.69 (s, 2H), 3.89 (s, 3H), 3.96 (s, 6H), 7.24 (s, 2H), 7.26 (d, 1H, J=4.9 Hz), 7.66 (s, 1H), 8.62 (d, 1H, J=4.9 Hz).

### PREPARATION EXAMPLE 24

### Synthesis of 1-[[2-(3,4,5-trimethoxyphenyl)pyridin-4-yl]methyl]-4-piperidone

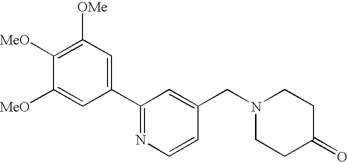

4-Piperidone hydrochloride monohydrate (3.07 g) and 4-chloromethyl-2-(3,4,5-trimethoxyphenyl)pyridine (2.94 g) were coupled by the same manner as described in Example 2 to give the title compound.

Yield: 3.55 g (99%).

### PREPARATION EXAMPLE 25

### Synthesis of

4-(methylamino)-1-[[2-(3,4,5-trimethoxyphenyl)pyridin-4-yl]methyl]piperidine:

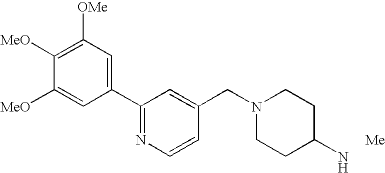

To a solution of 1-[[2-(3,4,5-trimethoxyphenyl)pyridin-4-yl]methyl]-4-piperidone (1.00 g) in 1,2-dichloroethane (60 mL) was added 30% solution of methylamine in ethanol (750 mg) and sodium triacetoxyborohydride (1.66 g). The mixture was stirred at room temperature for 3 hours, then small amount of water was added and evaporated. Water was added to the residue and extracted with chloroform. The organic layer was washed with brine, dried over anhydrous sodium sulfate and evaporated. The residue was subjected to silica gel column chromatography using chloroform-methanol (40:1) to give the title compound.

Yield: 640 mg (62%).

### PREPARATION EXAMPLE 26

### Synthesis of ethyl 3-(3,4,5-trimethoxyphenyl)benzoate

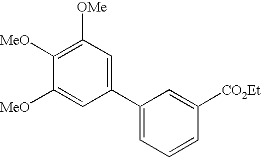

3,4,5-Trimethoyphenylboronic acid (3.7 g) and ethyl 3-bromobenzoate (4.02 g) were condensed in the same manner as described in Preparation Example 1 to give the title compound.

Yield: 5.09 g (92%).

¹H-NMR (400 MHz, CDCl₃) δ: 1.42 (t, 3H, J=7.1 Hz), 3.90 (s, 3H), 3.94 (s, 6H), 4.41 (q, 2H, J=7.1 Hz), 6.79 (s, 2H), 7.50 (t, 1H, J=7.8 Hz), 7.73 (dt, 1H, J=7.1 Hz, 1.5 Hz), 8.01 (dt, 1H, J=7.8 Hz, 1.4 Hz), 8.23 (t, 1H, J=1.8 Hz).

### PREPARATION EXAMPLE 27

### Synthesis of 3-(3,4,5-trimethoxyphenyl)benzoic Acid

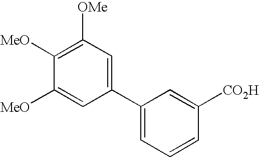

Ethyl 3-(3,4,5-trimethoxyphenyl)benzoate (1.19 g) was treated in the same manner as described in Preparation Example 17 to give the title compound.

Yield: 986 mg (91%).

### EXAMPLE 6

### Synthesis of 4-[N-methyl-N-[3-(3,4,5-trimethoxyphenyl)]benzoylamino]-1-[[2-(3,4,5-trimethoxyphenyl)pyridin-4-yl]methyl]piperidine Dihydrochloride

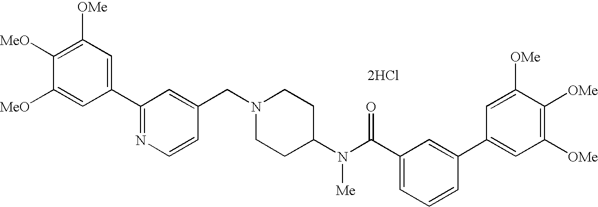

3-(3,4,5-trimethoxyphenyl)benzoic acid (1.03 g) and 4-(methylamino)-1-[[2-(3,4,5-trimethoxyphenyl)pyridin-4-yl]methyl]piperidine (1.32 g) were condensed in the same method as described in Example 1. The title compound was obtained after converting a free amine to a dihydrochloride.

Yield: 1.44 g (57%).

¹H-NMR (400 MHz, measured as a dihydrochloride, DMSO-d₆) δ: 1.89 (d, 2H, J=11.7 Hz), 2.54-2.62 (m, 2H), 2.89 (s, 3H), 3.09 (t, 2H, J=12.7 Hz), 3.43 (d, 2H, J=14.4 Hz), 3.76 (s, 3H), 3.78 (s, 3H), 3.88 (s, 6H), 3.91 (s, 6H), 4.34 (br, 3H), 6.91 (s, 2H), 7.33 (d, 1H, J=7.6 Hz), 7.47-7.51 (m, 2H), 7.54 (s, 2H), 7.60 (s, 1H), 7.71 (d, 1H, J=7.8 Hz), 8.55 (s, 1H), 8.68 (d, 1H, J=5.1 Hz).

### EXAMPLE 7

### Synthesis of 4-[N-methyl-N-[[5-(3,4,5-trimethoxyphenyl)pyridin-3-yl]methyl]amino]-1-[[2-(3,4,5-trimethoxyphenyl)pyridin-4-yl]methyl]piperidine Difumarate

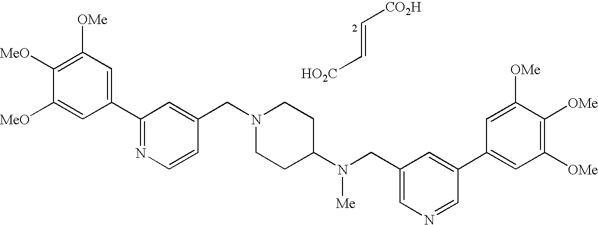

4-methylamino-1-[[2-(3,4,5-trimethoxyphenyl)pyridin-4-yl]methyl]piperidine (135 mg) and 3-chloromethyl-5-(3,4,5-trimethoxypyenyl)pyridine (107 mg) were condensed by the same method as described in Example 2. White powder of the title compound was obtained after converting a free base to a difumarate.

Yield: 180 mg (58%).

¹H-NMR (400 MHz, measured as a free base, CDCl₃) δ: 1.69-1.73 (m, 2H), 1.82-1.85 (m, 2H), 2.03-2.08 (m, 2H), 2.25 (s, 3H), 2.48-2.51 (m, 1H), 2.97-2.99 (m, 2H), 3.56 (s, 2H), 3.67 (s, 2H), 3.90 (s, 3H), 3.91 (s, 3H), 3.94 (s, 6H), 3.98 (s, 6H), 6.76 (s, 2H), 7.22 (d, 1H, J=5.1 Hz), 7.24 (s, 2H), 7.62 (s, 1H), 7.80 (s, 1H), 8.50 (d, 1H, J=2.0 Hz), 8.60 (d, 1H, J=4.3 Hz), 8.69 (d, 1H, J=5.1 Hz).

### PREPARATION EXAMPLE 28

### Synthesis of 1-bromo-4-chloro-3,5-dimethoxybenzene

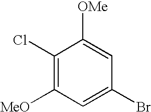

A solution of sodium nitrite (97 mg) in water (2.0 mL) was added dropwise to an ice-cold suspension of 4-bromo-2,6-dimethoxyaniline (232 mg) in 6.0 M hydrochloric acid (2.5 mL). After stirring in ice for 30 minutes, a solution of cupric chloride (495 mg) in concentrated hydrochloric acid (2.0 mL) was added. The reaction mixture was stirred at room temperature for 30 minutes, then at 100° C. for 2 hours, and extracted with ethyl acetate. The organic layer was washed with a saturated aqueous sodium hydrogencarbonate and water, dried over anhydrous sodium sulfate, and evaporated. The residue was subjected to a column of silica gel using hexane-ethyl acetate (10:1) as an eluent to give the title compound as white powder.

Yield: 230 mg (92%).

### PREPARATION EXAMPLE 29

### Synthesis of 4-chloro-3,5-dimethoxyphenylboronic Acid

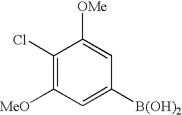

Under an argon atomsphere, to dry THF (2 mL) stirred in a dry ice-methanol bath was gradually added a 1.57 M solution of n-butyllithium in hexane (0.8 mL), followed by the dropwise addition of a solution of 1-bromo-4-chloro-3,5-dimethoxybenzene (160 mg) in dry THF (2 mL). After the mixture was stirred for 20 minutes in the dry ice-methanol bath, triisopropyl borate (0.18 mL) was added and the mixture was additionally stirred for 20 minutes. The reaction mixture was then stirred at room temperature for 1 hour and pH of the mixture was adjusted at 3 using 4 M hydrochloric acid. The mixture was stirred at 0° C. for 1 hour and extracted with ethyl acetate. The organic layer was washed with brine, dried over anhydrous sodium sulfate and evaporated. The residue was recrystallized from ethyl acetate-hexane giving the title compound as white powder.

Yield: 90 mg (66%).

### PREPARATION EXAMPLE 30

### Synthesis of ethyl 2-(4-chloro-3,5-dimethoxyphenyl)isonicotinate

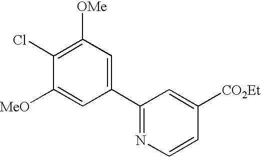

4-Chloro-3,5-dimethoxyphenylboronic acid (7.45 g) and ethyl 2-chloroisonicotinate (6.39 g) were condensedn in the same manner as described in Preparation Example 1 to give the title compound.

Yield: 8.55 g (77%).

¹H-NMR (400 MHz, CDCl₃) δ: 1.45 (t, 3H, J=7.3 Hz), 4.03 (s, 6H), 4.45 (q, 2H, J=7.3 Hz), 7.32 (s, 2H), 7.80 (d, 1H, J=5.1 Hz), 8.27 (s, 1H), 8.83 (d, 1H, J=5.0 Hz).

### PREPARATION EXAMPLE 31

### Synthesis of 2-(4-chloro-3,5-dimethoxyphenyl)isonicotinic Acid

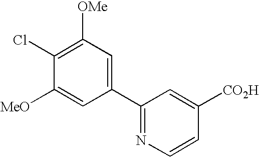

To a solution of ethyl 2-(4-chloro-3,5-diethoxyphenyl)isonicotinate (8.55 g) in ethanol (80 mL) was added 2 M sodium hydroxide (100 mL). The mixture was refluxed for 30 min and evaporated. The aqueous layer was neutralized by 1 M hydrochloric acid and precipitates were dissolved in a mixed solvent of ethyl acetate-THF (3:1). After drying over anhydrous sodium sulfate, the solvent was evaporated to give the title compound.

Yield: 7.20 g (92%).

¹H-NMR (400 MHz, CDCl₃) δ: 4.02 (s, 6H), 7.34 (s, 2H), 7.83 (d, 1H, J=4.9 Hz), 7.84 (s, 1H), 8.82 (d, 1H, J=4.9 Hz).

### PREPARATION EXAMPLE 32

### Synthesis of 2-(4-chloro-3,5-dimethoxyphenyl)-4-hydroxymethylpyridine

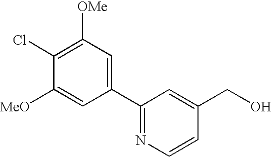

To an ice-cooled solution of 2-(4-chloro-3,5-dimethoxyphenyl)isonicotinic acid (7.20 g) and triethylamine (5.6 mL) in THF (70 mL) was added ethyl chloroformate (2.8 mL). The mixture was stirred at room temperature for 1 hour and filtered. To the filtrate was then added a solution of sodium borohydride (1.25 g) in water (4 mL). The mixture was stirred at room temperature for another hour and evaporated. Water was added to the residue and extracted with chloroform. The organic layer was washed with brine, dried over anhydrous sodium sulfate and evaporated. The residue was subjected to silica gel column chromatography using chloroform-methanol (20:1) and then chloroform-methanol (15:1) to give the title compound.

Yield: 4.10 g (60%).

¹H-NMR (400 Liz, CDCl₃+DMSO-d₆) δ: 4.01 (s, 6H), 4.76 (s, 2H), 7.20-7.35 (m, 3H), 7.78 (s, 1H), 8.62 (s, 1H).

### PREPARATION EXAMPLE 33

### Synthesis of 2-(4-chloro-3,5-dimethoxyphenyl)-4-chloromethylpyridine

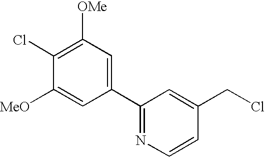

2-(4-Chloro-3,5-dimethoxyphenyl)-4-hydroxymethylpyridine (4.10 g) was treated in the same manner as described in Preparation Example 3 to give the title compound.

Yield: 4.20 g (96%).

¹H-NMR (400 MHz, CDCl₃) δ: 4.02 (s, 6H), 4.63 (s, 2H), 7.26 (s, 2H), 7.29 (d, 1H, J=4.9 Hz), 7.72 (s, 1H), 8.69 (d, 1H, J=4.9 Hz).

### PREPARATION EXAMPLE 34

### Synthesis of 1-[[2-(4-chloro-3,5-dimethoxyphenyl)pyridin-4-yl]methyl]piperidine-4-carboxamide

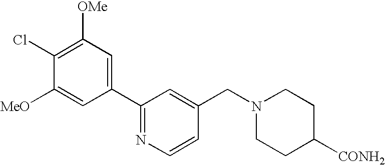

Piperidine-4-carboxamide (301 mg) and 2-(4-chloro-3,5-dimethoxyphenyl)-4-chloromethylpyridine (600 mg) were coupled in the same manner as described in Example 2 to give the title compound.

Yield: 743 mg (95%).

¹H-NMR (400 MHz, CDCl₃) δ: 1.75-1.90 (m, 4H), 2.07-2.25 (m, 3H), 2.94 (d, 2H, J=11.6 Hz), 3.57 (s, 2H), 4.02(s, 6H), 7.24-7.31 (m, 3H), 7.67 (s, 1H), 8.61 (d, 1H, J=5.1 Hz).

### PREPARATION EXAMPLE 35

### Synthesis of 1-[[2-(4-chloro-3,5-dimethoxyphenyl)pyridin-4-yl]methyl]-4-(ethoxycarbonylamino)piperidine

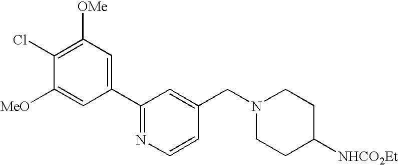

1-[[2-(4-chloro-3,5-dimethoxyphenyl)pyridin-4-yl]methyl]piperidine-4-carboxamide (743 mg) was treated in the same manner as described in Preparation Example 20 to give the title compound.

Yield: 887 mg (theoretical amount).

¹H-NMR (400 MHz, CDCl₃) δ: 1.24 (t, 3H, J=7.1 Hz), 1.43-1.59 (m, 2H), 1.96 (d, 2H, J=11.4 Hz), 2.19 (t, 2H, J=11.0 Hz), 2.82 (d, 2H, J=11.5 Hz), 3.56 (s, 2H), 4.02 (s, 6H), 4.10 (q, 2H, J=7.1 Hz), 7.26 (s, 2H), 7.66 (s, 1H), 7.71 (dd, 1H, J=5.6 Hz, 1.0 Hz), 8.6 (dd, 1H, J=4.9 Hz, 0.5 Hz).

### PREPARATION EXAMPLE 36

### Synthesis of 1-[[2-(4-chloro-3,5-dimethoxyphenyl)pyridin-4-yl]methyl]4-methylaminopiperidine

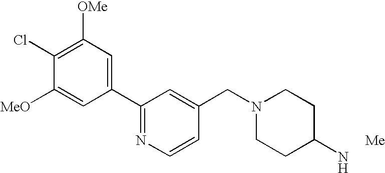

1-[[2-(4-chloro-3,5-diemthoxyphenyl)pyridin-4-yl]methyl]-4-(ethoxy-carbonylamino)piperidine (887 mg) was treated in the same manner as described in Preparation Example 21 to give the title compound.

Yield: 195 mg (27%).

¹H-NMR (400 MHz, CDCl₃) δ: 1.35-1.49 (m, 2H), 1.89 (d, 2H, J=12.3 Hz), 2.11 (t, 2H, J=9.4 Hz), 2.38-2.45 (m, 1H), 2.44 (s, 3H), 2.87 (d, 2H, J=10.7 Hz), 3.57 (s, 2H), 4.02 (s, 6H), 7.23-7.29 (m, 3H), 7.68 (s, 1H), 8.61 (d, 1H, J=4.9 Hz).

### EXAMPLE 8

### Synthesis of 1-[[2-(4-chloro-3,5-dimethoxyphenyl)pyridin-4-yl]methyl]-4-[N-[[2-(4-chloro-3,5-dimethoxyphenyl)pyridin-4-yl]methyl]-N-methylamino]piperidine Tetrahydrochloride

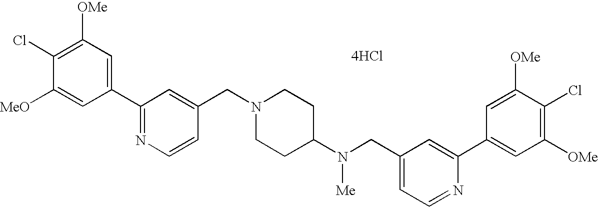

1-[[2-(4-chloro-3,5-dimethoxyphenyl)pyridin-4-yl]methyl]methylamino-piperidine (195 mg) and 2-(4-chloro-3,5-dimethoxyphenyl)-4-chloromethylpyridine (152 mg) were condensed in the same manner as described in Example 2. A free base obtained was converted to a tetrahydrochloride giving yellow powder.

Yield: 300 mg (75%).

¹H-NMR (400 MHz, CDCl₃) δ: 1.60-1.90 (m, 4H), 2.06 (t, 2H, J=11.7 Hz), 2.26 (s, 3H), 2.45-2.55 (m, 1H), 2.97 (d, 2H, J=11.3 Hz), 3.57 (s, 2H), 3.67 (s, 2H), 4.01 (s, 6H), 4.02 (s, 6H), 7.24-7.28 (m, 6H), 7.65 (s, 1H), 7.67 (s, 1H), 8.61 (d, 1H, J=5.4 Hz), 8.62 (d, 1H, J=5.4 Hz).

### PREPARATION EXAMPLE 37

### Synthesis of 4-(p-anisidino)-1-[[2-(3,4,5-trimethoxyphenyl)pyridin-4-yl]methyl]-piperidine

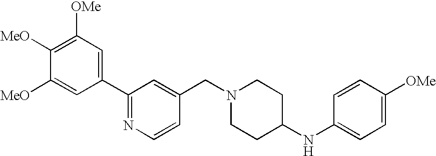

To a solution of 1-[[2-(3,4,5-trimethoxyphenyl)pyridin-4-yl]methyl]4-piperidone (2.17 g) in toluene (40 mL) was added p-anisidine (900 mg) and molecular sieves 4A (6.0 g). The mixture was refluxed overnight, then filtered and the filtrate was evaporated. The residual oil was dissolved in ethanol (40 mL) and sodium borohydride (276 mg) was added. The mixture was stirred at room temperature for 2 hours before concentration in vacuo. The residue was dissolved in ethyl acetate, washed with brine, dried over anhydrous sodium sulfate and evaporated. The residual oil was subjected to silica gel column chromatography using chloroform-methanol (50:1) to give the title compound as yellow amorphous.

Yield: 1.56 g (55%).

¹H-NMR (400 MHz, CDCl₃) δ: 1.48 (br, 2H), 2.05 (br, 2H), 2.20 (br, 2H), 2.86 (br, 2H), 3.23 (s, 1H), 3.58 (s, 2H), 3.74 (s, 3H), 3.91 (s, 3H), 3.97 (s, 6H), 6.58 (d, 2H, J=8.8 Hz), 6.77 (d, 2H, J=9.0 Hz), 7.22 (d, 1H, J=5.1 Hz), 7.26 (s, 2H), 7.64 (s, 1H), 8.59 (d, 1H, J=4.9 Hz).

### PREPARATION EXAMPLE 38

### Synthesis of ethyl 2-(3,4,5-trimethoxyphenyl)nicotinate

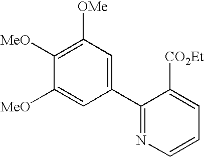

3,4,5-Trimethoxyphenylboronic acid (694 mg) and ethyl 2-chloronicotinate (608 mg) were reacted in the same manner as described in Preparation Example 1 to give the title compound.

Yield: 799 mg (77%).

¹H-NMR (400 MHz, CDCl₃) δ: 1.10 (t, 3H, J=7.2 Hz), 3.89 (s, 9H), 4.19 (q, 2H, J=7.2 Hz), 6.79 (s, 2H), 7.34 (dd, 1H, J=7.8 Hz, 4.8 Hz), 8.06 (dd, 1H, J=7.8 Hz, 1.7 Hz), 8.75 (dd, 1H, J=4.8 Hz, 1.7 Hz).

### PREPARATION EXAMPLE 39

### Synthesis of 3-hydroxymethyl-2-(3,4,5-trimethoxyphenyl)pyridine

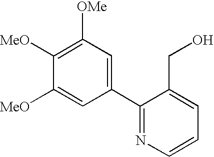

Ethyl 2-(3,4,5-trimethoxyphenyl)nicotinate (468 mg) was treated in the same manner as described in Preparation Example 2 to give the title compound.

Yield: 293 mg (72%).

¹H-NMR (400 MHz, CDCl₃) δ: 3.90 (s, 9H), 4.72 (s, 2H), 6.83 (s, 2H), 7.32 (dd, 1H, J=7.9 Hz, 4.8 Hz), 7.92 (dd, 1H, J=7.9 Hz, 1.7 Hz), 8.62 (dd, 1H, J=4.8 Hz, 1.7 Hz).

### PREPARATION EXAMPLE 40

### Synthesis of 3-chloromethyl-2-(3,4,5-trimethoxyphenyl)pyridine

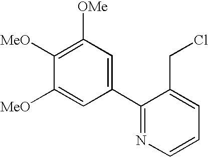

3-Hydroxymethyl-2-(3,4,5-trimethoxyphenyl)pyridine (293 mg) was treated in the same manner as described in the Preparation Example 3 to give the title compound.

Yield: 311 mg (theoretical amount).

### EXAMPLE 9

### Synthesis of 4-[N-(4-methoxyphenyl)-N-[[2-(3,4,5-trimethoxyphenyl)pyridin-3-yl]methyl]amino]-1-[[2-(3,4,5-trimethoxyphenyl)pyridin-4-yl]methyl]piperidine Trihydrochloride

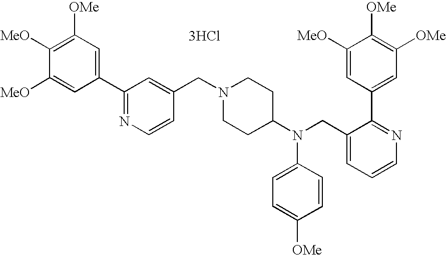

To a solution of 4-(p-anisidino)-1-[[2-(3,4,5-trimethoxyphenyl)pyridin-4-yl]methyl]piperidine (139 mg) and 3-chloromethyl-2-(3,4,5-trimethoxyphenyl)pyridine (114 mg) in acetonitrile (5 ml) was added potassium carbonate (83 mg) and potassium iodide (63 mg). The mixture was stirred at 70° C. overnight and evaporated. The residue was dissolved in chloroform, washed with water and brine, dried over anhydrous magnesium sulfate and evaporated. The residual oil was applied to a column of silica gel using diethylether-metanol (20:1) as an eluent. A free base obtained was converted to a trihydrochloride to give the title compound as yellow powder.

Yield: 16 mg, (8%).

¹H-NMR (400 MHz, measured as a free base, CDCl₃) δ: 1.60 (br, 2H), 1.77 (br, 21), 2.09 (br, 2H), 2.93 (br, 2H), 3.45 (br, 1H), 3.54 (s, 2H), 3.73 (s, 3H), 3.90 (s, 6H), 3.91 (s, 6H), 3.96 (s, 6H), 4.34 (s, 2H), 6.65 (d, 2H, J=9.0 Hz), 6.71 (s, 2H), 6.74 (d, 2H, J=9.0 Hz), 7.16-7.19 (m, 2H), 7.22 (s, 21), 7.55 (s, 1H), 7.79 (d, 1H, J=7.0 Hz), 8.50 (br, 1H), 8.58 (d, 1H, J=4.9 Hz).

### EXAMPLE 10

### Synthesis of 4-[N-(4-methoxyphenyl)-N-[[2-(3,4,5-trimethoxypheny)pyridin-4-yl]methyl]amino]-1-[[2-(3,4,5-trimethoxyphenyl)pyridin-4-yl]methyl]piperidine Trihydrochloride

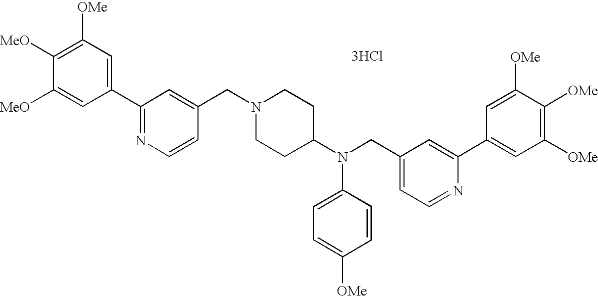

4-(p-Anisidino)-1-[[2-(3,4,5-trimethoxyphenyl)pyridin-4-yl]methyl]piperidine (1.56 g) and 4-chloromethyl-2-(3,4,5-trimethoxyphenyl)pyridine (1.08 g) were condensed by the same manner as described in Example 9. Yellow oil of a free base was converted to a trihydrochloride which gave the title compound as yellow powder.

Yield: 1.17 g (40%).

¹H-NMR (400 MHz, measured as a free base, CDCl₃) δ: 1.68-1.97 (m, 4H), 2.09-2.23 (m, 2H), 2.98 (br, 2H), 3.54-3.66 (m, 3H), 3.73 (s, 3H), 3.89 (s, 3H), 3.90 (s, 3H), 3.93 (s, 6H), 3.96 (s, 6H), 4.45 (s, 2H), 6.74 (d, 2H, J=9.2 Hz), 6.79 (d, 2H, J=9.2 Hz), 7.15 (s, 2H), 7.16-7.21 (m, 2H), 7.23 (s, 2H), 7.57 (s, 1H), 7.60 (s, 1H), 8.54 (d, 1H, J=5.1 Hz), 8.59 (d, 1H, J=4.9 Hz).

### PREPARATION EXAMPLE 41

### Synthesis of 3-(3,4,5-trimethoxyphenyl)benzyl Alcohol

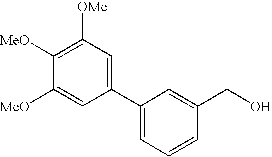

Ethyl 3-(3,4,5-trimethoxyphenyl)benzoate (5.09 g) was treated in the same manner as described in Preparation Example 2 to give the title compound.

Yield: 4.25 g (97%).

¹H-NMR (400 MHz, CDCl₃)δ: 1.87 (t, 1H, J=6.0 Hz), 3.89 (s, 3H), 3.92 (s, 6H), 4.76 (d, 1H, J=5.6 Hz), 6.77 (s, 2H), 7.34 (d, 1H, J=7.4 Hz), 7.42 (t, 1H, J=7.5 Hz), 7.48 (d, 1H, J=7.6 Hz), 7.55 (s, 1H).

### PREPARATION EXAMPLE 42

### Synthesis of 3-(3,4,5-trimethoxyphenyl)benzyl Chloride

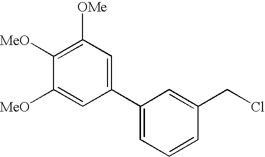

3-(3,4,5-Trimethoxyphenyl)benzyl alcohol (1.21 g) was treated in the same manner as described in Preparation Example 3 to give the title compound.

Yield: 893 mg (69%).

¹H-NMR (400 MHz, CDCl₃) δ: 3.87 (s, 3H), 3.90 (s, 6H), 4.62 (s, 2H), 6.75 (s, 2H), 7.33 (d, 1H, J=7.6 Hz), 7.39 (t, 1H, J=7.7 Hz), 7.48 (d, 1H, J=7.6 Hz), 7.54 (s, 1H).

### EXAMPLE 11

### Synthesis of 4-[N-(4-methoxyphenyl)-N-[3-(3,4,5-trimethoxyphenyl)benzyl]amino]-1-[[2-(3,4,5-trimethoxyphenyl)pyridin-4-yl]methyl]piperidine Dihydrochloride

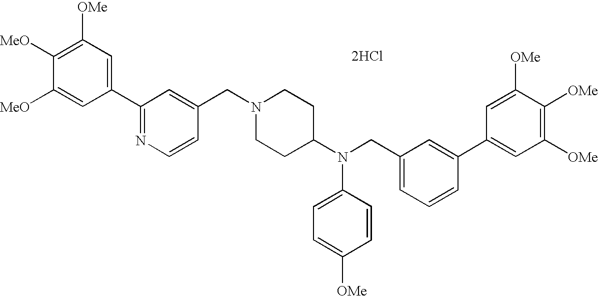

4-p-Anisidino)-1-[[2-(3,4,5-trimethoxyphenyl)pyridin-4-yl]methyl]piperidine (139 mg) and 3-(3,4,5-trimethoxyphenyl)benzyl chloride (114 mg) were condensed by the same manner as described in Example 9. Yellow oil of a free base was converted to a dihydrochloride which gave the title compound as yellow powder.

Yield: 52 mg (22%).

¹H-NMR (400 MHz, measured as a free base, CDCl₃) δ: 1.77-1.92 (m, 5H), 2.14-2.20 (m, 2H), 2.95-3.00 (m, 2H), 3.58 (s, 2H), 3.72 (s, 3H), 3.88 (s, 3H), 3.89 (s, 6H), 3.90 (s, 3H), 3.96 (s, 6H), 4.47 (s, 2H), 6.70 (s, 2H), 6.74-6.83 (m, 4H), 7.20 (d, 1H, J=7.4 Hz), 7.23 (s, 2H), 7.25-7.27 (m, 1H), 7.33 (t, 1H, J=7.4 Hz), 7.38 (d, 1H, J=8.7 Hz), 7.43 (s, 1H), 7.62 (s, 1H), 8.59 (d, 1H, J=5.1 Hz).

### PREPARATION EXAMPLE 43

### Synthesis of ethyl 6-(3,4,5-trimethoxyphenyl)nicotinate

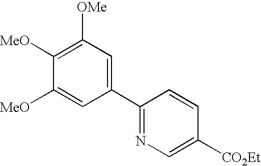

3,4,5-Trimethoxyphneylboronic acid (1.16 g) and ethyl 6-chloronitotinate (1.02 g) were coupled in the same manner as described in the Preparation Example 1 to give the title compound.

Yield: 1.42 g (82%)

¹H-NMR (400 MHz, CDCl₃) δ: 1.43 (t, 3H, J=7.2 Hz), 3.92 (s, 3H), 3.98 (s, 6H), 4.44 (q, 2H, J=7.2 Hz), 7.32 (s, 2H), 7.76 (d, 1H, J=8.3 Hz), 8.33 (dd, 1H, J=8.2 Hz, 2.2 Hz), 9.26 (d, 1H, J=2.2 Hz).

### PREPARATION EXAMPLE 44

### Synthesis of 5-hydroxymethyl-2-(3,4,5-trimethoxyphenyl)pyridine

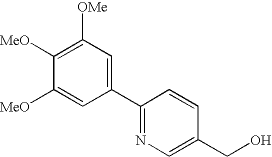

Ethyl 6-(3,4,5-trimethoxyphenyl)nicotinate (658 mg) was treated in the same manner as described in Preparation Example 2 to give the title compound.

Yield: 482 mg (85%).

¹H-NMR (400 MHz, CDCl₃) δ: 3.91 (s, 3H), 3.97 (s, 6H), 4.76 (s, 2H), 7.23 (s, 2H), 7.68 (d, 1H, J=7.4 Hz), 7.78 (dd, 1H, J=7.4 Hz, 2.3 Hz), 8.63 (d, 1H, J=2.3 Hz).

### PREPARATION EXAMPLE 45

### Synthesis of 5-chloromethyl-2-(3,4,5-trimethoxyphenyl)pyridine

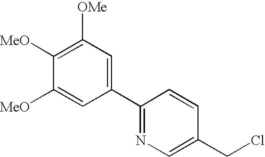

5-Hydroxymethyl-2-(3,4,5-trimethoxyphenyl)pyridine (685 mg) was treated in the same manner as described in Preparation Example 3 to give the title compound.

Yield: 717 mg (theoretical amount).

### EXAMPLE 12

### Synthesis of 4-[N-(4-methoxyphenyl)-N-[[2-(3,4,5-trimethoxyphenyl)pyridin-5-yl]methyl]amino]-1-[[2-(3,4,5-trimethoxyphenyl)pyridin-4-yl]methyl]piperidine Trihydrochloride

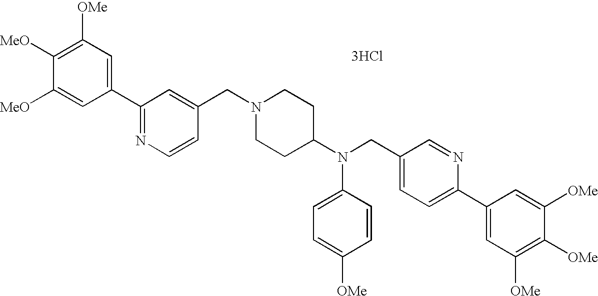

4-(p-Anisidino)-1-[[2-(3,4,5-trimethoxyphenyl)pyridin-4-yl]methyl]piperidine (139 mg) and 5-chloromethyl-2-(3,4,5-trimethoxyphenyl)pyridine (114 mg) were condensed by the same manner as described in Example 9. Yellow oil of a free base was converted to a trihydrochloride which gave the title compound as yellow powder.

Yield: 13 mg (5%).

¹H-NMR (400 MHz, measured as a free base, CDCl₃) δ: 1.76 (br, 2H), 1.88 (br, 2H), 2.14 (br, 2H), 2.97 (br, 2H), 3.51 (br, 1H), 3.57 (s, 2H), 3.73 (s, 3H), 3.89 (s, 3H), 3.90 (s, 3H), 3.94 (s, 6H), 3.96 (s, 6H), 4.42 (s, 2H), 6.78 (br, 4H), 7.20 (br, 3H), 7.23 (s, 2H), 7.57-7.70 (m, 3H), 8.58-8.60 (m, 2H).

### PREPARATION EXAMPLE 46

### Synthesis of Ethyl 5-(3,4,5-trimethoxyphenyl)nicotinate

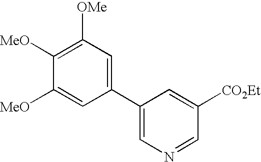

3,4,5-Trimethoxyphenylboronic acid (6.36 g) and ethyl 5-bromonicotinate (6.90 g) were reacted in the same manner as described in Preparation Example 1 to give the title compound.

Yield: 7.19 g (76%).

¹H-NMR (400 MHz, CDCl₃) δ: 1.44 (t, 3H, J=7.1 Hz), 3.91 (s, 3H), 3.95 (s, 6H), 4.46 (q, 2H, J=7.1 Hz), 6.79 (s, 2H), 8.44 (t, 1H, J=2.1 Hz), 8.96 (d, 1H, J=2.1 Hz), 9.18 (d, 1H, J=1.8 Hz).

### PREPARATION EXAMPLE 47

### Synthesis of 3-hydroxymethyl-5-(3,4,5-trimethoxyphenyl)pyridine

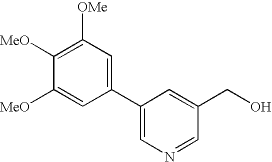

Ethyl 5-(3,4,5-trimethoxyphenyl)nicotinate (7.19 g) was treated in the same manner as described in the Preparation Example 2 to give the title compound.

Yield; 3.83 g (61%).

¹H-NMR (400 MHz, CDCl₃) δ: 3.88 (s, 3H), 3.89 (s, 6H), 4.39 (br, 1H), 4.80 (s, 2H), 6.72 (s, 2H), 7.89 (t, 1H, J=1.2 Hz), 8.47 (d, 1H, J=2.1 Hz), 8.63 (d, 1H, J=2.2 Hz).

### PREPARATION EXAMPLE 48

### Synthesis of 3-chloromethyl-5-(3,4,5-trimethoxyphenyl)pyridine

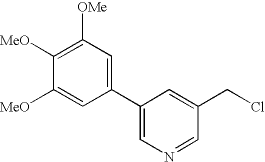

3-Hydroxymethyl-5-(3,4,5-trimethoxyphenyl)pyridine (2.85 g) was treated in the same manner as described in Preparation Example 3 to give the title compound.

Yield: 1.97 g (65%).

¹H-NMR (400 MHz, CDCl₃) δ: 3.90 (s, 3H), 3.94 (s, 6H), 4.67 (s, 2H), 6.75 (s, 2H), 7.87 (t 1H, J=2.1 Hz), 8.59 (d, 1H, J=2.0 Hz), 8.76 (d, 1H, J=2.1 Hz).

### EXAMPLE 13

### Synthesis of 4-[N-(4-methoxyphenyl)-N-[[5-(3,4,5-trimethoxyphenyl)pyridin-3-yl]methyl]amino]-1-[[2-(3,4,5-trimethoxyphenyl)pyridin-4-yl]methyl]piperidine Trihydrochloride

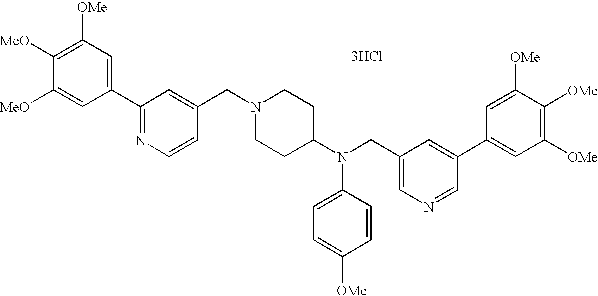

4-(p-Anisidino)-1-[[2-(3,4,5-trimethoxyphenyl)pyridin-4-yl]methyl]piperidine (139 mg) and 3-chloromethyl-5-(3,4,5-trimethoxyphenyl)pyridine (114 mg) were condensed by the same manner as described in Example 9. Yellow oil of a free base was converted to a trihydrochloride which gave the title compound as yellow powder.

Yield: 14 mg (5%).

¹H-NMR (400 MHz, measured as a free base, CDCl₃) δ: 1.73-1.75 (m, 2H), 1.88 (d, 2H, J=11.3 Hz), 2.13 (t, 2H, J=11.3 Hz), 2.96 (d, 2H, J=11.5 Hz), 3.50 (br, 1H), 3.55 (s, 2H), 3.72 (s, 3H), 3.88 (s, 3H), 3.89 (s, 9H), 3.96 (s, 6H), 4.45 (s, 2H), 6.65 (s, 2H), 6.76 (d, 2H, J=9.6 Hz), 6.80 (d, 2H, J=9.4 Hz), 7.20 (d, 1H, J=5.3 Hz), 7.22 (s, 2H), 7.59 (s, 1H), 7.67 (s, 1H), 8.50 (s, 1H), 8.59 (d, 1H, J=4.7 Hz), 8.62 (s, 1H).

### PREPARATION EXAMPLE 49

### Synthesis of 2,6-dimethoxy-4-iodophenol

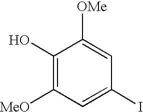

To a solution of 5-iodo-1,2,3-trimethoxybenzene (3.2 g) in 1,2-dichloroethane (40 mL) was added aluminum chloride (1.6 g). The mixture was stirred at 60° C. for 4 hours and evaporated. The residue was dissolved in 1 M aqueous sodium hydroxide solution and washed with ether. The aqueous layer was then acidified and extracted with chloroform. The organic layer was washed with brine, dried over anhydrous magnesium sulfate and evaporated to give the title compound as white crystalline powder.

Yield: 1.0 g (31%)

### PREPARATION EXAMPLE 50

### Synthesis of 1,3-dimethoxy-5-iodo-2-isopropoxybenzene

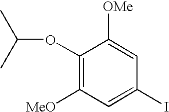

To a suspension of 2,6-dimethoxy-4-iodophenol (1.0 g) and potassium carbonate (938 mg) in DMF (10 mL) was added isopropyl iodide (507 μL). The mixture was stirred at 60° C. for 3 hours and evaporated. Ethyl acetate and water were added to the residue, the organic layer was separated, washed with brine, dried over anhydrous sodium sulfate and evaporated. The residue was applied to a column of silica gel using hexane-ethyl acetate (5:1) as an eluent to give the title compound.

Yield: 788 mg (72%).

### PREPARATION EXAMPLE 51

### Synthesis of 3,5-dimethoxy-4-isopropoxyphenylboronic Acid

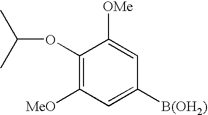

1,3-Dimethoxy-5-iodo-2-isopropoxybenzene (2.25 g) was treated in the same manner as described in Preparation Example 27 to give the title compound.

Yield: 1.23 g (74%).

### PREPARATION EXAMPLE 52

### Synthesis of ethyl 2-(3,5-dimethoxy-4-isopropoxyphenyl)isonicotinate

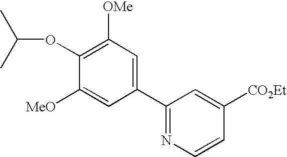

To a solution of 3,5-dimethoxy-4-isopropoxyphenylboronic acid (1.23 g) and ethyl 2-chloroisonicotinate (0.95 g) were condensed in the same manner as described in Preparation Example 1 to give the title compound.

Yield: 1.57 g (89%).

¹H-NMR (400 MHz, CDCl₃) δ: 1.33 (d, 6H, J=4.9 Hz), 1.44 (t, 3H, J=7.1 Hz), 3.95 (s, 6H), 4.42-4.49 (m, 3H), 7.29 (s, 2H), 7.75 (dd, 1H, J=4.9 Hz, 1.4 Hz), 8.24 (s, 1H), 8.80 (d, 1H, J=4.9 Hz).

### PREPARATION EXAMPLE 53

### Synthesis of 2-(3,5-dimethoxy-4-isopropoxyphenyl)-4-hydroxymethylpyridine

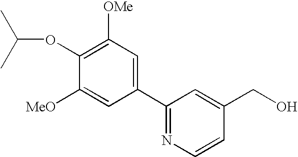

Ethyl 2-(3,5-dimethoxy-4-isopropoxyphenyl)isonicotinate (1.57 g) was treated in the same manner as described in the Preparation Example 2 to give the title compound.

Yield: 1.27 g (92%).

¹H-NMR (400 MHz, CDCl₃) δ: 1.32 (d, 6H, J=6.1 Hz), 3.93 (s, 6H), 4.45 (quint, 1H, J=6.1 Hz), 4.81 (s, 2H), 7.20 (d, 1H, J=5.1 Hz), 7.23 (s, 2H), 7.68 (s, 1H), 8.62 (d, 1H, J=5.1 Hz).

### PREPARATION EXAMPLE 54

### Synthesis of 4-chloromethyl-2-(3,5-dimethoxy-4-isopropoxyphenyl)pyridine

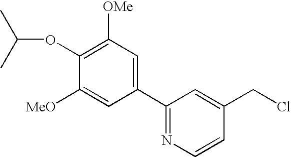

2-(3,5-Dimethoxy-4-isopropoxyphenyl)-4-hydroxymethylpyridine (1.49 g) was treated in the same manner as described in Preparation Example 3 to give the title compound.

Yield: 1.33 g (84%).

¹H-NMR (400 MHz, CDCl₃) δ: 1.32 (d, 6H, J=6.2 Hz), 3.94 (s, 6H), 4.45 (quint, 1H, J=6.1 HZ), 4.61 (s, 2H), 7.23-7.26 (m, 3H), 7.69 (s, 1H), 8.66 (d, 1H, J=5.1 Hz).

### PREPARATION EXAMPLE 55

### Synthesis of 1-[[2-(3,5-dimethoxy-4-isopropoxyphenyl)pyridin-4-yl]methyl]-4-piperidone Ethylene Ketal

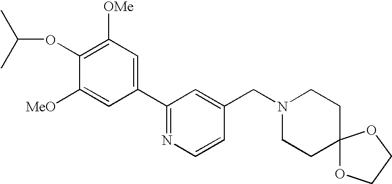

4-Chloromethyl-2-(3,5-dimethoxy-4-isopropoxyphenyl)pyridine (643 mg) and 4-piperidone ethylene ketal (287 mg) were coupled in the same manner as described in Example 2 to give the title compound.

Yield: 818 mg (95%).

¹H-NMR (400 MHz, CDCl₃) δ: 1.32 (d, 6H, J=6.1 Hz), 1.78 (t, 4H, J=5.7 Hz), 2.57 (br, 4H), 3.49 (s, 4H), 3.59 (s, 2H), 3.94 (s, 6H), 4.44 (quint, 1H, J=6.1 Hz), 7.21 (d, 1H, J=5.1 Hz), 7.23 (s, 2H), 7.65 (s, 1H), 8.59 (d, 1H, J=5.1 Hz).

### PREPARATION EXAMPLE 56

### Synthesis of 1-[[2-(3,5-dimethoxy-4-isopropoxyphenyl)pyridin-4-yl]methyl]-4-piperidone

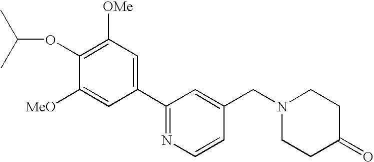

1-[[2-(3,5-Dimethoxy-4-isopropoxyphenyl)pyridin-4-yl]methyl]-4-piperidone ethylene ketal (818 mg) was treated in the same manner as described in Preparation Example 23 to give the title compound.

Yield: 717 mg (98%).

¹H-NMR (400 MHz, CDCl₃) δ: 1.32 (d, 6H, J=6.2 Hz), 2.50 (t, 4H, J=6.1 Hz), 2.81 (t, 4H, J=6.1 Hz), 3.69 (s, 2H), 3.95 (s, 6H), 4.45 (quint, 1H, J=6.2 Hz), 7.24 (s, 2H), 7.25-7.27 (m, 1H), 7.68 (s, 1H), 8.63 (d, 1H, J=5.1 Hz).

### PREPARATION EXAMPLE 57

### Synthesis of 4-(p-anisidino)-1-[[2-(3,5-dimethoxy-4-isopropoxyphenyl)pyridin-4-yl]methyl]piperidine

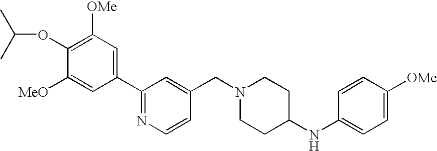

1-[[2-(3,5-dimethoxy-4-isopropoxyphenyl)pyridin-4-yl]methyl]-4-piperidone (350 mg) and p-anisidine (123 mg) were condensed in the same manner as described in Preparation Example 37 to give the title compound.

Yield: 307 mg (69%).

¹H-NMR (400 MHz, CDCl₃) δ: 1.32 (d, 6H, J=6.3 Hz), 1.46-1.52 (m, 2H), 2.00-2.24 (m, 2H), 2.22 (t, 2H, J=11.1 Hz), 2.86 (d, 2H, J=12.1 Hz), 3.18-3.28 (m, 1H), 3.58 (s, 2H), 3.74 (s, 3H), 3.94 (s, 6H), 4.40 (quint, 1H, J=6.3 Hz), 6.58 (d, 2H, J=6.6 Hz), 6.78 (d, 2H, J=6.6 Hz), 7.20 (d, 1H, J=5.1 Hz), 7.24 (s, 2H), 7.64 (s, 1H), 8.59 (d, 1H, J=5.1 Hz).

### EXAMPLE 14

### Synthesis of 1-[[2-(3,5-dimethoxy-4-isopropoxyphenyl)pyridin-4-yl]methyl]-4-[N-[[2-(3,5-dimethoxy-4-isopropoxyphenyl)pyridin-4-yl]methyl]-N-(4-methoxyphenyl) amino]piperidine Trihydrochloride

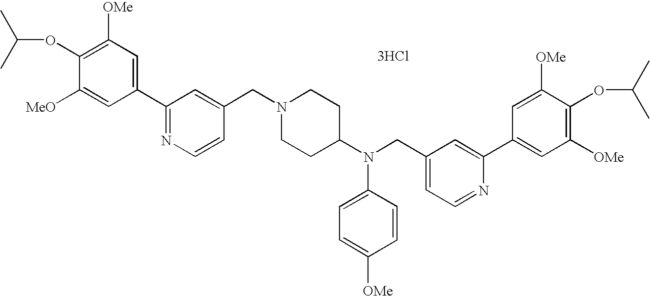

4-p-anisidino)-1-[[2-(3,5-dimethoxy-4-isopropoxyphenyl)pyridin-4-yl]methyl] piperidine (307 mg) and 4-chloromethyl-2-(3,5-dimethoxy-4-isopropoxyphenyl) pyridine (201 mg) were condensed in the same manner as described in Example 9. A free base obtained was converted to a trihydrochloride giving the title compound as yellow powder.

Yield: 230 mg (46%).

¹H-NMR (400 MHz, CDCl₃) δ: 1.31 (d, 6H, J=3.3 Hz), 1.32 (d, 6H, J=6.8 Hz), 1.70-1.92 (m, 4H), 2.10-2.20 (m, 2H), 2.92-3.01 (m, 2H), 3.56 (s, 2H), 3.73 (s, 3H), 3.85-3.95 (m, 1H), 3.90 (s, 6H), 3.93 (s, 6H), 4.39-4.49 (m, 4H), 6.73 (d, 2H, J=4.8 Hz), 6.78 (d, 2H, J=4.8 Hz), 7.14 (s, 2H), 7.15-7.20 (m, 2H), 7.23 (s, 2H), 7.58 (s, 1H), 7.60 (s, 1H), 8.53 (d, 1H, J=5.1 Hz), 8.58 (d, 1H, J=5.1 Hz).

### PREPARATION EXAMPLE 58

### Synthesis of 4-benzylamino-1-[[2-(3,4,5-trimethoxyphenyl)pyridin-4-yl]-methyl] Piperidine

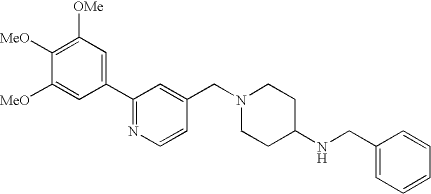

1-[[2-(3,4,5-Trimethoxyphenyl)pyridin-4-yl]methyl-4-piperidone (1.40 g) and benzylamine (0.51 g) was condensed in the same manner as described in Preparation Example 37 to give the title compound as yellow amorphous.

Yield: 1.20 g (68%).

¹H-NMR (400 MHz, CDCl₃) δ: 1.40-1.60 (m, 2H), 1.88-2.09 (m, 5H), 2.54 (br, 1H), 2.82-2.85 (m, 2H), 3.52 (s, 2H), 3.80 (s, 2H), 3.89 (s, 3H), 3.95 (s, 6H), 7.18-7.31 (m, 8H), 7.64 (s, 1H), 8.57 (d, 1H, J=5.1 Hz).

### EXAMPLE 15

### Synthesis of 4-[N-benzyl-N-[[2-(3,4,5-trimethoxypheny)pyridin-3-yl]-methyl]amino]-1-[[2-(3,4,5-trimethoxyphenyl)pyridinyl]methyl]piperidine Tetrahydrochloride

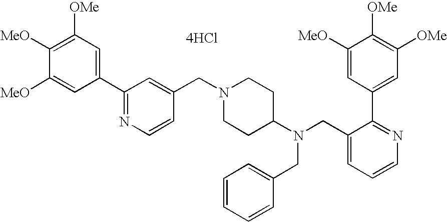

4-Benzylamino-1-[[2-(3,4,5-trimethoxyphenyl)pyridin-4-yl]methyl]piperidine (134 mg) and 3-chloromethyl-2-(3,4,5-trimethoxyphenyl)pyridine (114 mg) were condensed in the same manner as described in Example 9. A free base obtained was converted to a tetrahydrochloride to give the title compound as yellow powder.

Yield: 43 mg, (17%).

¹H-NMR (400 MHz, measured as a free base, CDCl₃) δ: 1.63 (br, 4H), 1.87 (br, 2H), 2.39 (br, 1H), 2.88 (br, 2H), 3.49 (s, 2H), 3.57 (s, 2H), 3.68 (s, 2H), 3.86 (s, 6H), 3.88 (s, 3H), 3.90 (s, 3H), 3.96 (s, 6H), 6.60 (s, 2H), 7.17 (d, 1H, J=5.1 Hz), 7.22-7.29 (m, 8H), 7.56 (s, 1H), 8.02 (d, 1H, J=8.0 Hz), 8.50 (d, 1H, J=6.4 Hz), 8.58 (d, 1H, J=5.1 Hz).

### EXAMPLE 16

### Synthesis of 4-[N-benzyl-N-[[2-(3,4,5-trimethoxyphenyl)pyridin-4-yl]methyl]amino]-1-[[2-(3,4,5-trimethoxyphenyl)pyridin-4-yl]methyl]piperidine Tetrahydrochloride

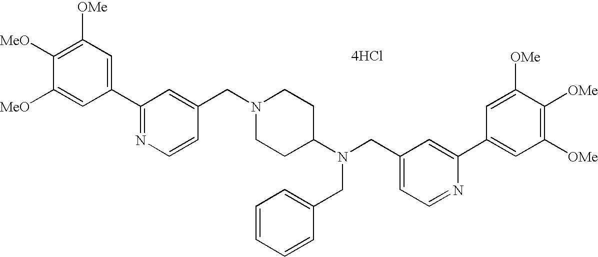

4-Benzylamino-1-[[2-(3,4,5-trimethoxyphenyl)pyridin-4-yl]methyl]piperidine (230 mg) and 4-chloromethyl-2-(3,4,5-trimethoxyphenyl)pyridine (158 mg) were condensed by the same manner as described in Example 9. Yellow oil of a free base was converted to a tetrahydrochloride which gave the title compound as yellow powder.

Yield: 172 mg (47%).

¹H-NMR (400 MHz, measured as a free base, CDCl₃) δ: 1.69-1.85 (m, 4H), 1.93-1;99 (m, 2H), 2.56 (br, 1H), 2.93-3.00 (m, 2H), 3.51 (s, 2H), 3.71 (s, 2H), 3.74 (s, 2H), 3.90 (s, 6H), 3.96 (s, 6H), 7.18-7.32 (m, 9H), 7.38 (d, 2H, J=7.1 Hz), 7.59 (s, 1H), 7.68 (s, 1H), 8.56 (d, 1H, J=5.1 Hz), 8.60 (d, 1H, J=5.1 Hz).

### EXAMPLE 17

### Synthesis of 4-[N-benzyl-N-[3-(3,4,5-trimethoxyphenyl)benzyl]amino]-1-[[2-(3,4,5-trimethoxyphenyl)pyridin-4-yl]methyl]piperidine Trihydrochloride

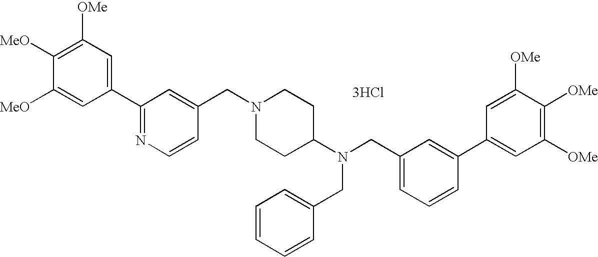

4-Benzylamino-1-[[2-(3,4,5-trimethoxyphenyl)pyridin-4-yl]methyl]piperidine (134 mg) and 3-(3,4,5-trimethoxyphenyl)benzyl chloride (114 mg) were condensed by the same manner as described in Example 9. Yellow oil of a free base was converted to a trihydrochloride which gave the title compound as yellow powder.

Yield: 47 mg (18%).

¹H-NMR (400 MHz, measured as a free base, CDCl₃) δ: 1.70-1.86 (m, 4H), 1.96 (br, 2H), 2.59 (br, 1H), 2.94 (br, 2H), 3.51 (s, 2H), 3.70 (s, 2H), 3.74 (s, 2H), 3.89 (s, 3H), 3.90 (s, 3H), 3.92 (s, 6H), 3.96 (s, 6H), 6.75 (s, 2H), 7.18-7.30 (m, 6H), 7.35-7.40 (m, 5H), 7.56 (s, 1H), 7.60 (s, 1H), 8.58 (d, 1H, J=5.1 Hz).

### EXAMPLE 18

### Synthesis of 4-[N-benzyl-N-[[2-(3,4,5-trimethoxyphenyl)pyridin-5-yl]methyl]amino]-1-[[2-(3,4,5-trimethoxyphenyl)pyridin-4-yl]methyl]piperidine Tetrahydrochloride

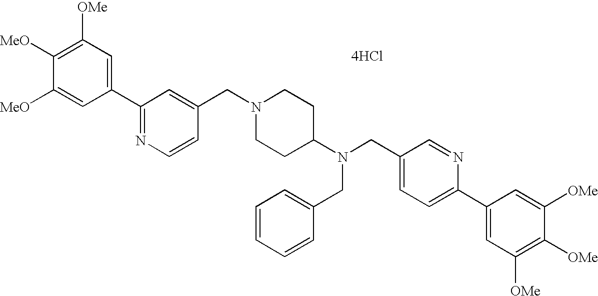

4-Benzylamino-1-[[2-(3,4,5-trimethoxyphenyl)pyridin-4-yl]methyl]piperidine (134 mg) and 5-chloromethyl-2-(3,4,5-trimethoxyphenyl)pyridine (114 mg) were condensed by the same manner as described in Example 9. Yellow oil of a free base was converted to a tetrahydrochloride which gave the title compound as yellow powder.

Yield: 44 mg (17%).

¹H-NMR (400 MHz, measured as a free base, CDCl₃) δ: 1.81 (br, 4H), 1.96 (br, 2H), 2.55 (br, 1H), 2.96 (br, 2H), 3.52 (s, 2H), 3.69 (s, 4H), 3.89 (s, 6H), 3.95 (s, 6H), 3.96 (s, 6H), 7.19-7.32 (m, 8H), 7.36-7.38 (m, 2H), 7.61 (d, 2H, J=7.6 Hz), 7.69-7.73 (m, 1H), 8.59 (d, 1H, J=4.9 Hz), 8.63 (s, 1H).

### EXAMPLE 19

### Synthesis of 4N-benzyl-N-[[5-(3,4,5-trimethoxypheny)pyridin-3-yl]methyl]amino]1-[[2-(3,4,5-trimethoxyphenyl)pyridin-4-yl]methyl]piperidine Tetrahydrochloride

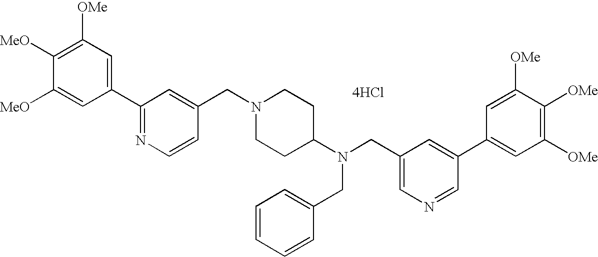

4-Benzylamino-1-[[2-(3,4,5-trimethoxyphenyl)pyridin-4-yl]methyl]piperidine (134 mg) and 3-chloromethyl-5-(3,4,5-trimethoxyphenyl)pyridine (114 mg) were condensed by the same manner as described in Example 9. Yellow oil of a free base was converted to a tetrahydrochloride which gave the title compound as yellow powder.

Yield: 26 mg (10%).

¹H-NMR (400 MHz, measured as a free base, CDCl₃) δ: 1.83 (br, 4H), 1.97 (br, 2H), 2.58 (br, 1H), 2.95 (br, 2H), 3.53 (s, 2H), 3.71 (s, 2H), 3.75 (s, 2H), 3.90 (s, 6H), 3.93 (s, 6H), 3.96 (s, 6H), 6.74 (s, 2H), 7.19-7.30 (m, 6H), 7.36 (d, 2H, J=6.8 Hz), 7.60 (s, 1H), 7.79 (s, 1H), 8.54 (s, 1H), 8.59 (d, 1H, J=5.1 Hz), 8.64 (s, 1H).

### PREPARATION EXAMPLE 59

### Synthesis of 1-(tert-butoxycarbonyl)-4-[N-[[2-(3,4,5-trmethoxyphenyl)pyridin-4-yl]methyl]aminomethyl]piperidine

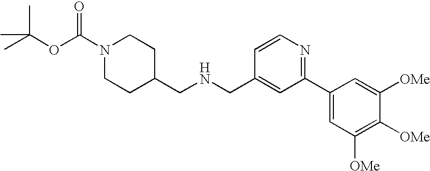

1-(tert-Butoxycarbonyl)-4-aminomethylpiperidine (200 mg) and 4-chloromethyl-2-(3,4,5-trimethoxyphenyl)pyridine (183 mg) were condensed in the same manner as described in Example 2 to give the title compound as yellow syrup.

Yield: 264 mg (90%).

¹H-NMR (400 MHz, CDCl₃) δ: 1.12-1.27 (m, 3H), 1.45 (s, 9H), 1.60 (br, 1H), 1.74 (d, 2H, J=12.9 Hz), 2.54 (d, 2H, J=6.6 Hz), 2.69 (br, 2H), 3.87 (s, 2H), 3.90 (s, 3H), 3.97 (s, 6H), 4.03-4.14 (m, 2H), 7.20 (d, 1H, J=3.9 Hz), 7.24 (s, 2H), 7.65 (s, 1H), 8.60 (d, 1H, J=4.9 Hz).

### PREPARATION EXAMPLE 60

### Synthesis of 1-(tert-butoxycarbonyl)-4-[N-methyl-N-[[2-(3,4,5-trimethoxyphenyl) pyridin-4-yl]methyl]aminomethyl]piperidine

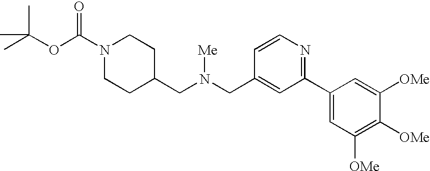

1-(tert-butoxycarbonyl)-4-[N-[[2-(3,4,5-trimethoxyphenyl)pyridin-4-yl]methyl]aminomethyl]piperidine (264 mg) was treated in the same manner as described in Preparation Example 11 to give the title compound as yellow syrup.

Yield: 157 mg (58%).

¹H-NMR (400 MHz, CDCl₃) δ: 1.00-1.09 (m, 2H), 1.43 (s, 9H), 1.65-1.70 (m, 1H), 1.79 (d, 2H, J=12.7 Hz), 2.21 (d, 2H, J=7.4 Hz), 2.23 (s, 3H), 2.69 (br, 2H), 3.52 (s, 2H), 3.89 (s, 3H), 3.96 (s, 6H), 4.07-4.13 (m, 2H), 7.20 (d, 1H, J=4.9 Hz), 7.24 (s, 2H), 7.64 (s, 1H), 8.58 (d, 1H, J=5.1 Hz).

### PREPARATION EXAMPLE 61

### Synthesis of 4-[N-methyl-N-[[2-(3,4,5-trimethoxyphenyl)pyridin-4-yl]methyl]aminomethyl] Piperidine

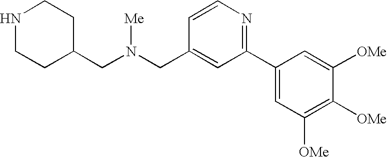

1-(tert-Butoxycarbonyl)-4-[N-methyl-N-[[2-(3,4,5-trimethoxyphenyl)pyridin-4-yl]methyl]aminomethyl]piperidine (152 mg) was treated in the same manner as described in Preparation Example 12 to give the title compound as yellow crystals.

Yield: 105 mg (88%).

¹H-NMR (400 MHz, CDCl₃) δ: 1.00-1.10 (m, 2H), 1.60-1.68 (m, 1H), 1.80 (d, 2H, J=12.5 Hz), 2.03 (br, 1H), 2.20 (d, 2H, J=8.4 Hz), 2.21 (s, 3H), 2.58 (dt, 2H, J=12.1 Hz, 2.1 Hz), 3.05 (d, 2H, J=12.1 Hz), 3.51 (s, 2H), 3.89 (s, 3H), 3.95 (s, 6H), 7.20 (d, 1H, J=5.1 Hz), 7.24 (s, 2H), 7.65 (s, 1H), 8.57 (d, 1H, J=5.9 Hz).

### EXAMPLE 20

### Synthesis of 4-[N-methyl-N-[[2-(3,4,5-trimethoxyphenyl)pyridin-4-yl]methyl]aminomethyl]-1-[[2-(3,4,5-trimethoxyphenyl)pyridin-4-yl]methyl]piperidine Dioxalate

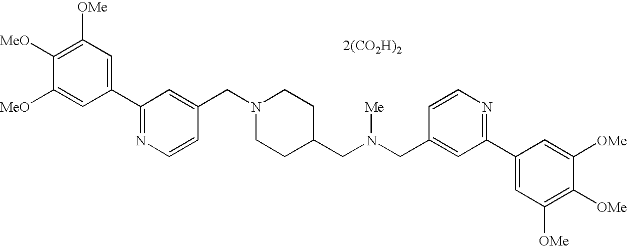

4-[N-methyl-N-[[2-(3,4,5-trimethoxyphenyl)pyridin-4-yl]methyl]amino-methyl] piperidine (96 mg) and 4-chloromethyl-2-(3,4,5-trimethoxyphenyl)pyridine (73 mg) were condensed in the same manner as described in Example 2. The title compound was obtained as white powder after converting a free base to a dioxalate.

Yield: 109 mg (40%).

¹H-NMR (400 MHz, measured as a free base, CDCl₃) δ: 1.19-1.27 (m, 2H), 1.56 (br, 1H), 1.81 (d, 2H, J=11.1 Hz), 1.99-2.04 (m, 2H), 2.23 (s, 5H), 2.88 (d, 2H, J=11.1 Hz), 3.53 (s, 4H), 3.89 (s, 3H), 3.90 (s, 3H), 3.94 (s, 6H), 3.96 (s, 6H), 7.20 (br, 2H), 7.23 (s, 4H), 7.61 (s, 1H), 7.64 (s, 1H), 8.58 (d, 2H, J=4.9 Hz).

### PREPARATION EXAMPLE 62

### Synthesis of 4-(3,5-dimethoxyphenylamino)-1-[[2-(3,4,5-trimethoxyphenyl)pyridin-4-yl]methyl]piperidine

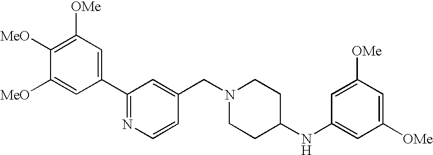

1-[[2-(3,4,5-Trimethoxyphenyl)pyridinyl]methyl-4-piperidone (1.40 g) and 3,5-dimethoxyaniline (722 mg) were treated in the same manner as described in Preparation Example 37 to give the title compound.

Yield: 800 mg (41%).

¹H-NMR (400 MHz, CDCl₃) δ: 1.40-1.90 (m, 2H), 1.95-2.50 (m, 4H), 2.93 (br, 2H), 3.31 (br, 1H), 3.65 (br, 2H), 3.72 (s, 6H), 3.88 (s, 3H), 3.96 (s, 6H), 5.76 (s, 2H), 5.85 (s, 1H), 7.20-7.35 (m, 3H), 7.73 (br, 1H), 8.60 (d, 1H, J=4.9 Hz).

### EXAMPLE 21

### Synthesis of 4-[N-(3,5-dimethoxyphenyl)-N-[[2-(3,4,5-trimethoxyphenyl)pyridin-3-yl]methyl]amino]-1-[[2-(3,4,5-trimethoxyphenyl)pyridin-4-yl]methyl]piperidine Trihydrochloride

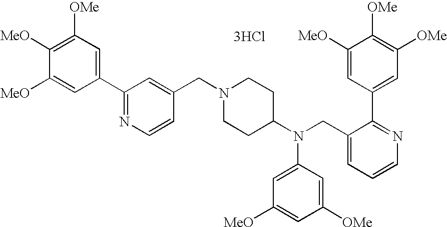

4-(3,5-Dimethoxyphenylamino)-1-[[2-(3,4,5-trimethoxyphenyl)pyridin-4-yl]m ethyl]piperidine (148 mg) and 3-chloromethyl-2-(3,4,5-trimethoxyphenyl)pyridine (114 mg) were condensed in the same manner as described in Example 9. Yellow syrup obtained was converted to a trihydrochloroide to give the title compound as yellow powder.

Yield: 29 mg, (11%).

¹H-NMR (400 MHz, measured as a free base, CDCl₃) δ: 1.60-1.63 (m, 2H), 1.79 (d, 2H, J=11.7 Hz), 2.13 (t, 2H, J=11.4 Hz), 2.94 (d, 2H, J=11.3 Hz), 3.54 (s, 2H), 3.71 (s, 6H), 3.78-3.84 (m, 1H), 3.90 (s, 3H), 3.91 (s, 6H), 3.92 (s, 3H), 3.96 (s, 6H), 4.41 (s, 2H), 5.84 (s, 2H), 6.72 (s, 2H), 7.09-7.24 (m, 5H), 7.53 (s, 1H), 7.71 (d, 1H, J=6.6 Hz), 8.51 (dd, 1H, J=4.7 Hz, 1.6 Hz), 8.59 (d, 1H, J=4.9 Hz).

### PREPARATION EXAMPLE 63

### Synthesis of ethyl 2-(3,4,5-trimethoxyphenyl)benzoate

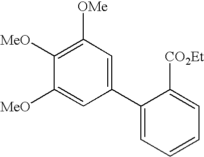

3,4,5-Trimethoxyphenylboronic acid (639 mg) and ethyl 2-bromobenzoate (479 mg) were condensed in the same manner as described in Preparation Example 1 to give the title compound.

Yield: 655 mg (69%).

¹H-NMR (400 MHz, CDCl₃) δ: 1.04 (t, 3H, J=7.2 Hz), 3.86 (s, 6H), 3.89 (s, 3H), 4.12 (q, 2H, J=7.2 Hz), 6.54 (s, 2H), 7.40-7.42 (m, 2H), 7.51 (t, 1H, J=7.8 Hz), 7.77 (d, 1H, J=6.8 Hz).

### PREPARATION EXAMPLE 64

### Synthesis of 2-(3,4,5-trimethoxyphenyl)benzyl Alcohol

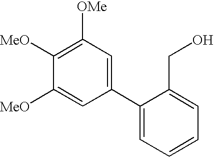

Ethyl 2-(3,4,5-trimethoxyphenyl)benzoate (655 mg) was treated in the same manner as described in Preparation Example 2 to give the title compound.

Yield: 630 mg (theoretical amount).

¹H-NMR (400 MHz, CDCl₃) δ: 3.85 (s, 6H), 3.90 (s, 3H), 4.61 (s, 2H), 6.61 (s, 2H), 7.26-7.39 (m, 3H), 7.53 (d, 1H, J=6.8 Hz).

### PREPARATION EXAMPLE 65

### Synthesis of 2-(3,4,5-trimethoxyphneyl)benzyl Chloride

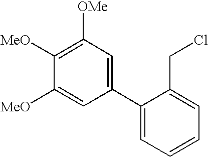

2-(3,4,5-Trimethoxyphenyl)benzyl alcohol (630 mg) was treated in the same manner as described in Preparation Example 3 to give the title compound.

Yield: 615 mg (theoretical amount).

¹H-NMR (400 MHz, CDCl₃) δ: 3.87 (s, 6H), 3.90 (s, 3H), 4.53 (s, 2H), 6.66 (s, 2H), 7.29-7.32 (m, 1H), 7.34-7.39 (m, 2H), 7.50-7.52 (m, 1H).

### EXAMPLE 22

### Synthesis of 4-[N-(3,5-dimethoxyphenyl)-N-[2-(3,4,5-trimethoxyphenyl)benzyl]amino]-1-[[2-(3,4,5-trimethoxyphenyl)pyridin-4-yl]methyl]piperidine Dihydrochloride

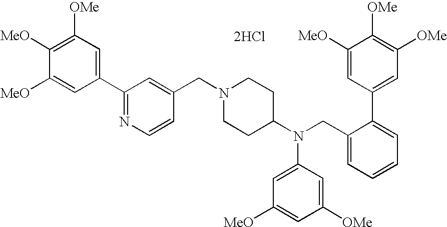

4-(3,5-Dimethoxyphenylamino)-1-[[2-(3,4,5-trimethoxyphenyl)pyridin-4-yl]methyl]piperidine (148 mg) and 2-(3,4,5-trimethoxyphenyl)benzyl chloride (114 mg) were condensed in the same manner as described in Example 9. A free base obtained was converted to a dihydrochloroide to give the title compound as yellow powder.

Yield: 20 mg, (8%).

¹H-NMR (400 MHz, measured as a free base, CDCl₃) δ: 1.50-1.90 (m, 4H), 2.05-2.20 (m, 2H), 2.92 (br, 2H), 3.52 (br, 3H), 3.68 (s, 6H), 3.85 (s, 6H), 3.88 (s, 3H), 3.89 (s, 3H), 3.94 (s, 6H), 4.31 (s, 2H), 5.85 (br, 3H), 6.52 (s, 2H), 7.05-7.27 (m, 6H), 7.34 (s, 1H), 7.51 (s, 1H), 8.56 (s, 1H).

### EXAMPLE 23

### Synthesis of 4-[N-(3,5-dimethoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine Trihydrochloride

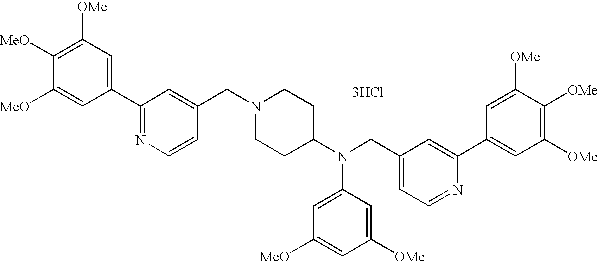

4-(3,5-Dimethoxyphenylamino)-1-[[2-(3,4,5-trimethoxyphenyl)pyridin-4-yl]methyl]piperidine (148 mg) and 4-chloromethyl-2-(3,4,5-trimethoxyphenyl)pyridine (114 mg) were condensed by the same manner as described in Example 9. Yellow oil of a free base was converted to a trihydrochloride which gave the title compound as yellow powder.

Yield: 40 mg (18%).

¹H-NMR (400 MHz, measured as a free base, CDCl₃) δ: 1.68-1.90 (m, 4H), 2.12-2.22 (m, 2H), 2.94-3.02 (m, 2H), 3.57 (s, 2H), 3.71 (s, 6H), 3.81-3.83 (m, 1H), 3.89 (s, 3H), 3.90 (s, 3H), 3.93 (s, 6H), 3.96 (s, 6H), 4.52 (s, 2H), 5.89-5.94 (m, 3H), 7.14 (d, 1H, J=5.3 Hz), 7.16 (s, 2H), 7.20 (d, 1H, J=3.7 Hz), 7.22 (s, 2H), 7.54-7.60 (m, 2H), 8.55 (d, 1H, J=5.1 Hz), 8.59 (d, 1H, J=5.1 Hz).

### EXAMPLE 24

### Synthesis of 4-[N-(3,5-dimethoxyphenyl)-N-[3-(3,4,5-trimethoxyphenyl)benzyl]amino]-1-[[2-(3,4,5-trimethoxyphenyl)pyridin-4-yl]methyl]piperidine Dihydrochloride

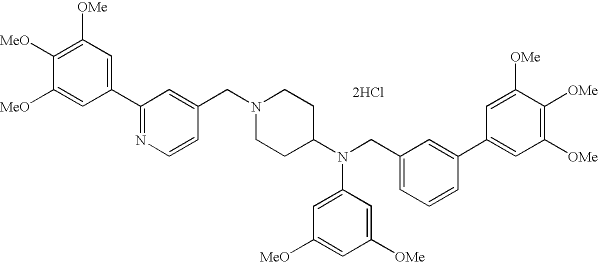

4-(3,5-Dimethoxyphenylamino)-1-[[2-(3,4,5-trimethoxyphenyl)pyridin-4-yl]methyl]piperidine (148 mg) and 3-(3,4,5-trimethoxyphenyl)benzyl chloride (114 mg) were condensed by the same manner as described in Example 9. Yellow oil of a free base was converted to a dihydrochloride which gave the title compound as yellow powder.

Yield: 41 mg (16%).

¹H-NMR (400 MHz, measured as a free base, CDCl₃) δ: 1.78-1.88 (m, 4H), 2.16 (t, 2H, J=10.7 Hz), 2.96 (d, 2H, J=11.3 Hz), 3.56 (s, 2H), 3.70 (s, 6H), 3.73-3.84 (m, 1H), 3.87 (s, 3H), 3.89 (s, 6H), 3.90 (s, 3H), 3.95 (s, 6H), 4.54 (s, 2H), 5.95 (s, 2H), 6.71 (s, 2H), 7.19-7.26 (m, 4H), 7.31-7.39 (m, 3H), 7.42 (s, 1H), 7.59 (s, 1H), 8.58 (d, 1H, J=4.9 Hz).

### EXAMPLE 25

### Synthesis of 4-[N-(3,5-dimethoxyphenyl)-N-[[2-(3,4,5-trimethoxyphenyl)pyridin-5-yl]methyl]amino]-1-[[2-(3,4,5-trimethoxyphenyl)pyridin-4-yl]methyl]piperidine Trihydrochloride

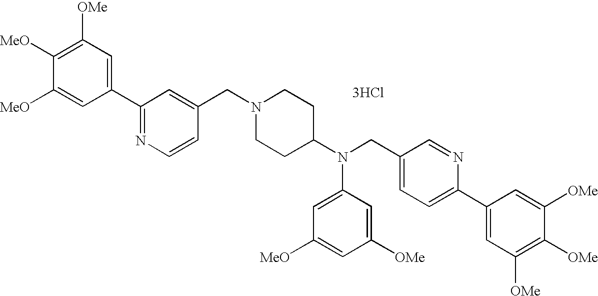

4-(3,5-Dimethoxyphenylamino)-1-[[2-(3,4,5-trimethoxyphenyl)pyridin-4-yl]methyl]piperidine (148 mg) and 5-chloromethyl-2-(3,4,5-trimethoxyphenyl)pyridine (114 mg) were condensed by the same manner as described in Example 9. Yellow oil of a free base was converted to a trihydrochloride which gave the title compound as yellow powder.

Yield: 23 mg (10%).

¹H-NMR (400 MHz, measured as a free base, CDCl₃) δ: 1.64 (br, 2H), 1.82 (br, 2H), 2.10 (br, 2H), 2.94 (br, 2H), 3.48-3.60 (m, 3H), 3.64 (s, 6H), 3.82 (s, 3H), 3.83 (s, 3H), 3.87 (s, 6H), 3.90 (s, 6H), 4.46 (s, 2H), 5.85 (br, 3H), 7.05-7.24 (m, 6H), 7.53-7.54 (m, 2H), 8.51 (s, 1H), 8.54 (br, 1H).

### PREPARATION EXAMPLE 66

### Synthesis of Ethyl 4-(3,4,5-trimethoxyphenyl)benzoate

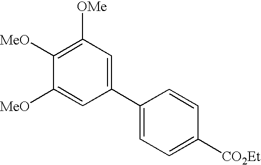

3,4,5-Trimethoxyphenylboronic acid (2.01 g) and ethyl 4-bromobenzoate (2.29 g) were condensed in the same manner as described in Preparation Example 1 to give the title compound.

Yield: 2.99 g (95%).

¹H-NMR (400 MHz, CDCl₃) δ: 1.42 (t, 3H, J=7.2 Hz), 3.90 (s, 3H), 3.94 (s, 6H), 4.38 (q, 2H, J=7.2 Hz), 6.81 (s, 2H), 7.62 (d, 2H, J=8.2 Hz), 8.10 (d, 2H, J=8.2 Hz).

### PREPARATION EXAMPLE 67

### Synthesis of 4-(3,4,5-trimethoxyphenyl)benzyl Alcohol

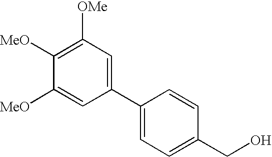

Ethyl 4-(3,4,5-trimethoxyphenyl)benzoate (2.99 g) was treated in the same manner as described in Preparation Example 2 to give the title compound.

Yield: 1.83 g (71%)

### PREPARATION EXAMPLE 68

### Synthesis of 4-(3,4,5-trimethoxyphenyl)benzyl Chloride

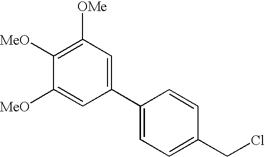

4-(3,4,5-Trimethoxyphenyl)benzyl alcohol (1.83 g) was treated in the same manner as describe in Preparation Example 3 to give the title compound.

Yield: 1.65 g (84%)

¹H-NMR (400 MHz, CDCl₃) δ: 3.90 (s, 3H), 3.93 (s, 6H), 4.65 (s, 2H), 6.77 (s, 2H), 7.46 (d, 2H, J=8.0 Hz), 7.55 (d, 2H, J=8.0 Hz).

### EXAMPLE 26

### Synthesis of 4-[N-(3,5-dimethoxyphenyl)-N-[4-(3,4,5-trimethoxypheny)benzyl]amino]-1-[[2-(3,4,5-trimethoxyphenyl)pyridin-4-yl]methyl]piperidine Dihydrochloride

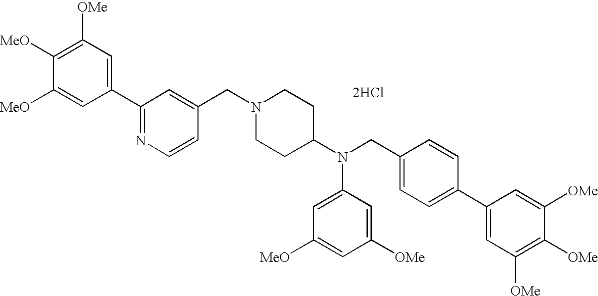

4-(3,5-Dimethoxyphenylamino)-1-[[2-(3,4,5-trimethoxyphenyl)pyridin-4-yl]methyl]piperidine (148 mg) and 4-(3,4,5-trimethoxyphenyl)benzyl chloride (114 mg) were condensed by the same manner as described in Example 9. Yellow oil of a free base was converted to a dihydrochloride which gave yellow powder of the title compound.

Yield: 35 mg (14%).

¹H-NMR (400 MHz, measured as a free base, CDCl₃) δ; 1.80-1.89 (m, 4H), 2.17 (br, 2H), 2.97 (d, 2H, J=10.5 Hz), 3.57 (s, 2H), 3.70 (s, 6H), 3.77-3.84 (m, 1H), 3.87 (s, 3H), 3.90 (s, 3H), 3.91 (s, 6H), 3.96 (s, 6H), 4.52 (s, 2H), 5.93 (s, 2H), 6.74 (s, 2H), 7.19-7.22 (m, 4H), 7.31 (d, 2H, J=8.2 Hz), 7.46 (d, 2H, J=8.2 Hz), 7.60 (s, 1H), 8.59 (d, 1H, J=5.1 Hz).

### PREPARATION EXAMPLE 69

### Synthesis of 4-(3,4-methylenedioxyphenylamino)-1-[[2-(3,4,5-trimethoxyphenyl) pyridin-4-yl]methyl]piperidine

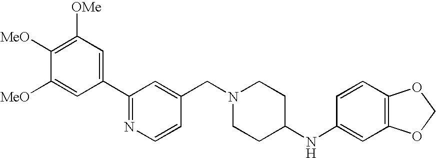

1-[[2-(3,4,5-Trimethoxyphenyl)pyridin-4-yl]methyl-4-piperidone (1.40 g) and 3,4-methylenedioxyaniline (646 mg) were treated in the same manner as described in Preparation Example 29 to give the title compound.

Yield: 810 mg (43%).

¹H-NMR (400 MHz, CDCl₃) δ: 1.63 (br, 2H), 2.02-2.60 (m, 4H), 2.80-3.15 (m, 2H), 3.25 (br, 1H), 3.70 (br, 2H), 3.88 (s, 3H), 3.96 (s, 6H), 5.83 (s, 2H), 6.02 (d, 1H, J=8.3 Hz), 6.22 (s, 1H), 6.61 (d, 1H, J=8.3 Hz), 7.18-7.28 (m, 3H), 7.64 (br, 1H), 8.60 (d, 1H, J=4.9 Hz).

### EXAMPLE 27

### Synthesis of 4-[N-(3,4-methylenedioxyphenyl)-N-[[2-(3,4,5-trimethoxypheny) pyridin-3-yl]methyl]amino]-1-[[2-(3,4,5-trimethoxyphenyl)pyridin-4-yl]methyl] piperidine Trihydrochloride

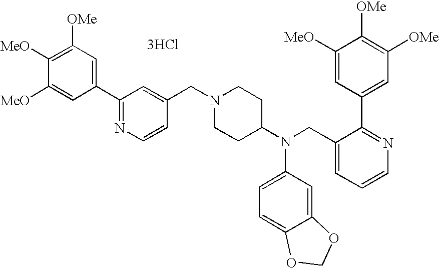

4-(3,4-Methylenedioxyphenylamino)-1-[[2-(3,4,5-trimethoxyphenyl)pyridin-4-yl]methyl]piperidine (119 mg) and 3-chloromethyl-2-(3,4,5-trimethoxyphenyl)pyridine (114 mg) were condensed in the same manner as described in Example 9. Yellow syrup obtained was converted to a trihydrochloroide to give the title compound as yellow powder.

Yield: 30 mg (14%).

¹H-NMR (400 MHz, measured as a free base, CDCl₃) δ: 1.45-2.25 (m, 6H), 2.90 (br, 2H), 3.40 (br, 1H), 3.55 (br, 2H), 3.87 (s, 3H), 3.88 (s, 9H), 3.93 (s, 6H), 4.28 (s, 2H), 5.82 (s, 2H), 6.10 (br, 1H), 6.28 (s, 1H), 6.58 (d, 1H, J=8.4 Hz), 6.67 (s, 2H), 7.12-7.30 (m, 4H), 7.52 (br, 1H), 7.75 (br, 1H), 8.51 (br, 1H), 8.57 (br, 1H).

### EXAMPLE 28

### Synthesis of 4-[N-(3,4-methylenedioxyphenyl)-N-[2-(3,4,5-trimethoxyphenyl) benzyl]amino]-1-[[2-(3,4,5-trimethoxyphenyl)pyridin-4-yl]metyl]piperidine Dihydrochloride

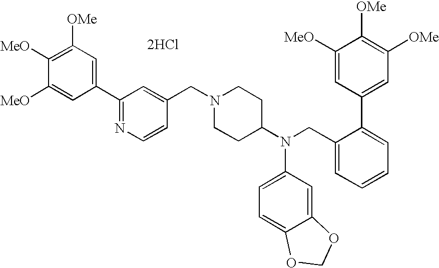

4-(3,4-Methylenedioxyphenylamino)-1-[[2-(3,4,5-trimethoxyphenyl)pyridin-4-yl]methyl]piperidine (119 mg) and 2-(3,4,5-trimethoxyphenyl)benzyl chloride (114 mg) were condensed in the same manner as described in Example 9. A free base obtained was converted to a dihydrochloroide to give the title compound as yellow powder.

Yield: 13 mg (6%).

¹H-NMR (400 MHz, measured as a free base, CDCl₃) δ: 1.61 (br, 2H), 1.78 (br, 2H), 2.10 (br, 2H), 2.91 (br, 2H), 3.50-3.54 (m, 3H), 3.87 (s, 6H), 3.90 (s, 3H), 3.92 (s, 3H), 3.99 (s, 6H), 4.26 (s, 2H), 5.82 (s, 2H), 6.12 (d, 1H, J=8.6 Hz), 6.32 (s, 1H), 6.53 (s, 2H), 6.62 (d, 1H, J=8.6 Hz), 7.17-7.26 (m, 6H), 7.42 (br, 1H), 7.55 (s, 1H), 8.58 (d, 1H, J=4.9 Hz).

### EXAMPLE 29

### Synthesis of 4-[N-(3,4-methylenedioxyphenyl)-N-[[2-(3,4,5-trimethoxyphenyl) pyridin-4-yl]methyl]amino]-1-[[2-(3,4,5-trimethoxyphenyl)pyridin-4-yl]methyl] piperidine Trihydrochloride

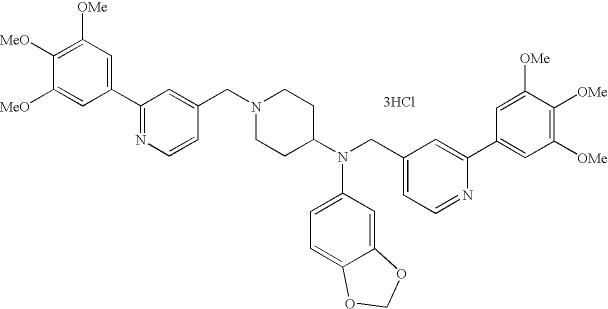

4-(3,4-Methylenedioxyphenylamino)-1-[[2-(3,4,5-trimethoxyphenyl)pyridin-4-yl]methyl]piperidine (119 mg) and 4-chloromethyl-2-(3,4,5-trimethoxyphenyl) pyridine (114 mg) were condensed by the same manner as described in Example 9. Yellow oil of a free base was converted to a trihydrochloride which gave the title compound as yellow powder.

Yield: 52 mg (25%).

¹H-NMR (400 MHz, measured as a free base, CDCl₃) δ: 1.60-1.95 (m, 4H), 2.20 (br, 2H), 3.00 (br, 2H), 3.58 (br, 3H), 3.86 (s, 3H), 3.87 (s, 3H), 3.91 (s, 6H), 3.94 (s, 6H), 4.41 (s, 2H), 5.82 (s, 2H), 6.17 (d, 1H, J=8.4 Hz), 6.39 (s, 1H), 6.62 (d, 1H, J=8.4 Hz), 7.12-7.13 (m, 3H), 7.18 (d, 1H, J=4.1 Hz), 7.23 (br, 2H), 7.54 (br, 2H), 8.51 (d, 1H, J=5.1 Hz), 8.57 (d, 1H, J=4.9 Hz).

### EXAMPLE 30

### Synthesis of 4-[N-(3,4-methylenedioxyphenyl)-N-[3-(3,4,5-trimethoxyphenyl) benzyl]amino]-1-[[2-(3,4,5-trimethoxyphenyl)pyridin-4-yl]methyl]piperidine Dihydrochloride

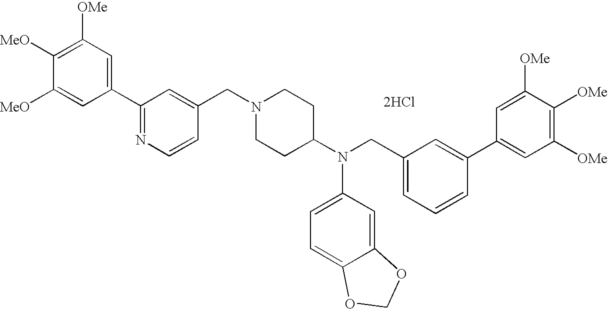

4-(3,4-Methylenedioxyphenylamino)-1-[[2-(3,4,5-trimethoxyphenyl)pyridin-4-yl]methyl]piperidine (119 mg) and 3-(3,4,5-trimethoxyphenyl)benzyl chloride (114 mg) were condensed by the same manner as described in Example 9. Yellow oil of a free base was converted to a dihydrochloride which gave the title compound as yellow powder.

Yield: 58 mg (29%).

¹H-NMR (400 MHz, measured as a free base, CDCl₃) δ: 1.60-1.97 (m, 4H), 2.15 (br, 2H), 3.00 (br, 2H), 3.58 (br, 3H), 3.86 (s, 3H), 3.88 (s, 9H), 3.94 (s, 6H), 4.43 (s, 2H), 5.81 (s, 2H), 6.21 (br, 1H), 6.42 (s, 1H), 6.62 (d, 1H, J=8.4 Hz), 6.69 (s, 2H), 7.18 (d, 1H, J=4.9 Hz), 7.22-7.39 (m, 6H), 7.60 (br, 1H), 8.57 (d, 1H, J=4.9 Hz).

### EXAMPLE 31

### Synthesis of 4-[N-(3,4-methylenedioxyphenyl)-N-[[2-(3,4,5-trimethoxyphenyl) pyridin-5-yl]methyl]amino]-1-[[2-(3,4,5-trimethoxyphenyl)pyridinyl]methyl] Piperidine Trihydrochloride

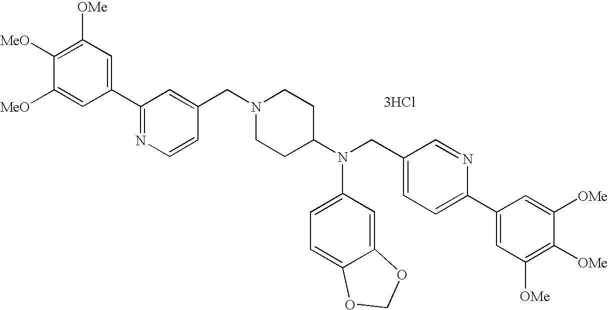

4-(3,4-Methylenedioxyphenylamino)-1-[[2-(3,4,5-trimethoxyphenyl)pyridin-4-yl]methyl]piperidine (119 mg) and 5-chloromethyl-2-(3,4,5-trimethoxyphenyl) pyridine (114 mg) were condensed by the same manner as described in Example 9. Yellow oil of a free base was converted to a trihydrochloride which gave the title compound as yellow powder.

Yield: 69 mg (27%).

¹H-NMR (400 MHz, measured as a free base, CDCl₃) δ: 1.71-1.88 (m, 4H), 2.14 (d, 2H, J=11.2 Hz), 2.97 (d, 2H, J=11.5 Hz), 3.45-3.52 (m, 1H), 3.56 (s, 2H), 3.89 (s, 3H), 3.90 (s, 3H), 3.94 (s, 6H), 3.96 (s, 6H), 4.12 (s, 2H), 5.85 (s, 2H), 6.24 (dd, 1H, J=8.5 Hz, 2.5 Hz), 6.45 (d, 1H, J=2.4 Hz), 6.64 (d, 1H, J=8.5 Hz), 7.20-7.21 (m, 1H), 7.21 (s, 2H), 7.23 (s, 2H), 7.58-7.65 (m, 3H), 8.57 (d, 1H, J=1.5 Hz), 8.59 (d, 1H, J=4.9 Hz).

### EXAMPLE 32

### Synthesis of 4-[N-(3,4-Methylenedioxyphenyl)-N-[4-(3,4,5-trimethoxyphenyl) benzyl]amino]-1-[[2-(3,4,5-trimethoxyphenyl)pyridin-4-yl]methyl]piperidine Dihydrochloride

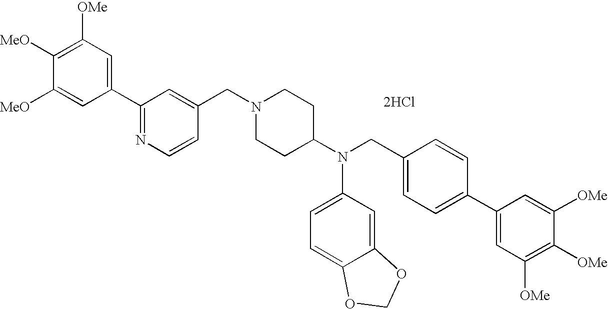

4-(3,4-Methylenedioxyphenylamino)-1-[[2-(3,4,5-trimethoxyphenyl)pyridin-4-yl]methyl]piperidine (119 mg) and 4-(3,4,5-trimethoxyphenyl)benzyl chloride (114 mg) were condensed by the same manner as described in Example 9. Yellow oil of a free base was converted to a dihydrochloride which gave the title compound as yellow powder.

Yield: 29 mg (14%).

¹H-NMR (400 MHz, measured as a free base, CDCl₃) δ; 1.62-2.00 (m, 4H), 2.20 (br, 2H), 2.99 (br, 2H), 3.58 (br, 3H), 3.86 (s, 3H), 3.87 (s, 3H), 3.88 (s, 6H), 3.89 (s, 6H), 4.41 (s, 2H), 5.82 (s, 2H), 6.19 (d, 1H, J=8.6 Hz), 6.39 (s, 1H), 6.63 (d, 1H, J=8.4 Hz), 6.72 (s, 2H), 7.18 (d, 1H, J=5.1 Hz), 7.23 (s, 2H), 7.29 (d, 2H, J=8.0 Hz), 7.43 (d, 2H, J=8.2 Hz), 7.60 (br, 1H), 8.57 (d, 1H, J=4.9 Hz).

### PREPARATION EXAMPLE 70

### Synthesis of 4-[N-methyl-N-[(2-nitrobenzene)sulfonyl]aminomethyl]-2-(3,4,5-trimethoxyphenyl)pyridine

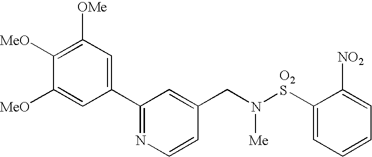

4-Chloromethyl-2-(3,4,5-trimethoxyphenyl)pyridine (232 mg), N-methyl-2-nitrobenzenesulfonamide (171 mg) and potassium carbonate (138 mg) were suspended in acetonitrile (10 mL). The mixture was stirred at room temperature overnight and evaporated. To the residue was added chloroform and water. The organic layer was separated, washed with saturated aqueous sodium hydrogencarbonate and brine, dried over anhydrous magnesium sulfate and evaporated to give the title compound.

Yield: 362 mg (97.0%).

### PREPARATION EXAMPLE 71

### Synthesis of 4-(methylaminomethyl)-2-(3,4,5-trimethoxyphenyl)pyridine

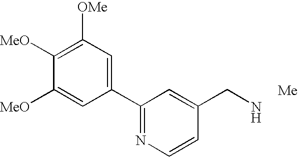

To a suspension of 4-[N-methyl-N-[(2-nitrobenzene)sulfonyl]aminomethyl]-2-(3,4,5-trimethoxyphenyl)pyridine (691 mg) and potassium carbonate (203 mg) in acetonitrile (20 mL) was added thiophenol (228 μL). The mixture was stirred at 50° C. overnight and evaporated. To the residue was added chloroform and water. The organic layer was separated, washed with saturated aqueous sodium hydrogencarbonate and brine, dried over anhydrous magnesium sulfate and evaporated. The residue was subjected to a column of silica gel using chloroform-methanol (40:1) and then chloroform-methanol (10:1) as eluents. Fractions containing the product were collected and evaporated to give the title compound.

Yield: 356 mg (84%).

### EXAMPLE 33

### Synthesis of 4-[N-methyl-N-[[2-(3,4,5-trimethoxyphenyl)pyridin-4-yl]methyl]aminocarbonyl]-1-[[2-(3,4,5-trimethoxyphenyl)pyridin-4-yl]methyl]piperidine Maleate

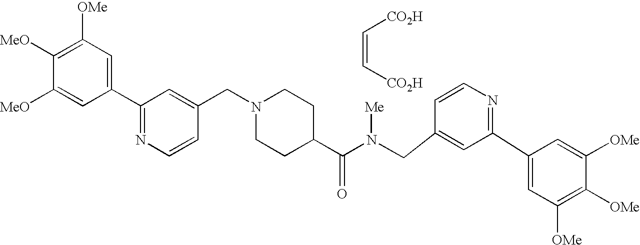

1-[[2-(3,4,5-trimethoxyphenyl)pyridin-4-yl]methyl]piperidine-4-caroxylic acid (98 mg) and 4-(methylaminomethyl)-2-(3,4,5-trimethoxyphenyl)pyridine (73 mg) were condensed by the same manner as described in Example 1 giving a maleate of the title compound as white powder.

Yield: 145 mg (75%).

¹H-NMR (400 MHz, measured as a maleate, DMSO-d₆)δ: 1.89-1.97 (m, 4H), 2.75-2.96 (m, 3H), 3.03 (s, 3H), 3.27 (d, 2H, J=12.0 Hz), 3.78 (s, 3H), 3.79 (s, 3H), 3.87 (s, 6H), 3.90 (s, 6H), 4.09 (s, 2H), 4.64 (s, 2H), 6.14 (s, 2H), 7.09 (d, 1H, J=5.0 Hz), 7.33 (s, 2H), 7.37 (d, 1H, J=5.0 Hz), 7.38 (s, 2H), 7.65 (s, 1H), 7.90 (s, 1H), 8.57 (d, 1H, J=5.0 Hz), 8.67 (d, 1H, J=5.0 Hz).

### PREPARATION EXAMPLE 72

### Synthesis of (3S)-1-(tert-butoxycarbonyl)-3-[N-[(2-nitrobenzene)sulfonyl]-N-[[2-(3,4,5-trimethoxyphenyl)pyridinyl]methyl]amino]pyrrolidine

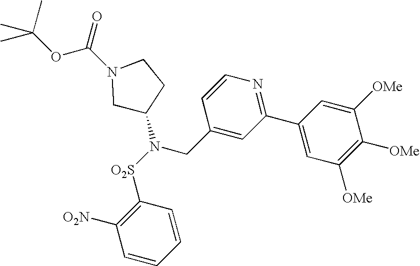

(3S)-1-(tert-Butoxycarbonyl)-3-[(2-nitrobenzene)sulfonylamino]pyrrolidine (72 mg) and 4-chloromethyl-2-(3,4,5-trimethoxyphenyl)pyridine (57 mg) were condensed in the same manner as described in Example 2 to give colorless amorphous of the title compound.

Yield: 103 mg (85%).

### PREPARATION EXAMPLE 73

### Synthesis of (3S)-3-[N-[(2-nitrobenzene)sulfonyl]-N-[[2-(3,4,5-trimethoxyphenyl)pyridin-4-yl]methyl]amino]pyrrolidine

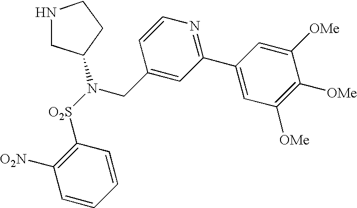

(3S)-1-(tert-butoxycarbonyl)-3-[N-[(2-nitrobenzene)sulfonyl]-N-[[2-(3,4,5-tri methoxyphenyl)pyridin-4-yl]methyl]amino]pyrrolidine (103 mg) was treated in the same manner as described in Preparation Example 12 to give yellow amorphous of the title compound.

Yield: 72 mg (84%).

¹H-NMR (400 MHz, CDCl₃)δ: 1.66-1.75 (m, 1H), 2.03-2.05 (m, 1H), 2.78-2.85 (m, 2H), 3.00-3.10 (m, 2H), 3.39 (br, 1H), 3.90 (s, 3H), 3.96 (s, 6H), 4.59-4.67 (m, 1H), 4.70 (s, 2H), 7.13-7.18 (m, 1H), 7.20 (s, 2H), 7.52-7.64 (m, 4H), 7.95 (dd, 1H, J=7.9 Hz, 1.1 Hz), 8.52 (d, 1H, J=5.1 Hz).

### PREPARATION EXAMPLE 74

### Synthesis of (3S)-3-[N-[(2-nitrobenzene)sulfonyl]-N-[[2-(3,4,5-trimethoxyphenyl)pyridin-4-yl]methyl]amino]-1-[[2-(3,4,5-trimethoxyphenyl)pyridin-4-yl]methyl]pyrrolidine

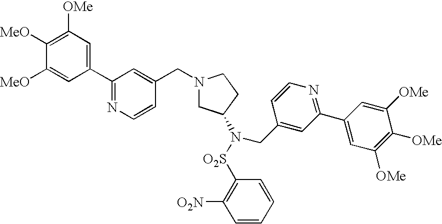

(3S)-3-[N-[(2-Nitrobenzene)sulfonyl]-N-[[2-(3,4,5-trimethoxyphenyl)pyridin-4-yl]methyl]amino]pyrrolidine (72 mg) and 4-chloromethyl-2-(3,4,5-trimethoxyphenyl)pyridine (40 mg) were treated in the same manner as described in Example 2 to give a yellow amorphous of the title compound.

Yield: 97 mg (91%).

¹H-NMR (400 MHz, CDCl₃)δ: 1.59 (br, 1H), 1.80-1.90 (m, 1H), 2.20-2.30 (m, 2H), 2.55 (dd, 1H, J=10.5 Hz, 8.2 Hz), 2.78 (dd, 1H, J=10.6 Hz, 3.2 Hz), 2.87 (t, 1H, J=7.2 Hz), 3.50 (d, 1H, J=13.7 Hz), 3.64 (d, 1H, J=13.7 Hz), 3.89 (s, 3H), 3.90 (s, 3H), 3.92 (s, 6H), 3.93 (s, 6H), 4.83 (d, 2H, J=4.5 Hz), 7.07 (d, 1H, J=5.1 Hz), 7.10 (d, 1H, J=4.9 Hz), 7.15 (s, 2H), 7.17 (s, 2H), 7.41-7.45 (m, 1H), 7.50-7.55 (m, 3H), 7.61 (s, 1H), 7.81 (d, 1H, J=7.4 Hz), 8.45 (d, 1H, J=4.9 Hz), 8.51 (d, 1H, J=5.1 Hz).

### EXAMPLE 34

### Synthesis of (3S)-1-[[2-(3,4,5-trimethoxyphenyl)pyridinyl]methyl]-3-[[2-(3,4,5-trimethoxyphenyl)pyridin-4-yl]methylamino]pyrrolidine Trihydrochloride

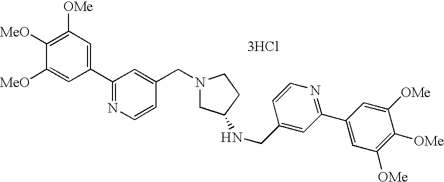

(3S)-3-[N-[(2-nitrobenzene)sulfonyl]-N-[[2-(3,4,5-trimethoxyphenyl)pyridin-4-yl]methyl]amino]-1-[[2-(3,4,5-trimethoxyphenyl)pyridin-4-yl]methyl]pyrrolidine (97 mg) was treated in the same manner as described in Preparation Example 11 to give yellow amorphous of the title compound, which was converted to a trihydrochloride.

Yield: 80 mg (89%).

¹H-NMR (400 MHz, measured as a free base, CDCl₃)δ: 1.71 (br, 2H), 2.19-2.21 (m, 1H), 2.52-2.55 (m, 2H), 2.73-2.77 (m, 2H), 3.39 (br, 1H), 3.66 (d, 1H, J=13.7 Hz), 3.71 (d, 1H, J=13.7 Hz), 3.82 (s, 2H), 3.90 (s, 6H), 3.95 (s, 12H), 7.18-7.21 (m, 2H), 7.23 (s, 2H), 7.24 (s, 2H), 7.63 (s, 2H), 8.59 (d, 1H, J=4.3 Hz), 8.60 (d, 1H, J=4.3 Hz).

### EXAMPLE 35

### Synthesis of 4-[3-(3,4,5-trimethoxyphenyl)benzoylamino]-1-[[2-(3,4,5-trimethoxyphenyl)pyridin-4-yl]methyl]piperidine Maleate

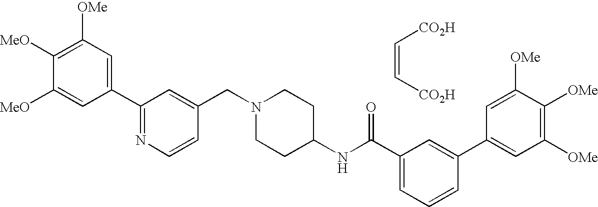

3-(3,4,5-trimethoxyphenyl)benzoic acid (69 mg) and 4-amino-1-[[2-(3,4,5-trimethoxyphenyl)pyridin-4-yl]methyl]piperidine (114 mg) were condensed in the same manner as described in Example 1. The title compound was obtained after converting the product to a maleate.

Yield: 100 mg (56%)

¹H-NMR (400 MHz, measured as a maleate, DMSO-d₆)δ: 1.85-2.10 (m, 4H), 2.77-2.93 (m, 2H), 3.20-3.31 (m, 2H), 3.77 (s, 3H), 3.79 (s, 3H), 3.89 (s, 6H), 3.91 (s, 6H), 3.98-4.07 (m, 1H), 4.13 (s, 2H), 6.15 (s, 2H), 6.94 (s, 2H), 7.40-7.52 (m, 4H), 7.73-7.80 (m, 2H), 8.02-8.10 (m, 3H), 8.67-8.68 (m, 1H).

### EXAMPLE 36

### Synthesis of 4-[N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine Tetrahydrochloride

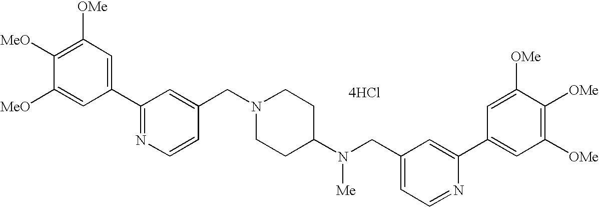

4-(methylamino)-1-[[2-(3,4,5-trimethoxyphenyl)pyridin-4-yl]methyl]piperidine (2.67 g) and 4-chloromethyl-2-(3,4,5-trimethoxyphenyl)pyridine (2.12 g) were condensed in the same manner as described in Example 2. The title compound was obtained after converting a free base to a tetrahydrochloride.

Yield: 2.55 g (46%).

¹H-NMR (400 MHz, measured as a free base, CDCl₃)δ: 1.66-1.74 (m, 2H), 1.82 (d, 2H, J=10.7 Hz), 2.04 (t, 2H, J=11.0 Hz), 2.25 (s, 3H), 2.45-2.51 (m, 1H), 2.98 (d, 2H, J=11.7 Hz), 3.55 (s, 2H), 3.66 (s, 2H), 3.90 (s, 3H), 3.91 (s, 3H), 3.96 (s, 6H), 3.97 (s, 6H), 7.21-7.23 (m, 2H), 7.24 (s, 2H), 7.25 (s, 2H), 7.62 (s, 1H), 7.63 (s, 1H), 8.59 (d, 1H, J=5.1 Hz), 8.60 (d, 1H, J=5.3 Hz).

### PREPARATION EXAMPLE 75

### Synthesis of 1-(ethoxycarbonyl)-4-[[2-(3,4,5-trimethoxyphenyl)pyridin-4-yl]methylamino]piperidine

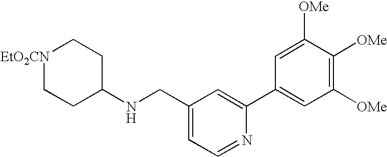

4-Amino-1-(ethoxycarbonyl)piperidine (341 mg) and 4-chloromethyl-2-(3,4,5-trimethoxyphenyl)pyridine (300 mg) were condensed in the same manner as described in Example 2 to give the title compound.

Yield: 438 mg (theoretical yield).

¹H-NMR (400 MHz, CDCl₃)δ: 1.25 (t, 3H, J=7.1 Hz), 1.27-1.34 (m, 2H), 1.60 (br, 1H), 1.90 (d, 2H, J=10.9 Hz), 2.67-2.72 (m, 1H), 2.87 (t, 2H, J=11.5 Hz), 3.90 (s, 3H), 3.91 (br, 2H), 3.96 (s, 6H), 4.09 (br, 2H), 4.12 (q, 2H, J=7.0 Hz), 7.21 (d, 1H, J=3.5 Hz), 7.24 (s, 2H), 7.65 (s, 1H), 8.59 (d, 1H, J=4.9 Hz).

### PREPARATION EXAMPLE 76

### Synthesis of 1-(ethoxycarbonyl)-4-[N-methyl-N-[[2-(3,4,5-trimethoxyphenyl)pyridin-4-yl]methyl]amino]piperidine

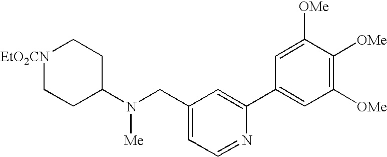

To a solution of 1-(ethoxycarbonyl)-4-[[2-(3,4,5-trimethoxyphenyl)pyridin-4-yl]methylamino]piperidine (438 mg) was treated in the same manner as described in Preparation Example 11 to give the title compound as yellow syrup.

Yield: 235 mg (52%).

¹H-NMR (400 MHz, CDCl₃)δ: 1.26 (t, 3H, J=7.1 Hz), 1.42-1.57 (m, 2H), 1.82 (d, 2H, J=11.9 Hz), 2.24 (s, 3H), 2.59-2.65 (m, 1H), 2.75 (t, 2H, J=12.0 Hz), 3.65 (s, 2H), 3.90 (s, 3H), 3.97 (s, 6H), 4.13 (q, 2H, J=7.0 Hz), 4.23 (br, 2H), 7,22 (dd, 1H, J=5.0 Hz, 1.3 Hz), 7.24 (s, 2H), 7.63 (s, 1H), 8.59 (d, 1H, J=4.5 Hz).

### PREPARATION EXAMPLE 77

### Synthesis of 4-[N-methyl-N-[[2-(3,4,5-trimethoxyphenyl)pyridin-4-yl]methyl]amino]piperidine

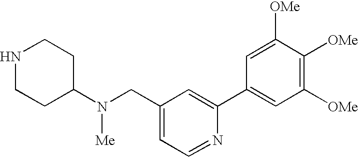

To a solution of 1-(ethoxycarbonyl)-4[N-methyl-N-[[2-(3,4,5-trimethoxyphenyl)pyridin-4-yl]methyl]amino]piperidine (100 mg) in ethanol (2 mL) was added 4 M sodium hydroxide (8 mL). The mixture was refluxed overnight and extracted with chloroform. The organic layer was washed with water and brine, dried over anhydrous sodium sulfate and evaporated. The residue was subjected to a column of silica gel and liquid chromatography was performed using chloroform-methanol (20:1) to give the title compound as yellow syrup.

Yield: 73 mg (88%).

¹H-NMR (400 MHz, CDCl₃)δ: 1.50-1.55 (m, 2H), 1.84 (d, 2H, J=12.0 Hz), 1.99 (br, 1H), 2.25 (s, 3H), 2.55-2.63 (m, 3H), 3.16 (d, 2H, J=12.2 Hz), 3.65 (s, 2H), 3.90 (s, 3H), 3.97 (s, 6H), 7.22 (d, 1H, J=6.1 Hz), 7.24 (s, 2H), 7.64 (s, 1H), 8.58 (d, 1H, J=5.1 Hz).

### EXAMPLE 37

### Synthesis of 4-[N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine Tetrahydrochloride

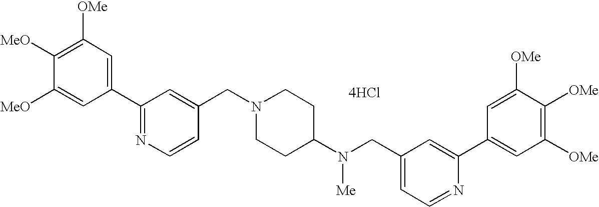

4-[N-methyl-N-[[2-(3,4,5-trimethoxyphenyl)pyridin-4-yl]methyl]amino]piperidine (73 mg) and 4-chloromethyl-2-(3,4,5-trimethoxyphenyl)pyridine (58 mg) were condensed in the same manner as described in Example 2. The title compound was obtained after converting a free base to a tetrahydrochloride.

Yield: 126 mg (84%).

### EXAMPLE 38

### Synthesis of 4-[N-methyl-N-[3-(3,4,5-trimethoxyphenyl)benzyl]amino]-1-[[2-(3,4,5-trimethoxyphenyl)pyridin-4-yl]methyl]piperidine Difurmarate

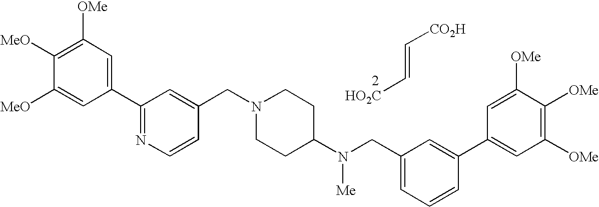

4-(methylamino)-1-[[2-(3,4,5-trimethoxyphenyl)pyridin-4-yl]methyl]piperidine (111 mg) and 3-(3,4,5-trimethoxyphenyl)benzyl chloride (88 mg) were condensed in the same manner as described in Example 2. The title compound was obtained as white powder after converting a free base to a difumarate.

Yield: 59 mg (46%).

¹H-NMR (400 MHz, measured as a free base, CDCl₃)δ: 1.70-1.77 (m, 2H), 1.85-1.87 (m, 2H), 2.03-2.08 (m, 2H), 2.27 (s, 3H), 2.55-2.59 (m, 1H), 2.98 (d, 2H, J=11.3 Hz), 3.56 (s, 2H), 3.69 (s, 2H), 3.89 (s, 3H), 3.90 (s, 3H), 3.93 (s, 6H), 3.98 (s, 6H), 6.79 (s, 2H), 7.22 (d, 1H, J=4.9 Hz), 7.28 (s, 2H), 7.31 (d, 1H, J=7.6 Hz), 7.38 (t, 1H, J=7.4 Hz), 7.45 (d, 1H, J=7.6 Hz), 7.51 (s, 1H), 7.63 (s, 1H), 8.60 (d, 1H, J=5.1 Hz).

### EXAMPLE 39

### Synthesis of 4-[N-methyl-N-[[2-(3,5-dimethoxy-4-hydroxyphenyl)pyridin-4-yl]methyl]amino]-1-[[2-(3,5-dimethoxy-4-hydroxyphenyl)pyridin-4-yl]methyl]piperidine Tetrahydrochloride

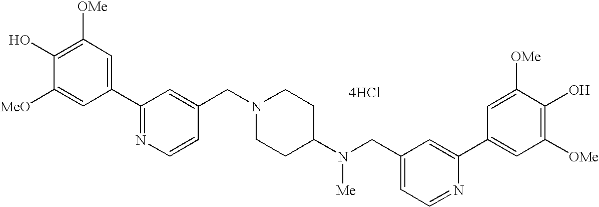

To an ice-cooled solution of 4-[N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine (100 mg) in dichloromethane (5 mL) was added iodotrimethylsilane (173 μL). The mixture was stirred at 0° C. for 2 hours and then at room temperature overnight. A small amount of water, ethyl acetate and saturated aqueous sodium hydrogencarbonate were added to the mixture at 0° C. and the organic layer was separated. The organic layer was washed with brine, dried over anhydrous magnesium sulfate and evaporated. The residue was applied to a preparative TLC using chloroform-ammonia saturated methanol (15:1) to give a free base of the title compound which was converted to a tetrahydrochloride by the conventional method.

Yield: 50 mg (52.3%).

¹H-NMR (400 MHz, measured as a free base, CDCl₃)δ: 1.68-1.89 (m, 4H), 2.03-2.12 (m, 2H), 2.26 (s, 3H), 2.48-2.60 (m, 1H), 2.98-3.05 (m, 2H), 3.57 (s, 2H), 3.65 (s, 2H), 3.94 (s, 6H), 3.95 (s, 6H), 7.16-7.19 (m, 2H), 7.26 (s, 2H), 7.27 (s, 2H), 7.62-7.68 (m, 2H), 8.56 (d, 1H, J=5.3 Hz), 8.58 (d, 1H, J=5.2 Hz).

### PREPARATION EXAMPLE 78

### Synthesis of 1-(ethoxycarbonyl)-4-[N-ethyl-N-[[2-(3,4,5-trimethoxyphenyl)pyridin-4-yl]methyl]amino]piperidine

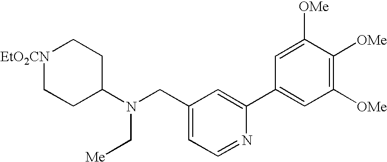

To a solution of 1-(ethoxycarbonyl)-4-[[2-(3,4,5-trimethoxyphenyl)pyridin-4-yl]methylamino]piperidine (400 mg) in acetonitrile (5 mL) was added potassium carbonate (13 mg) and iodoethane (145 mg). The mixture was placed in sealed vessel and stirred at 80° C. for 2 hours. After removing the solvent in vacuo, ethyl acetate was added, washed with water and brine, dried over anhydrous sodium sulfate and evaporated. The residue was subjected to a column of silica gel using chloroform-methanol (30:1) as an eluent. Fractions containing the product were collected and evaporated to give the title compound as yellow syrup.

Yield: 242 mg (57%).

¹H-NMR (400 MHz, CDCl₃)δ: 1.04 (t, 3H, J=7.1 Hz), 1.25 (t, 3H, J=7.1 Hz), 1.43-1.52 (m, 2H), 1.79 (d, 2H, J=11.5 Hz), 2.60 (q, 2H, J=7.0 Hz), 2.66-2.76 (m, 3H), 3.70 (s, 2H), 3.90 (s, 3H), 3.97 (s, 6H), 4.12 (q, 2H, J=7.0 Hz), 4.20 (br, 2H), 7.23 (s, 2H), 7.26 (d, 1H, J=5.7 Hz), 7.67 (s, 1H), 8.58 (d, 1H, J=4.9 Hz).

### PREPARATION EXAMPLE 79

### Synthesis of 4-[N-ethyl-N-[[2-(3,4,5-trimethoxyphenyl)pyridin-4-yl]methyl]amino]piperidine

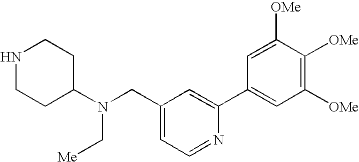

1-(ethoxycarbonyl)-4[N-ethyl-N-[[2-(3,4,5-trimethoxyphenyl)pyridin-4-yl]m ethyl]amino]piperidine (242 mg) was treated in the same manner as described in Preparation Example 77 to give the title compound as yellow syrup.

Yield: 150 mg (74%).

¹H-NMR (400 MHz, CDCl₃)δ: 1.03 (t, 3H, J=7.0 Hz), 1.43-1.52 (m, 2H), 1.70 (br, 1H), 1.79 (d, 2H, J=12.3 Hz), 2.53-2.67 (m, 5H), 3.13 (d, 2H, J=11.9 Hz), 3.71 (s, 2H), 3.90 (s, 3H), 3.97 (s, 6H), 7.24 (s, 2H), 7.27 (d, 1H, J=5.1 Hz), 7.68 (s, 1II), 8.57 (d, 1H, J=4.3 Hz).

### EXAMPLE 40

### Synthesis of 4-[N-ethyl-N-[[2-(3,4,5-trimethoxyphenyl)pyridin-4-yl]methyl]amino]-1-[[2-(3,4,5-trimethoxyphenyl)pyridinyl]methyl]piperidine Tetrahydrochloride

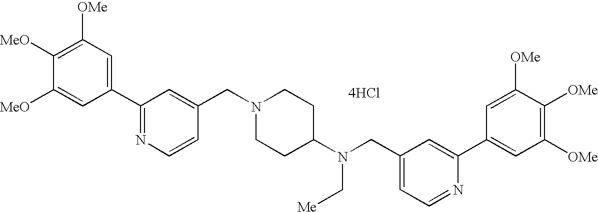

4-[N-ethyl-N-[[2-(3,4,5-trimethoxyphenyl)pyridin-4-yl]methyl]amino]piperidine (65 mg) and 4-chloromethyl-2-(3,4,5-trimethoxyphenyl)pyridine (50 mg) were condensed in the same manner as described in Example 2. The title compound was obtained after converting a free base to a tetrahydrochloride.

Yield: 121 mg (90%).

¹H-NMR (400 MHz, measured as a free base, CDCl₃)δ: 1.03 (t, 3H, J=7.1 Hz), 1.64-1.69 (m, 2H), 1.77 (d, 2H, J=10.7 Hz), 2.01 (t, 2H, J=10.8 Hz), 2.55-2.64 (m, 3H), 2.95 (d, 2H, J=11.1 Hz), 3.53 (s, 2H), 3.71 (s, 2H), 3.90 (s, 6H), 3.97 (s, 12H), 7.20-7.27 (m, 6H), 7.60 (s, 1H), 7.68 (s, 1H), 8.57 (d, 1H, J=4.9 Hz), 8.59 (d, 1H, J=5.1 Hz).

### PREPARATION EXAMPLE 80

### Synthesis of 4-(cyclohexylamino)-1-[[2-(3,4,5-trimethoxyphenyl)pyridin-4-yl]methyl]piperidine

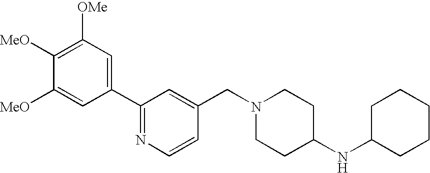

1-[[2-(3,4,5-trimethoxyphenyl)pyridin-4-yl]methyl]-4-pieridone (400 mg) and cyclohexylamine (134 mg) were reacted in the same manner as described in Preparation Example 37 to give the title compound.

Yield: 342 mg (69%).

¹H-NMR (400 MHz, CDCl₃) δ: 1.05-1.30 (m, 6H), 1.38-1.52 (m, 2H), 1.53-1.80 (m, 3H), 1.87 (br, 4H), 2.07 (t, 2H, J=10.7 Hz), 2.59(br, 2H), 2.86 (br, 2H), 3.54 (s, 2H), 3.90 (s, 3H), 3.97 (s, 6H), 7.19 (d, 1H, J=4.9 Hz), 7.24 (s, 2H), 7.64 (s, 1H), 8.58 (d, 1H, J=4.9 Hz).

### EXAMPLE 41

### Synthesis of 4-[N-cyclohexyl-N-[[2-(3,4,5-trimethoxyphenyl)pyridin-4-yl]methyl]amino]-1-[[2-(3,4,5-trimethoxyphenyl)pyridin-4-yl]methyl]piperidine Tetrahydrochloride

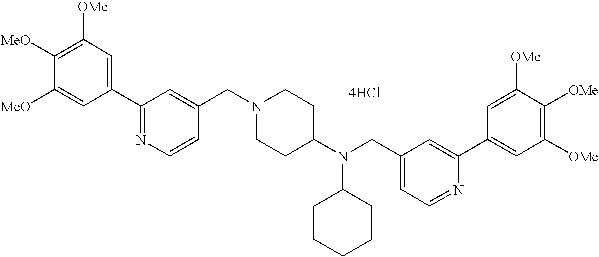

4-(Cyclohexylamino)-1-[[2-(3,4,5-trimethoxyphenyl)pyridin-4-yl]methyl]piperidine (342 mg) and 4-chloromethyl-2-(3,4,5-trimethoxyphenyl)pyridine (252 mg) were reacted in the same manner as described in Preparation Example 6. The title compound was obtained after converting the product to a tetrahydrochloride.

Yield: 55 mg (8%).

¹H-NMR (400 MHz, measured as a free base, CDCl₃)δ: 1.00-1.39 (m, 6H), 1.58-1.88 (m, 8H), 2.07 (br, 2H), 2.61 (br, 2H), 2.96 (br, 2H), 3.57 (br, 2H), 3.85 (s, 2H), 3.90 (s, 3H), 3.91 (s, 3H), 3.97 (s, 12H), 7.19-7.28 (m, 6H), 7.70 (br, 2H), 8.56 (d, 1H, J=5.1 Hz), 8.60 (d, 1H, J=5.1 Hz).

### PREPARATION EXAMPLE 81

### Synthesis of 4-anilino-1-[[2-(3,4,5-trimethoxyphenyl)pyridin-4-yl]methyl]piperidine

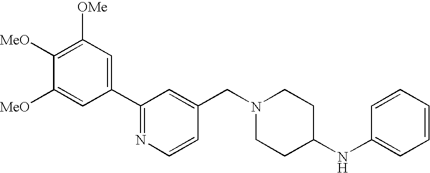

1-[[2-(3,4,5-trimethoxyphenyl)pyridin-4-yl]methyl]4-piperidone (1.1 g) and aniline (344 mg) were reacted in the same manner as described in Preparation Example 37 to give the title compound.

Yield: 1.09 g (81%).

¹H-NMR (400 MHz, CDCl₃)δ: 1.53 (br, 2H), 2.02-2.13 (m, 2H), 2.16-2.32 (m, 2H), 2.86 (br, 2H), 3.32 (br, 1H), 3.59 (s, 2H), 3.88 (s, 3H), 3.95 (s, 6H), 6.57 (d, 2H, J=8.6 Hz), 6.66 (t, 1H, J=7.3 Hz), 7.14 (t, 2H, J=7.9 Hz), 7.20-7.24 (m, 5H), 7.65 (br, 1H), 8.59 (d, 1H, J=5.1 Hz).

### EXAMPLE 42

### Synthesis of 4-[N-phenyl-N-[[2-(3,4,5-trimethoxyphenyl)pyridin-4-yl]methyl]amino]-1-[[2-(3,4,5-trimethoxyphenyl)pyridin-4-yl]methyl]piperidine Trihydrochloride

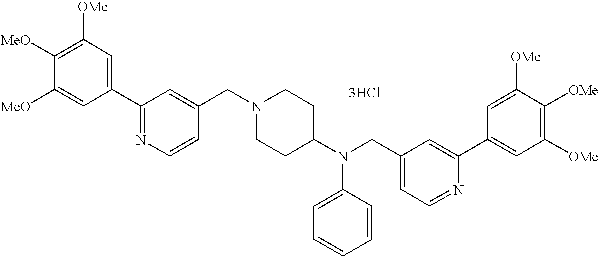

4-Anilino-1-[[2-(3,4,5-trimethoxyphenyl)pyridin-4-yl]methyl]piperidine (1.64 g) and 4-chloromethyl-2-(3,4,5-trimethoxyphenyl)pyridine (1.64 g) were reacted in the same manner as described in Preparation Example 9. The title compound was obtained after converting the product to a trihydrochloride.

Yield: 635 mg (20%).

¹H-NMR (400 MHz, measured as a free base, CDCl₃)δ: 1.60-2.00 (m, 4), 2.10-2.35 (m, 2H), 2.99 (br, 2H), 3.58 (br, 3H), 3.86 (s, 3H), 3.88 (s, 3H), 3.90 (s, 6H), 3.94 (s, 6H), 4.52 (s, 2H), 6.66-6.78 (m, 3H), 7.13-7.28 (m, 8H), 7.54 (br, 2H), 8.53 (d, 1H, J=5.1 Hz), 8.58 (d, 1H, J=4.9 Hz).

### PREPARATION EXAMPLE 82

### Synthesis of 1-[[2-(4-chloro-3,5-dimethoxyphenyl)pyridin-4-yl]methyl]-4-piperidone Ethylene Ketal

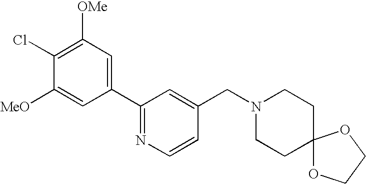

4-Piperidone ethylene ketal (573 mg) and 2-(4-chloro-3,5-dimetoxyphenyl)-4-chloromethylpyridine (1.19 g) were condensed in the same manner as described in Example 2 to give the title compound.

Yield: 1.67 g (theoretical amount).

¹H-NMR (400 MHz, CDCl₃)δ: 1.78 (t, 4H, J=5.6 Hz), 2.58 (br, 4H), 3.61 (s, 2H), 3.67 (s, 4H), 4.02 (s, 6H), 7.25-7.29 (m, 3H), 7.68 (s, 1H), 8.61 (d, 1H, J=4.9 Hz).

### PREPARATION EXAMPLE 83

### Synthesis of 1-[[2-(4-chloro-3,5-dimethoxyphenyl)pyridin-4-yl]methyl]-4-piperidone

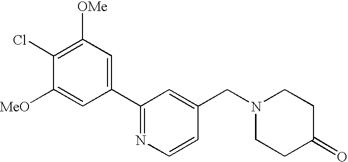

1-[[2-(4-Chloro-3,5-dimethoxyphenyl)pyridin-4-yl]methyl]-4-piperidone ethylene ketal (1.67 g) was treated in the same manner as described in Preparation Example 23 to give the title compound.

Yield: 1.29 g (89%).

¹H-NMR (400 MHz, CDCl₃) δ: 2.50 (t, 4H, J=5.8 Hz), 2.81 (t, 4H, J=5.8 Hz), 3.71 (s, 2H), 4.02 (s, 6H), 7.26 (s, 2H), 7.33 (d, 1H, J=4.3 Hz), 7.70 (s, 1H), 8.66 (d, 1H, J=4.9 Hz).

### PREPARATION EXAMPLE 84

### Synthesis of 4-anilino-1-[[2-(4-chloro-3,5-dimethoxyphenyl)pyridin-4-yl]methyl]piperidine

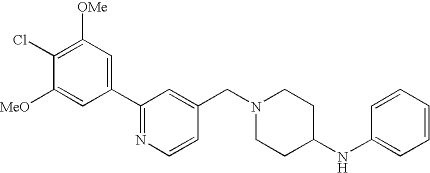

1-[[2-(4-Chloro-3,5-dimethoxyphenyl)pyridin-4-yl]methyl]-4-piperidone (600 mg) and aniline (0.18 mL) were reacted in the same manner as described in Preparation Example 37 to give the title compound.

Yield: 465 mg (63%).

¹H-NMR (400 MHz, CDCl₃)δ: 1.49-1.69 (m, 2H), 2.08 (d, 2H, J=7.8 Hz), 2.23 (t, 2H, J=9.3 Hz), 2.87 (d, 2H, J=7.8 Hz), 3.34 (br, 1H), 3.60 (s, 2H), 4.02 (s, 6H), 6.60 (d, 2H, J=7.6 Hz), 6.69 (t, 1H, J=7.3 Hz), 7.10-7.20 (m, 2H), 7.20-7.30 (m, 3H), 7.67 (s, 1H), 8.62 (d, 1H, J=5.2 Hz).

### EXAMPLE 43

### Synthesis of 1-[[2-(4-chloro-3,5-dimethoxyphenyl)pyridin-4-yl]methyl]-4-[N-[[2-(4-chloro-3,5-dimethoxyphenyl)pyridin-4-yl]methyl]-N-phenylamino] Piperidine Trihydrochloride

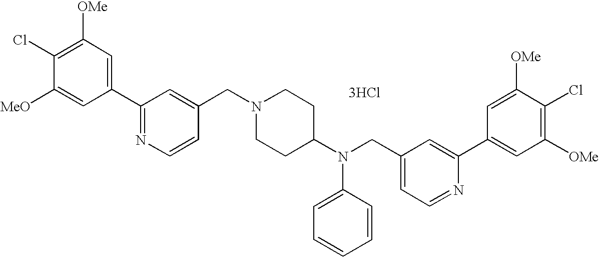

4-Anilino-1-[[2-(4-chloro-3,5-dimethoxyphenyl)pyridin-4-yl]methyl]piperidine (230 mg) and 2-(4-chloro-3,5-dimethoxyphenyl)-4-chloromethylpyridine (157 mg) were condensed in the same manner as described in Example 9. The title compound was obtained as yellow powder after converting a free base to a trihydrochloride.

Yield: 104 mg (24%).

¹H-NMR (400 MHz, measured as a free base, CDCl₃)δ: 1.70-1.85 (m, 4H), 2.20 (t, 2H, J=2.3 Hz), 3.00 (d, 2H, J=1.3 Hz), 3.59 (s, 2H), 3.96 (s, 6H), 4.00 (s, 6H), 4.56 (s, 2H), 6.65-6.78 (m, 3H), 7.16 (s, 2H), 7.18-7.28 (m, 6H), 7.59 (s, 1H), 7.62 (s, 1H), 8.57 (d, 1H, J=5.1 Hz), 8.57 (d, 1H, J=4.8 Hz).

### PREPARATION EXAMPLE 85

### Synthesis of 4-(p-anisidino)-1-[[2-(4-chloro-3,5-dimethoxyphenyl)pyridin-4-yl]methyl]piperidine

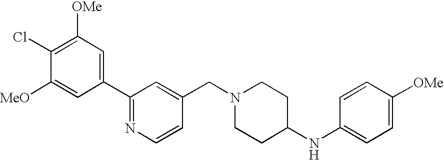

1-[[2-(4-Chloro-3,5-dimethoxyphenyl)pyridin-4-yl]methyl]-4-piperidone (690 mg) and p-anisidine (283 mg) were reacted in the same manner as described in Preparation Example 37 to give the title compound.

Yield: 646 mg (72%).

¹H-NMR (400 MHz, CDCl₃)δ: 1.45-1.55 (m, 2H), 2.05 (d, 2H, J=11.7 Hz), 2.20 (t, 241, J=11.2 Hz), 2.87 (d, 2H, J=11.7 Hz), 3.20-3.35 (m, 1H), 3.59 (s, 2H), 3.74 (s, 3H), 4.02 (s, 6H), 6.58 (d, 2H, J=8.7 Hz), 6.77 (d, 2H, J=8.7 Hz), 7.25-7.28 (m, 3H), 7.67 (s, 1H), 8.62 (d, 1H, J=4.9 Hz).

### EXAMPLE 44

### Synthesis of 1-[[2-(4-chloro-3,5-dimethoxyphenyl)pyridin-4-yl]methyl]4-[N-[[2-(4-chloro-3,5-dimethoxyphenyl)pyridin-4-yl]methyl]-N-(4-methoxyphenyl)amino] Piperidine Trihydrochloride

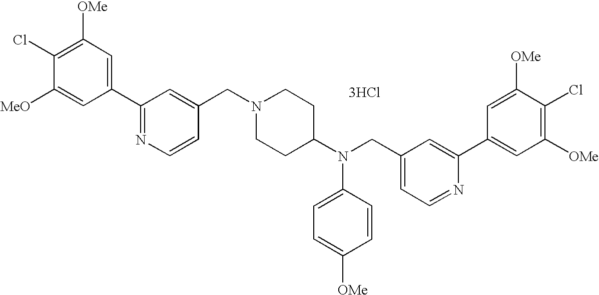

4-(p-Anisidino)-1-[[2-(4-chloro-3,5-dimethoxyphenyl)pyridin-4-yl]methyl]piperidine (271 mg) and 2-(4-chloro-3,5-dimethoxyphenyl)-4-chloromethylpyridine (173 mg) were condensed in the same manner as described in Example 9. The title compound was obtained as yellow powder after converting a free base to a trihydrochloride.

Yield: 324 mg (67%).

¹H-NMR (400 MHz, measured as a free base, CDCl₃)δ: 1.65-1.90 (m, 4H), 2.16 (t, 2H, J=10.4 Hz), 2.97 (d, 2H, J=7.5 Hz), 3.54-3.60 (m, 1H), 3.58 (s, 2H), 3.73 (s, 3H), 3.97 (s, 6H), 4.00 (s, 6H), 4.46 (s, 2H), 6.74 (d, 2H, J=9.4 Hz), 6.79 (d, 2H, J=9.4 Hz), 7.16 (s, 2H), 7.20-7.29 (m, 4H), 7.59 (s, 1H), 7.62 (s, 1H), 8.56 (d, 1H, J=4.8 Hz), 8.60 (d, 1H, J=4.8 Hz).

### PREPARATION EXAMPLE 86

### Synthesis of 4-(3-methylthioanilino)-1-[[2-(3,4,5-trimethoxyphenyl)pyridin-4-yl]methyl]piperidine

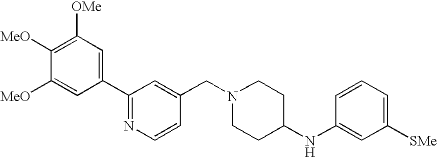

1-[[2-(3,4,5-trimethoxyphenyl)pyridin-4-yl]methyl]-4-piperidone (1.40 g) and 3-methylthioaniline (655 mg) were reacted in the same manner as described in Preparation Example 37 to give the title compound.

Yield: 1.01 g (54%).

¹H-NMR (400 MHz, CDCl₃)δ: 1.44-1.60 (m, 2H), 1.98-2.10 (m, 2H), 2.23 (br, 2H), 2.42 (s, 3H), 2.88 (br, 2H), 3.30 (br, 1H), 3.59 (s, 2H), 3.88 (s, 3H), 3.95 (s, 6H), 6.35 (d, 1H, J=7.6 Hz), 6.47 (s, 1H), 6.55 (d, 1H, J=8.6 Hz), 7.05 (t, 1H, J=7.9 Hz), 7.20 (d, 1H, J=4.9 Hz), 7.24 (s, 2H), 7.68 (br, 1H), 8.58 (d, 1H, J=4.9 Hz).

### EXAMPLE 45

### Synthesis of 4-[N-(3-methylthiophenyl)-N-[[2-(3,4,5-trimethoxyphenyl)pyridin-3-yl]methyl]amino]-1-[[2-(3,4,5-trimethoxyphenyl)pyridin-4-yl]methyl]piperidine Trihydrochloride

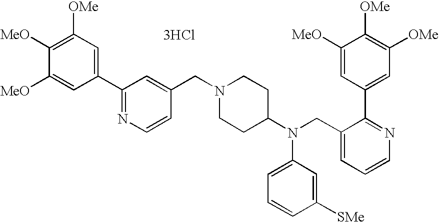

4-(3-Methylthioanilino)-1-[[2-(3,4,5-trimethoxyphenyl)pyridin-4-yl]methyl]piperidine (143 mg) and 3-chloromethyl-2-(3,4,5-timethoxyphenyl)pyridine (114 mg) were condensed in the same manner as described in Example 9. The title compound was obtained as yellow powder after converting a free base to a trihydrochloride.

Yield: 45 mg (18%).

¹H-NMR (400 MHz, measured as a free base, CDCl₃)δ: 1.58-1.71 (s, 2H), 1.79 (d, 2H, J=10.7 Hz), 2.16 (t, 2H, J=11.2 Hz), 2.38 (s, 3H), 2.96 (d, 2H, J=11.2 Hz), 3.56 (s, 3H), 3.68-3.97 (m, 1H), 3.90 (s, 3H), 3.92 (s, 9H), 3.96 (s, 9H), 4.42 (s, 2H), 6.45 (d, 1H, J=8.3 Hz), 6.52 (s, 1H), 6.61 (d, 1H, J=7.3 Hz), 6.74 (s, 2H), 7.11 (t; 1H, J=8.1 Hz), 7.15-7.26 (m, 4H), 7.54 (s, 1H), 7.68 (d, 1H, J=7.8 Hz), 8.53 (d, 1H, J=3.2 Hz), 8.59 (d, 1H, J=4.8 Hz).

### EXAMPLE 46

### Synthesis of 4-[N-(3-methylthiophenyl)-N-[2-(3,4,5-trimethoxyphenyl)benzyl]amino]-1-[[2-(3,4,5-trimethoxyphenyl)pyridin-4-yl]methyl]piperidine Dihydrochloride

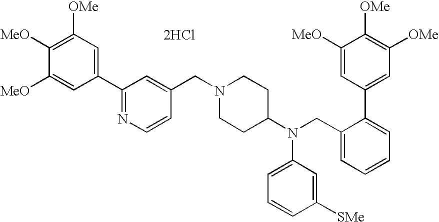

4-(3-Methylthioanilino)-1-[[2-(3,4,5-trimethoxyphenyl)pyridin-4-yl]methyl]piperidine (143 mg) and 2-(3,4,5-trimethoxyphenyl)benzyl chloride (114 mg) were condensed in the same manner as described in Example 9. The title compound was obtained as yellow powder after converting a free base to a dihydrochloride.

Yield: 51 mg (23%).

¹H-NMR (400 MHz, measured as a free base, CDCl₃)δ: 1.56-1.73 (m, 2H), 1.78-1.87 (m, 2H), 2.10-2.20 (m, 2H), 2.38 (s, 3H), 2.91-2.98 (m, 2H), 3.55 (s, 2H), 3.70-3.80 (m, 1H), 3.88 (s, 6H), 3.90 (s, 3H), 3.92 (s, 3H), 3.96 (s, 6H), 4.35 (s, 2H), 6.47 (d, 1H, J=8.2 Hz), 6.53-6.62 (m, 5H), 7.09 (t, 1H, J=8.0 Hz), 7.18-7.40 (m, 6H), 7.54 (s, 1H), 8.58 (d, 1H, J=4.7 Hz).

### EXAMPLE 47

### Synthesis of 4-[N-(3-methylthiophenyl)-N-[[2-(3,4,5-trimethoxyphenyl)pyridin-4-yl]methyl]amino]-1-[[2-(3,4,5-trimethoxyphenyl)pyridin-4-yl]methyl]piperidine Fumarate

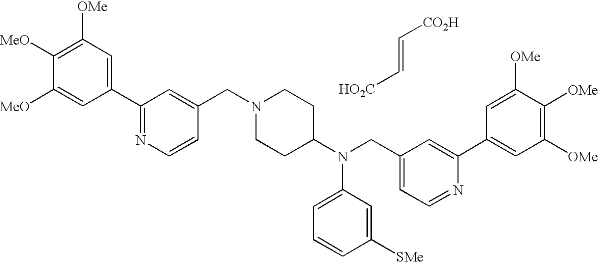

4-(3-Methylthioanilino)-1-[[2-(3,4,5-trimethoxyphenyl)pyridin-yl]methyl]piperidine (143 mg) and 4-chloromethyl-2-(3,4,5-trimethoxyphenyl)pyridine (114 mg) were condensed in the same manner as described in Example 9. The title compound was obtained as white powder after converting a free base to a fumarate.

Yield: 14 mg (5%).

¹H-NMR (400 MHz, measured as a free base, CDCl₃)δ: 1.76-1.86 (m, 5H), 2.17-2.23 (m, 2H), 2.39 (s, 3H), 2.97-3.00 (m, 2H), 3.58 (s, 2H), 3.89 (s, 3H), 3.90 (s, 3H), 3.93 (s, 6H), 3.96 (s, 6H), 4.54 (s, 2H), 6.47-6.50 (m, 1H), 6.63 (s, 1H), 6.64 (s, 1H), 7.10-7.15 (m, 2H), 7.15 (s, 2H), 7.20-7.21 (m, 1H), 7.22 (s, 2H), 7.55 (s, 1H), 7.59 (s, 1H), 8.56 (d, 1H, J=5.1 Hz), 8.59 (d, 1H, J=5.1 Hz).

### EXAMPLE 48

### Synthesis of 4-[N-(3-methylthiophenyl)-N-[3-(3,4,5-trimethoxyphenyl)benzyl]amino]-1-[[2-(3,4,5-trimethoxyphenyl)pyridin-4-yl]methyl]piperidine Dihydrochloride

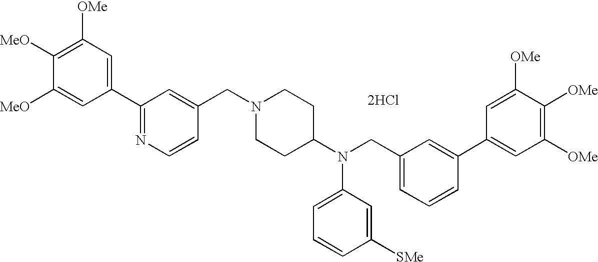

4-(3-Methylthioanilino)-1-[[2-(3,4,5-trimethoxyphenyl)pyridin-4-yl]methyl]piperidine (143 mg) and 3-(3,4,5-trimethoxyphenyl)benzyl chloride (114 mg) were condensed in the same manner as described in Example 9. The title compound was obtained as yellow powder after converting a free base to a dihydrochloride.

Yield: 60 mg (24%).

¹H-NMR (400 MHz, measured as a free base, CDCl₃)δ: 1.65-1.91 (m, 4H), 2.18 (t, 2H, J=10.5 Hz), 2.38 (s, 3H), 2.97 (d, 2H, J=10.9 Hz), 3.58 (s, 2H), 3.70-3.85 (m, 1H), 3.88 (s, 3H), 3.89 (s, 6H), 3.90 (s, 3H), 3.96 (s, 6H), 4.56 (s, 2H), 6.52 (d, 1H, J=8.4 Hz), 6.59 (d, 1H, J=7.6 Hz), 6.65 (s, 1H), 6.72 (s, 2H), 7.10 (t, 2H, J=8.0 Hz), 7.19-7.25 (m, 4H), 7.31-7.42 (m, 3H), 7.60 (s, 1H), 8.59 (d, 1H, J=7.8 Hz).

### EXAMPLE 49

### Synthesis of 4-[N-(3-methylthiophenyl)-N-[[2-(3,4,5-trimethoxyphenyl)pyridin-5-yl]methyl]amino]-1-[[2-(3,4,5-trimethoxyphenyl)pyridin-4-yl]methyl]piperidine Trihydrochloride

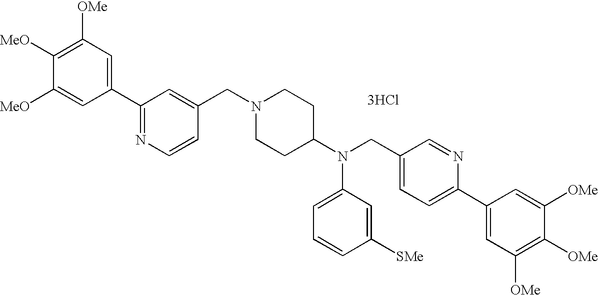

4-(3-Methylthioanilino)-1-[[2-(3,4,5-trimethoxyphenyl)pyridin-4-yl]methyl]piperidine (143 mg) and 5-chloromethyl-2-(3,4,5-trimethoxyphenyl)pyridine (114 mg) were condensed in the same manner as described in Example 9. The title compound was obtained as yellow powder after converting a free base to a trihydrochloride.

Yield: 22 mg (9%).

¹H-NMR (400 MHz, measured as a free base, CDCl₃)δ: 1.50-2.05 (m, 4H), 2.20 (br, 2H), 2.37 (s, 3H), 3.05 (br, 2H), 3.50-3.70 (br, 3H), 3.86 (s, 3H), 3.87 (s, 3H), 3.92 (s, 6H), 3.95 (s, 6H), 4.52 (s, 2H), 6.49 (d, 1H, J=8.3 Hz), 6.62 (br, 2H), 7.09 (t, 1H, J=8.2 Hz), 7.18-7.30 (m, 6H), 7.58 (s, 2H), 8.54 (br, 1H), 8.60 (br, 1H).

### EXAMPLE 50

### Synthesis of 4-[N-(3-methylthiophenyl)-N-[4-(3,4,5-trimethoxyphenyl)benzyl]amino]-1-[[2-(3,4,5-trimethoxyphenyl)pyridin-4-yl]methyl]piperidine Dihydrochloride

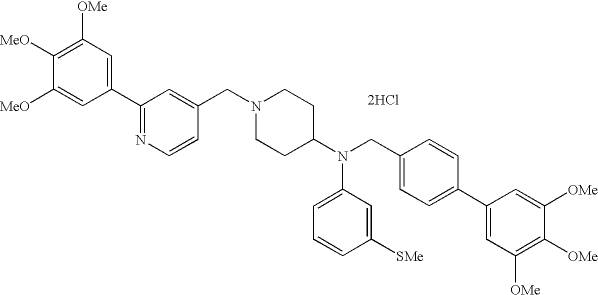

4-(3-Methylthioanilino)-1-[[2-(3,4,5-trimethoxyphenyl)pyridin-4-yl]methyl]piperidine (143 mg) and 4-(3,4,5-trimethoxyphenyl)benzyl chloride (114 mg) were condensed in the same manner as described in Example 9. The title compound was obtained as yellow powder after converting a free base to a dihydrochloride.

Yield: 57 mg (22%).

¹H-NMR (400 MHz, measured as a free base, CDCl₃)δ: 1.58-1.83 (m, 4H), 2.20 (t, 2H, J=11.3 Hz), 2.39 (s, 3H), 2.98 (d, 2H, J=111.1 Hz), 3.58 (s, 2H), 3.88 (s, 3H), 3.90 (s, 3H), 3.91 (s, 6H), 3.96 (s, 6H), 4.53 (s, 2H), 6.51 (dd, 1H, J=8.4 Hz, 2.4 Hz), 6.60 (d, 1H, J=8.0 Hz), 6.64 (s, 1H), 6.75 (s, 2H), 7.10 (t, 1H, J=8.1 Hz), 7.24-7.33 (m, 4H), 7.47 (d, 2H, J=8.0 Hz), 7.61 (s, 1H), 8.59 (d, 1H, J=5.0 Hz).

### PREPARATION EXAMPLE 87

### Synthesis of 4-propargylamino-1-[[2-(3,4,5-trimethoxyphenyl)pyridin-4-yl]methyl]piperidine

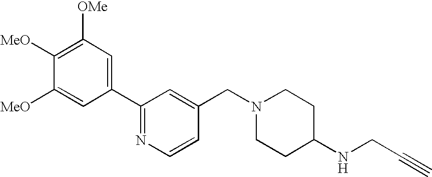

1-[[2-(3,4,5-trimethoxyphenyl)pyridin-4-yl]methyl]-4-piperidone (400 mg) and propargylamine (80 mg) were reacted in the same manner as described in Preparation Example 25 to give the title compound.

Yield: 227 mg (63%).

¹H-NMR (400 MHz, CDCl₃)δ: 1.38-1.51 (m, 2H), 1.83-1.86 (m, 3H), 2.10-2.15 (m, 2H), 2.21 (s, 1H), 2.74 (br, 1H), 2.83-2.87 (m, 2H), 3.45 (s, 2H), 3.56 (s, 2H), 3.89 (s, 3H), 3.96 (s, 6H), 7.19 (d, 1H, J=4.9 Hz), 7.24 (s, 2H), 7.65 (s, 1H), 8.58 (d, 1H, J=4.9 Hz).

### EXAMPLE 51

### Synthesis of 4-[N-propargyl-N-[[2-(3,4,5-trimethoxyphenyl)pyridin-4-yl]methyl]amino]-1-[[2-(3,4,5-trimethoxyphenyl)pyridin-4-yl]methyl]piperidine Tetrahydrochloride

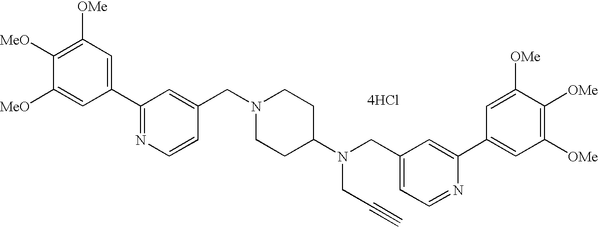

4-Propargylamino-1-[[2-(3,4,5-trimethoxyphenyl)pyridin-4-yl]methyl]piperidine (227 mg) and 4-chloromethyl-2-(3,4,5-trimethoxyphenyl)pyridine (226 mg) were condensed in the same manner as described in Example 9. The title compound was obtained as yellow powder after converting a free base to a tetrahydrochloride.

Yield: 128 mg (23%).

¹H-NMR (400 MHz, measured as a free base, CDCl₃)δ: 1.48-2.40 (m, 7H), 2.72 (br, 1H), 3.02 (br, 2H), 3.39 (s, 2H), 3.64 (br, 2H), 3.84 (s, 2H), 3.91 (s, 6H), 3.98 (s, 6H), 3.99 (s, 6H), 7.22-7.29 (m, 6H), 7.66 (br, 2H), 8.60 (d, 1H, J=4.9 Hz), 8.62 (d, 1H, J=4.9 Hz).

### PREPARATION EXAMPLE 88

### Synthesis of 4-(5-indanylamino)-1-[[2-(3,4,5-trimethoxyphenyl)pyridin-4-yl]methyl]piperidine

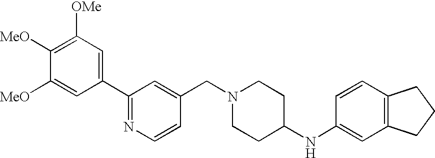

1-[[2-(3,4,5-trimethoxyphenyl)pyridin-4-yl]methyl]-4-piperidone (1.40 g) and 5-aminoindan (680 mg) were reacted in the same manner as described in Preparation Example 37 to give the title compound.

Yield: 1.22 g (59%).

¹H-NMR (400 MHz, CDCl₃)δ: 1.40-1.57 (m, 2H), 2.00-2.15 (m, 5H), 2.19-2.25 (m, 2H), 2.77-2.93 (m, 6H), 3.30 (br, 1H), 3.58 (s, 2H), 3.91 (s, 3H), 3.97 (s, 6H), 6.41 (d, 1H, J=8.0 Hz), 6.52 (s, 1H), 7.01 (d, 1H, J=8.0 Hz), 7.21-7.26 (m, 3H), 7.64 (s, 1H), 8.60 (d, 1H, J=4.9 Hz).

### EXAMPLE 52

### Synthesis of 4-[N-(indan-5-yl)-N-[[2-(3,4,5-trimethoxyphenyl)pyridin-3-yl]methyl]amino]-1-[[2-(3,4,5-trimethoxyphenyl)pyridin-4-yl]methyl]piperidine Trihydrochloride

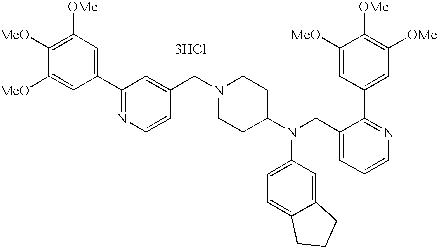

4-(5-Indanylamino)-1-[[2-(3,4,5-trimethoxyphenyl)pyridin-4-yl]methyl]piperidine (142 mg) and 3-chloromethyl-2-(3,4,5-trimethoxyphenyl)pyridine (114 mg) were condensed in the same manner as described in Example 9. The title compound was obtained as yellow powder after converting a free base to a trihydrochloride.

Yield: 90 mg (41%).

¹H-NMR (400 MHz, measured as a free base, CDCl₃)δ: 1.54-1.67 (m, 2H), 1.74-1.83 (m, 2H), 1.98-2.07 (m, 2H), 2.09-2.98 (m, 2H), 3.55 (s, 2H), 3.64-3.74 (m, 1H), 3.90 (s, 3H), 3.91 (s, 6H), 3.92 (s, 3H), 3.96 (s, 6H), 4.41 (s, 2H), 6.49 (dd, 1H, J=8.2 Hz, 2.4 Hz), 6.59 (s, 1H), 6.74 (s, 2H), 7.04 (d, 1H, J=8.2 Hz), 7.15-7.20 (m, 2H), 7.22 (s, 2H), 7.54 (s, 1H), 7.77 (dd, 1H, J=7.8 Hz, 1.4 Hz), 8.52 (dd, 1H, J=4.7 Hz, 1.8 Hz), 8.59 (d, 1H, J=5.1 Hz).

### EXAMPLE 53

### Synthesis of 4-[N-(indan-5-yl)-N-[2-(3,4,5-trimethoxyphenyl)benzyl]amino]-1-[[2-(3,4,5-trimethoxyphenyl)pyridin-4-yl]methyl]piperidine Dihydrochloride

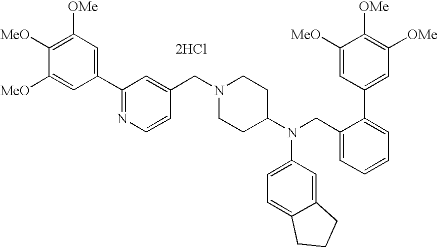

45-Indanylamino)-1-[[2-(3,4,5-trimethoxyphenyl)pyridin-4-yl]methyl]piperidine (142 mg) and 2-(3,4,5-trimethoxyphenyl)benzyl chloride (114 mg) were condensed in the same manner as described in Example 9. The title compound was obtained as yellow powder after converting a free base to a dihydrochloride.

Yield: 115 mg (47%).

¹H-NMR (400 MHz, measured as a free base, CDCl₃)δ: 1.56-1.66 (m, 2H), 1.80-1.83 (m, 2H), 2.00-2.05 (m, 2H), 2.11-2.18 (m, 2H), 2.77-2.83 (m, 4H), 2.92-2.95 (m, 2H), 3.55 (s, 2H), 3.72 (br, 1H), 3.87 (s, 6H), 3.90 (s, 3H), 3.92 (s, 3H), 3.96 (s, 6H), 4.34 (s, 2H), 6.49 (d, 1H, J=8.3 Hz), 6.56 (s, 2H), 6.60 (s, 1H), 7.02 (d, 1H, J=8.3 Hz), 7.17-7.27 (m, 5H), 7.42-7.45 (m, 1H), 7.54 (s, 1H), 8.58 (d, 1H, J=4.9 Hz).

### EXAMPLE 54

### Synthesis of 4-[N-(indan-5-yl)-N-[[2-(3,4,5-trimethoxyphenyl)pyridin-4-yl]methyl]amino]-1-[[2-(3,4,5-trimethoxyphenyl)pyridin-4-yl]methyl]piperidine Trihydrochloride

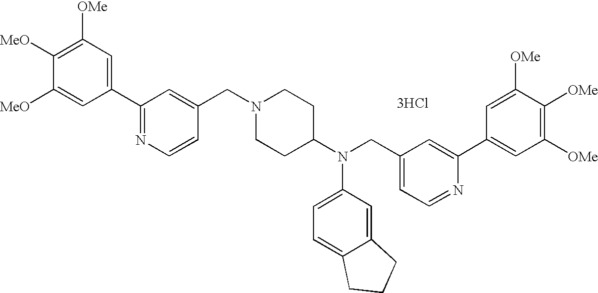

4-(5-Indanylamino)-1-[[2-(3,4,5-trimethoxyphenyl)pyridin-4-yl]methyl]piperidine (142 mg) and 4-chloromethyl-2-(3,4,5-trimethoxyphenyl)pyridine (114 mg) were condensed in the same manner as described in Example 9. The title compound was obtained as white powder after converting a free base to a trihydrochloride.

Yield: 23 mg (9%).

¹H-NMR (400 MHz, measured as a free base, CDCl₃)δ: 1.60-1.95 (m, 4H), 2.00 (quint, 2H, J=7.3 Hz), 2.20 (br, 2H), 2.75-2.81 (m, 4H), 2.99 (br, 2H), 3.58 (br, 2H), 3.77 (s, 1H), 3.86 (s, 3H), 3.87 (s, 3H), 3.91 (s, 6H), 3.94 (s, 6H), 4.49 (s, 2H), 6.51 (d, 1H, J=8.3 Hz), 6.62 (s, 1H), 7.02 (d, 1H, J=8.0 Hz), 7.16 (s, 2H), 7.18-7.22 (m, 4H), 7.57 (br, 2H), 8.52 (d, 1H, J=4.9 Hz), 8.57 (d, 1H, J=4.9 Hz).

### EXAMPLE 55

### Synthesis of 4-[N-(indan-5-yl)-N-[3-(3,4,5-trimethoxyphenyl)benzyl]amino]-1-[[2-(3,4,5-trimethoxyphenyl)pyridin-4-yl]methyl]piperidine Dihydrochloride

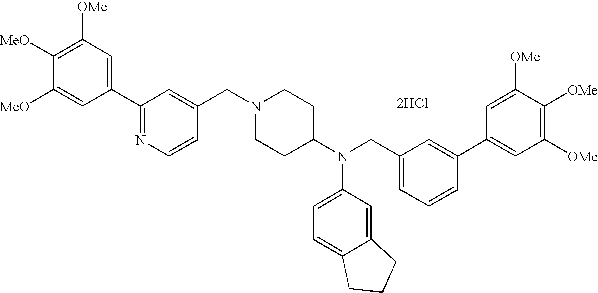

4-(5-Indanylamino)-1-[[2-(3,4,5-trimethoxyphenyl)pyridin-4-yl]methyl]piperidine (60 mg) and 3-(3,4,5-trimethoxyphenyl)benzyl chloride (114 mg) were condensed in the same manner as described in Example 9. The title compound was obtained as yellow powder after converting a free base to a dihydrochloride.

Yield: 18 mg (19%).

¹H-NMR (400 MHz, measured as a free base, CDCl₃)δ: 1.60-1.95 (m, 4H), 2.00 (quint, 2H, J=7.2 Hz), 2.20 (br, 2H), 2.75-2.81 (m, 4H), 2.95 (br, 2H), 3.60 (br, 2H), 3.85 (br, 1H), 3.86 (s, 3H), 3.87 (s, 6H), 3.88 (s, 3H), 3.94 (s, 6H), 4.51 (s, 2H), 6.54 (d, 1H, J=8.2 Hz), 6.66 (s, 1H), 6.70 (s, 2H), 7.01 (d, 1H, J=8.4 Hz), 7.19 (d, 1H, J=4.9 Hz), 7.19-7.42 (m, 6H), 7.60 (br, 1H), 8.59 (br, 1H).

### EXAMPLE 56

### Synthesis of 4-[N-(indan-5-yl)-N-[[2-(3,4,5-trimethoxyphenyl)pyridin-5-yl]methyl]amino]-1-[[2-(3,4,5-trimethoxyphenyl)pyridin-5-yl]methyl]piperidine Trihydrochloride

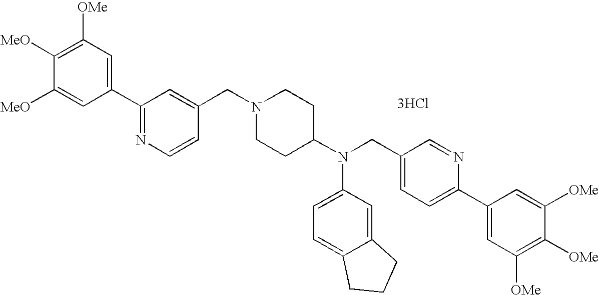

4-(5-Indanylamino)-1-[[2-(3,4,5-trimethoxyphenyl)pyridin-4-yl]methyl]piperidine (143 mg) and 5-chloromethyl-2-(3,4,5-trimethoxyphenyl)pyridine (114 mg) were condensed in the same manner as described in Example 9. The title compound was obtained as yellow powder after converting a free base to a trihydrochloride.

Yield: 138 mg (63%).

¹H-NMR (400 MHz, measured as a free base, CDCl₃)δ: 1.71-1.91 (m, 4H), 1.98-2.06 (m, 2H), 2.13-2.22 (m, 2H), 2.76-2.84 (m, 4H), 2.94-3.05 (m, 2H), 3.57 (s, 2H), 3.69-3.78 (m, 1H), 3.89 (s, 3H), 3.90 (s, 3H), 3.94 (s, 6H), 3.96 (s, 6H), 4.50 (s, 2H), 6.57 (dd, 1H, J=8.2 Hz, 2.3 Hz), 6.67 (s, 1H), 7.04 (d, 1H, J=8.4 Hz), 7.20-7.22 (m, 1H), 7.22 (s, 2H), 7.23 (s, 2H), 7.57-7.62 (m, 1H), 7.60 (s, 1H), 7.65 (dd, 1H, J=8.2 Hz, 2.2 Hz), 8.58-8.62 (m, 2H).

### EXAMPLE 57

### Synthesis of 4-[N-(indan-5-yl)-N-[4-(3,4,5-trmethoxyphenyl)benzyl]amino]-1-[[2-(3,4,5-trimethoxyphenyl)pyridin-4-yl]methyl]piperidine Dihydrochloride

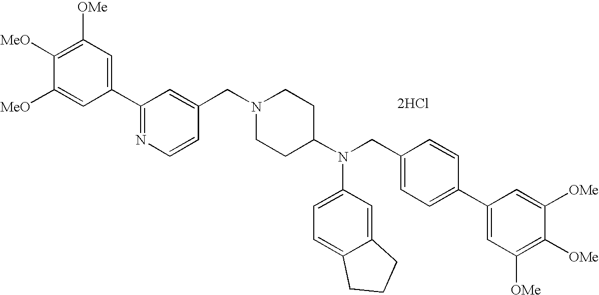

4-(5-Indanylamino)-1-[[2-(3,4,5-trimethoxyphenyl)pyridin-4-yl]methyl]piperidine (143 mg) and 4-(3,4,5-trimethoxyphenyl)benzyl chloride (114 mg) were condensed in the same manner as described in Example 9. The title compound was obtained as yellow powder after converting a free base to a dihydrochloride.

Yield: 95 mg (39%).

¹H-NMR (400 MHz, measured as a free base, CDCl₃)δ: 1.74-1.90 (m, 4H), 2.01-2.06 (m, 2H), 2.16-2.22 (m, 2H), 2.78-2.84 (m, 41), 2.96-2.99 (m, 2H), 3.58 (s, 2H), 3.72 (br, 1H), 3.88 (s, 3H), 3.90 (s, 3H), 3.91 (s, 6H), 3.96 (s, 6H), 4.51 (s, 2H), 6.55 (d, 1H, J=8.3 Hz), 6.67 (s, 1H), 6.72 (s, 2H), 7.04 (d, 1H, J=8.3 Hz), 7.20 (d, 1H, J=5.1 Hz), 7.23 (s, 2H), 7.35 (d, 2H, J=8.1 Hz), 7.47 (d, 2H, J=8.1 Hz), 7.61 (s, 1H), 8.59 (d, 1H, J=4.9 Hz).

### PREPARATION EXAMPLE 89

### Synthesis of 4-(4-butylanilino)-1-[[2-(3,4,5-trimethoxyphenyl)pyridin-4-yl]methyl]piperidine

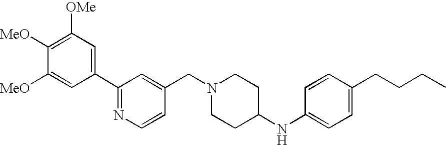

1-[[2-(3,4,5-trimethoxyphenyl)pyridin-4-yl]methyl]-4-piperidone (1.24 g) and 4-butylaniline (149 mg) were reacted in the same manner as described in Preparation Example 37 to give the title compound.

Yield: 1.23 g (72%).

¹H-NMR (400 MHz, CDCl₃)δ: 0.82 (t, 3H, J=7.3 Hz), 1.20-1.30 (m, 2H), 1.38-1.50 (m, 4H), 1.92-2.25 (m, 4H), 2.40 (t, 2H, J=7.7 Hz), 2.77 (br, 2H), 3.21 (br, 1H), 3.50 (s, 2H), 3.82 (s, 3H), 3.89 (s, 6H), 6.45 (d, 2H, J=7.8 Hz), 6.89 (d, 2H, J=8.0 Hz), 7.13 (d, 1H, J=4.9 Hz), 7.18 (s, 2H), 7.58 (s, 1H), 8.52 (d, 1H, J=4.9 Hz).

### EXAMPLE 58

### Synthesis of 4-[N-(4-butylphenyl)-N-[[2-(3,4,5-trimethoxyphenyl)pyridin-3-yl]methyl]amino]-1-[[2-(3,4,5-trimethoxyphenyl)pyridin-4-yl]methyl]piperidine Trihydrochloride

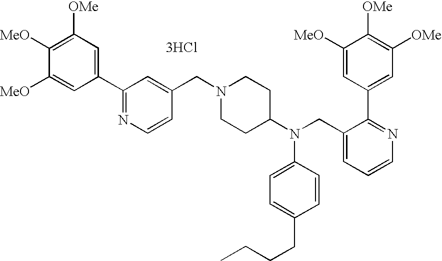

4-(4-Butylanilino)-1-[[2-(3,4,5-trimethoxyphenyl)pyridin-4-yl]methyl]piperidine (147 mg) and 3-chloromethyl-2-(3,4,5-trimethoxyphenyl)pyridine (114 mg) were condensed in the same manner as described in Example 9. The title compound was obtained as yellow powder after converting a free base to a trihydrochloride.

Yield: 58 mg (27%).

¹H-NMR (400 MHz, measured as a free base, CDCl₃)δ: 0.91 (t, 3H, J=7.3 Hz), 1.32-1.35 (m, 2H), 1.50-1.70 (m, 4H), 1.75 (br, 2H), 2.10-2.20 (m, 2H), 2.49 (t, 2H, J=7.6 Hz), 2.95 (br, 21), 3.55 (s, 2H), 3.70 (br, 1H), 3.90 (s, 3H), 3.91 (s, 6H), 3.92 (s, 3H), 3.96 (s, 6H), 4.41 (s, 2H), 6.59 (d, 2H, J=8.8 Hz), 6.74 (s, 2H), 7.00 (d, 2H, J=8.6 Hz), 7.16-7.17 (m, 1H), 7.19 (d, 1H, 3=4.9 Hz), 7.22 (s, 2H), 7.54 (s, 1H), 8.59 (d, 1H, J=7.5 Hz), 8.52 (br, 1H), 8.59 (d, 1H, J=4.9 Hz).

### EXAMPLE 59

### Synthesis of 4-[N-(4-butylphenyl)-N-[2-(3,4,5-trimethoxyphenyl)benzyl]amino]-1-[[2-(3,4,5-trimethoxyphenyl)pyridin-4-yl]methyl]piperidine Dihydrochloride

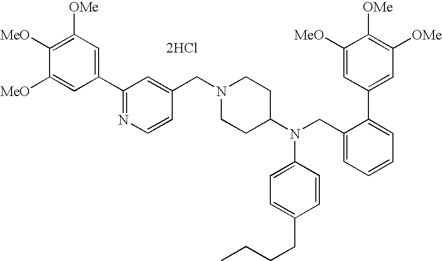

4-(4-Butylanilino)-1-[[2-(3,4,5-trimethoxyphenyl)pyridin-4-yl]methyl]piperidine (147 mg) and 2-(3,4,5-trimethoxyphenyl)benzyl chloride (114 mg) were condensed in the same manner as described in Example 9. The title compound was obtained as yellow powder after converting a free base to a dihydrochloride.

Yield: 59 mg (24%).

¹H-NMR (400 MHz, measured as a free base, CDCl₃)δ: 0.90 (t, 3H, J=7.4 Hz), 1.25-1.41 (m, 2H), 1.48-1.75 (m, 4H), 1.81 (d, 2H, J=11.7 Hz), 2.13 (t, 2H, J=11.2 Hz), 2.48 (t, 2H, J=7.5 Hz), 2.93 (d, 2H, J=11.2 Hz), 3.55 (s, 2H), 3.65-3.80 (m, 1H), 3.87 (s, 6H), 3.90 (s, 3H), 3.92 (s, 1H), 3.96 (s, 6H), 4.33 (s, 2H), 6.56 (s, 2H), 6.60 (d, 2H, J=8.5 Hz), 6.98 (d, 2H, J=8.5 Hz), 7.18 (d, 1H, J=4.9 Hz), 7.21 (s, 2H), 7.20-7.37 (m, 3H), 7.41 (br, 1H), 7.54 (s, 1H), 8.58 (d, 1H, J=4.9 Hz).

### EXAMPLE 60

### Synthesis of 4-[N-(4-buthylphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine Trihydrochloride

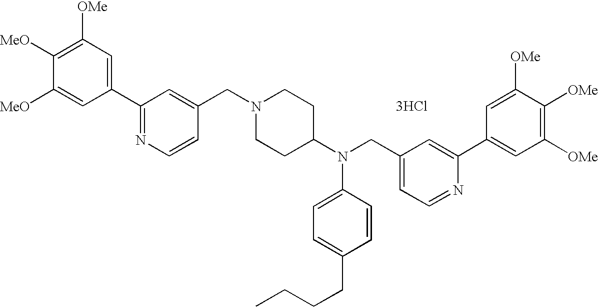

4-(4-Butylanilino)-1-[[2-(3,4,5-trimethoxyphenyl)pyridin-4-yl]methyl]piperidine (196 mg) and 4-chloromethyl-2-(3,4,5-trimethoxyphenyl)pyridine (129 mg) were condensed in the same manner as described in Example 9. The title compound was obtained as white powder after converting a free base to a trihydrochloride.

Yield: 20 mg (6%).

¹H-NMR (400 MHz, measured as a free base, CDCl₃)δ: 0.88 (t, 3H, J=7.3 Hz), 1.20-1.35 (m, 2H), 1.49-1.60 (it, 2H), 1.62-2.02 (m, 4H), 2.20 (br, 2H), 2.46 (t, 2H, J=7.3 Hz), 3.05 (br, 2H), 3.60 (br, 3H), 3.87 (s, 3H), 3.88 (s, 3H), 3.90 (s, 6H), 3.94 (s, 6H), 4.49 (s, 2H), 6.62 (d, 2H, J=8.3 Hz), 6.98 (d, 2H, J=8.3 Hz), 7.13 (s, 2H), 7.15-7.40 (m, 4H), 7.55 (br, 2H), 8.52 (d, 1H, J=4.9 Hz), 8.60 (br, 1H).

### EXAMPLE 61

### Synthesis of 4-[N-(4-butylphenyl)-N-[3-(3,4,5-trimethoxyphenyl)benzyl]amino]-1-[[2-(3,4,5-trimethoxyphenyl)pyridin-4-yl]methyl]piperidine Dihydrochloride

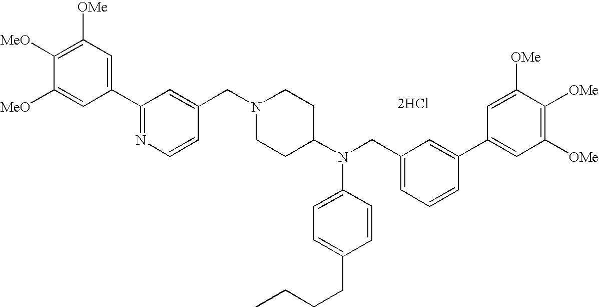

4-(4-Butylanilino)-1-[[2-(3,4,5-trimethoxyphenyl)pyridin-4-yl]methyl]piperidine (147 mg) and 3-(3,4,5-trimethoxyphenyl)benzyl chloride (114 mg) were condensed in the same manner as described in Example 9. The title compound was obtained as yellow powder after converting a free base to a dihydrochloride.

Yield: 102 mg (42%).

¹H-NMR (400 MHz, measured as a free base, CDCl₃)δ: 0.90 (t, 3H, J=7.4 Hz), 1.30-1.36 (m, 2H), 1.48-1.56 (m, 2H), 1.76-1.89 (m, 4H), 2.19 (br, 2H), 2.48 (t, 2H, J=7.8 Hz), 2.97 (br, 2H), 3.58 (s, 2H), 3.86 (br, 1H), 3.88 (s, 3H), 3.90 (s, 3H), 3.95 (s, 6H), 4.54 (s, 2H), 6.68 (d, 2H, J=8.6 Hz), 6.72 (s, 2H), 7.00 (d, 2H, J=8.6 Hz), 7.20-7.27 (m, 2H), 7.23 (s, 2H), 7.32-7.40 (m, 2H), 7.44 (s, 1H), 7.62 (s, 1H), 8.59 (d, 1H, J=5.1 Hz).

### EXAMPLE 62

### Synthesis of 4-[N-(4-butylphenyl)-N-[[2-(3,4,5-trimethoxyphenyl)pyridin-5-yl]methyl]amino]-1-[[2-(3,4,5-trimethoxyphenyl)pyridin-4-yl]methyl]piperidine Trihydrochloride

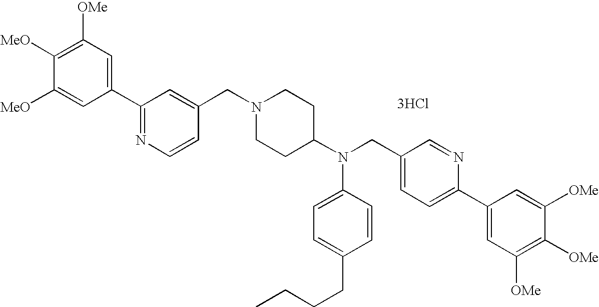

4-(4-Butylanilino)-1-[[2-(3,4,5-trimethoxyphenyl)pyridin-4-yl]methyl]piperidine (147 mg) and 5-chloromethyl-2-(3,4,5-trimethoxyphenyl)pyridine (114 mg) were condensed in the same manner as described in Example 9. The title compound was obtained as yellow powder after converting a free base to a trihydrochloride.

Yield: 65 mg (21%).

¹H-NMR (400 MHz, measured as a free base, CDCl₃)δ: 0.90 (t, 3H, J=7.3 Hz), 1.32-1.36 (m, 2H), 1.50-1.54 (m, 2H), 1.70-1.95 (m, 4H), 2.17 (br, 2H), 2.49 (t, 2H, J=7.7 Hz), 2.96 (br, 2H), 3.58 (s, 2H), 3.75 (br, 1H), 3.89 (s, 3H), 3.90 (s, 3H), 3.94 (s, 6H), 3.96 (s, 6H), 4.50 (s, 21), 6.68 (d, 2H, J=8.6 Hz), 7.00 (d, 2H, J=8.6 Hz), 7.20-7.22 (m, 3H), 7.23 (s, 2H), 7.58-7.66 (m, 3H), 8.59 (br, 1H), 8.60 (br, 1H).

### EXAMPLE 63

### Synthesis of 4-[N-(4-butylphenyl)-N-[4-(3,4,5-trimethoxyphenyl)benzyl]amino]-1-[[2-(3,4,5-trimethoxyphenyl)pyridin-4-yl]methyl]piperidine Dihydrochloride

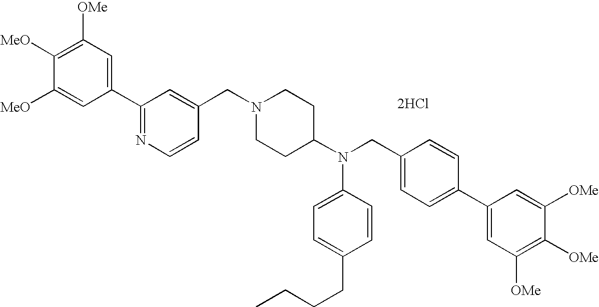

4-(4-Butylanilino)-1-[[2-(3,4,5-trimethoxyphenyl)pyridin-4-yl]methyl]piperidine (147 mg) and 4-(3,4,5-trimethoxyphenyl)benzyl chloride (114 mg) were condensed in the same manner as described in Example 9. The title compound was obtained as yellow powder after converting a free base to a dihydrochloride.

Yield: 82 mg (33%).

¹H-NMR (400 MHz, measured as a free base, CDCl₃)δ: 0.90 (t, 3H, J=7.3 Hz), 1.30-1.36 (m, 2H), 1.51-1.55 (m, 2H), 1.79-1.90 (m, 4H), 2.18 (br, 2H), 2.48 (t, 2H, J=7.7 Hz), 2.98 (d, 2H, J=10.7 Hz), 3.57 (s, 2H), 3.72-3.85 (m, 1H), 3.88 (s, 3H), 3.90 (s, 3H), 3.91 (s, 61), 3.96 (s, 6H), 4.50 (s, 2H), 6.66 (d, 2H, J=8.8 Hz), 6.75 (s, 2H), 7.00 (d, 2H, J=8.8 Hz), 7.20 (d, 1H, J=4.9 Hz), 7.22 (s, 2H), 7.33 (d, 2H, J=8.2 Hz), 7.47 (d, 2H, J=8.2 Hz), 7.61 (s, 1H), 8.59 (d, 1H, J=5.1 Hz).

### PREPARATION EXAMPLE 90

### Synthesis of 1-(4-pycolyl)-4-piperidone

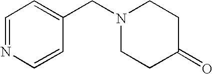

4-piperidone hydrochloride monohydrate (922 mg) and 4-picolyl chloride hydrochloride (820 mg) were reacted in the same manner as described in Example 9 to give the title compound.

Yield: 870 mg (92%).

¹H-NMR (400 MHz, CDCl₃)δ: 2.46 (t, 4H, J=5.9 Hz), 2.74 (t, 4H, J=6.2 Hz), 3.61 (s, 2H), 7.29 (d, 2H, J=6.2 Hz), 8.55 (dd, 2H, J=6.2 Hz, 1.1 Hz).

### PREPARATION EXAMPLE 91

### Synthesis of 1-(4-pycolyl)-4-(4-pycolylamino)piperidine Tetrahydrochloride

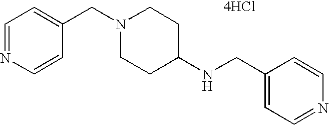

1-(4-pycolyl)-4-piperidone (870 mg) and 4 picolylamine (497 mg) were coupled in the same manner as described in Preparation Example 37. The title compound was obtained as pale brown powder after converting a free base to tetrahydrochloride.

Yield: 363 mg (19%).

¹H-NMR (400 MHz, measured as a free base, CDCl₃)δ: 1.37-1.51 (m, 2H), 1.82-1.90 (m, 2H), 2.04 (dt, 2H, J=11.6 Hz, 2.7 Hz), 2.44-2.55 (m, 1H), 2.76-2.82 (m, 2H), 3.47 (s, 2H), 3.82 (s, 2H), 7.23-7.26 (m, 4H), 8.50-8.53 (m, 4H).

### PREPARATION EXAMPLE 92

### Synthesis or 4-(p-anisidino)-1-(tert-butoxycarbonyl)piperidine

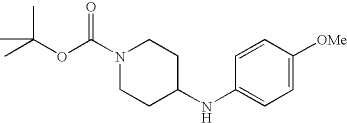

1-(tert-Butoxycarbonyl)-4-piperidone (116 g) and p-anisidine (68.3 g) were condensed in the same manner as described in Preparation Example 37 to give the title compound.

Yield: 125 g (74%).

¹H-NMR (400 MHz, CDCl₃) δ: 1.23-1.35 (m, 2H), 1.46 (s, 9H), 1.96-2.06 (m, 2H), 2.83-2.96 (m, 2H), 3.27-3.38 (m, 1H), 3.74 (s, 9H), 3.94-4.12 (m, 2H), 6.58 (d, 2H, J=9.0 Hz), 6.77 (d, 2H, J=9.0 Hz).

### PREPARATION EXAMPLE 93

### Synthesis of

1-(tert-butoxycarbonyl)-4-[N-(4-methoxyphenyl)-N-[3-(3,4,5-trimethoxyphenyl)benzoylamino]piperidine:

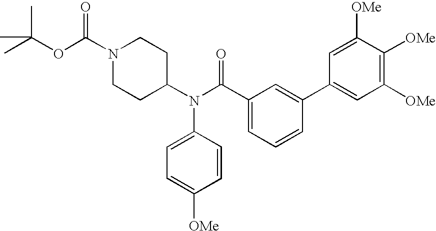

4-(p-Anisidino)-1-(tert-butoxycarbonyl)piperidine (613 mg) and 3-(3,4,5-trimethoxyphenyl)benzoic acid (577 mg) were condensed in the same manner as described in Example 1 to give the title compound.

Yield: 416 mg (36%).

### PREPARATION EXAMPLE 94

### Synthesis of

4-[N-(4-methoxyphenyl)-N-[3-(3,4,5-trimethoxyphenyl)benzoylamino]piperidine Hydrochloride:

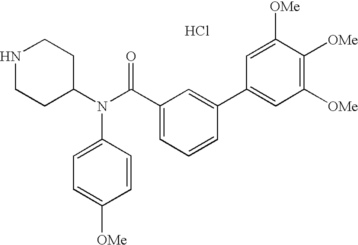

To a solution of 1-(tert-butoxycarbonyl)-4-[N-(4-methoxyphenyl)-N-[3-(3,4,5-trimethoxyphenyl)benzoylamino]piperidine (416 mg) in ethyl acetate (5 mL) was added 4 M hydrogen chloride in ethyl acetate (5 mL). The mixture was stirred at room temperature for 4 hr, resulting precipitates were collected and washed with ethyl acetate on a funnel to give the title compound.

Yield: 315 mg (85%)

### EXAMPLES 64 TO 66

These compounds were prepared by the condensation of 4-[N-(4-methoxyphenyl)-N-[3-(3,4,5-trimethoxyphenyl)benzoylamino]]piperidine hydrochloride with chloride derivatives obtained in Preparation Examples 3, 42 and 48. Free bases obtained were then converted to the corresponding hydrochlorides. Yields and NMR data of their free bases are listed below.

| Ex- | | | NMR data (400 MHz, measured |
|---|---|---|---|
| ample | Structure | Yield | as free bases, CDCl₃) δ |
| 64 | 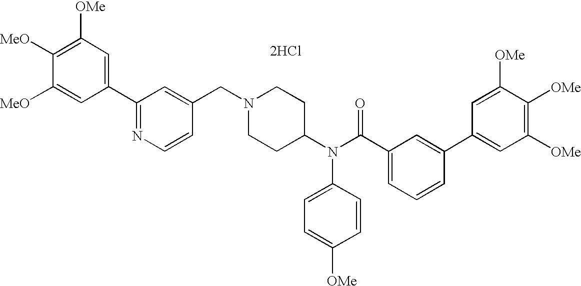 | 68% | 1.53-1.55(m, 2H), 1.89(d, 2H, J=12.0Hz), 2.23(t, 2H, J=12.0Hz), 2.91(d, 2H, J=11.0Hz), 3.51(s, 2H), 3.70(s, 3H), 3.84(s, 3H), 3.87(s, 9H), 3.92(s, 6H), 4.78(br, 1H), 6.54(s, 2H), 6.72(d, 2H, J=8.5Hz), 6.94(d, 2H, J=8.5Hz), 7.13-7.20(m, 4H), 7.18(s, 2H), 7.32(d, 1H, J=5.3Hz), 7.45(s, 1H), 8.19(d, 1H, J=4.9Hz). |
| 65 | 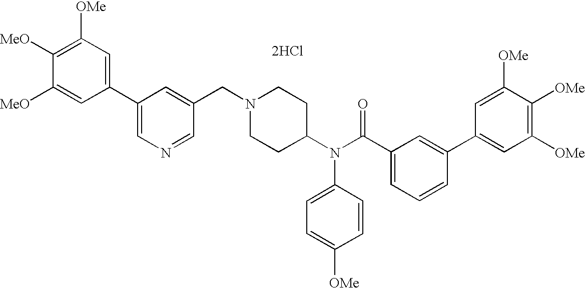 | 52% | 1.66-1.89(m, 4H), 2.05-2.17(m, 2H), 2.97(d, 2H, J=10.3Hz), 3.43-3.60(m, 1H), 3.57(s, 2H), 3.86(s, 3H), 3.87(s, 6H), 3.91(s, 6H), 4.42(s, 2H), 6.63(s, 2H), 6.72-6.79(m, 6H), 7.64(s, 1H), 7.78(br, 1H), 8.46(d, 2H, J=1.6Hz), 8.59(d, 1H, J=2.4Hz), 8.68(d, 1H, J=2.2Hz). |
| 66 | 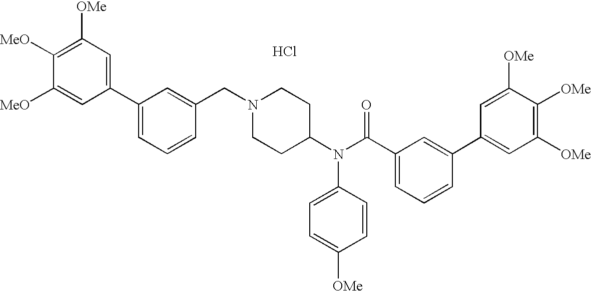 | 75% | 1.42-2.58(m, 2H), 1.85-1.92(m, 2H), 2.14-2.23(m, 2H), 2.93-3.03(m, 2H), 3.56(s, 2H), 3.70(s, 3H), 3.85(s, 3H), 3.87(s, 3H), 3.87(s, 6H), 3.90(s, 6H), 4.79(br, 1H), 6.54(s, 2H), 6.70(d, 2H, J=8.9Hz), 6.74(s, 2H), 6.93(d, 2H, J=8.9Hz), 7.17-7.23(m, 3H), 7.31-7.43(m, 5H). |

### PREPARATION EXAMPLE 95

### Synthesis of 1-(tert-butoxycarbonyl)-4-[N-(4-methoxyphenyl)-N-[[2-(3,4,5-trimethoxyphenyl)pyridin-4-yl]methyl]amino]piperidine

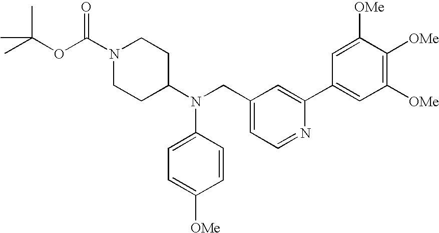

4-(p-Anisidino)-1-(tert-butoxycarbonyl)piperidine (2.21 g) and 4-chloromethyl-2-(3,4,5-trimethoxyphenyl)pyridine (2.12 g) were condensed in the same manner as described in Example 9 to give the title compound.

Yield: 3.76 g (93%)

¹H-NMR (400 MHz, CDCl₃) δ: 1.40-1.64 (m, 2H), 1.44 (s, 9H), 1.82-1.91 (m, 2H), 2.71-2.84 (m, 2H), 3.62-3.73 (m, 1H), 3.74 (s, 3H), 3.89 (s, 3H), 3.94 (s, 6H), 4.10-4.30 (m, 2H), 4.40 (s, 2H), 6.76 (d, 2H, J=9.4 Hz), 6.79 (d, 2H, J=9.8 Hz), 7.14-7.19 (m, 3H), 7.56 (s, 1H), 8.55 (d, 1H, J=5.1 Hz).

### PREPARATION EXAMPLE 96

### Synthesis of

4-[N-(4-methoxyphenyl)-N-[[2-(3,4,5-trimethoxyphenyl)pyridin-4-yl]methyl]amino]piperidine Dihydrochloride:

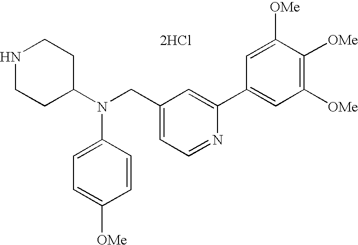

1-(tert-Butoxycarbonyl)-4-[N-(4-methoxyphenyl)-N-[[2-(3,4,5-trimethoxyphenyl)pyridin-4-yl]methyl]amino]piperidine (3.76 g) was treated in the same manner as described in Preparation Example 94 to give the title compound.

Yield: 3.77 g (theoretical yield).

### PREPARATION EXAMPLE 97

### Synthesis of

1-(tert-butoxycarbonyl)-4-[N-(4-methoxyphenyl)-N-[[3-(3,4,5-trimethoxyphenyl)pyridin-5-yl]methyl]amino]piperidine:

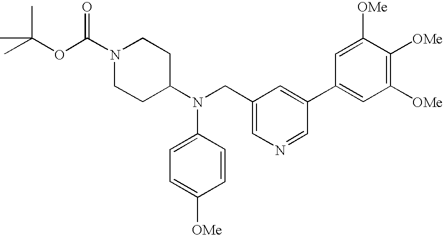

4-p-anisidino)-1-(tert-butoxycarbonyl)piperidine (613 mg) and 5-chloromethyl-3-(3,4,5-trimethoxyphenyl)pyridine (588 mg) was treated in the same manner as described in Preparation Example 9 to give pale yellow amorphous of the title compound.

Yield: 159 mg (14%).

¹H-NMR (400 MHz, CDCl₃) δ: 1.44 (s, 9H), 1.50-1.65 (m, 2H), 1.83-1.91 (m, 2H), 2.70-2.84 (m, 2H), 3.53-3.62 (m, 1H), 3.73 (s, 3H), 3.89 (s, 3H), 3.91 (s, 6H), 4.10-4.29 (m, 2H), 4.41 (s, 2H), 6.66 (s, 2H), 6.76-6.84 (m, 4H), 7.70 (s, 1H), 8.49 (s, 1H), 8.63 (d, 1H, J=2.1 Hz).

### PREPARATION EXAMPLE 98

### Synthesis of

4-[N-(4-methoxyphenyl)-N-[[3-(3,4,5-trimethoxyphenyl)pyridin-5-yl]methyl]amino]piperidine Dihydrochloride:

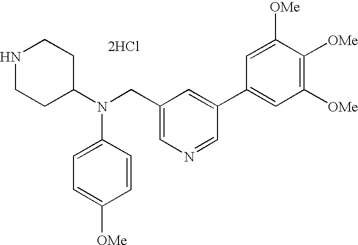

1-(tert-Butoxycarbonyl)-4-[N-(4-methoxyphenyl)-N-[[3-(3,4,5-trimethoxyphenyl)pyridin-5-yl]methyl]amino]piperidine (159 mg) was treated in the same manner as described in Preparation Example 94 to give pale yellow powder of the title compound.

Yield: 142 mg (94%).

### PREPARATION EXAMPLE 99

### Synthesis of

1-(tert-butoxycarbonyl)-4[N-(4-methoxyphenyl)-N-[3-(3,4,5-trimethoxyphenyl)benzyl]amino]piperidine:

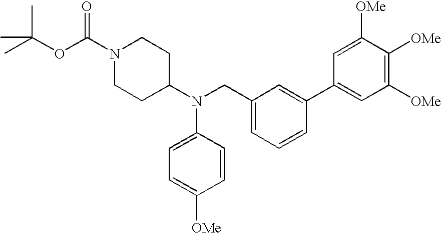

4-(p-Anisidino)-1-(tert-butoxycarbonyl)piperidine (613 mg) and 3-(3,4,5-trimethoxyphenyl)benzyl chloride (586 mg) was treated in the same manner as described in Example 9 to give pale yellow amorphous of the title compound.

Yield: 1.12 g (90%).

¹H-NMR (400 MHz, CDCl₃) δ: 1.44 (s, 9H), 1.50-1.63 (m, 2H), 1.82-1.91 (m, 2H), 2.71-2.83 (m, 2H), 3.69 (tt, 1H, J=11.5 Hz, 3.5 Hz), 3.73 (s, 3H), 3.88 (s, 3H), 3.90 (s, 6H), 4.10-4.28 (m, 2H), 4.42 (s, 2H), 6.71 (s, 2H), 6.78 (s, 4H), 7.24-7.28 (m, 1H), 7.31-7.40 (m, 2H), 7.42 (s, 1H).

### PREPARATION EXAMPLE 100

### Synthesis of

4-[N-(4-methoxyphenyl)-N-[3-(3,4,5-trimethoxyphenyl)benzyl]amino]piperidine Hydrochloride:

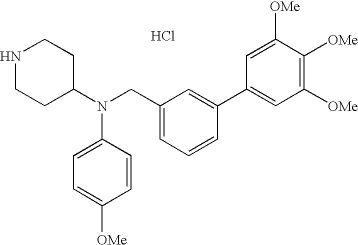

1-(tert-Butoxycarbonyl)-4-[N-(4-methoxyphenyl)-N-[3-(3,4,5-trimethoxyphenyl)benzyl]amino]piperidine (1.12 g) was treated in the same manner as described in Preparation Example 94 to give pale yellow powder of the title compound.

Yield: 980 mg (99%).

### EXAMPLES 67 TO 71

These compounds were obtained by the condensation of amines obtained in Preparation Examples 96, 98 and 100 with chloride derivatives obtained in Preparation Examples 42 and 48. Free bases obtained were then converted to the corresponding hydrochlorides. Yields and NMR data of their free bases are listed below.

| Ex- | | | NMR data (400 MHz, measured |
|---|---|---|---|
| ample | Structure | Yield | as free bases, CDCl₃) δ |
| 67 | 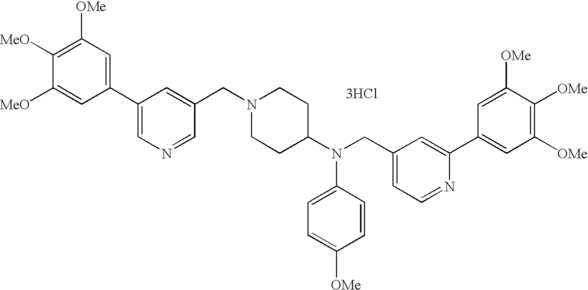 | 62% | 1.60-1.92(m, 4H), 2.08-2.22(m, 2H), 2.92-3.06(m, 2H), 3.54-3.64(m, 3H), 3.73(s, 3H), 3.89(s, 3H), 3.90(s, 3H), 3.93(s, 12H), 4.43(s, 2H), 6.70-6.81(m, 6H), 7.12-7.17(m, 3H), 7.56(s, 1H), 7.76(s, 1H), 8.49(d, 1H, J=1.8Hz), 8.53(d, 1H, J=5.1Hz), 8.70(s, 1H). |
| 68 | 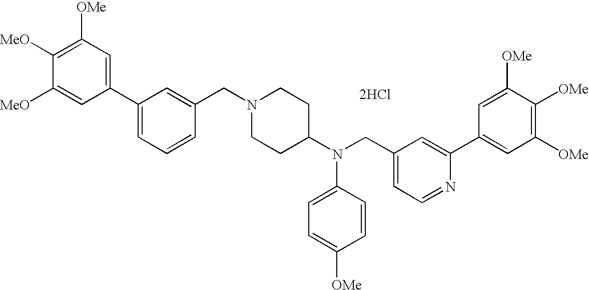 | 54% | 1.65-1.79(m, 2H), 1.81-1.90(m, 2H), 2.04-2.18(m, 2H), 2.94-3.06(m, 2H), 3.52-3.66(m, 3H), 3.72(s, 3H), 3.89(s, 6H), 3.92(s, 6H), 3.93(s, 6H), 4.44(s, 2H), 6.70-6.80(m, 6H), 7.13-7.17(m, 3H), 7.24-7.50(m, 4H), 7.55(s, 1H), 8.53(d, 1H, J=4.9Hz). |
| 69 | 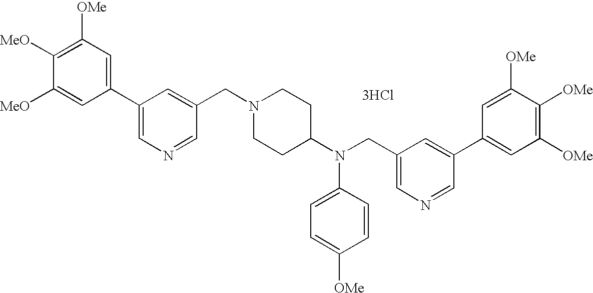 | 52% | 1.66-1.89(m, 4H), 2.05-2.17(m, 2H), 2.97(d, 2H, J=10.3Hz), 3.43-3.60(m, 1H), 3.57(s, 2H), 3.86(s, 3H), 3.87(s, 6H), 3.91(s, 6H), 4.42(s, 2H), 6.63(s, 2H), 6.72-6.79(m, 6H), 7.64(s, 1H), 7.78(br, 1H), 8.46(d, 2H, J=1.6Hz), 8.59(d, 1H, J=2.24Hz), 8.68(d, 1H, J=2.2Hz). |
| 70 | 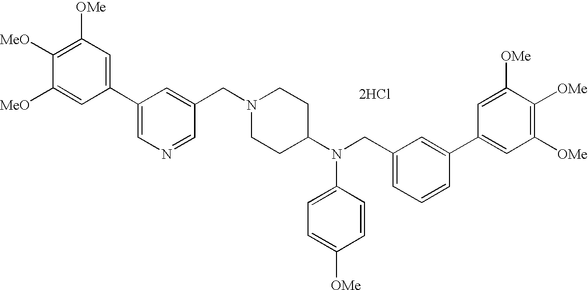 | 69% | 1.55-1.97(m, 4H), 2.06-2.21(m, 2H), 2.92-3.07(m, 2H), 3.53-3.68(m, 3H), 3.72(s, 3H), 3.87(s, 3H), 3.89(s, 6H), 3.94(s, 3H), 4.46(s, 2H), 6.69(s, 2H), 6.73-6.82(m, 6H), 7.22-7.29(m, 1H), 7.32(t, 1H, J=7.4Hz), 7.36(d, 1H, J=7.8Hz), 7.41(s, 1H), 7.79(br, 1H), 8.48(s, 1H), 8.71(br, 1H). |
| 71 | 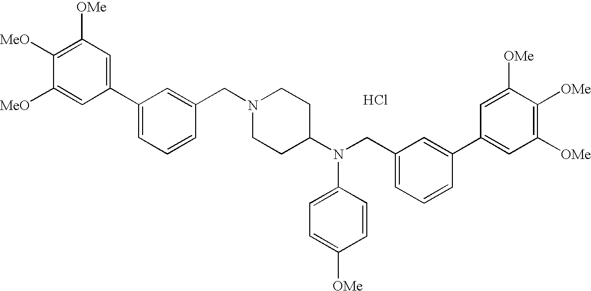 | 75% | 1.69-1.89(m, 4H), 2.06-2.15(m, 2H), 2.96-3.04(m, 2H), 3.56-3.66(m, 1H), 3.57(s, 2H), 3.72(s, 3H), 3.87(s, 3H), 3.89(s, 9H), 3.92(s, 6H), 4.46(s, 2H), 6.70(s, 2H), 6.71-6.79(m, 6H), 7.23-7.47(m, 8H). |

### PREPARATION EXAMPLE 101

### Synthesis of 1-(tert-butoxycarbonyl)-4-(4-ethoxyphenylamino)piperidine

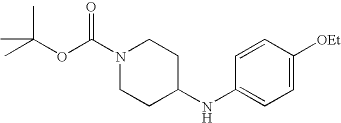

1-(tert-butoxycarbonyl)-4-piperidinone (5.00 g) and p-phenetidine (3.28 g) was treated in the same manner as described in Preparation Example 37 to give brown powder of the title compound.

Yield: 7.00 g (910%).

¹H-NMR (400 MHz, CDCl₃) δ: 1.21-1.31 (m, 2H), 1.37 (t, 3H, J=7.0 Hz), 1.46 (s, 9H), 1.97-2.05 (m, 2H), 2.84-2.95 (m, 2H), 3.28-3.37 (m, 1H), 3.96 (q, 2H, J=7.0 Hz), 3.99-4.10 (m, 2H), 6.57 (d, 2H, J=8.8 Hz), 6.77 (d, 2H, J=9.0 Hz).

### PREPARATION EXAMPLE 102

### Synthesis of 1-(tert-butoxycarbonyl)-4-[N-(4-ethoxyphenyl)-N-[[2-(3,4,5-trimethoxyphenyl)pyridin-4-yl]methyl]amino]piperidine

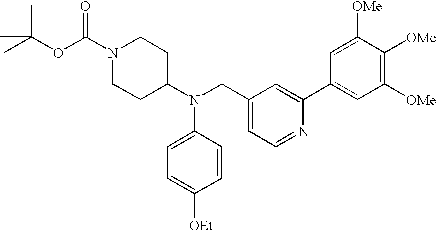

1-(tert-Butoxycarbonyl)-4-[(4-ethoxyphenyl)amino]piperidine (641 mg) and 4-chloromethyl-2-(3,4,5-trimethoxyphenyl)pyridine (588 mg) was treated in the same manner as described in Example 9 to give light yellow amorphous of the title compound.

Yield: 1.08 g (94%).

¹H-NMR (400 MHz, CDCl₃) δ: 1.36 (t, 3H, J=7.9 Hz), 1.44 (s, 9H), 1.49-1.58 (m, 2H), 1.82-1.92 (m, 2H), 2.70-2.85 (m, 2H), 3.62-3.72 (m, 1H), 3.89 (s, 3H), 3.94 (s, 6H), 4.12-4.29 (m, 2H), 4.39 (s, 2H), 6.75 (d, 2H, J=9.2 Hz), 6.78 (d, 2H, J=9.6 Hz), 7.14-7.18 (m, 3H), 7.55 (s, 1H), 8.54 (d, 1H, J=5.1 Hz).

### PREPARATION EXAMPLE 103

### Synthesis of

4-[N-(4-ethoxyphenyl)-N-[[2-(3,4,5-trimethoxyphenyl)pyridin-4-yl]methyl]amino]piperidine Dihydrochloride:

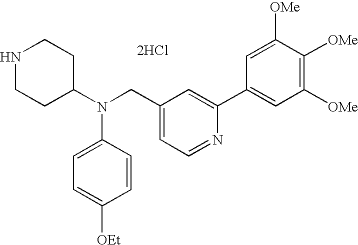

1-(tert-Butoxycarbonyl)-4-[N-(4-ethoxyphenyl)-N-[[2-(3,4,5-trimethoxyphenyl)pyridin-4-yl]methyl]amino]piperidine (1.08 g) was treated in the same manner as described in Preparation Example 94 to give light yellow powder of the title compound.

Yield: 1.01 g (98%).

### PREPARATION EXAMPLE 104

### Synthesis of 1-(tert-butoxycarbonyl)-4-[N-(4-ethoxyphenyl)-N-[[3-(3,4,5-trimethoxyphenyl)pyridin-5-yl]methyl]amino]piperidine

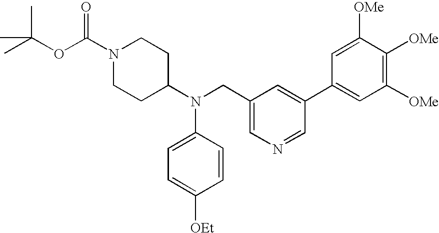

1-(tert-Butoxycarbonyl)-4-[(4-ethoxyphenyl)amino]piperidine (641 mg) and 5-chloromethyl-3-(3,4,5-trimethoxyphenyl)pyridine (588 mg) was treated in the same manner as described in Example 9 to give light yellow amorphous of the title compound.

Yield: 452 mg (39%).

¹H-NMR (400 MHz, CDCl₃) δ: 1.36 (t, 3H, J=6.8 Hz), 1.44 (s, 9H), 1.50-1.60 (m, 2H), 1.82-1.90 (m, 1H), 2.68-2.82 (m, 2H), 3.52-3.61 (m, 1H), 3.88 (s, 3H), 3.90 (s, 6H), 3.94 (q, 2H, J=7.0 Hz), 4.10-4.25 (m, 2H), 4.40 (s, 2H), 6.66 (s, 2H), 6.77 (d, 2H, J=9.2 Hz), 6.81 (d, 2H, J=9.2 Hz), 7.67 (s, 1H), 8.49 (d, 1H, J=2.0 Hz), 8.62 (d, 1H, J=2.1 Hz).

### PREPARATION EXAMPLE 105

### Synthesis of

4-[N-(4-ethoxyphenyl)-N-[[3-(3,4,5-trimethoxyphenyl)pyridin-5-yl]methyl]amino]piperidine Dihydrochloride:

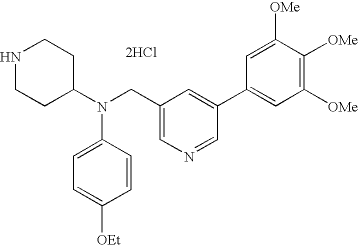

1-(tert-Butoxycarbonyl)-4-[N-(4-ethoxyphenyl)-N-[[3-(3,4,5-trimethoxyphenyl)pyridin-5-yl]methyl]amino]piperidine (452 mg) was treated in the same manner as described in Preparation Example 94 to give light yellow powder of the title compound.

Yield: 380 mg (88%).

### PREPARATION EXAMPLE 106

### Synthesis of 1-(tert-butoxycarbonyl)-4-[N-(4-ethoxyphenyl)-N-[3-(3,4,5-trimethoxyphenyl)benzyl]amino]piperidine

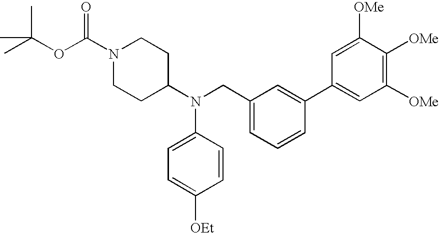

1-(tert-Butoxycarbonyl)-4-[(4-ethoxyphenyl)amino]piperidine (641 mg) and 3-(3,4,5-trimethoxyphenyl)benzyl chloride (586 mg) was treated in the same manner as described in Example 9 to give light yellow amorphous of the title compound.

Yield: 1.06 g (92%).

¹H-NMR (400 MHz, CDCl₃) δ: 1.36 (t, 3H, J=7.0 Hz), 1.44 (s, 9H), 1.53-1.59 (m, 2H), 1.83-1.91 (m, 2H), 2.70-2.83 (m, 2H), 3.64-3.73 (m, 1H), 3.88 (s, 3H), 3.90 (s, 6H), 3.94 (q, 2H, J=7.0 Hz), 4.10-4.29 (m, 2H), 4.41 (s, 2H), 6.71 (s, 2H), 6.76 (s, 4H), 7.26 (d, 1H, J=7.9 Hz), 7.33 (dd, 1H, J=7.4 Hz, 7.4 Hz), 7.38 (d, 1H, J=7.6 Hz), 7.42 (s, 1H).

### PREPARATION EXAMPLE 107

### Synthesis of

4-[N-(4-ethoxyphenyl)-N-[3-(3,4,5-trimethoxyphenyl)benzyl]amino]piperidine Hydrochloride:

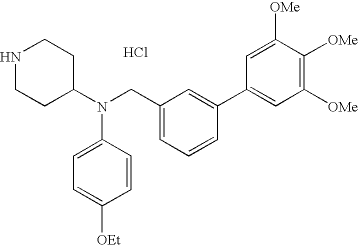

1-(tert-Butoxycarbonyl)-4>[N-(4-ethoxyphenyl)-N-[3-(3,4,5-trimethoxyphenyl)benzyl]amino]piperidine (1.06 g) was treated in the same manner as described in Preparation Example 94 to give light yellow powder of the title compound.

Yield: 913 mg (97%).

### EXAMPLES 72 TO 79

These compounds were obtained by the condensation of amines obtained in Preparation Examples 103, 105 and 107 with chloride derivatives obtained in Preparation Examples 3, 42 and 48. Free bases obtained were then converted to the corresponding hydrochlorides. Yields and NMR data of their free bases are listed below.

| Ex- | | | NMR data (400 MHz, measured as |
|---|---|---|---|
| ample | Structure | Yield | free bases, CDCl₃) δ |
| 72 | 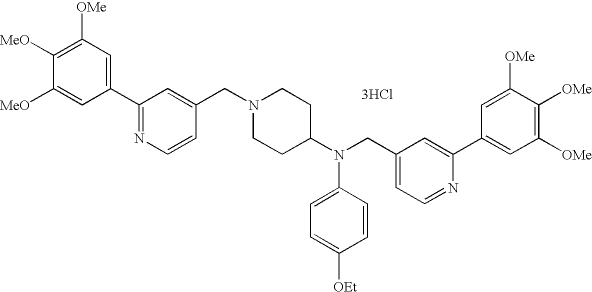 | 49% | 1.36(t, 3H, J=7.1Hz), 1.68-1.94(m, 4H), 2.10-2.24(m, 2H), 2.93-3.04(m, 2H), 3.54-3.65(m, 3H), 3.89(s, 3H), 3.90(s, 3H), 3.93(s, 6H), 3.96(s, 6H), 4.45(s, 2H), 6.72(d, 2H, J=9.2Hz), 6.78(d, 2H, J=9.3Hz), 7.15(s, 2H), 7.17(d, 1H, J=6.1Hz), 7.20(dd, 1H, J=4.9Hz, 1.0Hz), 7.23(s, 2H), 7.57(s, 1H), |
| #7.61(br, 1H), 8.54(d, 1H, J=5.2Hz), 8.59(d, 1H, J=4.9Hz). | | | |
| 73 | 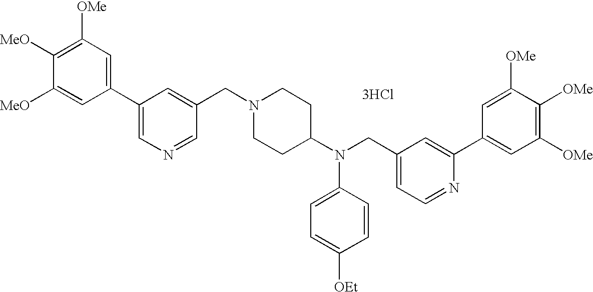 | 63% | 1.36(t, 3H, J=7.0Hz), 1.56-1.74(m, 2H), 1.80-1.90(m, 2H), 2.07-2.19(m, 2H), 2.92-3.02(m, 2H), 3.58(s, 2H), 3.88-3.95(m, 2H), 3.89(s, 3H), 3.93(s, 12H), 4.43(s, 2H), 6.69-6.79(m, 6H), 7.12-7.17(m, 3H), 7.55(s, 1H), 7.76(s, 1H), 8.49(d, 1H, J=1.8Hz), 8.53(d, 1H, J=5.1Hz), 8.69(s, 1H). |
| 74 | 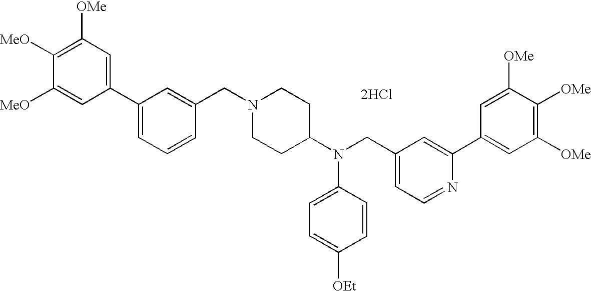 | 65% | 1.36(t, 3H, J=7.0Hz), 1.58-1.78(m, 2H), 1.80-1.89(m, 2H), 2.04-2.16(m, 2H), 2.95-3.05(m, 2H), 3.52-3.66(m, 1H), 3.57(s, 1H), 3.85-3.97(m, 2H), 3.89(s, 6H), 3.92(s, 6H), 3.93(s, 6H), 4.44(s, 2H), 6.67-6.80(m, 6H), 7.13-7.18(m, 3H), 7.25-7.31(m, 1H), 7.37(dd, 1H, J=7.6Hz, 7.6Hz), 7.41-7.48(m, |
| #2H), 7.55(s, 1H), 8.53(d, 1H, J=4.9Hz). | | | |
| 75 | 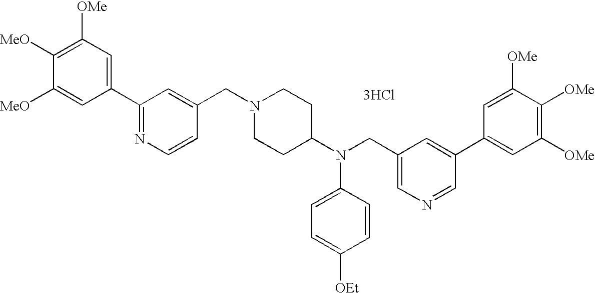 | 42% | 1.36(t, 3H, J=7.0Hz), 1.74-2.34(m, 6H), 2.96-3.10(m, 2H), 3.47-3.73(m, 3H), 3.87-3.98(m, 2H), 3.88(s, 3H), 3.90(s, 9H), 3.97(s, 6H), 4.44(s, 2H), 6.65(s, 2H), 6.74-6.82(m, 4H), 7.18-7.32(m, 4H), 7.67(s, 1H), 8.49(d, 1H, J=1.6Hz), 8.57-8.65(m, 2H). |
| 76 | 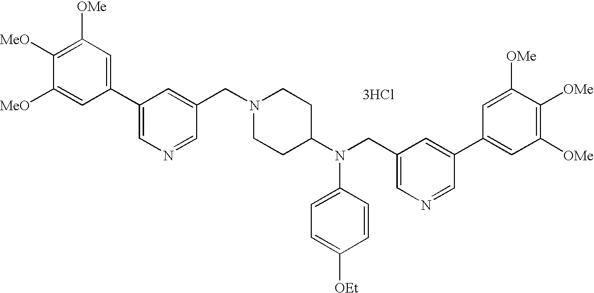 | 43% | 1.36(t, 3H, J=6.8Hz), 1.63-1.96(m, 4h), 2.00-2.26(m, 2H), 2.92-3.03(m, 2H), 3.44-3.66(m, 3H), 3.86-3.96(m, 2H), 3.88(s, 3H), 3.89(s, 6H), 3.90(s, 3H), 3.93(s, 6H), 4.44(s, 2H), 6.65(s, 2H), 6.72-6.80(m, 6H), 7.67(s, 1H), 7.77(br, 1H), 8.47-8.53(m, 2H), 8.62(d, 1H, J=1.9Hz), 8.70(s, 1H). |
| 77 | 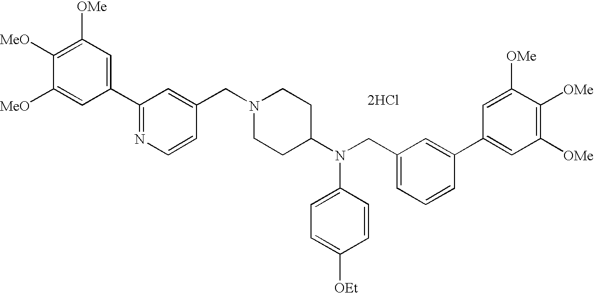 | 82% | 1.35(t, 3H, J=6.8Hz), 1.70-1.82(m, 2H), 1.84-1.92(m, 2H), 2.10-2.19(m, 2H), 2.92-3.00(m, 2H), 3.52-3.65(m, 3H), 3.88(s, 3H), 3.89(s, 6H), 3.90(s, 3H), 3.93(q, 2H, J=7.1Hz), 3.96(s, 6H), 4.47(s, 2H), 6.70(s, 2H), 6.73(d, 2H, J=9.3Hz), 6.77(d, 2H, J=9.3Hz), 7.18-7.28(m, 4H), 7.33(dd, 1H, |
| #J=7.3Hz, 7.3Hz), 7.37(d, 1H, J=7.6Hz), 7.43(s, 1H), 7.59(s, 1H), 8.58(d, 1H, J=4.9Hz). | | | |
| 78 | 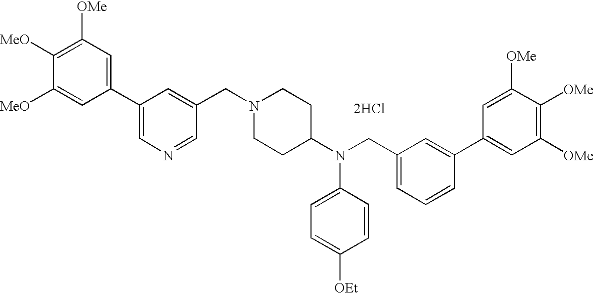 | 61% | 1.35(t, 3H, J=6.9Hz), 15.8-1.80(m, 2H), 1.82-1.91(m, 2H), 2.09-2.18(m, 2H), 2.93-3.20(m, 2H), 3.56-3.65(m, 1H), 3.58(s, 2H), 3.87(s, 3H), 3.89(s, 6H), 3.89(s, 3H), 3.91-3.94(m, 2H), 3.93(s, 6H), 4.45(s, 2H), 6.69(s, 2H), 6.71-6.78(m, 6H), 7.23-7.28(m, 1H), 7.32(t, 1H, J=7.5Hz), 7.36(d, |
| #1H, J=7.6Hz), 7.42(s, 1H), 7.77(s, 1H), 8.49(d, 1H, J=1.8Hz), 8.69(d, 1H, J=1.8Hz). | | | |
| 79 | 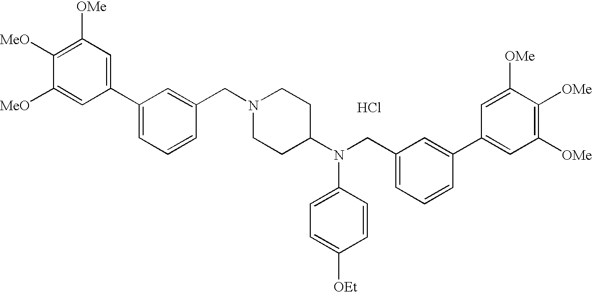 | 73% | 1.35(t, 3H, J=6.8Hz), 1.68-1.80(m, 2H), 1.81-1.89(m, 2H), 2.06-2.14(m, 2H), 2.96-3.03(m, 2H), 3.57(s, 2H), 3.57-3.65(m, 1H), 3.87(s, 3H), 3.89(s, 9H), 3.91-3.96(m, 2H), 3.92(s, 6H), 4.46(s, 2H), 6.69-6.79(m, 9H), 7.23-7.47(m, 7H). |

### PREPARATION EXAMPLE 108

### Synthesis of 1-(tert-butoxycarbonyl)-4-(4-butoxyphenylamino)piperidine

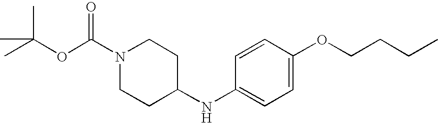

1-(tert-butoxycarbonyl)-4-piperidone (5.00 g) and 4-butoxyaniline (3.95 g) was treated in the same manner as described in Preparation Example 37 to give brown powder of the title compound.

Yield: 6.91 g (83%).

¹H-NMR (400 MHz, CDCl₃) δ: 0.96 (t, 3H, J=7.2 Hz), 1.23-1.35 (m, 2H), 1.42-1.53 (m, 2H), 1.46 (s, 9H), 1.68-1.76 (m, 2H), 1.97-2.05 (m, 2H), 2.84-2.95 (m, 2H), 3.28-3.37 (m, 1H), 3.88 (t, 2H, J=6.6 Hz), 3.96-4.12 (m, 2H), 6.57 (d, 2H, J=9.0 Hz), 6.77 (d, 2H, J=8.8 Hz).

### PREPARATION EXAMPLE 109

### Synthesis of

1-(tert-butoxycarbonyl)-4[N-(4-butoxyphenyl)-N-[[2-(3,4,5-trimethoxyphenyl)pyridin-4-yl]methyl]amino]piperidine:

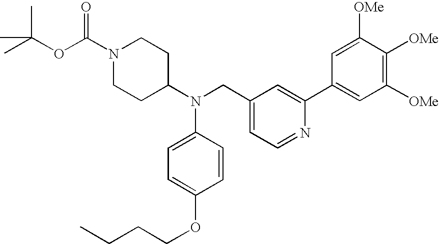

1-(tert-Butoxycarbonyl)-4-[(4-butoxyphenyl)amino]piperidine (696 mg) and 4-chloromethyl-2-(3,4,5-trimethoxyphenyl)pyridine (588 mg) was treated in the same manner as described in Example 9 to give light yellow amorphous of the title compound.

Yield: 980 mg (81%)

¹H-NMR (400 MHz, CDCl₃) δ: 0.95 (t, 3H, J=7.4 Hz), 1.40-1.50 (m, 2H), 1.44 (s, 9H), 1.67-1.76 (m, 2H), 1.82-1.90 (m, 2H), 1.82-1.90 (m, 2H), 2.70-2.82 (m, 2H), 3.61-3.71 (m, 1H), 3.84-3.90 (m, 5H), 3.94 (s, 6H), 4.10-4.28 (m, 2H), 4.39 (s, 2H), 6.74 (d, 2H, J=9.4 Hz), 6.78 (d, 2H, J=9.4 Hz), 7.14-7.18 (m, 3H), 7.56 (s, 1H), 8.54 (d, 1H, J=5.1 Hz).

### PREPARATION EXAMPLE 110

### Synthesis of

4-[N-(4-butoxyphenyl)-N-[[2-(3,4,5-trimethoxyphenyl)pyridin-4-yl]methyl]amino]piperidine Dihydrochloride:

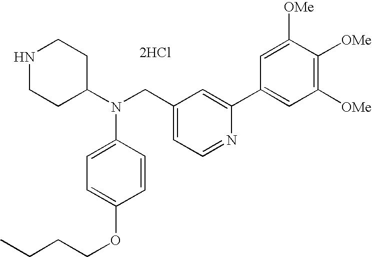

1-(tert-Butoxycarbonyl)-4-[N-(4-butoxyphenyl)-N-[[2-(3,4,5-trimethoxyphenyl)pyridinyl]methyl]amino]piperidine (980 mg) was treated in the same manner as described in Preparation Example 94 to give light yellow powder of the title compound.

Yield: 926 mg (99%).

### PREPARATION EXAMPLE 111

### Synthesis of

1-(tert-butoxycarbonyl)-4-[N-(4-butoxyphenyl)-N-[[3-(3,4,5-trimethoxyphenyl)pyridin-5-yl]methyl]amino]piperidine:

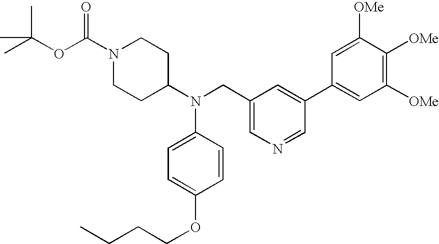

1-(tert-Butoxycarbonyl)-4-[(4-butoxyphenyl)amino]piperidine (697 mg) and 5-chloromethyl-3-(3,4,5-trimethoxyphenyl)pyridine (588 mg) was treated in the same manner as described in Example 9 to give light yellow amorphous of the title compound.

Yield: 485 mg (40%).

¹H-NMR (400 MHz, CDCl₃) δ: 0.95 (t, 3H, J=7.4 Hz), 1.40-1.57 (m, 2H), 1.44 (s, 9H), 1.67-1.75 (m, 2H), 1.82-1.90 (m, 2H), 2.69-2.81 (m, 2H), 3.51-3.60 (m, 1H), 3.87 (q, 2H, J=6.6 Hz), 3.88 (s, 3H), 3.90 (s, 6H), 4.06-4.23 (m, 2H), 4.39 (s, 2H), 6.66 (s, 2H), 6.77 (d, 2H, J=9.2 Hz), 6.81 (d, 2H, J=9.2 Hz), 6.81 (d, 2H, J=9.4 Hz), 7.67 (s, 1H), 8.49 (d, 1H, J=1.8 Hz), 8.62 (d, 1H, J=2.2 Hz).

### PREPARATION EXAMPLE 112

### Synthesis of

4-[N-(4-butoxyphenyl)-N-[[3-(3,4,5-trimethoxyphenyl)pyridin-5-yl]methyl]amino]piperidine Dihydrochloride:

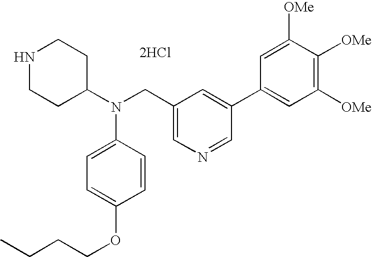

1 tert-Butoxycarbonyl)-4-[N-(4-butoxyphenyl)-N-[[3-(3,4,5-trimethoxyphenyl)pyridin-5-yl]methyl]amino]piperidine (485 mg) was treated in the same manner as described in Preparation Example 94 to give light yellow powder of the title compound.

Yield: 456 mg (98%).

### PREPARATION EXAMPLE 113

### Synthesis of

1-(tert-butoxycarbonyl)-4-[N-(4-butoxyphenyl)-N-[3-(3,4,5-trimethoxyphenyl)benzyl]amino]piperidine:

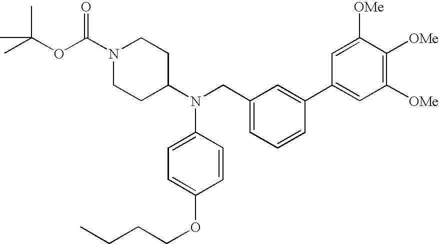

1-(tert-Butoxycarbonyl)-4-[(4-butoxyphenyl)amino]piperidine (697 mg) and 3-(3,4,5-trimethoxyphenyl)benzyl chloride (586 mg) was treated in the same manner as described in Example 9 to give light yellow amorphous of the title compound.

Yield: 1.17 g (97%).

¹H-NMR (400 MHz, CDCl₃) δ: 0.95 (t, 3H, J=7.3 Hz), 1.40-1.61 (m, 4H), 1.44 (s, 9H), 1.67-1.75 (m, 2H), 1.83-1.90 (m, 2H), 2.70-2.83 (m, 2H), 3.63-3.72 (m, 2H), 3.87 (q, 2H, J=6.6 Hz), 3.88 (s, 3H), 3.90 (s, 6H), 4.09-4.28 (m, 2H), 4.41 (s, 2H), 6.70 (s, 2H), 6.76 (s, 4H), 7.26 (d, 2H, J=8.0 Hz), 7.33 (t, 1H, J=7.6 Hz), 7.38 (d, 1H, J=7.3 Hz), 7.42 (s, 1H).

### PREPARATION EXAMPLE 114

### Synthesis of

4-[N-(4-butoxyphenyl)-N-[3-(3,4,5-trimethoxyphenyl)benzyl]amino]piperidine Hydrochloride:

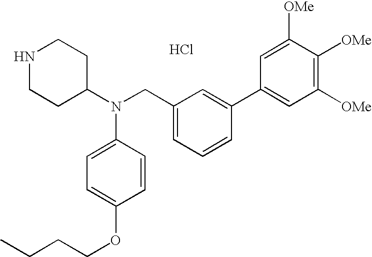

1-(tert-Butoxycarbonyl)-4-[N-(4-butoxyphenyl)-N-[3-(3,4,5-trimethoxyphenyl)benzyl]amino]piperidine (1.17 g) was treated in the same manner as described in Preparation Example 94 to give light yellow powder of the title compound.

Yield: 1.02 g (98%).

### EXAMPLE 80 to 87

These compounds were obtained by the condensation of amines obtained in Preparation Examples 110, 112 and 114 with chloride derivatives obtained in Preparation Examples 3, 42 and 48. Free bases obtained were then converted to the corresponding hydrochlorides. Yields and NMR data of their free bases are listed below.

| Ex- | | | NMR data (400 MHz, measured as |
|---|---|---|---|
| ample | Structure | Yield | free bases, CDCl₃) δ |
| 80 | 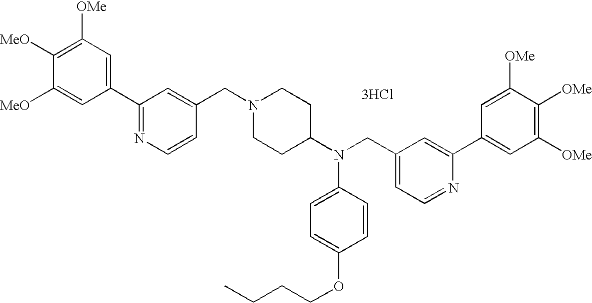 | 63 | 0.95(t, 3H, J=7.3Hz), 1.40-1.51(m, 2H), 1.66-1.79(m, 2H), 1.83-1.92(m, 2H), 2.10-2.21(m, 2H), 2.92-3.02(m, 2H), 3.53-3.63(m, 3H), 3.84-3.90(m, 2H), 3.89(s, 3H), 3.93(s, 6H), 3.96(s, 6H), 6.72(d, 2H, J=9.3Hz), 6.77(d, 2H, J=9.3Hz), 7.15(s, 2H), 7.17(d, 1H, J=5.1Hz), 7.20(d, 1H, J=6.1Hz), |
| #7.22(s, 2H), 7.57(s, 1H), 7.59(s, 1H), 8.54(d, 1H, J=4.9Hz), 8.59(d, 1H, J=5.1Hz). | | | |
| 81 | 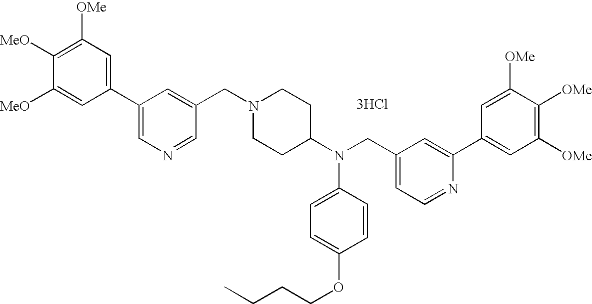 | 44% | 0.95(t, 3H, J=7.4Hz), 1.42-1.51(m, 2H), 1.67-1.76(m, 4H), 1.80-1.91(m, 2H), 2.08-2.20(m, 2H), 2.92-3.03(m, 2H), 3.84-3.96(m, 3H), 3.89(s, 3H), 3.90(s, 3H), 3.93(s, 12H), 4.43(s, 2H), 6.69-6.79(m, 6H), 7.14(s, 2H), 7.16(d, 1H, J=5.2Hz), 7.55(s, 1H), 7.76(s, 1H), 8.49(d, 1H, J=1.8Hz), 8.53(d, 1H, |
| #J=5.0Hz), 8.69(s, 1H). | | | |
| 82 | 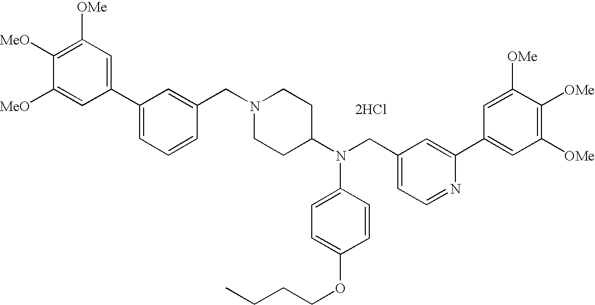 | 53% | 0.95(t, 3H, J=7.2Hz), 1.40-1.51(m, 2H), 1.65-1.78(m, 4H), 1.81-1.89(m, 2H), 2.05-2.18(m, 2H), 3.05-3.06(m, 2H), 3.54-3.65(m, 3H), 3.84-3.96(m, 20H), 4.44(s, 2H), 6.70(d, 2H, J=9.2Hz), 6.74-6.80(m, 4H), 7.11-7.19(m, 3H), 7.22-7.32(m, 1H), 7.34-7.50(m, 3H), 7.55(s, 1H), 8.53(d, |
| #1H, J=5.1Hz). | | | |
| 83 | 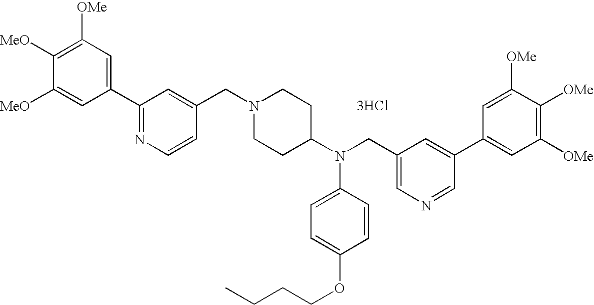 | 42% | 0.95(t, 3H, 7.4Hz), 1.40-1.51(m, 2H), 1.67-1.86(m, 6H), 2.03-2.30(m, 2H), 2.92-3.06(m, 2H), 3.46-3.56(m, 1H), 3.60(s, 2H), 3.84-3.91(m, 2H), 3.88(s, 3H), 3.89(s, 6H), 3.90(s, 3H), 3.96(s, 6H), 4.44(s, 2H), 6.65(s, 2H), 6.74-6.81(m, 4H), 7.20(d, 1H, J=4.9Hz), 7.25(s, 2H), 7.67(br, 2H), 8.50(d, |
| #1H, J=1.6Hz), 8.60(d, 1H, J=5.6Hz). | | | |
| 84 | 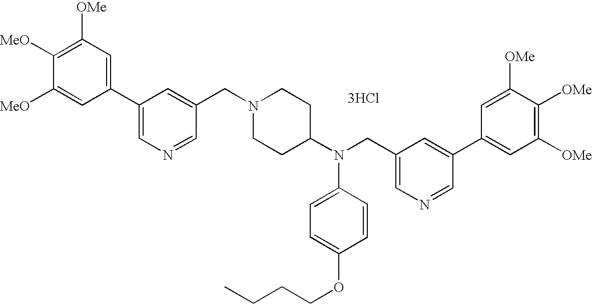 | 36% | 0.95(t, 3H, J=7.4Hz), 1.40-1.51(m, 2H), 1.66-1.79(m, 4H), 1.82-1.92(m, 2H), 2.00-2.22(m, 2H), 2.83-3.06(m, 2H), 3.44-3.67(m, 3H), 3.82-3.97(m, 2H), 3.88(s, 3H), 3.89(s, 6H), 3.90(s, 3H), 3.93(s, 6H), 4.44(s, 2H), 6.65(s, 2H), 6.72-6.80(m, 6H), 7.67(s, 1H), 7.76(br, 1H), 8.47-8.53(m, 2H), 8.62(d, 1H, |
| #J=2.2Hz), 8.70(s, 1H). | | | |
| 85 | 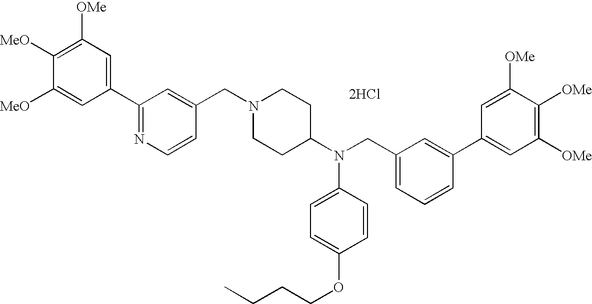 | 72% | 0.95(t, 3H, J=7.3Hz), 1.40-1.51(m, 2H), 1.66-1.82(m, 4H), 1.84-1.92(m, 2H), 2.10-2.20(m, 2H), 2.92-3.00(m, 2H), 3.53-3.66(m, 3H), 3.83-3.92(m, 2H), 3.88(s, 3H), 3.89(s, 6H), 3.90(s, 3H), 3.96(s, 6H), 4.47(s, 2H), 6.67(s, 2H), 6.73(d, 2H, J=9.2Hz), 6.77(d, 2H, J=9.5Hz), 7.18-7.29(m, 4H), 7.33(dd, |
| #1H, J=7.3Hz, 7.3Hz), 7.37(d, 1H, J=7.6Hz), 7.43(s, 1H), 7.60(s, 1H), 8.58(d, 1H, J=4.9Hz). | | | |
| 86 | 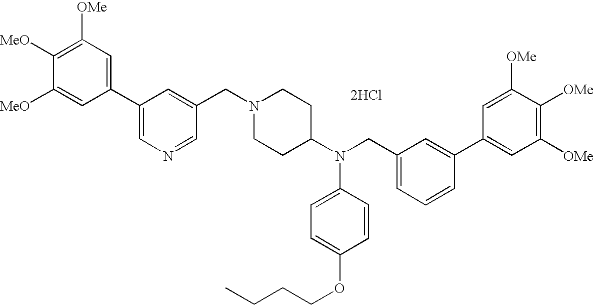 | 24% | 0.94(t, 3H, J=7.4Hz), 1.41-1.51(m, 2H), 1.61-1.80(m, 4H), 1.82-1.92(m, 2H), 2.08-2.19(m, 2H), 2.92-3.02(m, 2H), 3.55-3.65(m, 1H), 3.57(s, 2H), 3.84-3.91(m, 2H), 3.87(s, 3H), 3.88(s, 6H), 3.89(s, 3H), 3.93(s, 6H), 4.45(s, 2H), 6.69(s, 2H), 6.71-6.78(m, 4H), 6.75(s, 2H), 7.23-7.28(m, 1H), 7.32(t, |
| #1H, J=7.4Hz), 7.36(d, 1H, J=7.6Hz), 7.42(s, 1H), 7.77(s, 1H), 8.49(d, 1H, J=1.6Hz), 8.69(s, 1H). | | | |
| 87 | 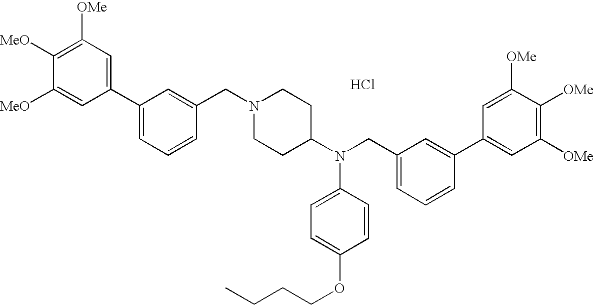 | 78% | 0.94(t, 3H, J=7.3Hz), 1.40-1.50(m, 2H), 1.66-1.88(m, 4H), 1.82-1.89(m, 2H), 2.04-2.16(m, 2H), 2.96-3.03(m, 2H), 3.55-3.65(m, 3H), 3.83-3.90(m, 2H), 3.87(s, 3H), 3.89(s, 9H), 3.92(s, 6H), 4.46(s, 2H), 6.69-6.79(m, 9H), 7.23-7.48(m, 7H). |

### PREPARATION EXAMPLE 115

### Synthesis of 4-(m-anisidino)-1-(tert-butoxycarbonyl)piperidine

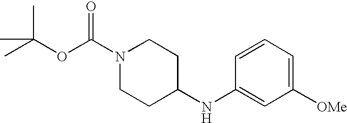

1-(tert-Butoxycarbonyl)-4-piperidone (4.78 g) and m-anisidine (2.96 g) were condensed in the same manner as described in Preparation Example 37 to give the title compound.

Yield: 4.83 g (66%).

¹H-NMR (400 MHz, CDCl₃) δ: 1.20-1.39 (m, 2H), 1.44 (s, 9H), 1.99-2.05 (m, 2H), 2.89 (dt, 2H, J=13.5 Hz, 2.2 Hz), 3.33-3.44 (m, 1H), 3.75 (s, 3H), 3.96-4.07 (m, 2H), 6.14 (t, 1H, J=2.2 Hz), 6.18-6.29 (m, 2H), 7.05 (t, 1H, J=8.1 Hz).

### PREPARATION EXAMPLE 116

### Synthesis of

1-(tert-butoxycarbonyl)-4-[N-(3-methoxyphenyl)-N-[[2-(3,4,5-trimethoxyphenyl)pyridin-4-yl]methyl]amino]piperidine:

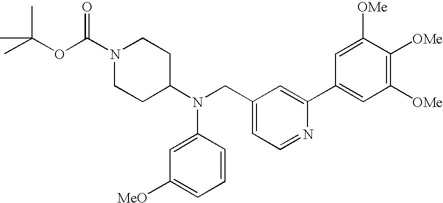

4-(m-Anisidino)-1-(tert-butoxycarbonyl)piperidine (613 mg) and 4-chloromethyl-2-(3,4,5-trimethoxyphenyl)pyridine (588 mg) was treated in the same manner as described in Example 9 to give light yellow amorphous of the title compound.

Yield: 789 mg (70%).

¹H-NMR (400 MHz, CDCl₃) δ: 1.45 (s, 9H), 1.50-1.67 (m, 2H), 1.82-1.91 (m, 2H), 2.74-2.87 (m, 2H), 3.74 (s, 3H), 3.88-3.98 (m, 1H), 3.89 (s, 3H), 3.94 (s, 6H), 4.14-4.32 (m, 2H), 4.48 (s, 2H), 6.28 (dd, 1H, J=2.2 Hz, 2.2 Hz), 6.31-6.37 (m, 2H), 7.10-7.15 (m, 2H), 7.16 (s, 2H), 7.55 (s, 1H), 8.56 (d, 1H, J=5.1 Hz).

### PREPARATION EXAMPLE 117

### Synthesis of 4-[N-(3-methoxyphenyl)-N-[[2-(3,4,5-trimethoxyphenyl)pyridin-4-yl]methyl]amino]piperidine Dihydrochloride:

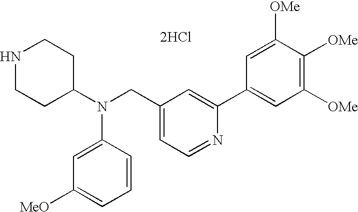

1-(tert-Butoxycarbonyl)-4-[N-(3-methoxyphenyl)-N-[[2-(3,4,5-trimethoxyphenyl)pyridin-4-yl]methyl]amino]piperidine (789 mg) was treated in the same manner as described in Preparation Example 94 to give light yellow powder of the title compound.

Yield: 710 mg (95%).

### PREPARATION EXAMPLE 118

### Synthesis of 1-(tert-butoxycarbonyl)-4-[N-(3-methoxyphenyl)-N-[[3-(3,4,5-trimethoxyphenyl)pyridin-5-yl]methyl]amino]piperidine

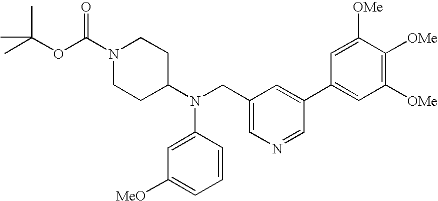

4-(m-Anisidino)-1-(tert-butoxycarbonyl)piperidine (613 mg) and 5-chloromethyl-3-(3,4,5-trimethoxyphenyl)pyridine (588 mg) was treated in the same manner as described in Example 9 to give light yellow amorphous of the title compound.

Yield: 396 mg (35%).

¹H-NMR (400 MHz, CDCl₃) δ: 1.45 (s, 9H), 1.54-1.66 (m, 2H), 1.81-1.91 (m, 2H), 2.73-2.87 (m, 2H), 3.74 (s, 3H), 3.87-3.93 (m, 1H), 3.88 (s, 3H), 3.90 (s, 6H), 4.14-4.29 (m, 2H), 4.51 (s, 2H), 6.30-6.35 (m, 2H), 6.38 (d, 1H, J=7.2 Hz), 6.68 (s, 2H), 7.12 (dd, 1H, J=8.8 Hz, 8.8 Hz), 7.66 (s, 1H), 8.49 (d, 1H, J=2.0 Hz), 8.66 (d, 1H, J=2.2 Hz).

### PREPARATION EXAMPLE 119

### Synthesis of 4-[N-(3-methoxyphenyl)-N-[[3-(3,4,5-trimethoxyphenyl)pyridin-5-yl]methyl]amino]piperidine Dihydrochloride

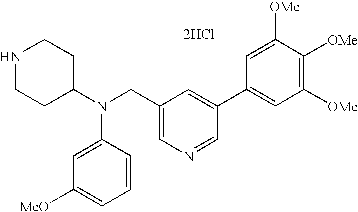

1-(tert-Butoxycarbonyl)-4-[N-(3-methoxyphenyl)-N-[[3-(3,4,5-trimethoxyphenyl)pyridin-5-yl]methyl]amino]piperidine (396 mg) was treated in the same manner as described in Preparation Example 94 to give light yellow powder of the title compound.

Yield: 348 mg (92%).

### PREPARATION EXAMPLE 120

### Synthesis of 1-(tert-butoxycarbonyl)-4-[N-(3-methoxyphenyl)-N-[3-(3,4,5-trimethoxyphenyl)benzyl]amino]piperidine

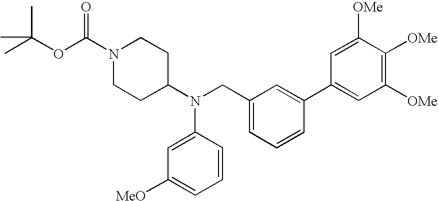

4-(m-Anisidino)-1-(tert-butoxycarbonyl)piperidine (613 mg) and 3-(3,4,5-trimethoxyphenyl)benzyl chloride (586 mg) was treated in the same manner as described in Preparation Example 9 to give light yellow amorphous of the title compound.

Yield: 1.01 g (90%).

¹H-NMR (400 MHz, CDCl₃) δ: 1.44 (s, 9H), 1.56-1.67 (m, 2H), 1.83-1.91 (m, 2H), 2.72-2.86 (m, 2H), 3.73 (s, 3H), 3.85-3.98 (m, 1H), 3.88 (s, 3H), 3.90 (s, 6H), 4.12-4.30 (m, 2H), 4.50 (s, 2H), 6.27-6.34 (m, 2H), 6.38 (dd, 1H, J=8.2 Hz, 2.4 Hz), 6.72 (s, 2H), 7.10 (dd, 1H, J=8.2 Hz, 8.2 Hz), 7.21-7.27 (m, 1H), 7.32-7.43 (m, 3H).

### PREPARATION EXAMPLE 121

### Synthesis of 4-[N-(3-methoxyphenyl)-N-[3-(3,4,5-trimethoxyphenyl)benzyl]amino]piperidine Hydrochloride

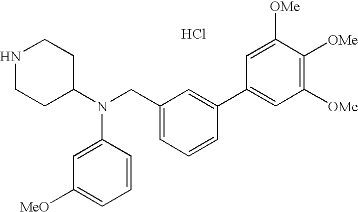

1-(tert-Butoxycarbonyl)-4[N-(3-methoxyphenyl)-N-[3-(3,4,5-trimethoxyphenyl)benzyl]amino]piperidine (1.01 g) was treated in the same manner as described in Preparation Example 94 to give light yellow powder of the title compound.

Yield: 820 mg (92%).

### EXAMPLES 88 TO 95

These compounds were obtained by the condensation of amines obtained in Preparation Examples 117, 119 and 121 with chloride derivatives obtained in Preparation Examples 3, 42 and 48. Free bases obtained were then converted to the corresponding hydrochlorides. Yields and NMR data of their free bases are listed below.

| Ex- | | | NMR data (400 MHz, measured as |
|---|---|---|---|
| ample | Structure | Yield | free bases, CDCl₃) δ |
| 88 | 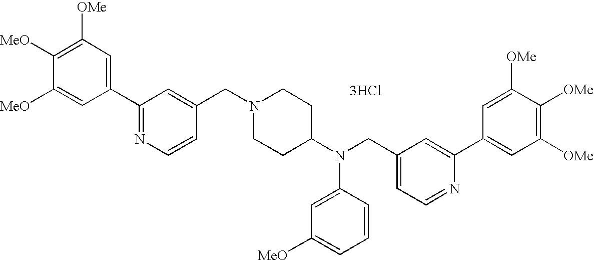 | 63% | 1.70-1.82(m, 2H), 1.83-1.90(m, 2H), 2.14-2.23(m, 2H), 2.94-3.01(m, 2H), 3.57(s, 2H), 3.73(s, 3H), 3.76-3.88(m, 1H), 3.89(s, 3H), 3.90(s, 3H), 3.93(s, 6H), 3.96(s, 6H), 4.53(s, 2H), 6.26-6.35(m, 3H), 7.11(dd, 1H, J=8.3Hz, 8.3Hz), 7.12-7.14(m, 1H), 7.15(s, 2H), 7.20(d, 1H, J=5.1Hz), |
| #7.22(s, 2H), 7.55(s, 1H), 7.58(s, 1H), 8.55(d, 1H, J=4.9Hz), 8.59(d, 1H, J=4.9Hz). | | | |
| 89 | 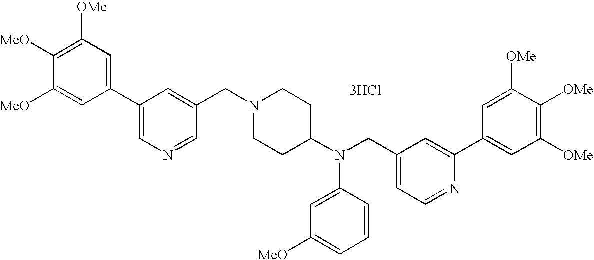 | 72% | 1.67-1.90(m, 4H), 2.13-2.22(m, 2H), 2.94-3.04(m, 2H), 3.59(s, 2H), 3.74(s, 3H), 3.77-3.87(m, 1H), 3.89(s, 3H), 3.89(s, 3H), 3.92(s, 6H), 3.93(s, 6H), 4.52(s, 2H), 6.27(dd, 1H, J=2.4Hz, 2.4Hz), 6.29-6.34(m, 2H), 6.75(s, 2H), 7.08-7.17(m, 4H), 7.54(s, 1H), 7.75(s, 1H), 8.50(d, 1H, J=1.8Hz), |
| #8.54(d, 1H, J=5.1Hz), 8.69(d, 1H, J=2.0Hz). | | | |
| 90 | 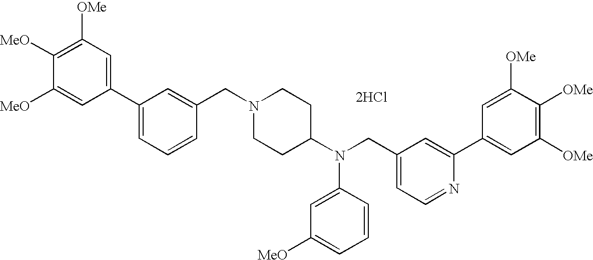 | 60% | 1.68-1.90(m, 4H), 2.09-2.19(m, 2H), 2.97-3.06(m, 2H), 3.58(s, 2H), 3.73(s, 3H), 3.76-3.87(m, 1H), 3.89(s, 6H), 3.92(s, 6H), 3.92(s, 6H), 4.52(s, 2H), 6.25-6.35(m, 3H), 6.76(s, 2H), 6.78-7.17(m, 4H), 7.25-7.32(m, 1H), 7.37(dd, 1H), J=7.4Hz, 7.4Hz), 7.41-7.47(m, 2H), 7.54(s, 1H), 8.54(d, 1H, |
| #J=5.1Hz). | | | |
| 91 | 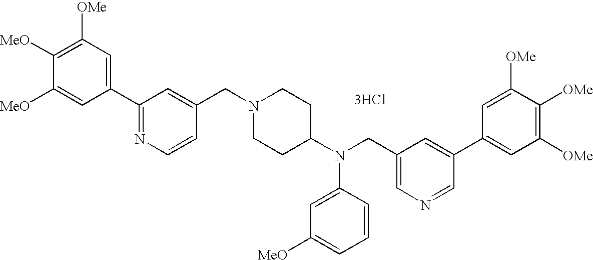 | 50% | 1.80-1.93(m, 4H), 2.13-2.32(m, 2H), 2.87-3.10(m, 2H), 3.60(s, 1H), 3.69-3.85(m, 1H), 3.73(s, 3H), 3.88(s, 3H), 3.89(s, 6H), 3.90(s, 3H), 3.96(s, 6H), 4.57(s, 2H), 6.29-6.34(m, 2H), 6.37(dd, 1H, J=8.2Hz, 8.1Hz), 6.67(s, 2H), 7.11(dd, 1H, J=8.6Hz, 8.6Hz), 7.20-7.28(m, 3H), 7.58-7.72(m, 1H), |
| #7.68(s, 1H), 8.50(d, 1H, J=1.8Hz), 8.60(d, 1H, J=4.7Hz), 8.65(d, 1H, J=2.0Hz). | | | |
| 92 | 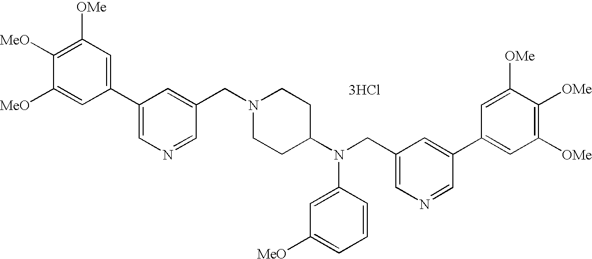 | 35% | 1.70-1.90(m, 4H), 2.12-2.25(m, 2H), 2.95-3.03(m, 2H), 3.59(s, 2H), 3.72-3.97(m, 1H), 3.73(s, 3H), 3.88(s, 3H), 3.89(s, 6H), 3.90(s, 3H), 3.93(s, 6H), 4.54(s, 2H), 6.25-6.38(m, 2H), 6.36(d, 1H, J=8.4Hz, 8.4Hz), 6.67(s, J=8.4Hz), 7.66(s, 1H), 8.49(s, 1H), 8.50(d, 1H, J=1.8Hz), 8.64(d, 1H, J=2.0Hz), |
| #8.70(d, 1H, J=1.9Hz). | | | |
| 93 | 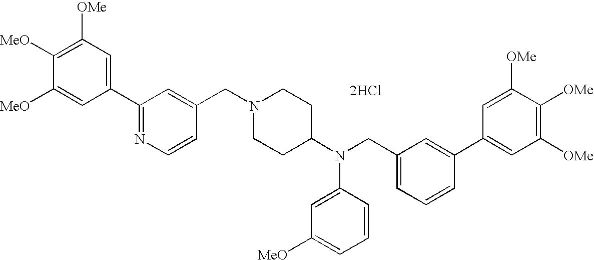 | 86% | 1.73-1.93(m, 4H), 2.13-2.23(m, 2H), 2.94-3.02(m, 2H), 3.57(s, 2H), 3.73(s, 3H), 3.77-3.87(m, 1H), 3.88(s, 3H), 3.88(s, 6H), 3.90(s, 3H), 3.96(s, 6H), 4.56(s, 2H), 6.27(dd, 1H, J=8.0Hz, 2.2Hz), 6.31(dd, 1H, J=2.2Hz, 2.2Hz), 6.36(dd, 1H, J=8.2Hz, 2.2Hz), 6.71(s, 2H), 7.09(dd, 1H, J=8.1Hz, 8.1Hz), |
| #7.18-7.28(m, 4H), 7.34(dd, 1H, J=7.4Hz, 7.4Hz), 7.38(d, 1H, J=7.6Hz), 7.42(s, 1H), 7.59(s, 2H), 8.59(d, 1H, J=4.9Hz). | | | |
| 94 | 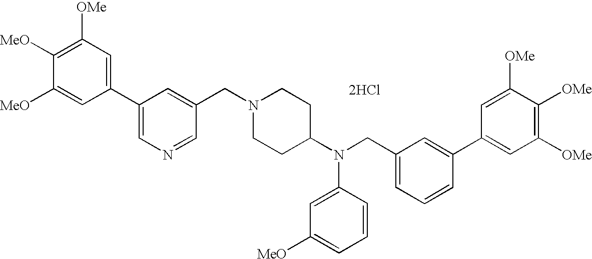 | 56% | 1.72-1.92(m, 4H), 2.10-2.23(m, 2H), 2.92-3.60(m, 2H), 3.59(s, 2H), 3.72(s, 3H), 3.77-3.89(m, 1H), 3.87(s, 3H), 3.88(s, 6H), 3.93(s, 6H), 4.55(s, 2H), 6.27(dd, 1H, J=8.0Hz, 2.2Hz), 6.31(dd, 1H, J=2.1Hz, 2.1Hz), 6.36(dd, 1H, J=8.4Hz, 2.4Hz), 6.70(s, 2H), 6.75(s, 2H), 7.09(dd, 1H, J=8.2Hz, 8.2Hz), |
| #7.22(d, 1H, J=7.4Hz), 7.33(dd, 1H, J=7.4Hz, 7.4Hz), 7.38(d, 1H, J=7.8Hz), 7.40(s, 1H), 7.77(s, 1H), 8.50(d, 1H, J=1.8Hz), 8.69(d, 1H, J=1.8Hz). | | | |
| 95 | 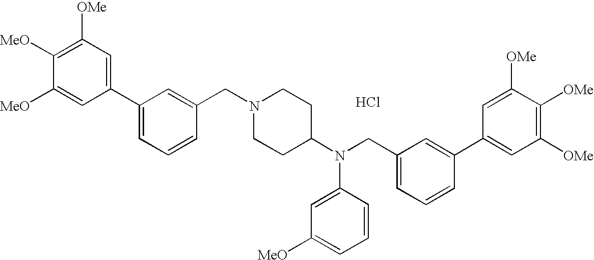 | 77% | 1.66-1.89(m, 4H), 2.08-2.18(m, 2H), 2.95-3.05(m, 2H), 3.58(s, 2H), 3.72(s, 3H), 3.75-3.84(m, 1H), 3.87(s, 3H), 3.88(s, 6H), 3.89(s, 3H), 3.92(s, 6H), 4.55(s, 2H), 6.26(dd, 1H, J=8.0Hz, 2.2Hz), 6.30(dd, 1H, J=2.2Hz, 2.2Hz), 6.36(dd, 1H, J=8.3Hz, 2.2Hz), 6.70(s, 2H), 6.76(s, 2H), 7.08(dd, 1H, |
| #J=8.3Hz, 8.3Hz), 7.22(d, 1H, J=7.3Hz), 7.27-7.47(m, 7H). | | | |

### PREPARATION EXAMPLE 122

### Synthesis of 4-(o-anisidino)-1-(tert-butoxycarbonyl)piperidine

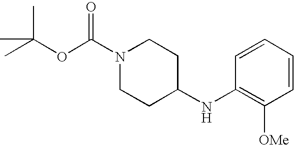

1-(tert-Butoxycarbonyl)-4-piperidone (4.78 g) and o-anisidine (2.96 g) were condensed in the same manner as described in Preparation Example 37 to give the title compound.

Yield: 2.61 g (36%).

¹H-NMR (400 MHz, CDCl₃) δ: 1.31-1.41 (m, 2H), 1.47 (s, 9H), 2.00-2.08 (m, 2H), 2.90-3.01 (m, 2H), 3.38-3.47 (m, 1H), 3.83 (s, 3H), 4.00-4.21 (m, 2H), 6.60-6.69 (m, 2H), 6.76-6.89 (m, 2H).

### PREPARATION EXAMPLE 123

### Synthesis of 1-(tert-butoxycarbonyl)-4-[N-(2-methoxyphenyl)-N-[[2-(3,4,5-trimethoxyphenyl)pyridin-4-yl]methyl]amino]piperidine

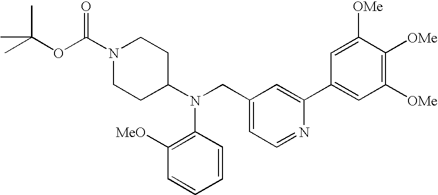

4-(o-Anisidino)-1-(tert-butoxycarbonyl)piperidine (613 mg) and 4-chloromethyl-2-(3,4,5-trimethoxyphenyl)pyridine (588 mg) was treated in the same manner as described in Example 9 to give light yellow amorphous of the title compound.

Yield: 763 mg (68%).

¹H-NMR (400 MHz, CDCl₃) δ: 1.41-1.58 (m, 2H), 1.44 (s, 9H), 1.81-1.91 (m, 2H), 2.62-2.78 (m, 2H), 3.29 (tt, 1H, J=7.6 Hz, 3.7 Hz), 3.86 (s, 3H), 3.89 (s, 3H), 3.95 (s, 6H), 4.06-4.16 (m, 2H), 4.37 (s, 2H), 6.80 (ddd, 1H, J=7.6 Hz, 7.6 Hz, 1.2 Hz), 6.87 (dd, 1H, J=8.5 Hz, 1.0 Hz), 7.00-7.06 (m, 2H), 7.14 (s, 2H), 7.20 (dd, 1H, J=4.9 Hz, 1.0 Hz), 7.61 (s, 1H), 8.49 (d, 1H, J=4.9 Hz).

### PREPARATION EXAMPLE 124

### Synthesis of

4-[N-(2-methoxyphenyl)-N-[[2-(3,4,5-trimethoxyphenyl)pyridin-4-yl]methyl]amino]piperidine Dihydrochloride:

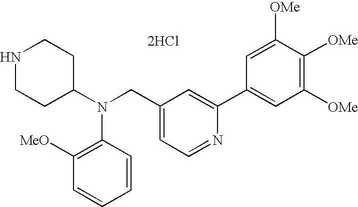

1-(tert-Butoxycarbonyl)-4-[N-(2-methoxyphenyl)-N-[[2-(3,4,5-trimethoxyphenyl)pyridin-4-yl]methyl]amino]piperidine (763 mg) was treated in the same manner as described in Preparation Example 94 to give light yellow powder of the title compound.

Yield: 701 mg (97%).

### PREPARATION EXAMPLE 125

### Synthesis of 1-(tert-butoxycarbonyl)-4-[N-(2-methoxyphenyl)-N-[[3-(3,4,5-trimethoxyphenyl)pyridin-5-yl]methyl]amino]piperidine

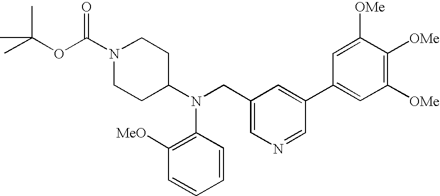

4-(o-Anisidino)-1-(tert-butoxycarbonyl)piperidine (613 mg) and 5-chloromethyl-3-(3,4,5-trimethoxyphenyl)pyridine (588 mg) was treated in the same manner as described in Example 9 to give light yellow amorphous of the title compound.

Yield: 353 mg (31%).

¹H-NMR (400 MHz, CDCl₃) δ: 1.44 (s, 9H), 1.46-1.53 (m, 2H), 1.82-1.91 (m, 2H), 2.62-2.78 (m, 2H), 3.24-3.33 (m, 1H), 3.83 (s, 3H), 3.89 (s, 3H), 3.91 (s, 6H), 4.03-4.16 (m, 2H), 4.37 (s, 2H), 6.64 (s, 2H), 6.79 (ddd, 1H, J=7.6 Hz, 7.6 Hz, 1.2 Hz), 6.84 (dd, 1H, J=7.0 Hz, 1.2 Hz), 6.97-7.06 (m, 2H), 7.68 (dd, 1H, J=1.3 Hz, 1.3 Hz), 8.49 (d, 1H, J=2.0 Hz), 8.56 (d, 1H, J=2.2 Hz).

### PREPARATION EXAMPLE 126

### Synthesis of 4-[N-(2-methoxyphenyl)-N-[[3-(3,4,5-trimethoxyphenyl)pyridin-5-yl]methyl]amino]piperidine Dihydrochloride

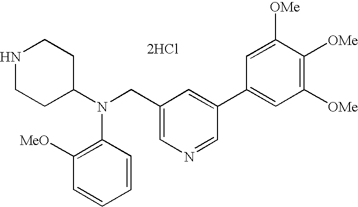

1-(tert-Butoxycarbonyl)-4-[N-(2-methoxyphenyl)-N-[[3-(3,4,5-trimethoxyphenyl)pyridin-5-yl]methyl]amino]piperidine (353 mg) was treated in the same manner as described in Preparation Example 94 to give light yellow powder of the title compound.

Yield: 312 mg (93%).

### PREPARATION EXAMPLE 127

### Synthesis of 1-(tert-butoxycarbonyl)-4-[N-(2-methoxyphenyl)-N-[3-(3,4,5-trimethoxyphenyl)benzyl]amino]piperidine

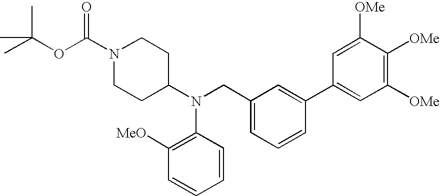

4-(o-Anisidino)-1-(tert-butoxycarbonyl)piperidine (613 mg) and 3-(3,4,5-trimethoxyphenyl)benzyl chloride (586 mg) was treated in the same manner as described in Example 9 to give light yellow amorphous of the title compound.

Yield: 1.12 g (100%).

¹H-NMR (400 MHz, CDCl₃) δ: 1.43 (s, 9H), 1.46-1.57 (m, 2H), 1.81-1.90 (m, 2H), 2.62-2.76 (m, 2H), 3.31 (tt, 1H, J=11.1 Hz, 3.3 Hz), 3.84 (s, 3H), 3.88 (s, 3H), 3.91 (s, 6H), 4.00-4.16 (m, 2H), 4.36 (s, 2H), 6.67 (s, 2H), 6.78 (t, 1H, J=7.3 Hz), 6.85 (d, 1H, J=7.9 Hz), 6.96-7.03 (m, 2H), 7.24-7.34 (m, 3H), 7.43 (s, 1H).

### PREPARATION EXAMPLE 128

### Synthesis of

4-[N-(2-methoxyphenyl)-N-[3-(3,4,5-trimethoxyphenyl)benzyl]amino]piperidine Hydrochloride:

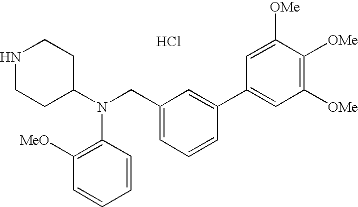

1-(tert-Butoxycarbonyl)-4-[N-(2-methoxyphenyl)-N-[3-(3,4,5-trimethoxyphenyl)benzyl]amino]piperidine (1.12 g) was treated in the same manner as described in Preparation Example 94 to give light yellow powder of the title compound.

Yield: 987 mg (99%).

### EXAMPLE 96 TO 101

These compounds were obtained by the condensation of amines obtained in Preparation Examples 124, 126 and 128 with chloride derivatives obtained in Preparation Examples 3 and 48. Free bases obtained were then converted to the corresponding hydrochlorides. Yields and NMR data of their free bases are listed below.

| Ex- | | | NMR data (400 MHz, measured as |
|---|---|---|---|
| ample | Structure | Yield | free bases, CDCl₃) δ |
| 96 | 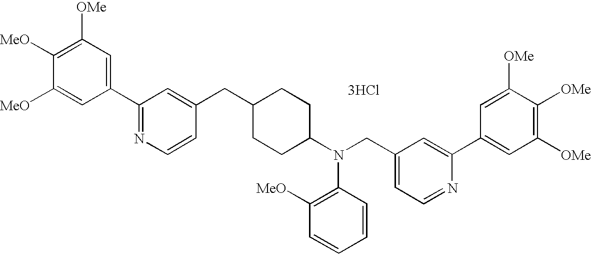 | 73% | 1.62-1.74(m, 2H), 1.82-1.90(m, 2H), 1.98-2.08(m, 2H), 2.86-2.94(m, 2H), 3.13-3.22(m, 1H), 3.52(s, 2H), 3.85(s, 3H), 3.89(s, 3H), 3.90(s, 3H), 3.94(s, 6H), 3.96(s, 6H), 4.40(s, 2H), 6.80(ddd, 1H, J=7.6Hz, 7.6Hz, 1.2Hz), 6.86(dd, 1H, J=8.1Hz, 1.2Hz), 6.98-7.05(m, 1H), 7.14(s, 2H), 7.18(dd, 1H, |
| #J=4.9Hz, 1.2Hz), 7.20-7.24(m, 1H), 7.22(s, 2H), 7.58(s, 1H), 7.62(s, 1H), 8.49(d, 1H, J=4.9Hz), 8.57(d, 1H, J=5.2Hz). | | | |
| 97 | 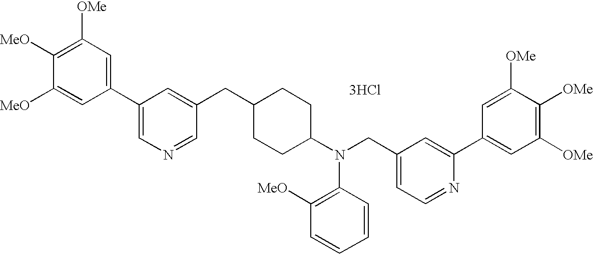 | 55% | 1.60-1.73(m, 4H), 1.82-1.93(m, 2H), 1.98-2.07(m, 2H), 2.87-2.97(m, 2H), 3.12-3.22(m, 1H), 3.54(s, 2H), 3.85(s, 3H), 3.89(s, 3H), 3.90(s, 3H), 3.93(s, 6H), 3.94(s, 6H), 4.39(s, 2H), 6.75(s, 2H), 6.79(dd, 1H, J=7.4Hz, 7.4Hz), 6.86(d, 1H, J=7.8Hz), 6.97-7.05(m, 2H), 7.13(s, 2H), |
| #7.20(d, 1H, J=4.7Hz), 7.61(s, 1H), 7.75(s, 1H), 8.46-8.50(m, 2H), 8.68(d, 1H, J=2.0Hz). | | | |
| 98 | 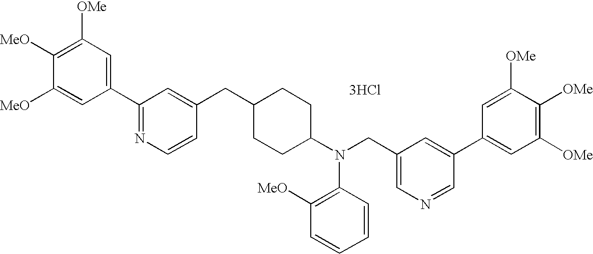 | 29% | 1.64-1.82(m, 2H), 1.84-1.97(m, 2H), 2.00-2.15(m, 2H), 2.84-3.01(m, 2H), 3.13-3.27(m, 1H), 3.56(s, 2H), 3.82(s, 3H), 3.88(s, 3H), 3.90(s, 3H), 3.91(s, 6H), 3.96(s, 6H), 4.40(s, 2H), 6.63(s, 2H), 6.75-6.88(m, 2H), 6.97-7.04(m, 2H), 7.19(d, 1H, J=4.3Hz), 7.25(s, 2H), 7.58-7.73(m, 2H), |
| #8.50(d, 1H, J=1.6Hz), 8.56(d, 1H, J=2.2Hz), 8.58(d, 1H, J=4.9Hz). | | | |
| 99 | 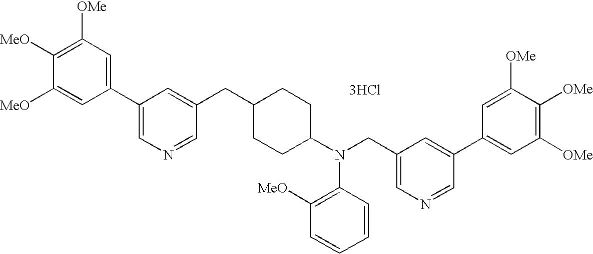 | 30% | 1.62-1.75(m, 2H), 1.83-1.94(m, 2H), 1.95-2.11(m, 2H), 2.84-3.01(m, 2H), 3.12-3.23(m, 1H), 3.55(s, 2H), 3.82(s, 3H), 3.88(s, 3H), 3.90(s, 3H), 3.90(s, 6H), 3.93(s, 6H), 4.39(s, 2H), 6.63(s, 2H), 6.70-6.86(m, 4H), 6.94-7.06(m, 2H), 7.68(s, 1H), 7.76(s, 1H), 8.47(d, 1H, J=1.7Hz), |
| #8.49(d, 1H, J=1.7Hz), 8.55(d, 1H, J=2.2Hz), 8.69(s, 1H). | | | |
| 100 | 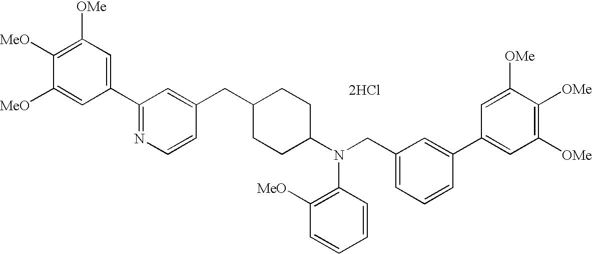 | 67% | 1.64-1.79(m, 2H), 1.85-1.93(m, 2H), 1.99-2.09(m, 2H), 2.86-2.95(m, 2H), 3.16-3.26(m, 1H), 3.52(s, 2H), 3.84(s, 3H), 3.88(s, 3H), 3.90(s, 6H), 3.96(s, 6H), 4.40(s, 2H), 6.67(s, 2H), 6.78(dd, 1H, J=7.4Hz, 7.4Hz), 6.85(d, 1H, J=8.2Hz), 6.97(dd, 1H, J=7.8Hz, 7.8Hz), 7.02(dd, 1H, |
| #J=7.8, 1.6Hz), 7.17-7.33(m, 6H), 7.44(s, 1H), 7.59(s, 1H), 8.57(d, 1H, J=5.1Hz). | | | |
| 101 | 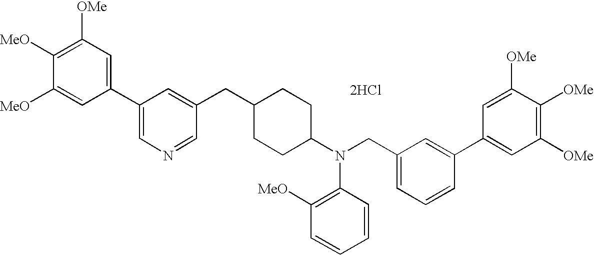 | 55% | 1.62-1.77(m, 2H), 1.82-1.94(m, 2H), 1.98-2.08(m, 2H), 2.86-2.96(m, 2H), 3.16-3.26(m, 1H), 3.54(s, 2H), 3.83(s, 3H), 3.87(s, 3H), 3.90(s, 9H), 3.93(s, 6H), 4.39(s, 2H), 6.66(s, 2H), 6.73-6.80(m, 3H), 6.84(d, 1H, J=7.8Hz), 6.97(dd, 1H, J=7.8Hz, 7.8Hz), 7.01(d, 1H, J=7.8Hz), |
| #7.23-7.32(m, 3H), 7.43(s, 1H), 7.77(s, 1H), 8.47(d, 1H, J=1.4Hz), 8.68(d, 1H, J=1.8Hz). | | | |

### PREPARATION EXAMPLE 129

### Synthesis of 1-(tert-butoxycarbonyl)-4-(2,3-dimethoxyphenylamino)piperidine

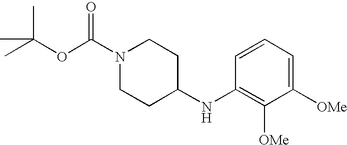

1-(tert-Butoxycarbonyl)-4-piperidone (4.78 g) and 2,3-dimethoxyaniline (3.68 g) were condensed in the same manner as described in Preparation Example 37 to give the title compound.

Yield: 3.18 g (39%).

¹H-NMR (400 MHz, CDCl₃) δ: 1.29-1.42 (m, 2H), 1.45 (s, 9H), 1.97-2.03 (m, 2H), 2.92 (dt, 2H, J=13.5 Hz, 2.2 Hz), 3.38 (dt, 1H, J=13.8 Hz, 4.1 Hz), 3.77 (s, 3H), 3.82 (s, 3H), 3.99-4.03 (m, 2H), 4.17 (m, 1H), 6.27-6.32 (m, 2H), 6.88 (t, 1H, J=8.4 Hz).

### PREPARATION EXAMPLE 130

### Synthesis of

1-(tert-butoxycarbonyl)-4-[N-(2,3-dimethoxyphenyl)-N-[[2-(3,4,5-trimethoxyphenyl)pyridin-4-yl]methyl]amino]piperidine:

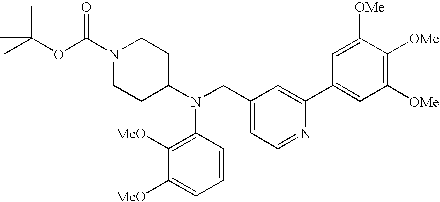

1-(tert-Butoxycarbonyl)-4-(2,3-dimethoxyphenylamino)piperidine (673 mg) and 4-chloromethyl-2-(3,4,5-trimethoxyphenyl)pyridine (588 mg) was treated in the same manner as described in Example 9 to give light yellow amorphous of the title compound.

Yield: 613 mg (52%).

¹H-NMR (400 MHz, CDCl₃) δ: 1.45 (s, 9H), 1.56-1.70 (m, 2H), 1.84-1.91 (m, 2H), 2.62-2.76 (m, 2H), 3.58 (tt, 1H, J=11.8 Hz, 3.6 Hz), 3.83 (s, 3H), 3.89 (s, 6H), 3.93 (s, 6H), 4.08-4.25 (m, 2H), 4.35 (s, 2H), 6.56-6.63 (m, 2H), 6.86 (t, 1H, J=8.3 Hz), 7.14 (s, 2H), 7.17 (dd, 1H, J=5.1 Hz, 1.2 Hz), 7.62 (s, 1H), 8.50 (d, 1H, J=5.1 Hz).

### PREPARATION EXAMPLE 131

### Synthesis of

4-[N-(2,3-dimethoxyphenyl)-N-[[2-(3,4,5-trimethoxyphenyl)pyridin-4-yl]methyl]amino]piperidine Dihydrochloride:

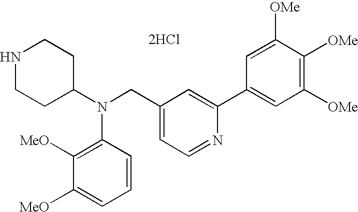

1-(tert-Butoxycarbonyl)-4-[N-(2,3-dimethoxyphenyl)-N-[[2-(3,4,5-trimethoxy phenyl)pyridin-4-yl]methyl]amino]piperidine (613 mg) was treated in the same manner as described in Preparation Example 94 to give light yellow powder of the title compound.

Yield: 512 mg (88%).

### EXAMPLE 102

### Synthesis of 4-[N-(2,3-dimethoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine Trihydrochloride

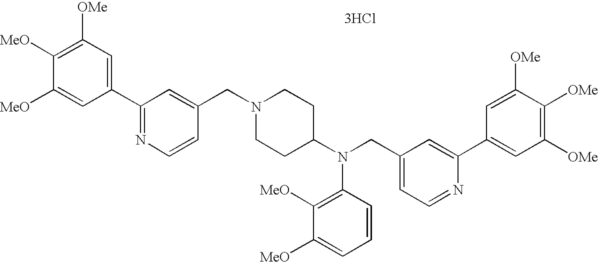

4-[N-(2,3-Dimethoxyphenyl)-N-[[2-(3,4,5-trimethoxyphenyl)pyridinyl]methyl]amino]piperidine dihydrochloride (113 mg) and 4-chloromethyl-2-(3,4,5-trimethoxyphenyl)pyridine (59 mg) were condensed in the same manner as described in Example 9. The title compound was obtained as light yellow powder after converting a free base to a trihydrochloride.

Yield: 21 mg (12%).

¹H-NMR (400 MHz, measured as a free base, CDCl₃) δ: 1.76-1.96 (m, 4H), 2.00-2.13 (m, 2H), 2.86-3.00 (m, 2H), 3.42-3.60 (m, 1H), 3.54 (s, 2H), 3.82 (s, 3H), 3.88 (s, 3H), 3.90 (s, 3H), 3.97 (s, 6H), 4.41 (s, 2H), 6.57 (d, 1H, J=8.0 Hz), 6.62 (d, 1H, J=8.2 Hz), 6.85 (dd, 1H, J=8.4 Hz, 8.4 Hz), 7.11-7.29 (m, 6H), 7.59 (s, 1H), 7.63 (s, 1H), 8.50 (d, 1H, J=4.9 Hz), 8.59 (d, 1H, J=4.9 Hz).

### PREPARATION EXAMPLE 132

### Synthesis of 1-(tert-butoxycarbonyl)-4-[[4-(trifluoromethoxy)phenyl]amino]piperidine

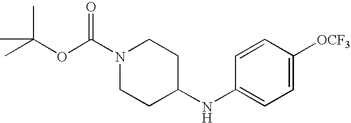

1-tert-Butoxycarbonyl)-4-piperidone (5.00 g) and 4-(trifluoromethoxy)aniline (4.23 g) was treated in the same manner as described in Preparation Example 37 to give white powder of the title compound.

Yield: 5.22 g (60%).

¹H-NMR (400 MHz, CDCl₃) δ: 1.25-1.40 (m, 2H), 1.47 (s, 9H), 1.98-2.08 (m, 2H), 2.83-2.98 (m, 2H), 3.34-3.43 (m, 1H), 3.97-4.12 (m, 2H), 6.58 (d, 2H, J=8.8 Hz), 7.03 (d, 2H, J=8.8 Hz).

### PREPARATION EXAMPLE 133

### Synthesis of

1-(tert-butoxycarbonyl)-4[N-[4-(trifluoromethoxy)phenyl]-N-[[2-(3,4,5-trimethoxyphenyl)pyridin-4-yl]methyl]amino]piperidine:

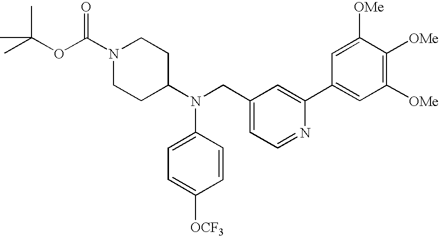

1-(tert-Butoxycarbonyl)-4-[[4-(trifluoromethoxy)phenyl]amino]piperidine (721 mg) and 4-chloromethyl-2-(3,4,5-trimethoxyphenyl)pyridine (588 mg) was treated in the same manner as described in Example 9 to give light yellow amorphous of the title compound.

Yield: 543 mg (44%).

¹H-NMR (400 MHz, CDCl₃) δ: 1.45 (s, 9H), 1.52-1.66 (m, 2H), 1.81-1.91 (m, 2H), 2.73-2.88 (m, 2H), 3.88-3.99 (m, 1H), 3.89 (s, 3H), 3.93 (s, 6H), 4.15-4.34 (m, 2H), 4.48 (s, 2H), 6.68 (d, 2H, J=9.2 Hz), 7.07 (d, 2H, J=8.6 Hz), 7.12 (dd, 1H, J=5.2 Hz, 1.3 Hz), 7.15 (s, 2H), 7.52 (s, 1H), 8.58 (d, 1H, J=5.2 Hz).

### PREPARATION EXAMPLE 134

### Synthesis of

4-[N-[4-(trifluoromethoxy)phenyl]-N-[[2-(3,4,5-trimethoxyphenyl)pyridin-4-yl]methyl]amino]piperidine Dihydrochloride:

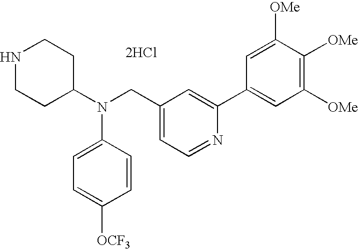

1-(tert-Butoxycarbonyl)-4-[N-[4-(trifluoromethoxy)phenyl]-N-[[2-(3,4,5-trimethoxyphenyl)pyridin-4-yl]methyl]amino]piperidine (543 mg) was treated in the same manner as described in Preparation Example 94 to give light yellow powder of the title compound.

Yield: 481 mg (93%).

### PREPARATION EXAMPLE 135

### Synthesis of 1-(tert-butoxycarbonyl)-4-[N-[4-(trifluoromethoxy)phenyl]-N-[[3-(3,4,5-trimethoxyphenyl)pyridin-5-yl]methyl]amino]piperidine

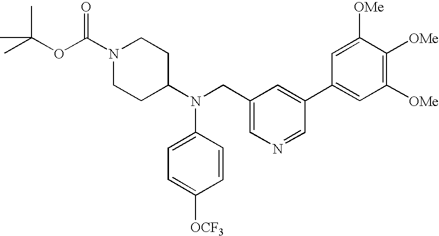

1-(tert-Butoxycarbonyl)-4-[[4-(trifluoromethoxy)phenyl]amino]piperidine (721 mg) and 5-chloromethyl-3-(3,4,5-trimethoxyphenyl)pyridine (588 mg) was treated in the same manner as described in Example 9 to give light yellow amorphous of the title compound.

Yield: 201 mg (16%).

¹H-NMR (400 MHz, CDCl₃) δ: 1.45 (s, 9H), 1.54-1.67 (m, 2H), 1.82-1.90 (m, 2H), 2.74-2.86 (m, 2H), 3.84-3.91 (m, 1H), 3.88 (s, 3H), 3.89 (s, 6H), 4.16-4.30 (m, 2H), 4.52 (s, 2H), 6.67 (s, 2H), 6.72 (d, 2H, J=9.4 Hz), 7.06 (d, 2H, J=8.4 Hz), 7.64 (t, 1H, J=2.1 Hz), 8.49 (d, 1H, J=2.2 Hz), 8.68 (d, 1H, J=2.1 Hz).

### PREPARATION EXAMPLE 136

### Synthesis of

4-[N-[4-(trifluoromethoxy)phenyl]-N-[[3-(3,4,5-trimethoxyphenyl)pyridin-5-yl]methyl]amino]piperidine Dihydrochloride:

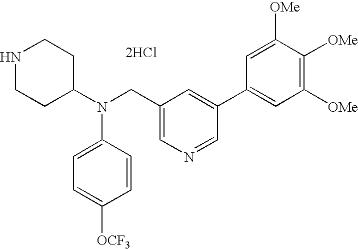

1-(tert-Butoxycarbonyl)-4-[N-[4-(trifluoromethoxy)phenyl]-N-[[3-(3,4,5-trimethoxyphenyl)pyridin-5-yl]methyl]amino]piperidine (201 mg) was treated in the same manner as described in Preparation Example 94 to give light yellow powder of the title compound.

Yield: 185 mg (96%).

### PREPARATION EXAMPLE 137

### Synthesis of 1-(tert-butoxycarbonyl)-4-[N-[4-(trifluoromethoxy)phenyl]-N-[3-(3,4,5-trimethoxyphenyl)benzyl]amino)piperidine

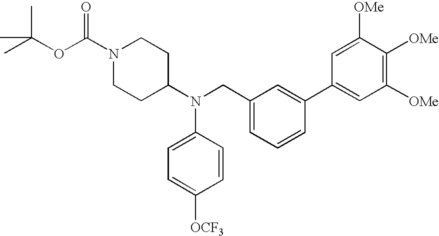

1-(tert-Butoxycarbonyl)-4-[[4-(trifluoromethoxy)phenyl]amino]piperidine (721 mg) and 3-(3,4,5-trimethoxyphenyl)benzyl chloride (586 mg) was treated in the same manner as described in Example 9 to give light yellow amorphous of the title compound.

Yield: 1.06 mg (86%).

¹H-NMR (400 MHz, CDCl₃) δ: 1.45 (s, 9H), 1.56-1.68 (m, 2H), 1.83-1.90 (m, 2H), 2.71-2.86 (m, 2H), 3.87-3.90 (m, 1H), 3.88 (s, 3H), 3.89 (s, 6H), 4.16-4.29 (m, 2H), 4.51 (s, 2H), 6.70 (d, 2H, J=9.3 Hz), 6.70 (s, 2H), 7.04 (d, 2H, J=8.5 Hz), 7.22 (d, 1H, J=7.8 Hz), 7.34-7.44 (m, 3H).

### PREPARATION EXAMPLE 138

### Synthesis of 4-[N-[4-(trifluoromethoxy)phenyl]-N-[3-(3,4,5-trimethoxyphenyl)benzyl]amino]piperidine Hydrochloride:

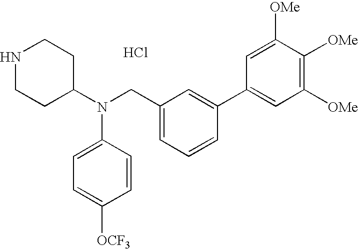

1-(tert-Butoxycarbonyl)-4-[N-[4-(trifluoromethoxy)phenyl]-N-[3-(3,4,5-trimethoxyphenyl)benzyl]amino]piperidine (1.06 g) was treated in the same manner as described in Preparation Example 94 to give light yellow powder of the title compound.

Yield: 795 mg (84%).

### EXAMPLE 103 TO 110

These compounds were obtained by the condensation of amines obtained in Preparation Examples 134, 136 and 138 with chloride derivatives obtained in Preparation Examples 3, 42 and 48. Free bases obtained were then converted to the corresponding hydrochlorides. Yields and NMR data of their free bases are listed below.

| Ex- | | | NMR data (400 MHz, measured as |
|---|---|---|---|
| ample | Structure | Yield | free bases, CDCl₃) δ |
| 103 | 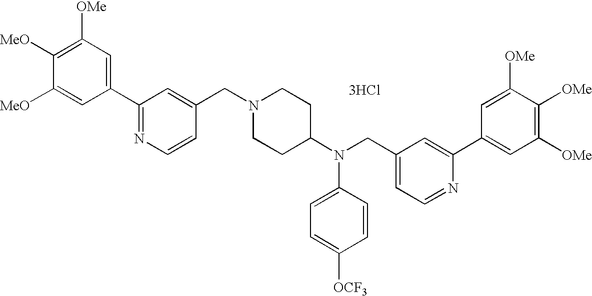 | 70% | 1.71-1.90(m, 4H), 2.15-2.23(m, 2H), 2.95-3.02(m, 2H), 3.58(s, 2H), 3.76-3.85(m, 1H), 3.89(s, 3H), 3.90(s, 3H), 3.92(s, 6H), 3.96(s, 6H), 4.54(s, 2H), 6.66(d, 2H, J=9.3Hz), 7.05(d, 2H, J=8.5Hz), 7.13(dd, 1H, J=5.1Hz, 1.2Hz), 7.14(s, 2H), 7.20(dd, 1H, J=4.9Hz, 1.2Hz), |
| #7.22(s, 2H), 7.53(s, 1H), 7.59(s, 1H), 8.57(d, 1H, J=4.9Hz), 8.59(d, 1H, J=5.2Hz). | | | |
| 104 | 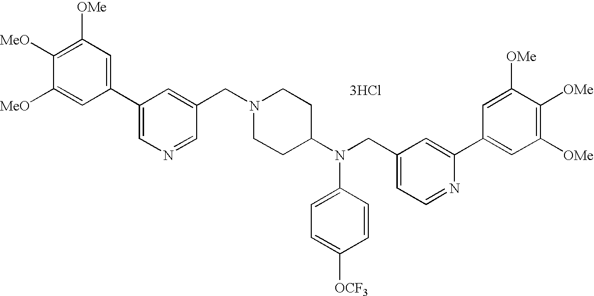 | 48% | 1.68-1.92(m, 4H), 2.13-2.25(m, 2H), 2.95-3.06(m, 2H), 3.60(s, 2H), 3.75-3.87(m, 1H), 3.89(s, 3H), 3.90(s, 3H), 3.91(s, 6H), 3.93(s, 6H), 4.52(s, 2H), 6.65(d, 2H, J=9.4Hz), 6.75(s, 2H), 7.05(d, 2H, J=9.2Hz), 7.12(d, 1H, J=5.1Hz), 7.14(s, 2H), 7.52(s, 1H), 7.76(s, 1H), |
| #8.51(d, 1H, J=1.8Hz), 8.57(d, 1H, J=5.1Hz), 8.70(d, 1H, J=2.1Hz). | | | |
| 105 | 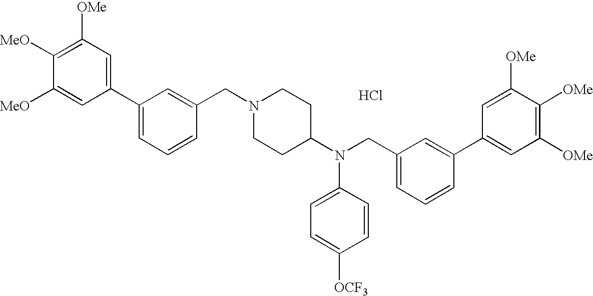 | 69% | 1.70-1.89(m, 4H), 2.10-2.19(m, 2H), 2.98-3.08(m, 2H), 3.59(s, 2H), 3.72-3.84(m, 1H), 3.89(s, 6H), 3.92(s, 6H), 3.92(s, 6H), 4.52(s, 2H), 6.65(d, 2H, J=9.4Hz), 6.76(s, 2H), 7.04(d, 2H, J=8.6Hz), 7.11(d, 1H, J=5.1Hz), 7.14(s, 2H), 7.25-7.33(m, 1H), 7.37(dd, 1H, |
| #J=7.4Hz, 7.4Hz), 7.41-7.48(m, 2H), 7.51(s, 1H), 8.56(d, 1H, J=5.1Hz). | | | |
| 106 | 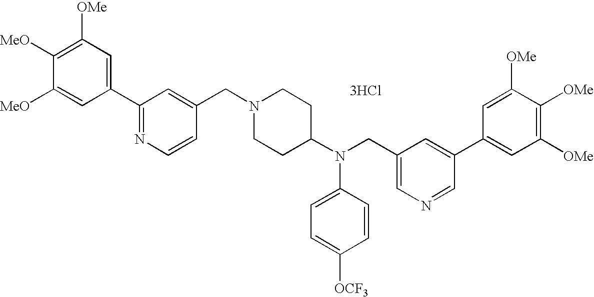 | 41% | 1.73-1.93(m, 4H), 2.12-2.26(m, 2H), 2.93-3.07(m, 2H), 3.53-3.65(m, 2H), 3.74-3.84(m, 1H), 3.88(s, 9H), 3.90(s, 3H), 3.96(s, 6H), 4.58(s, 2H), 6.66(s, 2H), 6.69(d, 2H, J=9.2Hz), 7.05(d, 2H, J=8.8Hz), 7.18-7.29(m, 3H), 7.59(br, 1H), 7.64(s, 1H), |
| #8.49(s, 1H), 8.60(d, 1H, J=5.3Hz), 8.67(d, 1H, J=2.0Hz). | | | |
| 107 | 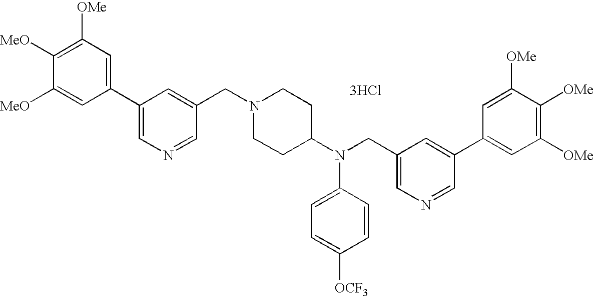 | 28% | 1.72-1.91(m, 4H), 2.12-2.28(m, 2H), 2.94-3.06(m, 2H), 3.60(s, 2H), 3.76-3.82(m, 1H), 3.88(s, 9H), 3.90(s, 3H), 3.93(s, 6H), 4.56(s, 2H), 6.65(s, 2H), 6.69(d, 2H, J=9.2Hz), 6.75(s, 2H), 7.05(d, 2H, J=8.8Hz), 7.63(s, 1H), 7.76(s, 1H), 8.48(d, 1H, J=1.8Hz), 8.51(d, |
| #1H, J=1.8Hz), 8.66(d, 1H, J=2.2Hz), 8.70(d, 1H, J=2.2Hz). | | | |
| 108 | 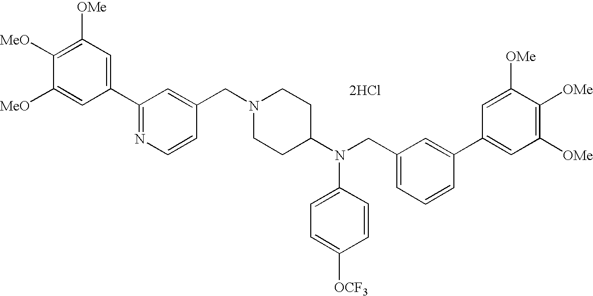 | 78% | 1.76-1.91(m, 4H), 2.14-2.23(m, 2H), 2.94-3.03(m, 2H), 3.57(s, 2H), 3.75-3.84(m, 1H), 3.87(s, 9H), 3.90(s, 3H), 3.96(s, 6H), 4.56(s, 2H), 6.65-6.72(m, 4H), 7.03(d, 2H, J=8.8Hz), 7.18-7.24(m, 4H), 7.33-7.43(m, 3H), 7.59(s, 1H), 8.59(d, 1H, J=4.9Hz). |
| 109 | 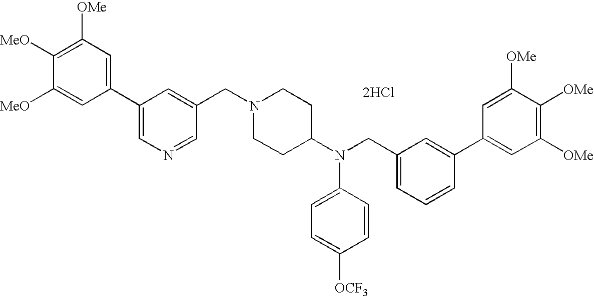 | 5% | 1.72-1.90(m, 4H), 2.12-2.21(m, 2H), 2.94-3.03(m, 2H), 3.59(s, 2H), 3.73-3.86(m, 1H), 3.87(s, 9H), 3.90(s, 3H), 3.93(s, 6H), 4.54(s, 2H), 6.66-6.70(m, 4H), 6.75(s, 2H), 7.03(d, 2H, J=9.0Hz), 7.21(d, 1H, J=7.2Hz), 7.32-7.41(m, 3H), 7.76(s, 1H), .50(d, 1H, J=1.6Hz), |
| #8.69(d, 1H, J=1.6Hz). | | | |
| 110 | 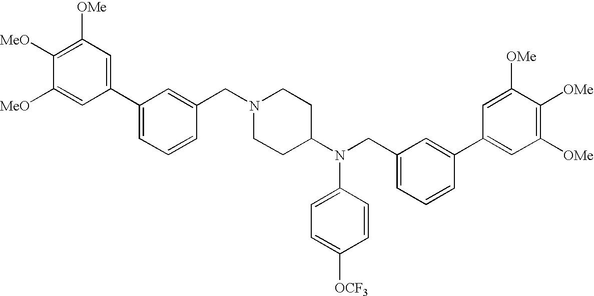 | 62% | 1.72-1.89(m, 4H), 2.08-2.20(m, 2H), 2.97-3.07(m, 2H), 3.59(s, 2H), 3.73-3.83(m, 1H), 3.87(s, 9H), 3.89(s, 3H), 3.92(s, 6H), 4.55(s, 2H), 6.67(d, 2H, J=9.3Hz), 6.69(s, 2H), 6.76(s, 2H), 7.02(d, 2H, J=8.6Hz), 7.20(d, 1H, J=7.6Hz), 7.25-7.47(m, 7H). |

### PREPARATION EXAMPLE 139

### Synthesis of 1-(tert-butoxycarbonyl)-4-[[4-(methylthio)phenyl]amino]piperidine

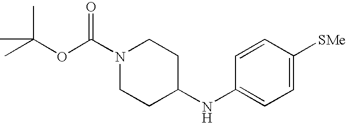

1-(tert-Butoxycarbonyl)-4-piperidone (5.00 g) and 4-(methylthio)aniline (3.33 g) was treated in the same manner as described in Preparation Example 37 to give white powder of the title compound.

Yield: 3.80 g (49%).

¹H-NMR (400 MHz, CDCl₃) δ: 1.26-1.38 (m, 2H), 1.46 (s, 9H), 1.98-2.06 (m, 2H), 2.41 (s, 3H), 2.88-2.97 (m, 2H), 3.36-3.45 (m, 2H), 3.48-3.56 (br, 1H), 3.96-4.12 (m, 2H), 6.55 (d, 2H, J=8.8 Hz), 7.21 (d, 2H, J=8.8 Hz).

### PREPARATION EXAMPLE 140

### Synthesis of 1-(tert-butoxycarbonyl)-4-[N-[4-(methylthio)phenyl]-N-[[2-(3,4,5-trimethoxyphenyl)pyridin-4-yl]methyl]amino]piperidine

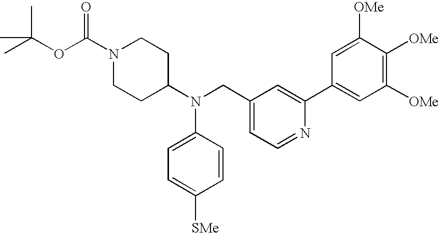

1-(tert-Butoxycarbonyl)-4-[[4-(methylthio)phenyl]amino]piperidine (644 mg) and 4-chloromethyl-2-(3,4,5-trimethoxyphenyl)pyridine (588 mg) was treated in the same manner as described in Example 9 to give light yellow amorphous of the title compound.

Yield: 671 mg (58%).

¹H-NMR (400 MHz, CDCl₃) δ: 1.45 (s, 9H), 1.50-1.66 (m, 2H), 1.81-1.89 (m, 2H), 2.40 (s, 3H), 2.74-2.87 (m, 2H), 3.88-3.94 (m, 1H), 3.90 (s, 3H), 3.94 (s, 6H), 4.15-4.29 (m, 2H), 4.48 (s, 2H), 6.67 (d, 2H, J=9.0 Hz), 7.11-7.18 (m, 1H), 7.16 (s, 2H), 7.22 (d, 2H, J=6.6 Hz), 7.54 (s, 1H), 8.57 (d, 1H, J=5.1 Hz).

### PREPARATION EXAMPLE 141

### Synthesis of 4-[N-[4-(methylthio)phenyl]-N-[[2-(3,4,5-trimethoxyphenyl)pyridin-4-yl]methyl]amino]piperidine Dihydrochloride

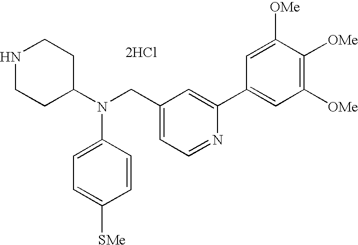

1-(tert-Butoxycarbonyl)-4[N-[4-(methylthio)phenyl]-N-[[2-(3,4,5-trimethoxy phenyl)pyridin-4-yl]methyl]amino]piperidine (671 mg) was treated in the same manner as described in Preparation Example 94 to give light yellow powder of the title compound.

Yield: 602 mg (94%).

### PREPARATION EXAMPLE 142

### Synthesis of 1-(tert-butoxycarbonyl)-4-[N-[4-(methylthio)phenyl]-N-[[3-(3,4,5-trimethoxyphenyl)pyridin-5-yl]methyl]amino]piperidine

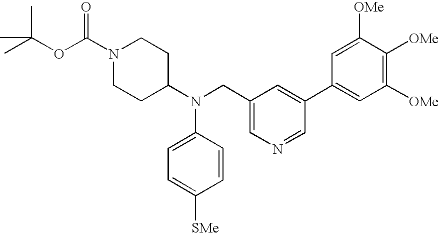

1-(tert-Butoxycarbonyl)-4-[[4-(methylthio)phenyl]amino]piperidine (645 mg) and 5-chloromethyl-3-(3,4,5-trimethoxyphenyl)pyridine (588 mg) was treated in the same manner as described in Example 9 to give light yellow amorphous of the title compound.

Yield: 312 mg (27%).

¹H-NMR (400 MHz, CDCl₃) δ: 1.45 (s, 9H), 1.53-1.63 (m, 2H), 1.83-1.89 (m, 2H), 2.40 (s, 3H), 2.73-2.85 (m, 2H), 3.87-3.91 (m, 1H), 3.88 (s, 3H), 3.90 (s, 6H), 4.16-4.30 (m, 2H), 4.50 (s, 2H), 6.67 (s, 2H), 6.71 (d, 2H, J=9.0 Hz), 7.21 (d, 2H, J=9.0 Hz), 7.64 (s, 1H), 8.48 (d, 1H, J=2.2 Hz), 8.66 (d, 1H, J=2.1 Hz).

### PREPARATION EXAMPLE 143

### Synthesis of

4-[N-[4-(methylthio)phenyl]-N-[[3-(3,4,5-trimethoxyphenyl)pyridin-5-yl]methyl]amino]piperidine Dihydrochloride:

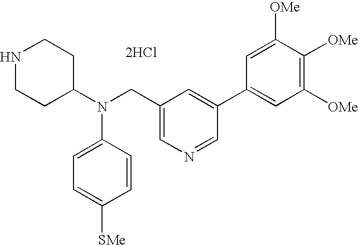

1-(tert-Butoxycarbonyl)-4-[N-[4-(methylthio)phenyl]-N-[[3-(3,4,5-trimethoxy phenyl)pyridin-5-yl]methyl]amino]piperidine (312 mg) was treated in the same manner as described in Preparation Example 94 to give light yellow powder of the title compound.

Yield: 251 mg (84%).

### PREPARATION EXAMPLE 144

### Synthesis of 1-(tert-butoxycarbonyl)-4-[N-[4-(methylthio)phenyl]-N-[3-(3,4,5-trimethoxyphenyl)benzyl]amino]piperidine:

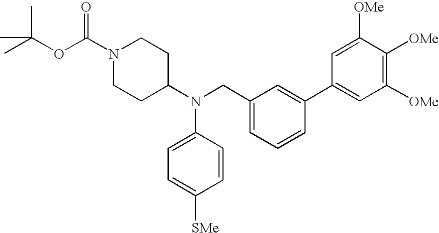

1-(tert-Butoxycarbonyl)-4-[[4-(methylthio)phenyl]amino]piperidine (645 mg) and 3-(3,4,5-trimethoxyphenyl)benzyl chloride (586 mg) was treated in the same manner as described in Example 9 to give light yellow amorphous of the title compound.

Yield: 1.10 g (95%).

¹H-NMR (400 MHz, CDCl₃) δ: 1.45 (s, 9H), 1.55-1.68 (m, 2H), 1.81-1.90 (m, 2H), 2.39 (s, 3H), 2.73-2.86 (m, 2H), 3.87-3.91 (m, 1H), 3.88 (s, 3H), 3.89 (s, 6H), 4.15-4.29(m, 2H), 4.50 (s, 2H), 6.68-6.73 (m, 4H), 7.19-7.24 (m, 3H), 7.33-7.43 (m, 3H).

### PREPARATION EXAMPLE 145

### Synthesis of 4-[N-[4-(methylthio)phenyl]-N-[3-(3,4,5-trimethoxyphenyl)benzyl]amino]piperidine Hydrochloride

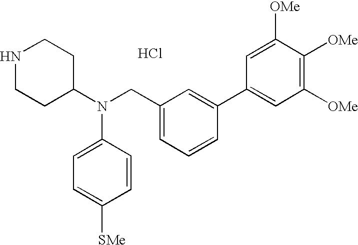

1-(tert-Butoxycarbonyl)-4-[N-[4-(methylthio)phenyl]-N-[3-(3,4,5-trimethoxyphenyl)benzyl]amino]piperidine (1.10 g) was treated in the same manner as described in Preparation Example 94 to give light yellow powder of the title compound.

Yield: 866 mg (89%).

### EXAMPLES 111 TO 118

These compounds were obtained by the condensation of amines obtained in Preparation Examples 141, 143 and 145 with chloride derivatives obtained in Preparation Examples 3, 42 and 48. Free bases obtained were then converted to the corresponding hydrochlorides. Yields and NMR data of their free bases are listed below.

| Ex- | | | NMR data (400 MHz, measured |
|---|---|---|---|
| ample | Structure | Yield | as free bases, CDCl₃) δ |
| 111 | 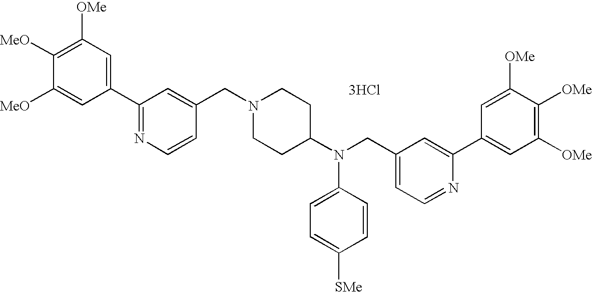 | 40% | 1.70-1.90(m, 4H), 2.14-2.26(m, 2H), 2.40(s, 3H), 2.94-3.04(m, 2H), 3.58(s, 2H), 3.76-3.88(m, 1H), 3.89(s, 3H), 3.90(s, 3H), 3.93(s, 6H), 3.96(s, 6H), 4.53(s, 2H), 6.66(d, 2H, J=9.0Hz), 7.11-7.24(m, 8H), 7.54(s, 1H), 7.59(s, 1H), 8.56(d, 1H, J=5.1Hz), 8.59(d, 1H, J=5.1Hz). |
| 112 | 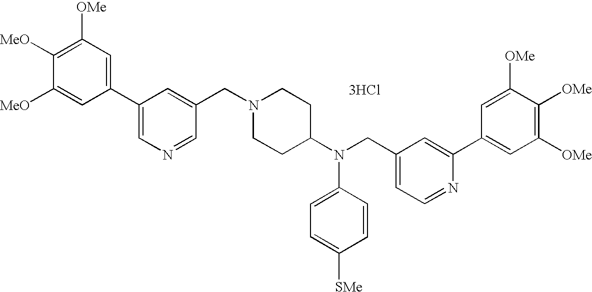 | 53% | 1.66-1.90(m, 4H), 2.12-2.24(m, 2H), 2.40(s, 3H), 2.94-3.05(m, 2H), 3.59(s, 2H), 3.73-3.88(m, 1H), 3.89(s, 3H), 3.90(s, 3H), 3.92(s, 6H), 3.93(s, 6H), 4.51(s, 2H), 6.65(d, 2H, J=8.8Hz), 6.75(s, 2H), 7.12(d, 1H, J=4.9Hz), 7.14(s, 2H), 7.21(d, 2H, J=8.8Hz), 7.53(s, 1H), |
| #7.76(s, 1H), 8.50(d, 1H, J=1.9Hz), 6.55(d, 1H, J=4.9Hz), 8.69(d, 1H, J=1.4Hz). | | | |
| 113 | 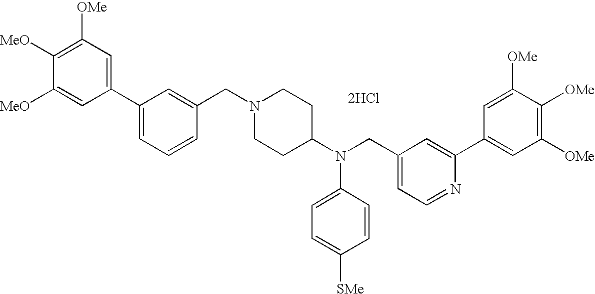 | 53% | 1.68-1.89(m, 4H), 2.10-2.20(m, 2H), 2.39(s, 3H), 2.98-3.07(m, 2H), 3.58(s, 2H), 3.75-3.87(m, 1H), 3.89(s, 6H), 3.92(s, 6H), 3.92(s, 6H), 4.51(s, 2H), 6.65(d, 2H, J=9.0Hz), 6.76(s, 2H), 7.11(d, 1H, J=5.1Hz), 7.14(s, 2H), 7.21(d, 2H, J=8.8Hz), 7.29(d, 1H, J=7.4Hz), |
| #7.37(dd, 1H, J=7.6Hz, 7.6Hz), 7.42-7.49(m, 2H), 7.52(s, 1H), 8.54(d, 1H, J=4.9Hz). | | | |
| 114 | 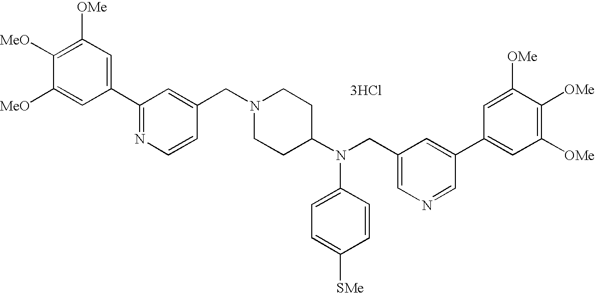 | 50% | 1.57-2.00(m, 4H), 2.12-2.30(m, 2H), 2.39(s, 3H), 2.90-3.13(m, 2H), 3.50-3.74(m, 2H), 3.75-3.86(m, 1H), 3.88(s, 3H), 2.89(s, 3H), 3.90(s, 6H), 3.97(s, 6H), 4.57(s, 2H), 6.66(s, 2H), 6.70(d, 2H, J=9.0Hz), 7.17-7.30(m, 5H), 7.66(br, 2H), 8.48(s, 1H), 8.58-8.70(m, 2H). |
| 115 | 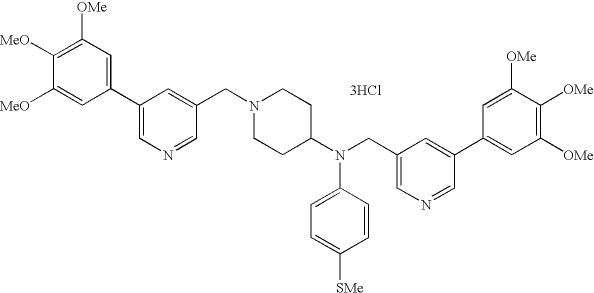 | 59% | 1.68-1.92(m, 4H), 2.12-2.27(m, 2H), 2.39(s, 3H), 2.94-3.08(m, 2H), 3.60(s, 2H), 3.74-3.83(m, 1H), 3.88(s, 3H), 3.89(s, 6H), 3.90(s, 3H), 3.93(s, 6H), 4.55(s, 2H), 6.66(s, 2H), 6.69(d, 2H), J=8.8Hz), 6.73-6.80(m, 2H), 7.20(d, 2H, J=8.8Hz), 7.64(s, 1H), 7.77(br, 1H), |
| #8.48(s, 1H), 8.50(s, 1H), 8.65(s, 1H), 8.71(s, 1H). | | | |
| 116 | 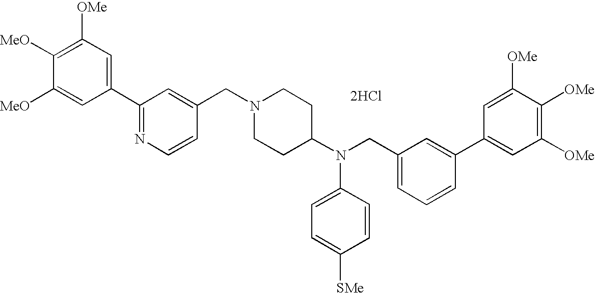 | 85% | 1.76-1.93(m, 4H), 2.14-2.24(m, 2H), 2.39(s, 3H), 2.94-3.03(m, 2H), 3.57(s, 2H), 3.76-3.86(m, 1H), 3.88(s, 6H), 3.90(s, 3H), 3.96(s, 6H), 4.55(s, 2H), 6.67-6.73(m, 4H), 7.18-7.29(m, 6H), 7.34(dd, 1H, J=7.6Hz, 7.6Hz), 7.37-7.44(m, 2H), 7.59(s, 1H), 8.59(d, 1H, J=4.9Hz). |
| 117 | 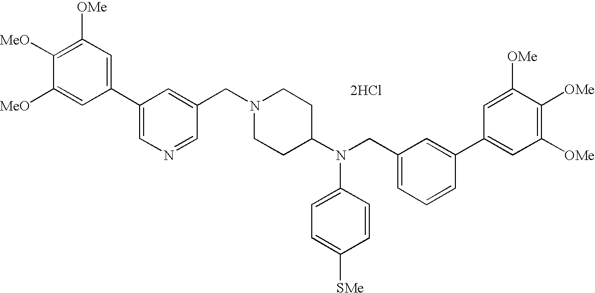 | 53% | 1.72-1.90(m, 4H), 2.12-2.22(m, 2H), 2.39(s, 3H), 2.95-3.05(m, 2H), 3.59(s, 2H), 3.74-3.85(m, 1H), 3.87(s, 3H), 3.88(s, 6H), 3.89(s, 3H), 3.93(s, 6H), 4.54(s, 2H), 6.67-6.70(m, 4H), 6.75(s, 2H), 7.19-7.23(m, 3H), 7.33(dd, 1H, J=7.4Hz, 7.4Hz), 7.36-7.40(m, 2H), 7.76(s, 1H), |
| #8.50(d, 1H, J=1.8Hz), 8.69(s, 1H). | | | |
| 118 | 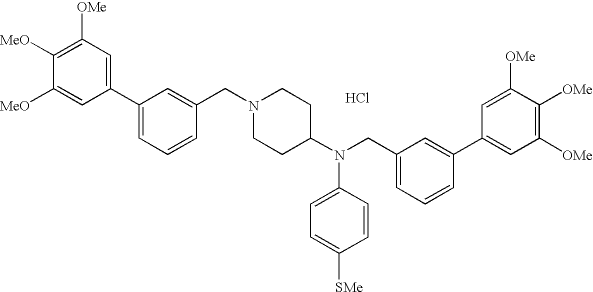 | 83% | 1.72-1.90(m, 4H), 2.09-2.20(m, 2H), 2.38(s, 3H), 2.97-3.06(m, 2H), 3.58(s, 2H), 3.73-3.84(m, 1H), 3.87(s, 3H), 3.88(s, 3H), 3.89(s, 6H), 3.92(s, 6H), 4.54(s, 2H), 6.66-6.71(m, 4H), 6.76(s, 2H), 7.18-7.24(m, 3H), 7.26-7.48(m, 7H). |

### PREPARATION EXAMPLE 146

### Synthesis of 1-(tert-butoxycarbonyl)-4-[(4-methylphenyl)amino]piperidine

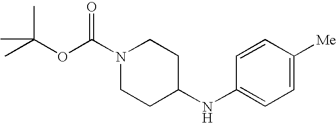

1-(tert-Butoxycarbonyl)-4-piperidone (5.00 g) and p-toluidine (2.56 g) was treated in the same manner as described in Preparation Example 37 to give white powder of the title compound.

Yield: 5.79 g (83%).

¹H-NMR (400 MHz, CDCl₃) δ: 1.25-1.36 (m, 2H), 1.46 (s, 9H), 1.99-2.06 (m, 2H), 2.23 (s, 3H), 2.86-2.96 (m, 2H), 3.30-3.43 (m, 2H), 3.96-4.10 (m, 2H), 6.53 (d, 2H, J=8.4 Hz), 6.98 (d, 2H, J=8.0 Hz).

### PREPARATION EXAMPLE 147

### Synthesis of 1-(tert-butoxycarbonyl)-4-[N-(4-methylphenyl)-N-[[2-(3,4,5-trimethoxyphenyl)pyridin-4-yl]methyl]amino]piperidine

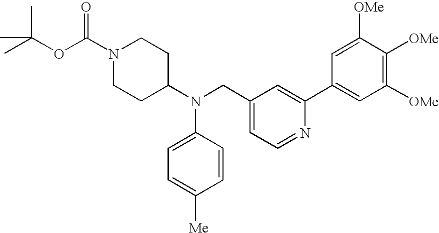

1-(tert-Butoxycarbonyl)-4-[(4-methylphenyl)amino]piperidine (581 mg) and 4-chloromethyl-2-(3,4,5-trimethoxyphenyl)pyridine (588 mg) was treated in the same manner as described in Example 9 to give light yellow amorphous of the title compound.

Yield: 1.00 g (91%).

¹H-NMR (400 MHz, CDCl₃) δ: 1.45 (s, 9H), 1.55-1.59 (m, 2H), 1.81-1.90 (m, 2H), 2.23 (s, 3H), 2.72-2.86 (m, 2H), 3.81-3.94 (m, 1H), 3.89 (s, 3H), 3.93 (s, 6H), 4.14-4.30 (m, 2H), 4.45 (s, 2H), 6.66 (d, 2H, J=8.6 Hz), 7.02 (d, 2H, J=8.2 Hz), 7.13-7.16 (m, 3H), 7.55 (s, 1H), 8.55 (d, 1H, J=8.1 Hz).

### PREPARATION EXAMPLE 148

### Synthesis of 4-[N-(4-methylphenyl)-N-[[2-(3,4,5-trimethoxyphenyl)pyridin-4-yl]methyl]amino]piperidine Dihydrochloride

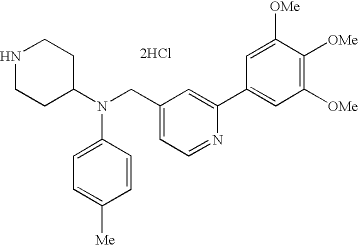

1-(tert-Butoxycarbonyl)-4-[N-(4-methylphenyl)-N-[[2-(3,4,5-trimethoxyphenyl)pyridin-4-yl]methyl]amino]piperidine (1.00 g) was treated in the same manner as described in Preparation Example 94 to give light yellow powder of the title compound.

Yield: 924 mg (97%).

### PREPARATION EXAMPLE 149

### Synthesis of 1-(tert-butoxycarbonyl)-4-[N-(4-methylphenyl)-N-[[3-(3,4,5-trimethoxyphenyl)pyridin-5-yl]methyl]amino]piperidine

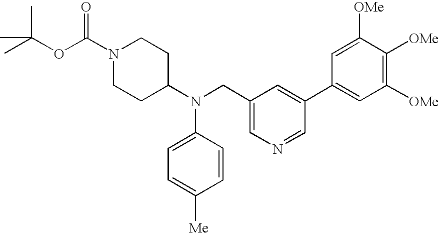

1-(tert-Butoxycarbonyl)-4-[(4-methylphenyl)amino]piperidine (581 mg) and 5-chloromethyl-3-(3,4,5-trimethoxyphenyl)pyridine (588 mg) was treated in the same manner as described in Example 9 to give light yellow amorphous of the title compound.

Yield: 426 mg (39%).

¹H-NMR (400 MHz, CDCl₃) δ: 1.45 (s, 9H), 1.52-1.70 (m, 2H), 1.82-1.90 (m, 2H), 2.23 (s, 3H), 2.72-2.86 (m, 2H), 3.77-3.86 (m, 1H), 3.88 (s, 3H), 3.90 (s, 6H), 4.10-4.28 (m, 2H), 4.47 (s, 2H), 6.67 (s, 2H), 6.70 (d, 2H, J=8.6 Hz), 7.01 (d, 2H, J=8.2 Hz), 7.67 (dd, 1H, J=2.1 Hz, 2.1 Hz), 8.50 (d, 1H, J=2.0 Hz), 8.64 (d, 1H, J=2.2 Hz).

### PREPARATION EXAMPLE 150

### Synthesis of 4-[N-(4-methylphenyl)-N-[[3-(3,4,5-trimethoxyphenyl)pyridin-5-yl]methyl]amino]piperidine Dihydrochloride

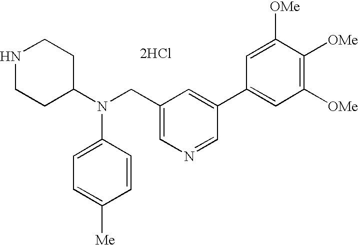

1-(tert-Butoxycarbonyl)-4-[N-(4-methylphenyl)-N-[[3-(3,4,5-trimethoxyphenyl)pyridin-5-yl]methyl]amino]piperidine (426 mg) was treated in the same manner as described in Preparation Example 94 to give light yellow powder of the title compound.

Yield: 400 mg (99%).

### PREPARATION EXAMPLE 151

### Synthesis of 1-(tert-butoxycarbonyl)-4-[N-(4-methylphenyl)-N-[3-(3,4,5-trimethoxyphenyl)benzyl]amino]piperidine

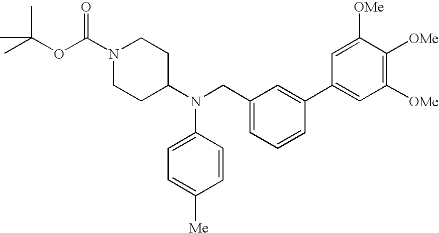

1-(tert-Butoxycarbonyl)-4-[(4-methylphenyl)amino]piperidine (581 mg) and 3-(3,4,5-trimethoxyphenyl)benzyl chloride (586 mg) was treated in the same manner as described in Example 9 to give light yellow amorphous of the title compound.

Yield: 1.03 g (94%).

¹H-NMR (400 MHz, CDCl₃) δ: 1.44 (s, 9H), 1.50-1.66 (m, 2H), 1.83-1.90 (m, 2H), 2.23 (s, 3H), 2.72-2.85 (m, 2H), 3.82-3.92 (m, 1H), 3.88 (s, 3H), 3.89 (s, 6H), 4.11-4.30 (m, 2H), 4.47 (s, 2H), 6.68 (d, 2H, J=8.6 Hz), 6.71 (s, 2H), 7.00 (d, 2H, J=8.8 Hz), 7.23-7.27 (m, 1H), 7.32-7.44 (m, 3H).

### PREPARATION EXAMPLE 152

### Synthesis of 4-[N-(4-methylphenyl)-N-[3-(3,4,5-trimethoxyphenyl)benzyl]amino]piperidine Hydrochloride

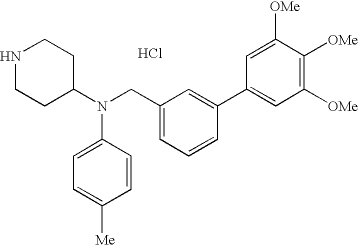

1-(tert-Butoxycarbonyl)-4-(4-methylphenyl)-N-[3-(3,4,5-trimethoxyphenyl)benzyl]amino]piperidine (1.03 g) was treated in the same manner as described in Preparation Example 94 to give light yellow powder of the title compound.

Yield: 882 mg (97%).

### EXAMPLES 119 TO 126

These compounds were obtained by the condensation of amines obtained in Preparation Examples 148, 150 and 152 with chloride derivatives obtained in Preparation Examples 3, 42 and 48. Free bases obtained were then converted to the corresponding hydrochlorides. Yields and NMR data of their free bases are listed below.

| Ex- | | | NMR data (400 MHz, measured |
|---|---|---|---|
| ample | Structure | Yield | as free bases, CDCl₃) δ |
| 119 | 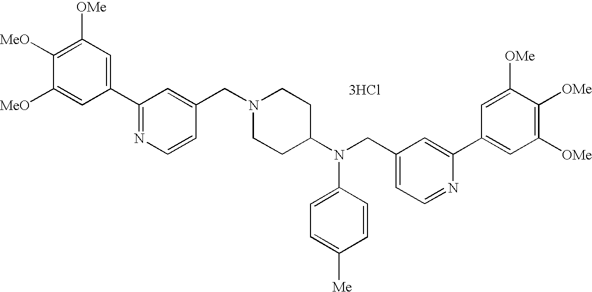 | 66% | 1.70-1.82(m, 2H), 1.83-1.91(m, 2H), 2.13-2.25(m, 2H), 2.23(s, 3H), 2.96-3.02(m, 2H), 3.57(s, 2H), 3.73-3.83(m, 1H), 3.89(s, 3H), 3.90(s, 3H), 3.93(s, 6H), 3.96(s, 6H), 4.50(s, 2H), 6.64(d, 2H, J=8.8Hz), 7.01(d, 2H, J=8.5Hz), 7.13-7.17(m, 3H), 7.20(d, 1H, J=4.9Hz), 7.22(s, 2H), 7.56(s, 1H), 7.59(s, 1H), 8.54(d, |
| #1H, J=5.1Hz), 8.59(d, 1H, J=4.9Hz). | | | |
| 120 | 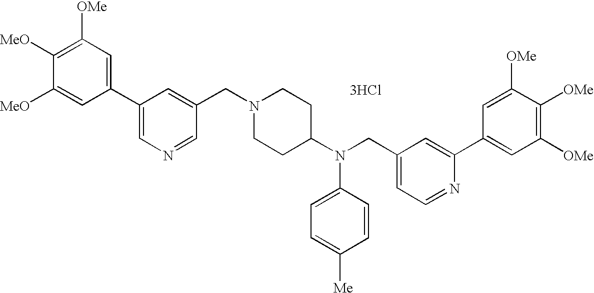 | 41% | 1.60-1.91(m, 4H), 2.12-2.24(m, 2H), 2.23(s, 3h), 2.95-3.08(m, 2H), 3.59(s, 2H), 3.73-3.83(m, 1H), 3.89(s, 3H), 3.89(s, 3H), 3.92(s, 6H), 3.93(s, 6H), 4.49(s, 2H), 6.63(d, 2H, J=8.6Hz), 6.75(s, 2H), 7.00(d, 2H, J=8.6Hz), 7.13-7.16(m, 3H), 7.55(s, 1H), 7.76(s, 1H), 8.50(d, 1H, J=1.8Hz), 8.53(d, 1H, J=5.1Hz), 8.70(s, 1H). |
| 121 | 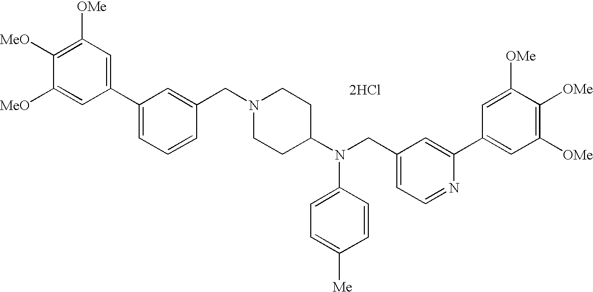 | 69% | 1.67-1.80(m, 2H), 1.81-1.89(m, 2H), 2.09-2.20(m, 2H), 2.22(s, 3H), 2.98-3.06(m, 2H), 3.58(s, 2H), 3.72-3.81(m, 1H), 3.88(s, 3H), 3.89(s, 3H), 3.92(s, 6H), 3.92(s, 6H), 4.49(s, 2H), 6.63(d, 2H, J=8.4Hz), 6.76(s, 2H), 7.00(d, 2H, J=8.6Hz), 7.12-7.15(m, 3H), 7.26-7.32(m, 1H), 7.37(dd, 1H, J=7.6Hz, 7.6Hz), |
| #7.41-7.48(m, 2H), 7.55(s, 1H), 8.53(d, 1H, J=5.0Hz). | | | |
| 122 | 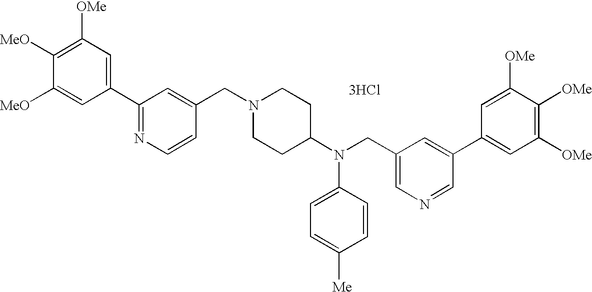 | 47% | 1.55-2.00(m, 4H), 2.12-2.31(m, 2H), 2.22(s, 3H), 2.93-3.10(m, 2H), 3.60(br, 2H), 3.69-3.80(m, 1H), 3.88(s, 3H), 3.89(s, 6H), 3.90(s, 3H), 3.96(s, 6H), 4.53(s, 2H), 6.66(s, 2H), 6.69(d, 2H, J=8.6Hz), 7.00(d, 2H, J=8.6Hz), 7.19-7.27(m, 4H), 7.68(s, 1H), 8.50(s, 1H), 8.60(d, 1H, J=4.9Hz), 8.64(d, 1H, J=2.2Hz). |
| 123 | 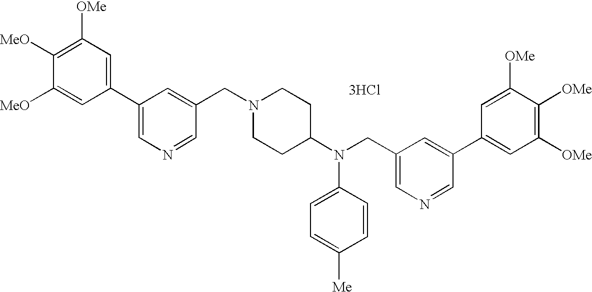 | 34% | 1.67-1.98(m, 4H), 2.10-2.38(m, 2H), 2.22(s, 3H), 2.85-3.10(m, 2H), 3.53-3.67(s, 2H), 3.67-3.79(m, 1H), 3.88(s, 3H), 3.89(s, 6H), 3.90(s, 3H), 3.93(s, 6H), 4.51(s, 2H), 6.66(s, 2H), 6.68(d, 2H, J=8.8Hz), 6.76(s, 2H), 7.00(d, 2H, J=8.2Hz), 7.67(s, 1H), 7.77(br, 1H), 8.47-8.53(m, 2H), 8.63(d, 1H, |
| #J=2.0Hz), 8.70(s, 1H). | | | |
| 124 | 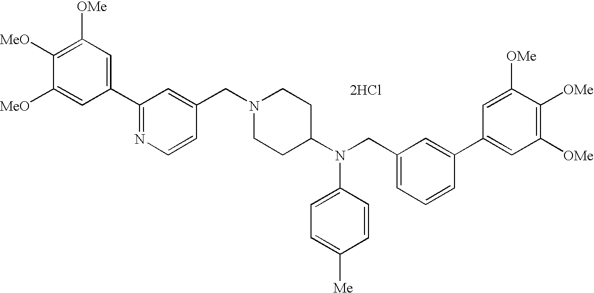 | 91% | 1.73-1.92(m, 4H), 2.12-2.26(m, 2H), 2.21(s, 3H), 2.92-3.02(m, 2H), 3.57(s, 2H), 3.72-3.82(m, 1H), 3.87(s, 3H), 3.88(s, 6H), 3.90(s, 3H), 3.95(s, 6H), 4.53(s, 2H), 6.67(d, 2H, J=7.8Hz), 6.70(s, 2H), 6.99(d, 2H, J=8.0Hz), 7.18-7.25(m, 4H), 7.33(dd, 1H, J=7.4Hz), 7.4Hz), 7.38(d, 1H, J=7.2Hz), 7.42(s, 1H), 7.59(s, 1H), |
| #8.58(d, 1H, J=4.7Hz). | | | |
| 125 | 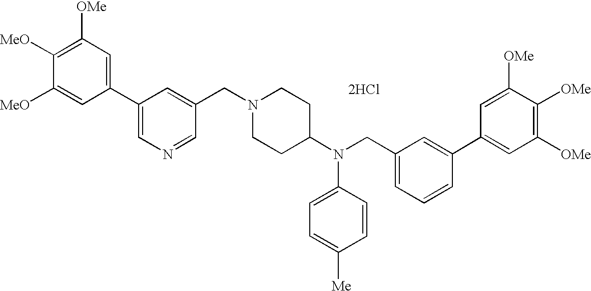 | 74% | 1.70-1.92(m, 4H), 2.10-2.28(m, 2H), 2.21(s, 3H), 2.92-3.06(m, 2H), 3.58(s, 2H), 3.72-3.82(m, 1H), 3.87(s, 3H), 3.88(s, 6H), 3.89(s, 3H), 3.93(s, 6H), 4.51(s, 2H), 6.66(d, 2H, J=8.6Hz), 6.70(s, 2H), 6.75(s, 2H), 7.23(d, 1H, J=7.0Hz), 7.32(dd, 1H, J=7.6Hz, 7.6Hz), 7.37(d, 1H, J=7.8Hz), 7.41(s, 1H), 7.77(s, 1H), 8.49(s, 1H), |
| #8.69(s, 1H). | | | |
| 126 | 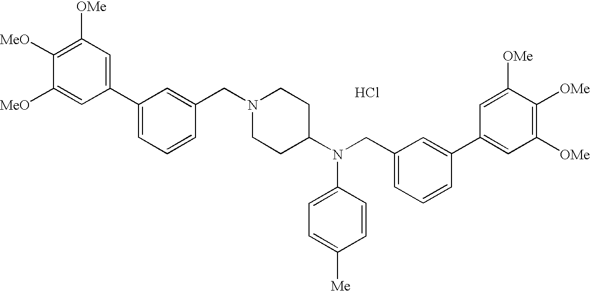 | 84% | 1.71-1.88(m, 4H), 2.08-2.18(m, 2H), 2.21(s, 3H), 2.96-3.04(m, 2H), 3.58(s, 2H), 3.71-3.83(m, 1H), 3.87(s, 3H), 3.88(s, 6H), 3.89(s, 3H), 3.92(s, 6H), 4.52(s, 2H), 6.66(d, 2H, J=8.6Hz), 6.70(s, 2H), 6.76(s, 2H), 6.98(d, 2H, J=8.3Hz), 7.22-7.47(m, 8H). |

### PREPARATION EXAMPLE 153

### Synthesis of 1-(tert-butoxycarbonyl)-4-[[4-(trifluoromethyl)phenyl]amino]piperidine

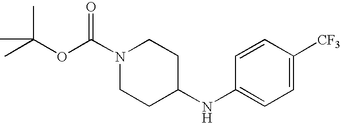

1-(tert-Butoxycarbonyl)-4-piperidone (5.00 g) and 4-(trifluoromethyl)aniline (3.85 g) was treated in the same manner as described in Preparation Example 37 to give white powder of the title compound.

Yield: 3.30 g (40%).

¹H-NMR (400 MHz, CDCl₃) δ: 1.30-1.41 (m, 2H), 1.47 (s, 9H), 2.00-2.07 (m, 2H), 2.88-2.99 (m, 2H), 3.32-3.52 (m, 1H), 3.83-3.89 (m, 1H), 4.00-4.14 (m, 2H), 6.59 (d, 2H, J=8.4 Hz), 7.39 (d, 2H, J=8.4 Hz).

### PREPARATION EXAMPLE 154

### Synthesis of 1-(tert-butoxycarbonyl)-4-[N-[4-(trifluoromethyl)phenyl]-N-[[2-(3,4,5-trimethoxyphenyl)pyridin-4-yl]methyl]amino]piperidine

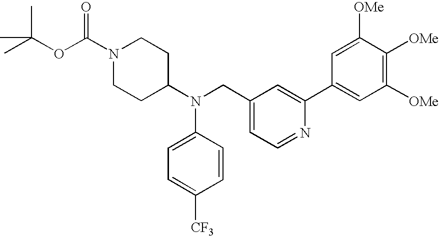

1-(tert-Butoxycarbonyl)-4-[[4-(trifluoromethyl)phenyl]amino]piperidine (688 mg) and 4-chloromethyl-2-(3,4,5-trimethoxyphenyl)pyridine (588 mg) was treated in the same manner as described in Example 9 to give light yellow amorphous of the title compound.

Yield: 412 mg (34%).

¹H-NMR (400 MHz, CDCl₃) δ: 1.45 (s, 9H), 1.54-1.68 (m, 2H), 1.81-1.90 (m, 2H), 2.77-2.90 (m, 2H), 3.89 (s, 3H), 3.92 (s, 6H), 3.98-4.07 (m, 1H), 4.18-4.33 (m, 2H), 4.55 (s, 2H), 6.73 (d, 2H, J=8.8 Hz), 7.09 (d, 1H, J=3.7 Hz), 7.13 (s, 2H), 7.44 (d, 2H, J=8.8 Hz), 7.49 (s, 1H), 8.58 (d, 1H, J=5.1 Hz).

### PREPARATION EXAMPLE 155

### Synthesis of 4-[N-[4-(trifluoromethyl)phenyl]-N-[[2-(3,4,5-trimethoxyphenyl)pyridin-4-yl]methyl]amino]piperidine Dihydrochloride

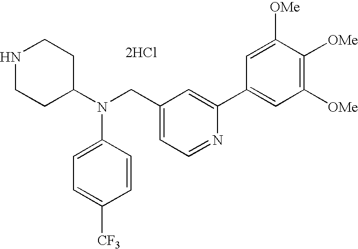

1-(tert-Butoxycarbonyl)-4-[N-[4-(trifluoromethyl)phenyl]-N-[[2-(3,4,5-trimethoxyphenyl)pyridin-4-yl]methyl]amino]piperidine (412 mg) was treated in the same manner as described in Preparation Example 94 to give light yellow powder of the title compound.

Yield: 359 mg (91%).

### PREPARATION EXAMPLE 156

### Synthesis of 1-(tert-butoxycarbonyl)-4-N-[4-(trifluoromethyl)phenyl]-N-[3-(3,4,5-trimethoxyphenyl)benzyl]amino]piperidine:

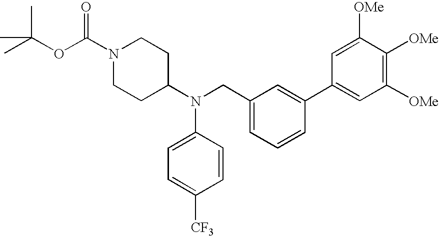

1-(tert-Butoxycarbonyl)-4-[[4-(trifluoromethyl)phenyl]amino]piperidine (689 mg) and 3-(3,4,5-trimethoxyphenyl)benzyl chloride (586 mg) was treated in the same manner as described in Example 9 to give light yellow amorphous of the title compound.

Yield: 522 mg (44%).

¹H-NMR (400 MHz, CDCl₃) δ: 1.45 (s, 9H), 1.58-1.70 (m, 2H), 1.83-1.90 (m, 2H), 2.76-2.87 (m, 2H), 3.87 (s, 6H), 3.88 (s, 3H), 3.96-4.06 (m, 1H), 4.15-4.30 (m, 2H), 4.58 (s, 2H), 6.68 (s, 2H), 6.76 (d, 2H, J=8.8 Hz), 7.19 (s, 1H, J=7.4 Hz), 7.33-7.44 (m, 5H).

### PREPARATION EXAMPLE 157

### Synthesis of 4-[N-[4-(trifluoromethyl)phenyl]-N-[3-(3,4,5-trimethoxyphenyl)benzyl]amino]piperidine Hydrochloride

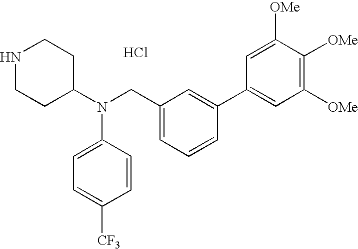

1-(tert-Butoxycarbonyl)-4-[N-[4-(trifluoromethyl)phenyl]-N-[3-(3,4,5-trimethoxyphenyl)benzyl]amino]piperidine (522 mg) was treated in the same manner as described in Preparation Example 94 to give light yellow powder of the title compound.

Yield: 460 mg (99%).

### EXAMPLE 127 TO 132

These compounds were obtained by the condensation of amines obtained in Preparation Examples 155 and 157 with chloride derivatives obtained in Preparation Examples 3, 42 and 48. Free bases obtained were then converted to the corresponding hydrochlorides. Yields and NMR data of their free bases are listed below.

| Ex- | | | NMR data (400 MHz, measured as |
|---|---|---|---|
| ample | Structure | Yield | free bases, CDCl₃) δ |
| 127 | 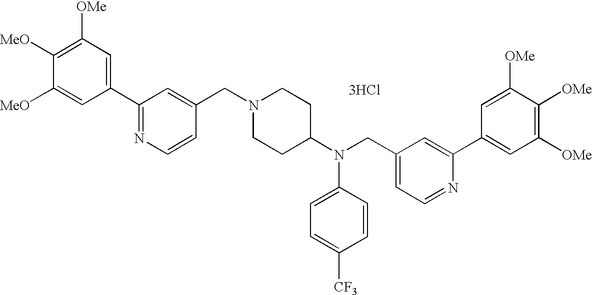 | 72% | 1.74-1.92(m, 4H), 2.17-2.26(m, 2H), 2.96-3.04(m, 2H), 3.59(s, 2H), 3.89(s, 3H), 3.90(s, 3H), 3.91(s, 6H), 3.96(s, 6H), 4.60(s, 2H), 6.72(d, 2H, J=8.8Hz), 7.10(d, 1H, J=4.9Hz), 7.13(s, 2H), 7.20(d, 1H, J=5.1Hz), 7.43(d, 2H, J=8.8Hz), 7.50(s, 1H), 7.59(s, 1H), 8.56(d, 1H, J=4.9Hz), 8.58(d, 1H, J=5.1Hz). |
| 128 | 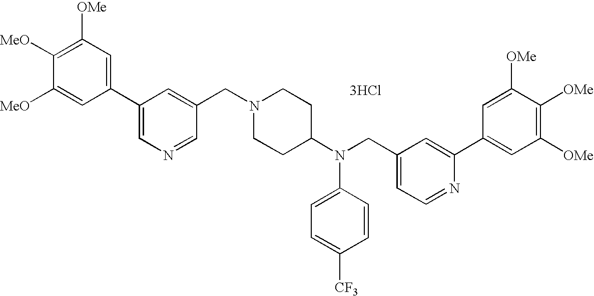 | 51% | 1.70-1.90(m, 4H), 2.14-2.28(m, 2H), 2.96-3.08(m, 2H), 3.61(s, 2H), 3.87-3.96(m, 1H), 3.89(s, 3H), 3.90(s, 3H), 3.91(s, 6H), 3.93(s, 6H), 4.59(s, 2H), 6.71(d, 2H, J=8.8Hz), 6.75(s, 2H), 7.07-7.15(m, 3H), 7.43(d, 2H, J=8.8Hz), 7.49(s, 1H), 7.76(s, 1H), 8.51(d, 1H, J=1.8Hz), 8.57(d, 1H, J=5.1Hz), 8.70(s, 1H). |
| 129 | 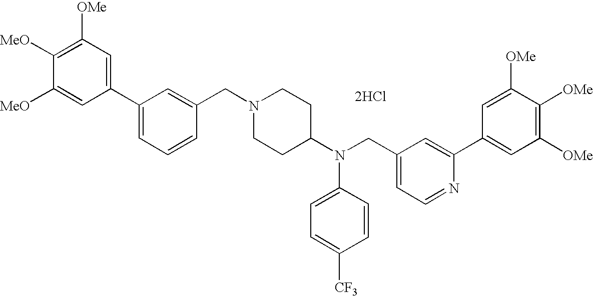 | 59% | 1.72-1.88(m, 4H), 2.11-2.24(m, 2H), 2.98-3.10(m, 2H), 3.59(s, 2H), 3.87-3.95(m, 1H), 3.88(s, 3H), 3.89(s, 3H), 3.90(s, 6H), 3.92(s, 6H), 4.59(s, 2H), 6.71(d, 2H, J=9.0Hz), 6.76(s, 2H), 7.08(d, 1H, J=5.1Hz), 7.12(s, 2H), 7.29(d, 1H, J=7.4Hz), 7.37(dd, 1H, J=7.6Hz, 7.6Hz), 7.40-7.52(m, 5H), 8.56(d, 1H, J=5.1Hz). |
| 130 | 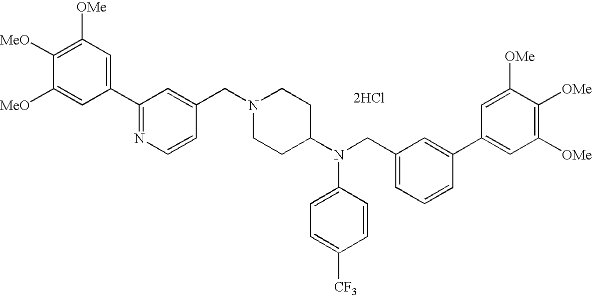 | 81% | 1.78-1.94(m, 4H), 2.15-2.27(m, 2H), 2.94-3.08(m, 2H), 3.58(s, 2H), 3.86(s, 6H), 3.87(s, 3H), 3.90(s, 3H), 3.96(s, 6H), 4.63(s, 2H), 6.67(s, 2H), 6.74(d, 2H, J=8.8Hz), 7.17-7.24(m, 4H), 7.34-7.45(m, 5H), 7.59(s, 1H), 8.59(d, 1H, J=5.1Hz). |
| 131 | 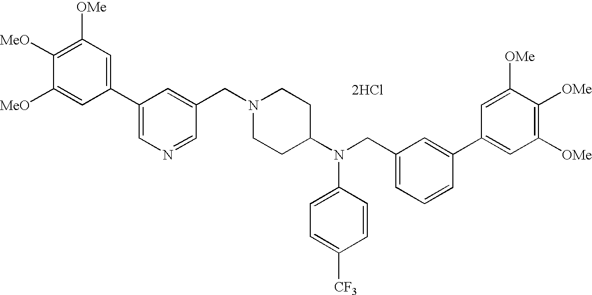 | 54% | 1.75-1.90(m, 4H), 2.14-2.24(m, 2H), 2.95-3.04(m, 2H), 3.60(s, 2H), 3.84-3.88(m, 1H), 3.86(m, 1H), 3.87(s, 3H), 3.90(s, 3H), 3.93(s, 6H), 4.61(s, 2H), 6.67(s, 2H), 6.72-6.77(m, 4H), 7.18(d, 1H, J=7.4Hz), 7.33-7.43(m, 5H), 7.76(s, 1H), 8.50(d, 1H, J=1.9Hz), 8.69(d, 1H, J=1.9Hz). |
| 132 | 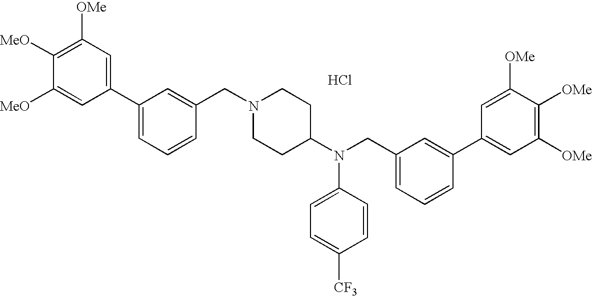 | 67% | 1.76-1.88(m, 4H), 2.11-2.19(m, 2H), 2.98-3.06(m, 2H), 3.59(s, 2H), 3.86(s, 6H), 3.87(s, 3H), 3.89(s, 3H), 3.92(s, 6H), 4.61(s, 2H), 6.67(s, 2H), 6.73(d, 2H, J=8.8Hz), 6.76(s, 2H), 7.18(d, 1H, J=7.3Hz), 7.29(d, 1H, J=7.6Hz), 7.32-7.47(m, 8H). |

### PREPARATION EXAMPLE 158

### Synthesis of 4-(4-bromophenyl)amino-1-(tert-butoxycarbonyl)piperidine

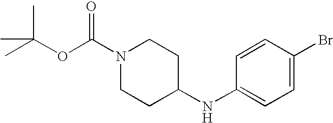

1-(tert-Butoxycarbonyl)-4-piperidone (5.00 g) and 4-bromoaniline (4.11 g) was treated in the same manner as described in Example 37 to give white crystalline powder of the title compound.

Yield: 3.09 g (36%).

¹H-NMR (400 MHz, CDCl₃) δ: 1.25-1.37 (m, 2H), 1.46 (s, 9H), 1.97-2.05 (m, 2H), 2.86-2.96 (m, 2H), 3.33-3.42 (m, 2H), 3.47-3.57 (m, 1H), 3.96-4.12 (m, 2H), 6.47 (d, 2H, J=8.8 Hz), 7.24 (d, 2H, J=9.0 Hz).

### PREPARATION EXAMPLE 159

### Synthesis of 4-[N-(4-bromophenyl)-N-[[2-(3,4,5-trimethoxyphenyl)pyridin-4-yl]methyl]amino]-1-(tert-butoxycarbonyl)piperidine

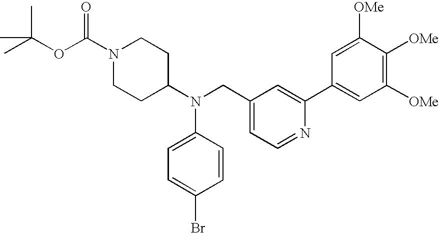

4-(4-Bromophenyl)amino-1-(tert-butoxycarbonyl)piperidine (711 mg) and 4-chloromethyl-2-(3,4,5-trimethoxyphenyl)pyridine (588 mg) was treated in the same manner as described in Example 9 to give light yellow amorphous of the title compound.

Yield: 607 mg (50%).

¹H-NMR (400 MHz, CDCl₃) δ: 1.45 (s, 9H), 1.50-1.64 (m, 2H), 1.81-1.88 (m, 2H), 2.74-2.88 (m, 2H), 3.86-3.94 (m, 1H), 3.89 (s, 3H), 3.93 (s, 6H), 4.14-4.32 (m, 2H), 4.46 (s, 2H), 6.59 (d, 2H, J=9.1 Hz), 7.10 (d, 1H, J=5.2 Hz), 7.14 (s, 2H), 7.28 (d, 2H, J=9.1 Hz), 7.50 (s, 1H), 8.57 (d, 1H, J=5.0 Hz).

### PREPARATION EXAMPLE 160

### Synthesis of 4-[N-(4-bromophenyl)-N-[[2-(3,4,5-trimethoxyphenyl)pyridin-4-yl]methyl]amino]piperidine Dihydrochloride

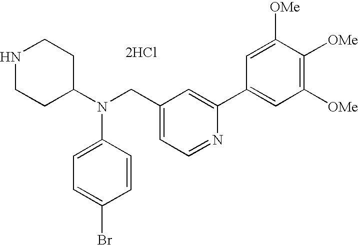

4-[N-(4-Bromophenyl)-N-[[2-(3,4,5-trimethoxyphenyl)pyridinyl]methyl]amino]-1-(tert-butoxycarbonyl)piperidine (607 mg) was treated in the same manner as described in Preparation Example 94 to give light yellow powder of the title compound.

Yield: 541 mg (93%).

### PREPARATION EXAMPLE 161

### Synthesis of 4-[N-(4-bromophenyl)-N-[[3-(3,4,5-trimethoxyphenyl)pyridin-5-yl]methyl]amino]-1-(tert-butoxycarbonyl)piperidine

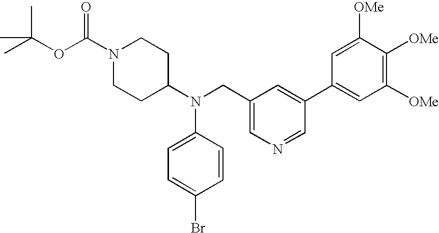

4-(4-Bromophenyl)amino-1-(tert-butoxycarbonyl)piperidine (711 mg) and 5-chloromethyl-3-(3,4,5-trimethoxyphenyl)pyridine (588 mg) was treated in the same manner as described in Example 9 to give light yellow amorphous of the title compound.

Yield: 347 mg (28%).

¹H-NMR (400 MHz, CDCl₃) δ: 1.45 (s, 9H), 1.52-1.67 (m, 2H), 1.80-1.89 (m, 2H), 2.72-2.87 (m, 2H), 3.82-3.92 (m, 1H), 3.89 (s, 3H), 3.90 (s, 6H), 4.14-4.33 (m, 2H), 4.50 (s, 2H), 6.63 (d, 2H, J=9.2 Hz), 6.65 (s, 2H), 7.28 (d, 2H, J=9.4 Hz), 7.61 (s, 1H), 8.47 (d, 1H, J=2.0 Hz), 8.67 (d, 1H, J=2.2 Hz).

### PREPARATION EXAMPLE 162

### Synthesis of 4-[N-(4-bromophenyl)-N-[[3-(3,4,5-trimethoxyphenyl)pyridin-5-yl]methyl]amino]piperidine Dihydrochloride

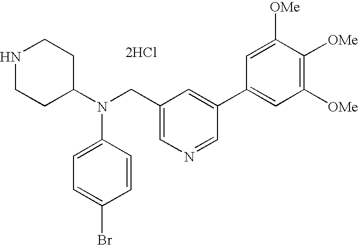

4-N-(4-Bromophenyl)-N-[[3-(3,4,5-trimethoxyphenyl)pyridin-5-yl]methyl]amino]-1-(tert-butoxycarbonyl)piperidine (347 mg) was treated in the same manner as described in Preparation Example 94 to give light yellow powder of the title compound.

Yield: 302 mg (91%).

### PREPARATION EXAMPLE 163

### Synthesis of 4-[N-(4-bromophenyl)-N-[3-(3,4,5-trimethoxyphenyl)benzyl]amino]-1-(tert-butoxycarbonyl)piperidine

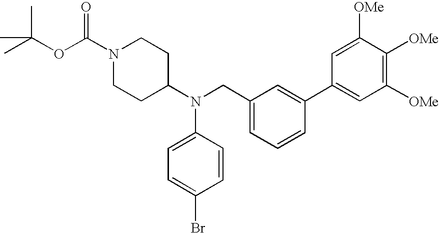

4-(4-Bromophenyl)amino-1-(tert-butoxycarbonyl)piperidine (711 mg) and 3-(3,4,5-trimethoxyphenyl)benzyl chloride (586 mg) was treated in the same manner as described in Example 9 to give light yellow amorphous of the title compound.

Yield: 1.14 g (93%).

¹H-NMR (400 MHz, CDCl₃) δ: 1.45 (s, 9H), 1.52-1.67 (m, 2H), 1.80-1.89 (m, 2H), 2.72-2.86 (m, 2H), 3.84-3.91 (m, 1H), 3.88 (s, 3H), 3.89 (s, 6H), 4.11-4.32 (m, 2H), 4.49 (s, 2H), 6.62 (d, 2H, J=9.2 Hz), 6.69 (s, 2H), 7.19 (d, 1H, J=7.6 Hz), 7.25 (d, 2H, J=5.5 Hz), 7.32-7.42 (m, 3H).

### PREPARATION EXAMPLE 164

### Synthesis of 4-[N-(4-bromophenyl)-N-[3-(3,4,5-trimethoxyphenyl)benzyl]amino]piperidine Hydrochloride

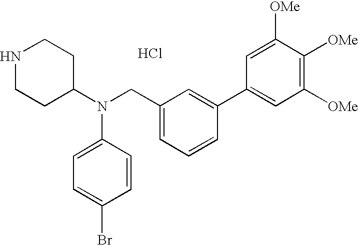

4-[N-(4-Bromophenyl)-N-[3-(3,4,5-trimethoxyphenyl)benzyl]amino]-1-(tert-butoxycarbonyl)piperidine (1.14 g) was treated in the same manner as described in Preparation Example 94 to give light yellow powder of the title compound.

Yield: 973 mg (84%).

### EXAMPLES 133 TO 140

These compounds were obtained by the condensation of amines obtained in Preparation Examples 160, 162 and 164 with chloride derivatives obtained in Preparation Examples 3, 42 and 48. Free bases obtained were then converted to the corresponding hydrochlorides. Yields and NMR data of their free bases are listed below.

| | | | NMR data (400 MHz, |
|---|---|---|---|
| | | | measured as free |
| Example | Structure | Yield | bases, CDCl₃) δ |
| 133 | 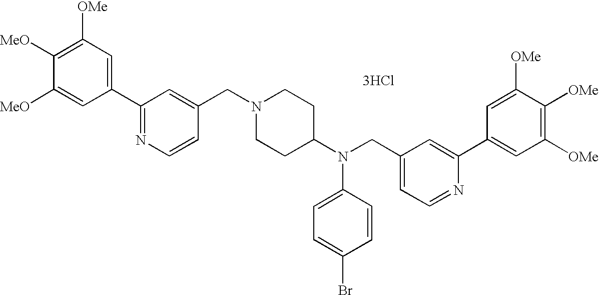 | 52% | 1.70-1.90 (m, 4H), 2.14-2.25 (m, 2H), 2.94-3.04 (m, 2H), 3.58 (s, 2H), 3.73-3.84 (m, 1H), 3.89 (s, 3H), 3.90 (s, 3H), 3.92 (s, 6H), 3.96 (s, 6H), 4.52 (s, 2H), 6.57 (d, 2H, J=8.8 Hz), 7.10 (d, 1H, J=4.9 Hz), 7.14 (s, 2H), 7.20 (d, 1H, J=4.9 Hz), 7.22 (s, 2H), 7.26 (d, 2H, J=8.5 Hz), 7.51 (s, 1H), 7.59 (s, 1H), 8.56 (d, 1H, J=4.9 Hz), 8.59 (d, 1H, J=4.9 Hz). |
| 134 | 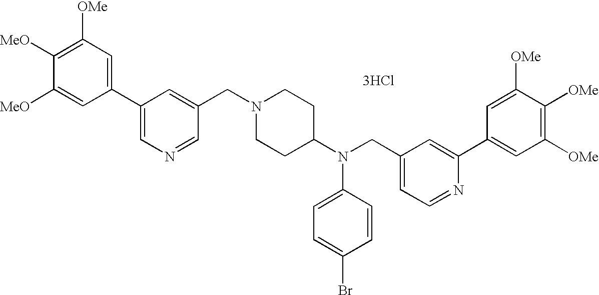 | 56% | 1.68-1.88 (m, 4H), 2.12-2.24 (m, 2H), 2.95-3.04 (m, 2H), 3.59 (s, 2H), 3.72-3.84 (m, 1H), 3.89 (s, 3H), 3.90 (s, 3H), 3.92 (s, 6H), 3.93 (s, 6H), 4.50 (s, 2H), 6.57 (d, 2H, J=9.2 Hz), 6.74 (s, 2H), 7.09 (d, 1H, J=3.9 Hz), 7.13 (s, 2H), 7.26 (d, 2H, J=8.8 Hz), 7.50 (s, 1H), 7.75 (s, 1H), 8.50 (d, 1H, J=2.0 Hz), 8.55 (d, 1H, J=5.0 Hz), 8.69 (d, 1H, J=2.0 Hz). |
| 135 | 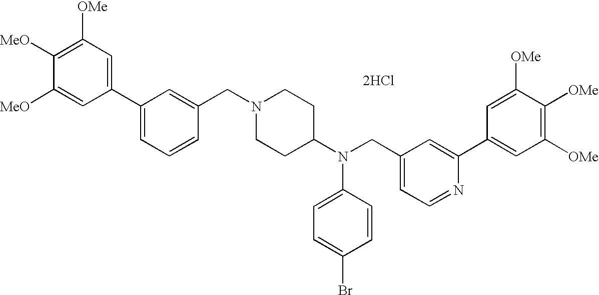 | 65% | 1.70-1.86 (m, 4H), 2.10-2.20 (m, 2H), 2.97-3.08 (m, 2H), 3.59 (s, 2H), 3.72-3.82 (m, 1H), 3.89 (s, 6H), 3.92 (s, 6H), 3.92 (s, 6H), 4.50 (s, 2H), 6.56 (d, 2H, J=9.2 Hz), 6.76 (s, 2H), 7.09 (d, 1H, J=5.1 Hz), 7.13 (s, 2H), 7.23-7.33 (m, 3H), 7.37 (dd, 1H, J=7.4 Hz), 7.41-7.48 (m, 2H), 7.49 (s, 1H), 8.54 (d, 1H, J=5.1 Hz). |
| 136 | 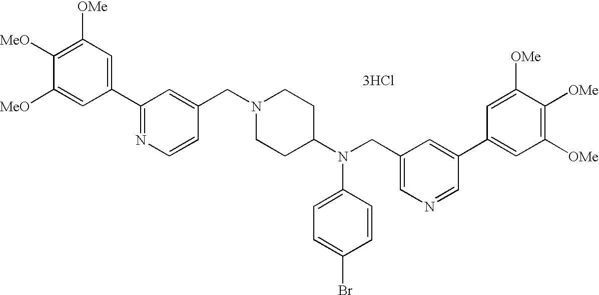 | 49% | 1.77-1.93 (m, 4H), 2.12-2.30 (m, 2H), 2.94-3.10 (m, 2H), 3.60 (s, 2H), 3.73-3.83 (m, 1H), 3.88 (s, 3H), 3.89 (s, 6H), 3.90 (s, 3H), 3.96 (s, 6H), 4.55 (s, 2H), 6.61 (d, 2H, J=9.2 Hz), 6.65 (s, 2H), 7.19-7.29 (m, 5H), 7.62(br, 2H), 8.47 (d, 1H, J=1.6 Hz), 8.60 (d, 1H, J=4.9 Hz), 8.66 (d, 1H, J=2.0 Hz). |
| 137 | 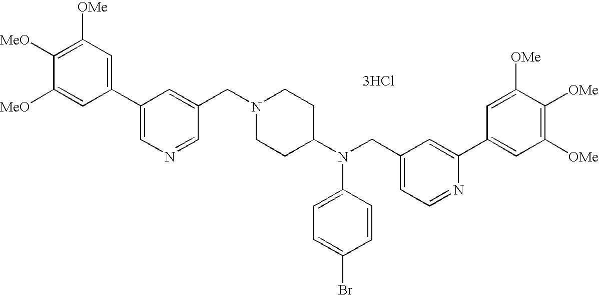 | 50% | 1.70-1.92 (m, 4H), 2.12-2.27 (m, 2H), 2.93-3.07 (m, 2H), 3.60 (s, 3H), 3.89 (s, 6H), 3.90 (s, 3H), 3.93 (s, 6H), 4.54 (s, 2H), 6.60 (d, 2H, J=9.0 Hz), 6.64 (s, 2H), 6.73-6.80 (m, 2H), 7.25 (s, 2H), 7.61 (s, 1H), 7.77(br, 1H), 8.45 (d, 1H, J=1.7 Hz), 8.50 (d, 1H, J=1.7 Hz), 8.65 (d, 1H, J=2.0 Hz). |
| 138 | 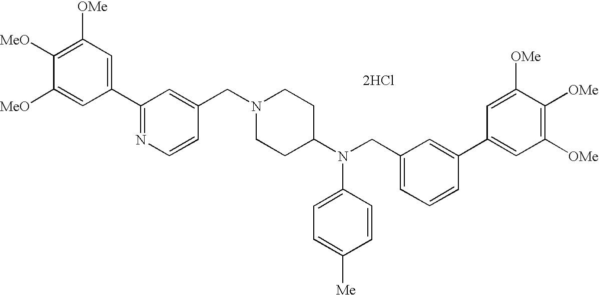 | 81% | 1.75-1.90 (m, 4H), 2.17-2.24 (m, 2H), 2.94-3.02 (m, 2H), 3.57 (s, 2H), 3.72-3.83 (m, 1H), 3.88 (s, 3H), 3.88 (s, 6H), 3.90 (s, 3H), 3.95 (s, 6H), 4.54 (s, 2H), 6.60 (d, 2H, J=9.2 Hz), 6.69 (s, 2H), 7.18-7.27 (m, 6H), 7.32-7.42 (m, 3H), 7.60 (s, 1H), 8.58 (d, 1H, J=4.9 Hz). |
| 139 | 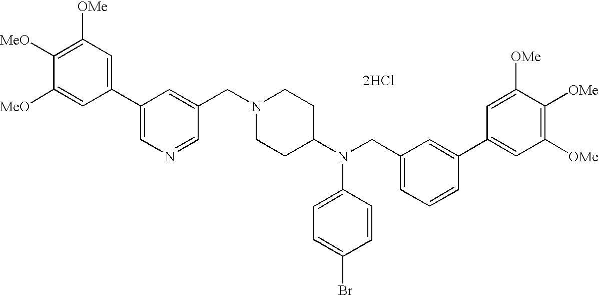 | 80% | 1.72-1.90 (m, 4H), 2.13-2.21 (m, 2H), 2.94-3.05 (m, 2H), 3.59 (s, 2H), 3.72-3.82 (m, 1H), 3.87 (s, 3H), 3.88 (s, 6H), 3.89 (s, 3H), 3.93 (s, 6H), 4.53 (s, 2H), 6.60 (d, 2H, J=9.0 Hz), 6.68 (s, 2H), 6.75 (s, 2H), 7.19 (d, 1H, J=7.2 Hz), 7.24 (d, 2H, J=9.0 Hz), 7.31-7.41 (m, 3H), 7.76 (s, 1H), 8.50 (d, 1H, J=1.8 Hz), 8.70 (s, 1H). |
| 140 | 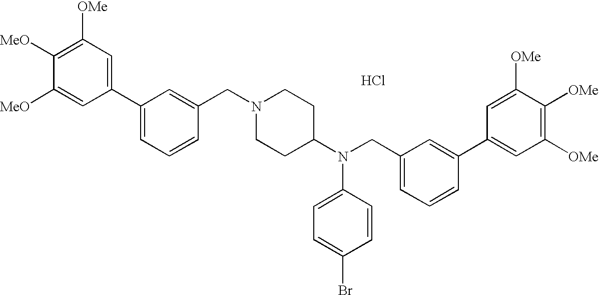 | 78% | 1.72-1.88 (m, 4H), 2.08-2.18 (m, 2H), 2.97-3.06 (m, 2H), 3.58 (s, 2H), 3.71-3.82 (m, 1H), 3.87 (s, 3H), 3.88 (s, 6H), 3.89 (s, 3H), 3.92 (s, 6H), 4.53 (s, 2H), 6.59 (d, 2H, J=9.3 Hz), 6.68 (s, 2H), 6.76 (s, 2H), 7.18 (d, 1H, J=7.3 Hz), 7.21-7.47 (m, 9H) |

### PREPARATION EXAMPLE 165

### Synthesis of 1-(tert-butoxycarbonyl)-4-[(4-chlorophenyl)amino]piperidine

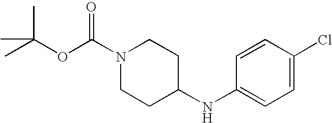

1-(tert-Butoxycarbonyl)-4-piperidone (5.00 g) and 4-chloroaniline (3.05 g) was treated in the same manner as described in Preparation Example 37 to give white powder of the title compound.

Yield: 3.80 g (49%).

¹H-NMR (400 MHz, CDCl₃) δ: 1.24-1.38 (m, 2H), 1.46 (s, 9H), 1.97-2.05 (m, 2H), 2.86-2.96 (m, 2H), 3.32-3.42 (m, 2H), 3.51 (br, 1H), 6.52 (d, 2H, J=9.0 Hz), 7.11 (d, 2H, J=9.0 Hz).

### PREPARATION EXAMPLE 166

### Synthesis of 1-(tert-butoxycarbonyl)-4-[N-(4-chlorophenyl)-N-[[2-(3,4,5-trimethoxyphenyl)pyridin-4-yl]methyl]amino]piperidine

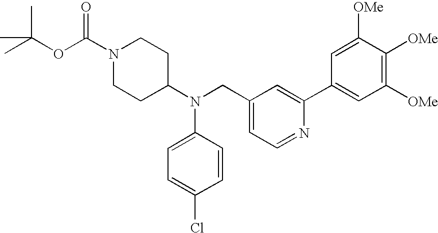

1-(tert-Butoxycarbonyl)-4-[(4-chlorophenyl)amino]piperidine (621 mg) and 4-chloromethyl-2-(3,4,5-trimethoxyphenyl)pyridine (588 mg) was treated in the same manner as described in Example 9 to give light yellow amorphous of the title compound.

Yield: 789 mg (69%).

¹H-NMR (400 MHz, CDCl₃) δ: 1.45 (s, 9H), 1.51-1.68 (m, 2H), 1.80-1.89 (m, 2H), 2.72-2.86 (m, 2H), 3.87-3.90 (m, 1H), 3.89 (s, 3H), 3.93 (s, 6H), 4.64 (s, 2H), 6.64 (d, 2H, J=9.0 Hz), 7.14 (d, 1H, J=5.3 Hz), 7.15 (d, 2H, J=9.0 Hz), 7.51 (s, 2H), 8.57 (d, 2H, J=5.1 Hz).

### PREPARATION EXAMPLE 167

### Synthesis of 4-[N-(4-chlorophenyl)-N-[[2-(3,4,5-trimethoxyphenyl)pyridin-4-yl]methyl]amino]piperidine Dihydrochloride

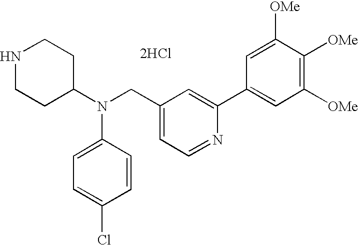

1-(tert-Butoxycarbonyl)-4-[N-(4-chlorophenyl)-N-[[2-(3,4,5-trimethoxyphenyl)pyridin-4-yl]methyl]amino]piperidine (789 mg) was treated in the same manner as described in Preparation Example 94 to give light yellow powder of the title compound.

Yield: 673 mg (90%).

### PREPARATION EXAMPLE 168

### Synthesis of 1-(tert-butoxycarbonyl)-4-[N-(4-chlorophenyl)-N-[[3-(3,4,5-trimethoxyphenyl)pyridin-5-yl]methyl]amino]piperidine

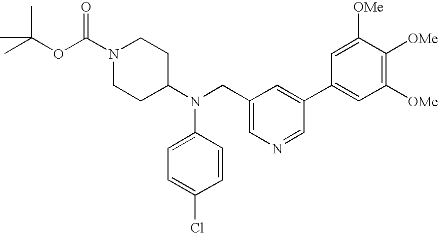

1-(tert-Butoxycarbonyl)-4-[(4-chlorophenyl)amino]piperidine (621 mg) and 5-chloromethyl-3-(3,4,5-trimethoxyphenyl)pyridine (588 mg) was treated in the same manner as described in Example 9 to give light yellow amorphous of the title compound.

Yield: 268 mg (24%).

¹H-NMR (400 MHz, CDCl₃) δ: 1.45 (s, 9H), 1.56-1.76 (m, 2H), 1.80-1.90 (m, 2H), 2.76-2.83 (m, 2H), 3.86-3.90 (m, 1H), 3.89 (s, 3H), 3.90 (s, 6H), 4.15-4.30 (m, 2H), 4.50 (s, 2H), 6.66 (s, 2H), 6.68 (d, 2H, J=9.2 Hz), 7.15 (d, 2H, J=9.0 Hz), 7.63 (s, 1H), 8.47 (d, 1H, J=2.0 Hz), 8.66 (d, 1H, J=2.0 Hz).

### PREPARATION EXAMPLE 169

### Synthesis of 4-[N-(4-chlorophenyl)-N-[[3-(3,4,5-trimethoxyphenyl)pyridin-5-yl]methyl]amino]piperidine Dihydrochloride

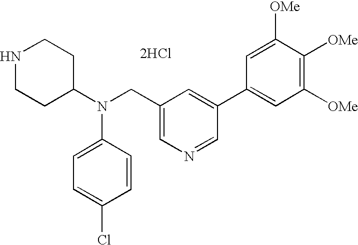

1-(tert-Butoxycarbonyl)+[N-(4-chlorophenyl)-N-[[3-(3,4,5-trimethoxyphenyl)pyridin-5-yl]methyl]amino]piperidine (268 mg) was treated in the same manner as described in Preparation Example 94 to give light yellow powder of the title compound.

Yield: 233 mg (91%).

### PREPARATION EXAMPLE 170

### Synthesis of 1-(tert-butoxycarbonyl)-4-[N-(4-chlorophenyl)-N-[3-(3,4,5-trimethoxyphenyl)benzyl]amino]piperidine

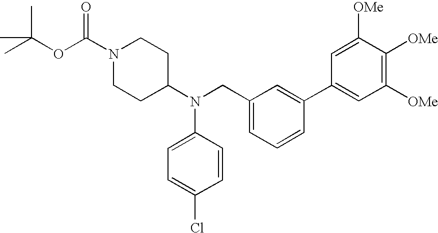

1-(tert-Butoxycarbonyl)-4-[4-(chlorophenyl)amino]piperidine (622 mg) and 3-(3,4,5-trimethoxyphenyl)benzyl chloride (586 mg) was treated in the same manner as described in Example 9 to give light yellow amorphous of the title compound.

Yield: 1.04 g (92%).

¹H-NMR (400 MHz, CDCl₃) δ: 1.45 (s, 9H), 1.58-1.67 (m, 2H), 1.82-1.91 (m, 2H), 2.74-2.86 (m, 2H), 3.85-3.92 (m, 1H), 3.88 (s, 3H), 3.89 (s, 6H), 4.35-4.31 (m, 2H), 4.49 (s, 2H), 6.66 (d, 2H, 3=9.2 Hz), 6.70 (s, 2H), 7.12 (d, 2H, J=9.0 Hz), 7.20 (d, 2H, J=7.3 Hz), 7.33-7.43 (m, 3H).

### PREPARATION EXAMPLE 171

### Synthesis of 4-[N-(4-chlorophenyl)-N-[3-(3,4,5-trimethoxyphenyl)benzyl]amino]piperidine Hydrochloride

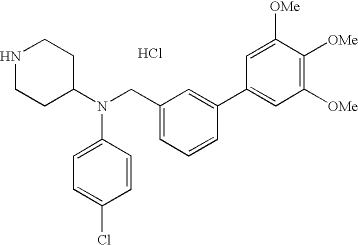

1-(tert-Butoxycarbonyl)-4-[N-(4-chlorophenyl)-N-[3-(3,4,5-trimethoxyphenyl) benzyl]amino]piperidine (1.04 g) was treated in the same manner as described in Preparation Example 94 to give light yellow powder of the title compound.

Yield: 899 mg (97%).

### EXAMPLE 141 TO 148

These compounds were obtained by the condensation of amines obtained in Preparation Examples 167, 169 and 171 with chloride derivatives obtained in Preparation Examples 3, 42 and 48. Free bases obtained were then converted to the corresponding hydrochlorides. Yields and NMR data of their free bases are listed below.

| | | | NMR data (400 MHz, |
|---|---|---|---|
| | | | measured as free |
| Example | Structure | Yield | bases, CDCl₃) δ |
| 141 | 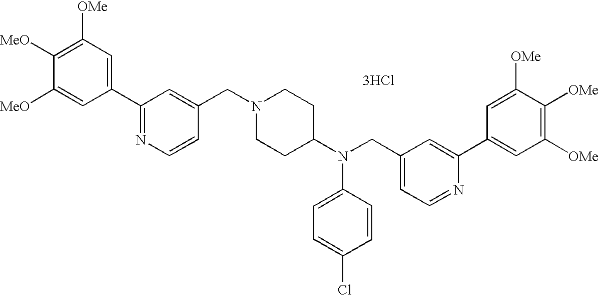 | 66% | 1.71-1.90 (m. 4H), 2.15-2.24 (m, 2H), 2.95-3.05 (m, 2H), 3.58 (s, 2H), 3.73-3.84 (m, 1H), 3.89 (s, 3H), 3.90 (s, 3H), 3.93 (s, 6H), 3.96 (s, 6H), 4.52 (s, 2H), 6.62 (d, 2H, J=9.0 Hz), 7.10-7.16 (m, 5H), 7.19-7.24 (m, 3H), 7.52 (s, 1H), 7.59 (s, 1H), 8.56 (d, 1H, J=4.9 Hz ,8.59 (d, 1H, J=4.9 Hz). |
| 142 | 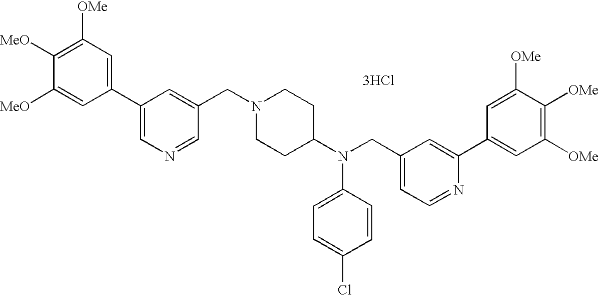 | 67% | 1.69-1.90 (m, 1H), 2.12-2.25 (m, 2H), 2.93-3.06 (m, 2H), 3.59 (s, 2H), 3.72-3.83 (m, 1H), 3.89 (s, 3H), 3.90 (s, 3H), 3.92 (s, 6H), 3.93 (s, 6H), 4.50 (s, 2H), 6.62 (d, 2H, J=9.2 Hz), 6.75 (s, 2H), 7.10 (d, 1H, J=5.3 Hz), 7.13 (s, 2H), 7.13 (d, 2H, J=9.0 Hz), 7.50 (s, 1H), 7.76 (s, 1H), 8.50 (d, 1H, J=1.8 Hz), 8.55 (d, 1H, J=5.1 Hz), 8.70 (d, 1H, J=1.8 Hz). |
| 143 | 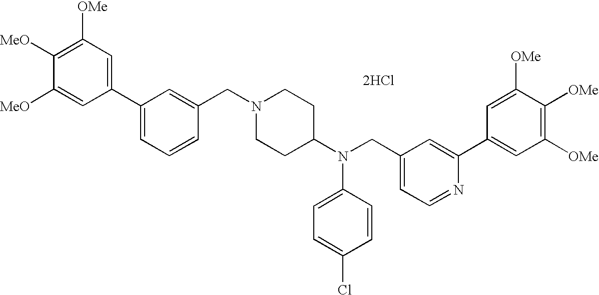 | 70% | 1.65-1.88 (m, 4H), 2.08-2.20 (m, 2H), 2.97-3.07 (m. 2H), 3.59 (s, 2H), 3.71-3.82 (m, 1H), 3.88 (s, 3H), 3.89 (s, 3H), 3.90-3.93 (m, 3H), 4.50 (s, 2H), 6.61 (d, 2H, J=8.2 Hz), 6.76 (s, 2H), 7.07-7.14 (m, 5H), 7.28 (d, 1H, J=6.6 Hz), 7.37 (dd, 1H, J=7.4 Hz), 7.40-7.47 (m, 2H), 7.50 (s, 1H), 8.54 (d, 1H, J=5.1 Hz). |
| 144 | 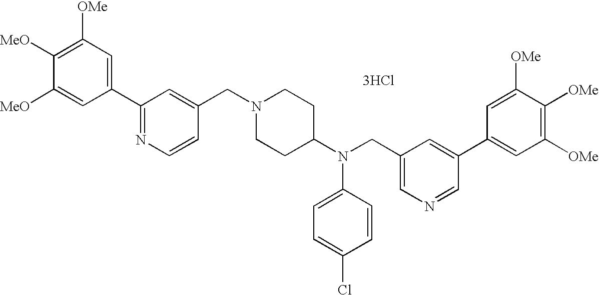 | 57% | 1.56-1.93 (m, 4H), 2.12-2.30 (m, 2H), 2.92-3.10 (m, 2H), 3.53-3.68 (m, 2H), 3.70-3.82 (m, 1H), 3.88 (s, 3H), 3.89 (s, 6H), 3.90 (s, 3H), 3.96 (s, 6H), 4.56 (s, 2H), 6.64-6.70 (m, 4H), 7.13 (d, 2H, J=9.0 Hz), 7.20-7.30 (m, 3H), 7.63 (br, 2H), 8.48 (s, 1H), 8.60 (d, 1H, J=5.1 Hz), 8.66 (d, 1H, J=2.2 Hz. |
| 145 | 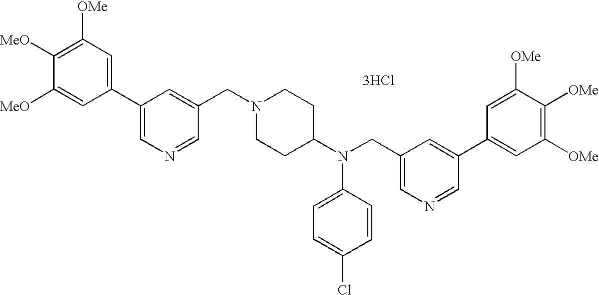 | 70% | 1.71-1.92 (m, 4H), 2.12-2.27 (m, 2H), 2.94-3.07 (m, 2H), 3.59 (s, 2H), 3.69-3.81 (s, 1H), 3.88 (s, 3H), 3.89 (s, 6H), 3.90 (s, 3H), 3.93 (s, 6H), 4.54 (s, 2H), 6.63-6.68 (m, 4H), 6.75 (s, 2H), 7.13 (d, 2H, J=9.0 Hz), 7.62 (s, 1H), 7.76 (s, 1H), 8.47 (d, 1H, J=1.8 Hz), 8.50 (d, 1H, J=1.8 Hz), 8.65 (d, 1H, J=2.0 Hz), 8.70 (s, 1H). |
| 146 | 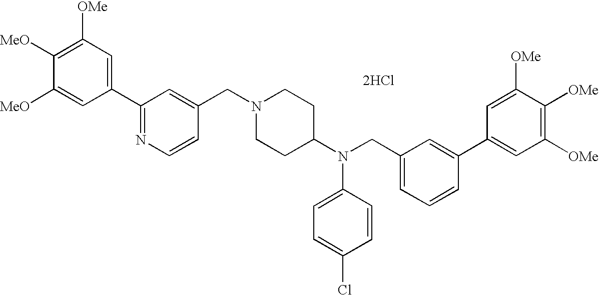 | 78% | 1.75-1.91 (m, 4H), 2.13-2.23 (m, 2H), 2.94-3.02 (m, 2H), 3.57 (s, 2H), 3.73-3.82 (m, 1H), 3.88 (s, 3H), 3.88 (s, 6H), 3.90 (s, 3H), 3.96 (s, 6H), 4.55 (s, 2H), 6.65 (d, 2H, J=9.0 Hz), 6.68 (s, 2H), 7.11 (d, 2H, J=8.5 Hz), 7.18-7.24 (m, 4H), 7.32-7.42 (m, 3H), 7.59 (s, 1H), 8.59 (d, 1H, J=4.9 Hz). |
| 147 | 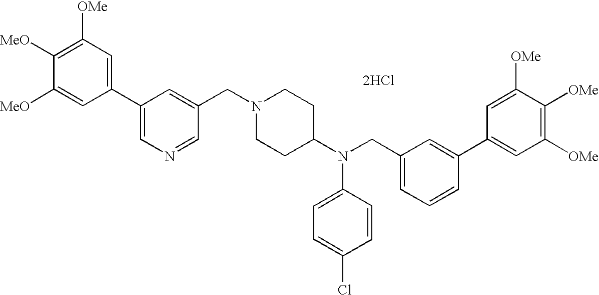 | 63% | 1.72-1.89 (m, 4H), 2.12-2.21 (m, 2H), 2.94-3.03 (m, 2H), 3.59 (s, 2H), 3.72-3.82 (m, 1H), 3.87 (s, 3H), 3.88 (s, 6H), 3.90 (s, 3H), 3.93 (s, 6H), 4.53 (s, 2H), 6.64 (d, 2H, J=9.2 Hz), 6.68 (s, 2H), 6.75 (s, 2H), 7.11 (d, 2H), 7.19 (d, 1H, J=7.6 Hz), 7.32-7.40 (m, 3H), 7.76 (s, 1H), 8.50 (d, 1H, J=1.8 Hz), 8.69 (d, 1H, J=2.2 Hz). |
| 148 | 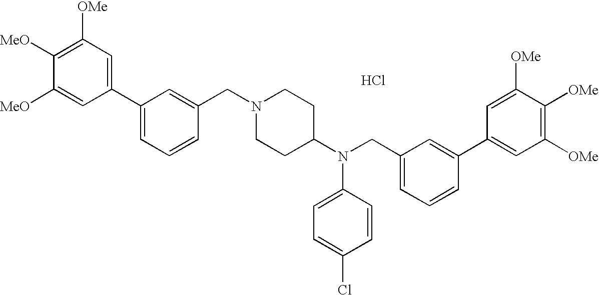 | 68% | 1.72-1.87 (m, 4H), 2.08-2.18 (m, 2H), 2.97-3.05 (m, 2H), 3.58 (s, 2H), 3.71-3.82 (m, 1H), 3.87 (s, 3H), 3.88 (s, 6H), 3.89 (s, 3H), 3.92 (s, 6H), 4.53 (s, 2H), 6.64 (dt, 2H, J=9.3 Hz, 2.9 Hz), 6.68 (s, 2H), 6.76 (s, 2H), 7.10 (dt, 2H, J=9.0 Hz, 2.8 Hz), 7.19 (d, 1H, J=7.6 Hz, 7.24-7.47 (m, 7H). |

### PREPARATION EXAMPLE 172

### Synthesis of 1-(tert-butoxycarbonyl)-4-[(3,4-difluorophenyl)amino]piperidine

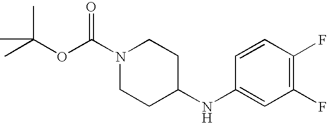

1-(tert-Butoxycarbonyl)-4-piperidone (5.00 g) and 3,4-difluoroaniline (3.09 g) was treated in the same manner as described in Preparation Example 37 to give light brown prism crystal of the title compound.

Yield: 4.66 g (62%).

¹H-NMR (400 MHz, CDCl₃) δ: 1.24-1.37 (m, 2H), 1.46 (s, 9H), 1.97-2.05 (m, 2H), 2.85-2.96 (m, 2H), 3.26-3.36 (m, 1H), 3.38-3.52 (m, 1H), 3.96-4.14 (m, 2H), 6.22-6.28 (m, 1H), 6.38 (ddd, 1H, J=12.7 Hz, 6.6 Hz, 2.9 Hz), 6.94 (dd, 1H, J=19.1 Hz, 9.0 Hz).

### PREPARATION EXAMPLE 173

### Synthesis of 1-(tert-butoxycarbonyl)-4-[N-(3,4-difluorophenyl)-N-[[2-(3,4,5-trimethoxyphenyl)pyridin-4-yl]methyl]amino]piperidine

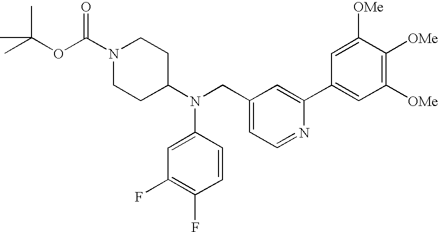

1-(tert-Butoxycarbonyl)-4[(3,4-difluorophenyl)amino]piperidine (625 mg) and 4-chloromethyl-2-(3,4,5-trimethoxyphenyl)pyridine (588 mg) was treated in the same manner as described in Example 9 to give light yellow amorphous of the title compound.

Yield: 534 mg (47%).

¹H-NMR (400 MHz, CDCl₃) δ: 1.45 (s, 9H), 1.50-1.70 (m, 2H), 1.82-1.90 (m, 2H), 2.73-2.88 (m, 2H), 3.90 (s, 3H), 3.94 (s, 6H), 4.15-4.30 (m, 2H), 4.43 (s, 2H), 6.33-6.39 (m, 1H), 6.52 (ddd, 1H, J=13.6 Hz, 6.4 Hz, 3.1 Hz), 6.98 (dd, 1H, J=19.1 Hz, 9.2 Hz), 7.11 (dd, 1H, J=5.0 Hz, 1.3 Hz), 7.16 (s, 2H), 7.51 (s, 1H), 8.58 (d, 1H, J=5.1 Hz).

### PREPARATION EXAMPLE 174

### Synthesis of 4-[N-(3,4-difluorophenyl)-N-[[2-(3,4,5-trimethoxyphenyl)pyridin-4-yl]methyl]amino]piperidine Dihydrochloride

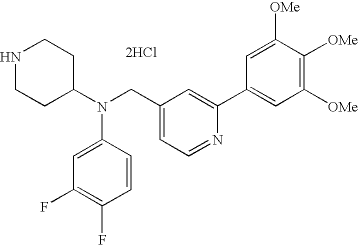

1-(tert-Butoxycarbonyl)-4-[N-(3,4-difluorophenyl)-N-[[2-(3,4,5-trimethoxyphenyl)pyridinyl]methyl]amino]piperidine (534 mg) was treated in the same manner as described in Preparation Example 94 to give light yellow powder of the title compound.

Yield: 442 mg (87%).

### PREPARATION EXAMPLE 175

### Synthesis of 1-(tert-butoxycarbonyl)-4[N-(3,4-difluorophenyl)-N-[[3-(3,4,5-trimethoxyphenyl)pyridin-5-yl]methyl]amino]piperidine

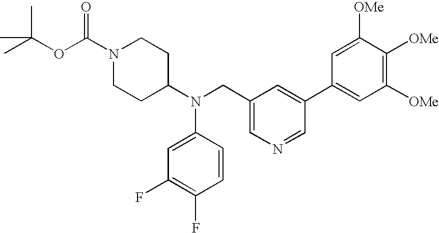

1-(tert-Butoxycarbonyl)-4-[(3,4-difluorophenyl)amino]piperidine (625 mg) and 5-chloromethyl-3-(3,4,5-trimethoxyphenyl)pyridine (588 mg) was treated in the same manner as described in Example 9 to give light yellow amorphous or the title compound.

Yield: 350 mg (31%).

### PREPARATION EXAMPLE 176

### Synthesis of 4-[N-(3,4-difluorophenyl)-N-[[3-(3,4,5-trimethoxyphenyl)pyridin-5-yl]methyl]amino]piperidine Dihydrochloride

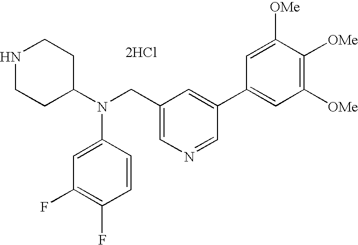

1-(tert-Butoxycarbonyl)-4-[N-(3,4-difluorophenyl)-N-[[3-(3,4,5-trimethoxyphenyl)pyridin-5-yl]methyl]amino]piperidine (350 mg) was treated in the same manner as described in Preparation Example 94 to give light yellow powder of the title compound.

Yield: 305 mg (92%).

### PREPARATION EXAMPLE 177

### Synthesis of 1-(tert-butoxycarbonyl)-4-[N-(3,4-difluorophenyl)-N-[3-(3,4,5-trimethoxyphenyl)benzyl]amino]piperidine

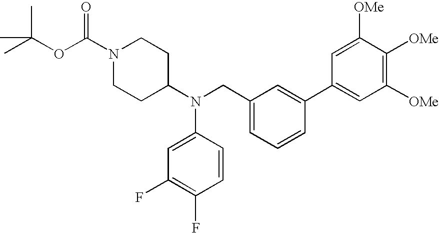

1-(tert-Butoxycarbonyl)-4-[(3,4-difluorophenyl)amino]piperidine (625 mg) and 3-(3,4,5-trimethoxyphenyl)benzyl chloride (586 mg) was treated in the same manner as described in Example 9 to give light yellow amorphous of the title compound.

Yield: 980 mg (86%).

¹H-NMR (400 MHz, CDCl₃) δ: 1.45 (s, 9H), 1.52-1.66 (m, 2H), 1.81-1.89 (m, 2H), 2.72-2.85 (m, 2H), 3.78 (tt, 1H, J=11.8 Hz, 3.8 Hz), 3.88 (s, 3H), 3.90 (s, 6H), 4.12-4.30 (m, 2H), 4.45 (s, 2H), 6.36-6.42 (m, 1H), 6.54 (ddd, 1H, J=13.9 Hz, 6.8 Hz, 2.9 Hz), 6.71 (s, 2H), 6.95 (dd, 1H, J=19.2 Hz, 9.2 Hz), 7.20 (d, 1H, J=7.4 Hz), 7.36-7.43 (m, 3H).

### PREPARATION EXAMPLE 178

### Synthesis of 4-[N-(3,4-difluorophenyl)-N-[3-(3,4,5-trimethoxyphenyl)benzyl]amino]piperidine Hydrochloride

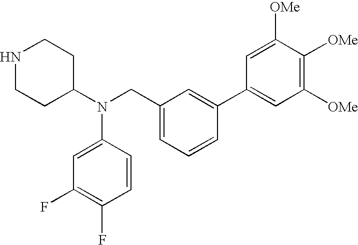

1-(tert-Butoxycarbonyl)-4-[N-(3,4-difluorophenyl)-N-[3-(3,4,5-trimethoxyphenyl)benzyl]amino]piperidine (980 mg) was treated in the same manner as described in Preparation Example 94 to give light yellow powder of the title compound.

Yield: 819 mg (94%).

### EXAMPLE 149 TO 156

These compounds were obtained by the condensation of amines obtained in Preparation Examples 174, 176 and 178 with chloride derivatives obtained in Preparation Examples 3, 42 and 48. Free bases obtained were then converted to the corresponding hydrochlorides. Yields and NMR data of their free bases are listed below.

| | | | NMR data (400 MHz, |
|---|---|---|---|
| | | | measured as free |
| Example | Structure | Yield | bases, CDCl₃) δ |
| 149 | 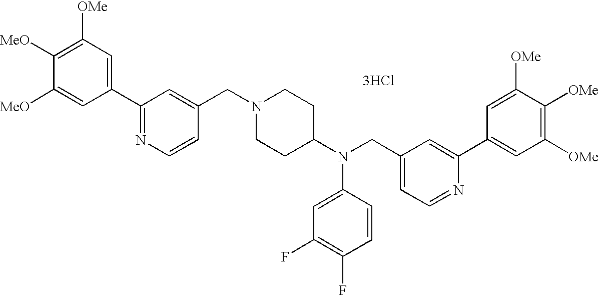 | 67% | 1.70-1.90 (m, 4H), 2.16-2.23 (m, 2H), 2.95-3.03 (m, 2H), 3.58 (s, 2H), 3.64-3.74 (m, 1H), 3.89 (s, 3H), 3.90 (s, 3H), 3.93 (s, 6H), 3.96 (s, 6H), 4.49 (s, 2H), 6.31-6.37 (m, 1H), 6.51 (ddd, 1H, J=13.9 Hz, 6.6 Hz, 3.1 Hz), 6.96 (dd, 1H, J=19.2 Hz, 9.8 Hz), 7.11 (d, 1H, J=5.1 Hz), 7.15 (s, 2H), 7.20 (d, 1H, J=5.1 Hz), 7.22 (s, 2H), 7.52 (s, 1H), 7.59 (s, 1H), 8.57 (d, 1H, J=5.1 Hz), 8.59 (d, |
| # 1H, J=5.1 Hz). | | | |
| 150 | 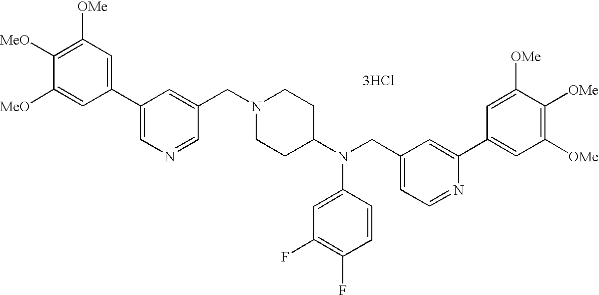 | 47% | 1.67-1.79 (m, 2H), 1.81-1.89 (m, 2H), 2.13-2.20 (m, 2H), 2.95-3.05 (m, 2H), 3.59 (s, 2H), 3.63-3.75 (m, 1H), 3.89 (s, 3H), 3.90 (s, 3H), 3.93 (s, 12H), 4.47 (s, 2H), 6.30-6.36 (m, 1H), 6.50 (ddd, 1H, J=13.9 Hz, 6.6 Hz, 3.1 Hz), 6.75 (s, 2H), 6.96 (d, 1H, J=19.0 Hz, 9.4 Hz), 7.10 (d, 1H, J=4.1 Hz), 7.15 (s, 2H), 7.51 (s, 1H), 7.75 (s, 1H), 8.50 (d, 1H, J=1.8 Hz), 8.56 (d, 1H, J=5.1 |
| # 1H, J=5.1 Hz), 8.70 (s, 1H). | | | |
| 151 | 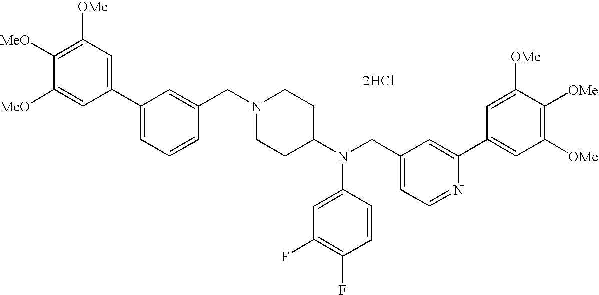 | 53% | 1.68-1.87 (m, 4H), 2.09-2.18 (m, 2H), 2.98-3.06 (m, 2H), 3.58 (s, 2H), 3.63-3.72 (m, 1H), 3.89 (s, 3H), 3.89 (s, 3H), 3.92 (s, 6H), 3.93 (s, 6H), 4.47 (s, 2H), 6.33-6.35 (m, 1H), 6.50 (ddd, 1H, J=13.9 Hz, 6.4 Hz, 2.9 Hz), 6.76 (s, 2H), 6.95 (dd, 1H, J=19.2 Hz, 9.4 Hz), 7.09 (d, 1H, J=5.1 Hz), 7.15 (s, 2H), 7.25-7.30 (m, 1H), 7.37 (dd, 1H, J=7.3 Hz, 7.3 Hz), 7.42-7.46 (m, 2H), 7.50 (s, 1H), |
| # 8.56 (d, 1H, J=5.1 Hz). | | | |
| 152 | 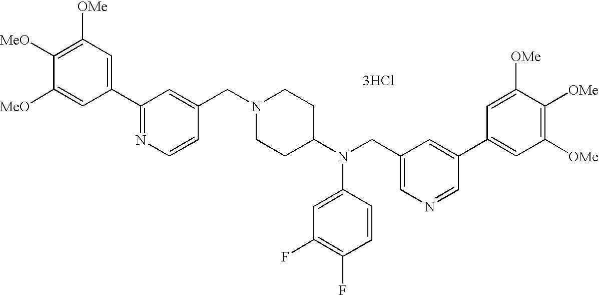 | 50% | 1.72-1.96 (m, 4H), 2.12-2.28 (m, 2H), 2.94-3.08 (m, 2H), 3.59 (s, 2H), 3.62-3.72 (m, 1H), 3.89 (s, 3H), 3.90 (s, 9H), 3.96 (s, 6H), 4.52 (s, 2H), 6.36-6.43 (m, 1H), 6.55 (ddd, 1H, J=13.7 Hz, 6.6 Hz, 2.9 Hz), 6.67 (s, 2H), 6.96 (dd, 1H, J=19.1 Hz, 9.2 Hz), 7.21 (dd, 1H, J=5.1 Hz, 1.2 Hz), 7.24 (s, 2H), 7.61(br, 1H), 7.64 (s, 1H), 8.47 (d, 1H, J=2.0 Hz), 8.60 (d, 1H, J=4.9 Hz), 8.67 (d, 1H, |
| # J=2.0 Hz). | | | |
| 153 | 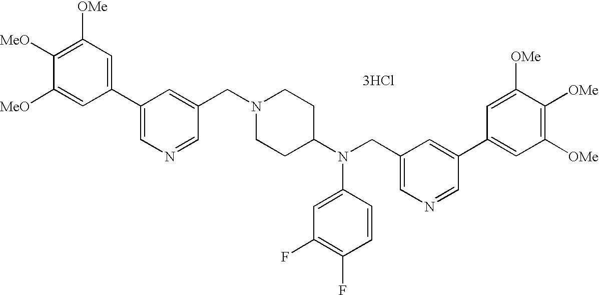 | 61% | 1.71-1.90 (m, 4H), 2.12-2.25 (m, 2H), 2.95-3.05 (m, 2H), 357-375 (m, 1H), 3.59 (m, 2H), 3.88 (s, 3H), 3.90 (s, 9H), 3.93 (s, 6H), 4.50 (s, 2H), 6.32-6.43 (m, 1H), 6.54 (ddd, 1H, J=13.6 Hz, 6.4 Hz, 2.7 Hz), 6.67 (s, 2H), 6.73-6.78 (m, 3H), 6.96 (dd, 1H, J=18.9 Hz, 9.6 Hz), 7.63 (s, 1H), 7.76 (s, 1H), 8.46 (s, 1H), 8.50 (d, 1H, J=1.6 Hz), 8.66 (d, 1H, J=1.8 Hz), 8.70 (d, 1H, J=2.0 Hz). |
| 154 | 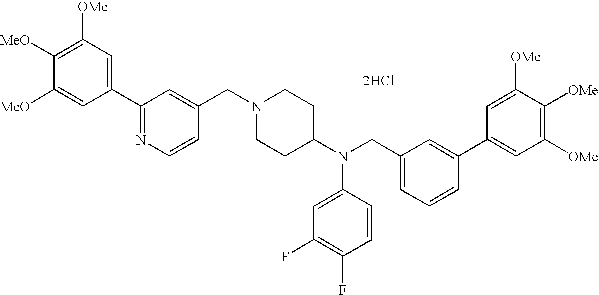 | 82% | 1.74-1.90 (m, 4H), 2.13-2.22 (m, 2H), 2.95-3.01 (m, 2H), 3.57 (s, 2H), 3.63-3.73 (m, 1H), 3.88 (s, 3H), 3.89 (s, 6H), 3.90 (s, 3H), 3.96 (s, 6H), 4.51 (s, 2H), 6.34-6.40 (m, 1H), 6.52 (ddd, 1H, J=14.1 Hz, 6.6 Hz, 3.1 Hz), 6.70 (s, 2H), 6.94 (dd, 1H, J=19.2 Hz, 9.4 Hz), 7.17-7.26 (m, 4H), 7.32-7.42 (m, 3H), 7.59 (s, 1H), 8.59 (d, 1H, J=5.1 Hz. |
| 155 | 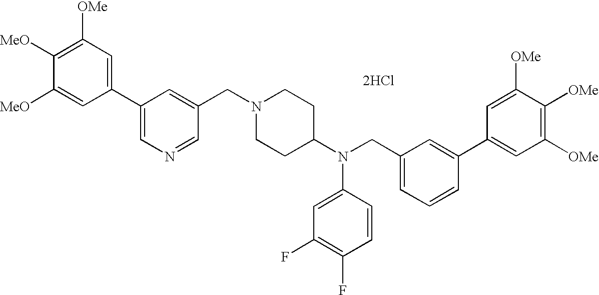 | 75% | 1.74-1.90 (m, 4H), 2.13-2.21 (m, 2H), 2.95-3.04 (m, 2H), 3.59 (s, 2H), 3.63-3.72 (m, 1H), 3.88 (s, 3H), 3.89 (s, 6H), 3.89 (s, 3H), 3.93 (s, 6H), 4.49 (s, 2H), 6.33-6.39 (m, 1H), 6.52 (ddd, 1H, J=14.3 Hz, 3.7 Hz, 2.9 Hz), 6.69 (s, 2H), 6.75 (s, 2H), 6.94 (dd, 1H, J=19.1 Hz, 9.8 Hz), 7.19 (d, 1H, J=7.8 Hz), 7.32-7.41 (m, 3H), 7.76 (s, 1H), 8.50 (d, 1H, J=1.5 Hz), 8.69 (s, 1H). |
| 156 | 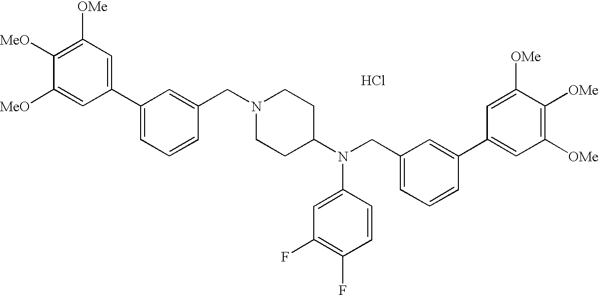 | 79% | 1.72-1.88 (m, 4H), 2.08-2.18 (m, 2H), 2.98-3.05 (m, 2H), 3.58 (s, 2H), 3.62-3.72 (m, 1H), 3.88 (s, 3H), 3.89 (s, 9H), 3.92 (s, 6H), 4.45 (s, 2H), 6.33-6.39 (m, 1H), 6.51 (ddd, 1H, J=13.9 Hz, 6.6 Hz, 3.0 Hz), 6.69 (s, 2H), 6.76 (s, 2H), 6.93 (dd, 1H, J=19.3 Hz, 9.5 Hz), 7.19 (d, 1H, J=7.6 Hz), 7.25-7.47 (m, 7H). |

### PREPARATION EXAMPLE 179

### Synthesis of 1-(tert-butoxycarbonyl)-4-[(4-fluorophenyl)amino]piperidine

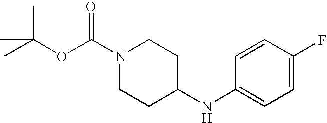

1-(tert-Butoxycarbonyl)-4-piperidone (5.00 g) and 4-fluoroaniline (2.66 g) was treated in the same manner as described in Preparation Example 37 to give white crystalline powder of the title compound.

Yield: 4.99 g (71%).

¹H-NMR (400 MHz, CDCl₃) δ: 1.23-1.36 (m, 2H), 1.46 (s, 9H), 1.97-2.05 (m, 2H), 2.84-2.96 (m, 2H), 3.30-3.39 (m, 2H), 3.96-4.14 (m, 2H), 6.51-6.57 (m, 2H), 6.84-6.91 (m, 2H).

### PREPARATION EXAMPLE 180

### Synthesis of 1-(tert-butoxycarbonyl)-4[N-(4-fluorophenyl)-N-[[2-(3,4,5-trimethoxyphenyl)pyridin-4-yl]methyl]amino]piperidine

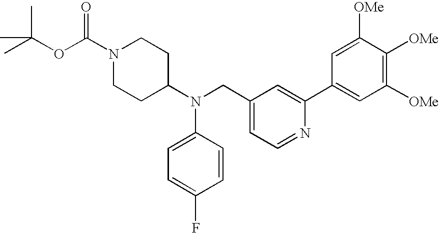

1-(tert-Butoxycarbonyl)-4-[(4-fluorophenyl)amino]piperidine (589 mg) and 4-chloromethyl-2-(3,4,5-trimethoxyphenyl)pyridine (588 mg) was treated in the same manner as described in Example 9 to give light yellow amorphous of the title compound.

Yield: 702 mg (64%).

¹H-NMR (400 MHz, CDCl₃) δ: 1.45 (s, 9H), 1.48-1.64 (m, 2H), 1.81-1.90 (m, 2H), 2.72-2.85 (m, 2H), 3.69-3.98 (m, 1H), 3.89 (m, 3H), 3.94 (m, 6H), 4.16-4.28 (m, 2H), 4.43 (s, 2H), 6.66-6.73 (m, 2H), 6.91 (dd, 2H, J=9.2 Hz, 9.2 Hz), 7.12-7.16 (m, 3H), 7.53 (s, 1H).

### PREPARATION EXAMPLE 181

### Synthesis of 4-[N-(4-fluorophenyl)-N-[[2-(3,4,5-trimethoxyphenyl)pyridin-4-yl]methyl]amino]piperidine Dihydrochloride

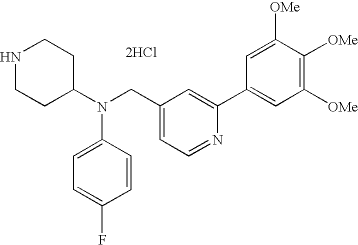

1-(tert-Butoxycarbonyl)-4-[N-(4-fluorophenyl)-N-[[2-(3,4,5-trimethoxyphenyl)pyridin-4-yl]methyl]amino]piperidine (702 mg) was treated in the same manner as described in Preparation Example 94 to give light yellow powder of the title compound.

Yield: 561 mg (84%).

### PREPARATION EXAMPLE 182

### Synthesis of 1-(tert-butoxycarbonyl)-4-[N-(4-fluorophenyl)-N-[[3-(3,4,5-trimethoxyphenyl)pyridin-5-yl]methyl]amino]piperidine

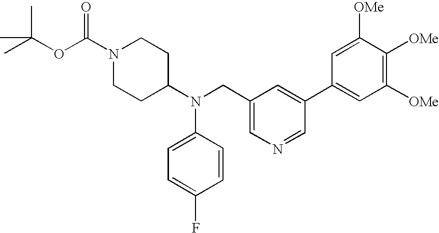

1-(tert-Butoxycarbonyl)-4-[(4-fluorophenyl)amino]piperidine (589 mg) and 5-chloromethyl-3-(3,4,5-trimethoxyphenyl)pyridine (588 mg) was treated in the same manner as described in Example 9 to give light yellow amorphous of the title compound.

Yield: 190 mg (17%).

¹H-NMR (400 MHz, CDCl₃) δ: 1.45 (s, 9H), 1.50-1.73 (m, 2H), 1.82-1.90 (m, 2H), 2.71-2.85 (m, 2H), 3.71 (tt, 1H, J=111.7 Hz, 3.1 Hz), 3.89 (s, 3H), 3.90 (s, 6H), 4.12-4.30 (m, 2H), 4.45 (s, 2H), 6.66 (s, 2H), 6.73-6.78 (m, 2H), 6.91 (dd, 2H, J=9.2 Hz, 8.2 Hz), 7.65 (s, 1H), 8.49 (d, 1H, J=2.0 Hz), 8.65 (d, 1H, J=2.0 Hz).

### PREPARATION EXAMPLE 183

### Synthesis of 4-[N-(4-fluorophenyl)-N-[[3-(3,4,5-trimethoxyphenyl)pyridin-5-yl]methyl]amino]piperidine Dihydrochloride

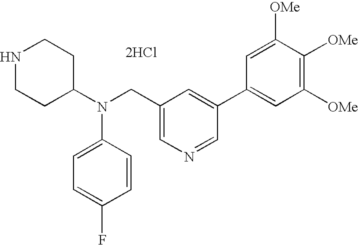

1-(tert-Butoxycarbonyl)-4-[N-(4-fluorophenyl)-N-[[3-(3,4,5-trimethoxyphenyl)pyridin-5-yl]methyl]amino]piperidine (190 mg) was treated in the same manner as described in Preparation Example 94 to give light yellow powder of the title compound.

Yield: 165 mg (91%).

### PREPARATION EXAMPLE 184

### Synthesis of 1-(tert-butoxycarbonyl)-4-[N-(4-fluorophenyl)-N-[3-(3,4,5-trimethoxyphenyl)benzyl]amino]piperidine

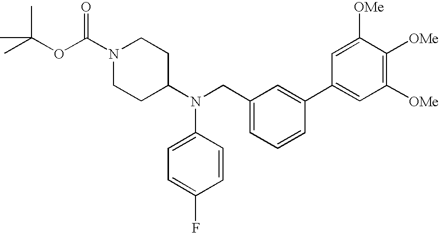

1-(tert-Butoxycarbonyl)-4-[(4-fluorophenyl)amino]piperidine (589 mg) and 3-(3,4,5-trimethoxyphenyl)benzyl chloride (586 mg) was treated in the same manner as described in Example 9 to give light yellow amorphous of the title compound.

Yield: 1.01 g (92%).

¹H-NMR (400 MHz, CDCl₃) δ: 1.44 (s, 9H), 1.51-1.65 (m, 2H), 1.82-1.90 (m, 2H), 2.82-2.84 (m, 2H), 3.78 (tt, 1H, J=11.7 Hz, 3.5 Hz), 3.88 (s, 3H), 3.90 (s, 6H), 4.10-4.30 (m, 2H), 4.45 (s, 2H), 6.68-6.73 (m, 4H), 6.89 (dd, 2H, J=9.2 Hz, 8.2 Hz), 7.21-7.25 (m, 1H), 7.32-7.41 (m, 3H).

### PREPARATION EXAMPLE 185

### Synthesis of 4-[N-(4-fluorophenyl)-N-[3-(3,4,5-trimethoxyphenyl)benzyl]amino]piperidine Hydrochloride:

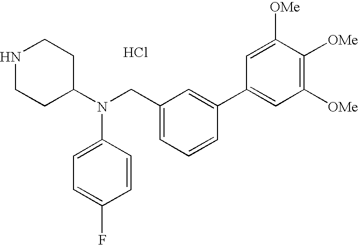

1-(tert-Butoxycarbonyl)-4-[N-(4-fluorophenyl)-N-[3-(3,4,5-trimethoxyphenyl) benzyl]amino]piperidine (1.01 g) was treated in the same manner as described in Preparation Example 94 to give light yellow powder of the title compound.

Yield: 790 mg (88%).

### EXAMPLE 157 TO 164

These compounds were obtained by the condensation of amines obtained in Preparation Examples 181, 183 and 185 with chloride derivatives obtained in Preparation Examples 3, 42 and 48. Free bases obtained were then converted to the corresponding hydrochlorides. Yields and NMR data of their free bases are listed below.

| | | | NMR data (400 MHz, |
|---|---|---|---|
| | | | measured as free |
| Example | Structure | Yield | bases, CDCl₃) δ |
| 157 | 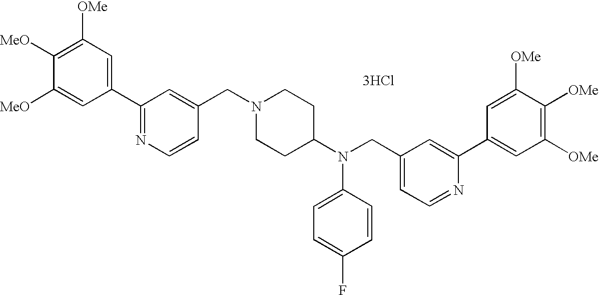 | 62% | 1.60-1.82 (m, 2H), 1.83-1.91 (m, 2H), 2.13-2.23 (m, 2H), 2.95-3.03 (m, 2H), 3.57 (s, 2H), 3.64-3.75 (m, 1H), 3.89 (s, 3H), 3.90 (s, 3H), 3.93 (s, 6H), 3.96 (s, 6H), 4.48 (s, 2H), 6.65-6.70 (m, 2H), 6.90 (dd, 2H, J=8.8 Hz, 8.8 Hz), 7.13-7.16 (m, 3H), 7.20 (d, 1H, J=5.1 Hz), 7.22 (s, 2H), 7.54 (s, 1H), 7.59 (s, 1H), 8.55 (d, 1H, J=5.1 Hz), 8.59 (d, 1H, J=4.9 Hz). |
| 158 | 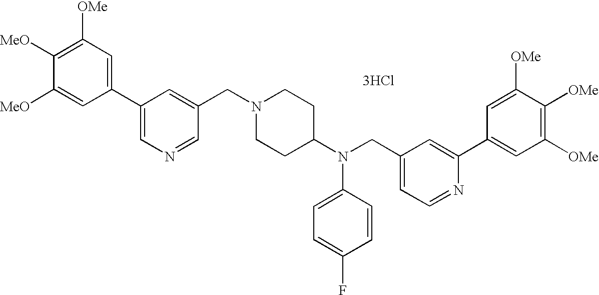 | 53% | 1.66-1.95 (m, 4H), 2.12-2.24 (m, 2H), 2.95-3.07 (m, 2H), 3.60 (s, 2H), 3.64-3.76 (m, 1H), 3.89 (s, 3H), 3.90 (s, 3H), 3.92 (s, 6H), 3.93 (s, 6H), 4.47 (s, 2H), 6.63-6.70 (m, 1H), 6.75 (s, 2H), 6.90 (dd, 1H, J=9.2 Hz, 9.2 Hz), 7.11-7.16 (m, 3H), 7.53 (s, 1H), 7.77 (s, 1H), 8.50 (d, 1H, J=2.0 Hz), 8.55 (d, 1H, J=4.9 Hz), 8.70 (d, 1H, J=5.9 Hz). |
| 159 | 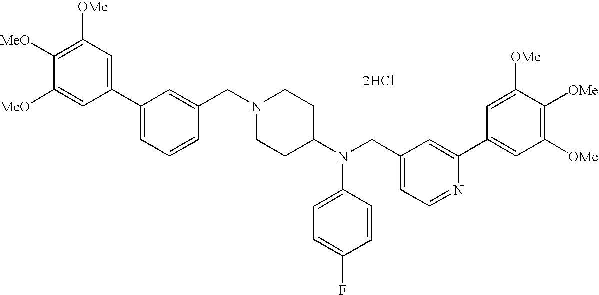 | 51% | 1.64-1.90 (m, 4H), 2.07-2.20 (m, 4H), 2.97-3.08 (m, 2H), 3.59 (s, 2H), 3.64-3.76 (m, 1H), 3.89 (s, 6H), 3.92 (s, 6H), 3.93 (s, 6H), 4.47 (s, 2H), 6.62-6.70 (m, 2H), 6.77 (s, 2H), 6.86-6.93 (m, 2H), 7.11-7.16 (m, 3H), 7.25-7.31 (m, 3H), 7.37 (dd, 1H, J=7.4 Hz, 7.4 Hz), 7.42-7.49 (m, 2H), 7.53 (s, 1H), 8.54 (d, 1H, J=5.1 Hz). |
| 160 | 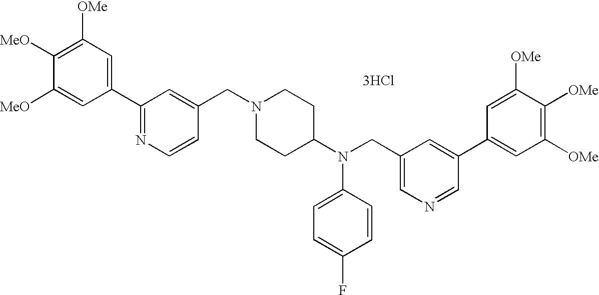 | 49% | 1.74-1.98 (m, 4H), 2.10-2.30 (m, 2H), 2.90-3.12 (m, 2H), 3.53-3.73 (m, 3H), 3.88 (s, 3H), 3.89 (s, 6H), 3.90 (s, 3H), 3.96 (s, 6H), 4.50 (s, 2H), 6.66 (s, 2H), 6.70-6.76 (m, 2H), 6.90 (dd, 2H, J=8.8 Hz, 8.8 Hz), 7.19-7.28 (m, 3H), 7.65 (br, 2H), 8.49 (d, 1H, J=1.8 Hz), 8.60 (d, 1H, J=4.9 Hz), 8.64 (d, 1H, J=2.2 Hz). |
| 161 | 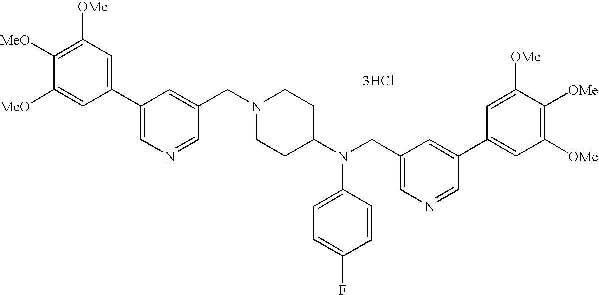 | 26% | 1.67-1.97 (m, 4H), 2.10-2.27 (m, 2H), 2.94-3.06 (m, 2H), 3.56-3.68 (m, 3H), 3.88 (s, 3H), 3.89 (s, 6H), 3.90 (s, 3H), 3.93 (s, 6H), 4.49 (s, 2H), 6.65 (s, 2H), 6.69-6.80 (m, 4H), 6.84-6.93 (m, 2H), 7.64 (s, 1H), 7.77 (br, 1H), 8.48 (d, 1H, J=1.7 Hz), 8.50 (d, 1H, J=1.7 Hz), 8.64 (d, 1H, J=1.9 Hz), 8.70 (s, 1H). |
| 162 | 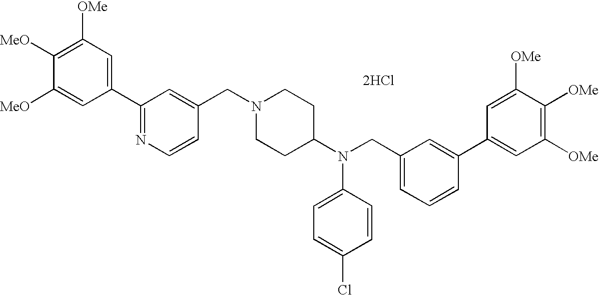 | 83% | 1.72-1.92 (m, 4H), 2.12-2.21 (m, 2H), 2.94-3.02 (m, 2H), 3.57 (s, 2H), 3.64-3.74 (m, 1H), 3.88 (s, 3H), 3.89 (s, 6H), 3.90 (s, 3H), 3.96 (s, 6H), 4.51 (s, 1H), 6.66-6.71 (m, 4H), 6.88 (dd, 2H, J=8.6 Hz, 8.6 Hz), 7.18-7.27 (m, 4H), 7.34 (dd, 1H, J=7.4 Hz, 7.4 Hz), 7.39 (d, 2H, J=5.4 Hz), 7.59 (s, 1H), 8.59 (d, 1H, J=5.1 Hz). |
| 163 | 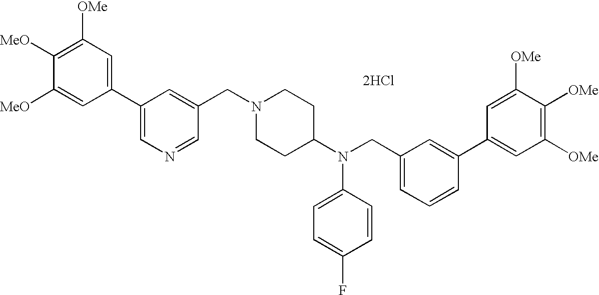 | 68% | 1.68-1.87 (m, 4H), 2.10-2.22 (m, 2H), 3.65-3.74 (m, 1H), 3.87 (s, 3H), 3.88 (s, 6H), 3.90 (s, 3H), 3.93 (s, 6H), 4.49 (s, 2H), 6.66-6.70 (m, 6H), 6.88 (dd, 2H, J=8.8 Hz, 8.8 Hz), 7.19-7.40 (m, 4H), 7.77 (s, 1H), 8.49 (d, 1H, J=1.8 Hz), 8.70 (s, 1H). |
| 164 | 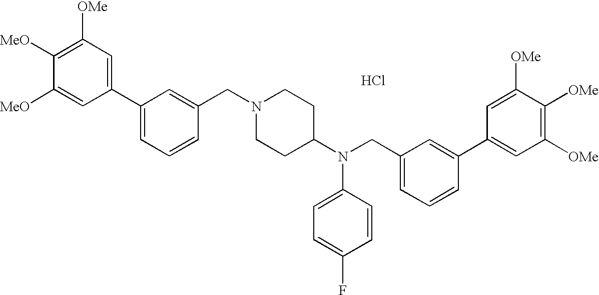 | 74% | 1.70-1.90 (m, 4H), 2.08-2.18 (m, 2H), 2.95-3.05 (m, 2H), 3.58 (s, 2H), 3.63-3.73 (m, 1H), 3.87 (s, 3H), 3.88 (s, 6H), 3.89 (s, 3H), 3.92 (s, 6H), 4.50 (s, 2H), 6.65-6.72 (m, 2H), 6.69 (s, 2H), 6.76 (s, 2H), 6.87 (dd, 2H, J=9.0 Hz, 9.0 Hz), 7.22 (d, 1H, J=7.6 Hz), 7.25-7.48 (m, 9H). |

### PREPARATION EXAMPLE 186

### Synthesis of 1-(tert-butoxycarbonyl)-4-phenylaminopiperidine

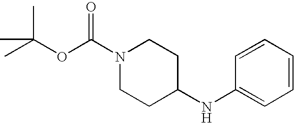

1-(tert-Butoxycarbonyl)-4-piperidone (5.00 g) and aniline (2.23 g) was treated in the same manner as described in Preparation Example 37 to give white needles of the title compound.

Yield: 3.77 g (57%).

¹H-NMR (400 MHz, CDCl₃) δ: 1.25-1.38 (m, 2H), 1.47 (s, 9H), 2.00-2.07 (m, 2H), 2.87-2.97 (m, 2H), 3.38-3.53 (m, 2H), 3.96-4.14 (m, 2H), 6.57-6.52 (m, 2H), 6.70 (tt, 1H, J=6.2 Hz, 1.0 Hz), 7.17 (dd, 2H, J=8.6 Hz, 7.2 Hz).

### PREPARATION EXAMPLE 187

### Synthesis of 1-(tert-butoxycarbonyl)-4-[N-phenyl-N-[[2-(3,4,5-trimethoxyphenyl)pyridin-4-yl]methyl]amino]piperidine

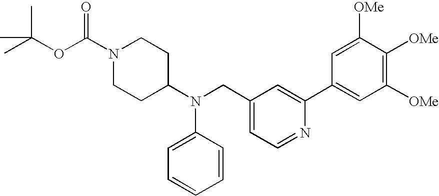

1-(tert-Butoxycarbonyl)-4-phenylaminopiperidine (553 mg) and 4-chloromethyl-2-(3,4,5-trimethoxyphenyl)pyridine (588 mg) was treated in the same manner as described in Example 9 to give light yellow amorphous of the title compound.

Yield: 760 mg (71%).

¹H-NMR (400 MHz, CDCl₃) δ: 1.45 (s, 9H), 1.53-1.63 (m, 2H), 1.83-1.91 (m, 2H), 2.76-2.90 (m, 2H), 3.86-3.97 (m, 1H), 3.89 (s, 3H), 3.93 (s, 6H), 4.14-4.32 (m, 2H), 4.49 (s, 2H), 6.71-6.78 (m, 3H), 7.14 (s, 1H), 7.15 (s, 2H), 7.21 (dd, 2H, J=8.8 Hz, 7.4 Hz), 7.55 (s, 1H), 8.56 (d, 1H, J=5.1 Hz).

### PREPARATION EXAMPLE 188

### Synthesis of 4-[N-phenyl-N-[[2-(3,4,5-trimethoxyphenyl)pyridin-4-yl]methyl]amino]piperidine Dihydrochloride

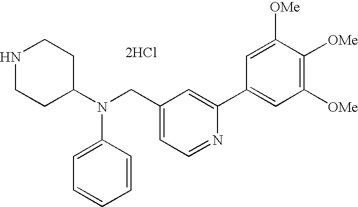

1-(tert-Butoxycarbonyl)-4-[N-phenyl-N-[[2-(3,4,5-trimethoxyphenyl)pyridin-4-yl]methyl]amino]piperidine (760 mg) was treated in the same manner as described in Preparation Example 94 to give light yellow powder of the title compound.

Yield: 652 mg (90%).

### PREPARATION EXAMPLE 189

### Synthesis of 1-(tert-butoxycarbonyl)-4-[N-phenyl-N-[[3-(3,4,5-trimethoxyphenyl)pyridin-5-yl]methyl]amino]piperidine

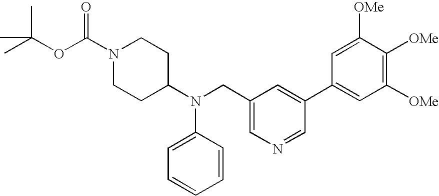

1-(tert-Butoxycarbonyl)-4-phenylaminopiperidine (553 mg) and 5-chloromethyl-3-(3,4,5-trimethoxyphenyl)pyridine (588 mg) was treated in the same manner as described in Example 9 to give light yellow amorphous of the title compound.

Yield: 222 mg (21%).

¹H-NMR (400 MHz, CDCl₃) δ: 1.45 (s, 9H), 1.52-1.67 (m, 2H), 1.82-1.91 (m, 2H), 2.74-2.87 (m, 2H), 3.88-3.90 (m, 1H), 3.88 (s, 3H), 3.89 (s, 6H), 4.14-4.31 (m, 2H), 4.53 (s, 2H), 6.67 (s, 2H), 6.74-6.80 (m, 3H), 7.21 (dd, 2H, J=8.8 Hz, 7.2 Hz), 7.67 (s, 1H), 8.50 (d, 1H, J=5.3 Hz, 2.2 Hz), 8.66 (d, 1H, J=2.1 Hz).

### PREPARATION EXAMPLE 190

### Synthesis of 4-[N-phenyl-N-[[3-(3,4,5-trimethoxyphenyl)pyridin-5-yl]methyl]amino]piperidine Dihydrochloride

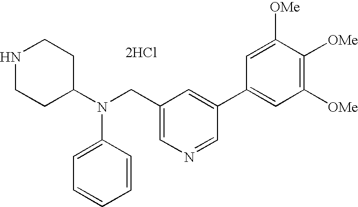

1-(tert-Butoxycarbonyl)-4-[N-phenyl-N-[[3-(3,4,5-trimethoxyphenyl)pyridin-5-yl]methyl]amino]piperidine (222 mg) was treated in the same manner as described in Preparation Example 94 to give light yellow powder of the title compound.

Yield: 197 mg (94%).

### PREPARATION EXAMPLE 191

### Synthesis of 1-(tert-butoxycarbonyl)-4-[N-phenyl-N-[3-(3,4,5-trimethoxyphenyl)benzyl]amino]piperidine

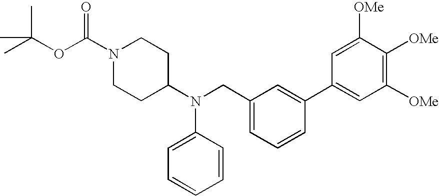

1-(tert-Butoxycarbonyl)-4-phenylaminopiperidine (553 mg) and 3-(3,4,5-trimethoxyphenyl)benzyl chloride (586 mg) was treated in the same manner as described in Example 9 to give light yellow amorphous of the title compound.

Yield: 1.06 g (100%).

¹H-NMR (400 MHz, CDCl₃) δ: 1.45 (s, 9H), 1.52-1.68 (m, 2H), 1.83-1.92 (m, 2H), 2.73-2.86 (m, 2H), 3.88 (s, 3H), 3.89 (s, 6H), 3.94 (tt, 1H, J=11.7 Hz, 3.3 Hz), 4.14-4.30 (m, 2H), 4.52 (s, 2H), 6.69-6.78 (m, 6H), 7.17-7.27 (m, 2H), 7.32-7.42 (m, 3H).

### PREPARATION EXAMPLE 192

### Synthesis of 4-[N-phenyl-N-[3-(3,4,5-trimethoxyphenyl)benzyl]amino]piperidine Hydrochloride

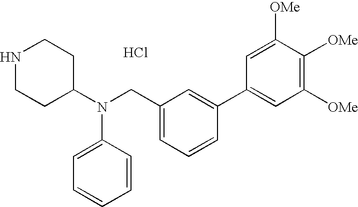

1-(tert-Butoxycarbonyl)-4-[N-phenyl-N-[3-(3,4,5-trimethoxyphenyl)benzyl]amino]piperidine (1.06 g) was treated in the same manner as described in Preparation Example 94 to give light yellow powder of the title compound.

Yield: 909 mg (97%).

### EXAMPLE 165 TO 169

These compounds were obtained by the condensation of amines obtained in Preparation Examples 188, 190 and 192 with chloride derivatives obtained in Preparation Examples 3 and 48. Free bases obtained were then converted to the corresponding hydrochlorides. Yields and NMR data of their free bases are listed below.

| | | | NMR data (400 MHz, |
|---|---|---|---|
| | | | measured as free |
| Example | Structure | Yield | bases, CDCl₃) δ |
| 165 | 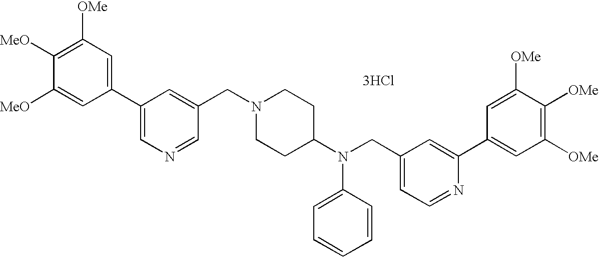 | 53% | 1.63-1.81 (m, 4H), 1.82-1.92 (m, 2H), 2.14-2.24 (m, 2H), 2.95-3.05 (m, 2H), 3.59 (s, 2H), 3.80-4.02 (m, 1H), 3.89 (s, 3H), 3.90 (s, 3H), 3.92 (s, 6H), 3.93 (s, 6H), 4.53 (s, 2H), 6.69-6.77 (m, 5H), 7.13-7.17 (m, 3H), 7.20 (dd, 2H, J=7.6 Hz, 7.6 Hz), 7.55 (s, 1H), 7.76 (s, 1H), 8.51 (d, 1H, J=1.8 Hz), 8.55 (d, 1H, J=5.1 Hz), 8.70 (s, 1H). |
| 166 | 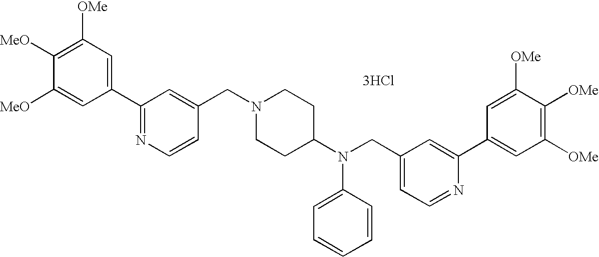 | 50% | 1.85-2.04 (m, 4H), 2.20-2.40 (m, 2H), 2.92-3.25 (m, 2H), 3.60-3.77 (m, 3H), 3.88 (s, 3H), 3.89 (s, 6H), 3.90 (s, 3H), 3.97 (s, 6H), 4.59 (s, 2H), 6.67 (s, 2H), 6.72-6.81 (m, 4H), 7.17-7.30 (m, 4H), 7.68 (s, 1H), 8.50 (s, 1H), 8.62 (d, 1H, J=4.9 Hz), 8.65 (d, 1H, J=2.0 Hz). |
| 167 | 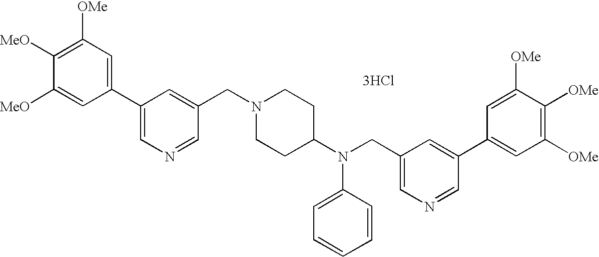 | 43% | 1.72-1.92 (m, 4H), 2.13-2.26 (m, 2H), 2.95-3.04 (m, 2H), 3.59 (s, 2H), 3.78-4.01 (m, 1H), 3.88 (s, 9H), 3.90 (s, 3H), 3.93 (s, 6H), 4.56 (s, 2H), 6.66 (s, 2H), 6.70-6.78 (m, 5H), 7.19 (dd, 2H, J=8.2 Hz, 8.2 Hz), 7.66 (s, 1H), 7.77 (s, 1H), 8.50 (d, 1H, J=2.3 Hz), 8.51 (d, 1H, J=2.2 Hz), 8.65 (d, 1H, J=1.9 Hz), 8.70 (d, 1H, J=2.2 Hz). |
| 168 | 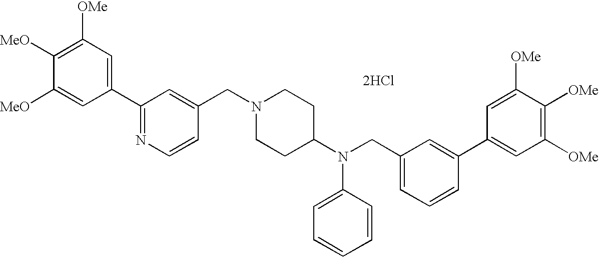 | 82% | 1.75-1.92 (m, 4H), 2.14-2.23 (m, 2H), 2.94-3.01 (m, 2H), 3.57 (s, 2H), 3.80-3.94 (m, 1H), 3.87 (s, 3H), 3.88 (s, 6H), 3.90 (s, 3H), 3.96 (s, 6H), 4.57 (s, 2H), 6.67-6.77 (m, 5H), 7.15-7.27 (m, 5H), 7.34 (dd, 1H, J=7.4 Hz, 7.4 Hz), 7.39 (d, 1H, 7.6 Hz), 7.42 (s, 1H), 7.59 (s, 1H), 8.59 (d, 1H, J=5.1 Hz). |
| 169 | 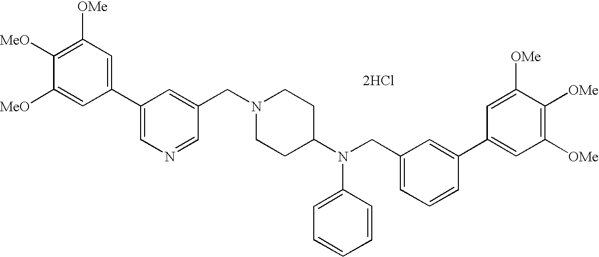 | 65% | 1.72-1.91 (m, 4H), 2.13-2.22 (m, 2H), 2.95-3.03 (m, 2H), 3.59 (s, 2H), 3.79-4.00 (m, 1H), 3.87 (s, 3H), 3.87 (s, 6H), 3.90 (s, 3H), 3.93 (s, 6H), 4.56 (s, 2H), 6.66-6.77 (m, 7H), 7.18 (dd, 2H, J=7.4 Hz, 7.4 Hz), 7.24 (d, 1H, J=7.4 Hz), 7.33 (dd, 1H, J=7.4 Hz, 7.4 Hz), 7.38 (d, 1H, J=7.6 Hz), 7.41 (s, 1H), 7.76 (s, 1H), 8.50 (d, 1H, J=1.6 Hz), 8.69 (d, 1H, J=2.2 Hz). |

### PREPARATION EXAMPLE 193 TO 203

These compounds were prepared by the same procedure as described in on Example from 1 to 3. Structures and NMR data are listed below.

| Preparation | | |
|---|---|---|
| Example | Structure | NMR data (400 MHz, CDCl₃) δ |
| 193 | 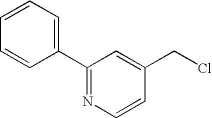 | 4.61 (s, 2H), 7.25 (d, 1H, J=1.2 Hz), 7.41-7.52 (m, 3H), 7.75 (d, 1H, J=0.8 Hz), 7.98-8.02 (m, 2H), 8.69 (d, 1H, J=4.9 Hz). |
| 194 | 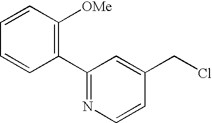 | 3.87 (s, 3H), 4.60 (s, 2H), 7.01 (d, 1H, J=8.4 Hz), 7.08 (t, 1H, J=7.4 Hz), 7.24 (dd, 1H, J=5.1 Hz, 1.4 Hz), 7.38 (dt, 1H, J=7.4 Hz, 1.8 Hz), 7.77 (dd, 1H, J=7.6 Hz, 1.8 Hz), 7.84 (s, 1H), 8.69 (d, 1H, J=5.1 Hz) |
| 195 | 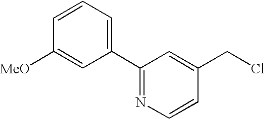 | 3.90 (s, 3H), 4.60 (s, 2H), 6.87-7.03 (1H, m), 7.39 (t, 1H, 7.8 Hz), 7.50-7.66 (m, 2H), 7.73 (s, 1H), 8.68 (d, 1H, J=5.1 Hz) |
| 196 | 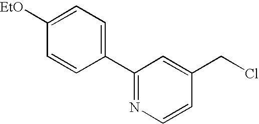 | 1.45 (t, 3H, J=7.0 Hz), 4.12 (q, 2H, J=7.0 Hz), 4.59 (s, 2H), 6.99 (d, 2H, J=8.8 Hz), 7.18 (d, 1H, J=5.1 Hz), 7.20-7.29 (m, 1H), 7.68 (s, 1H), 7.95 (d, 2H, J=8.8 Hz), 8.63 (d, 1H, J=5.1 Hz) |
| 197 | 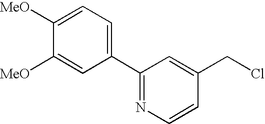 | 3.95 (s, 3H), 4.00 (s, 3H), 4.60 (s, 2H), 6.96 (d, 1H, J=8.4 Hz), 7.21 (d, 1H, J=4.1 Hz), 7.53 (dd, 1H, J=8.4 Hz, 2.0 Hz), 7.67 (d, 1H, J=2.0 Hz), 7.70 (s, 1H), 8.65 (d, 1H, J=5.1 Hz) |
| 198 | 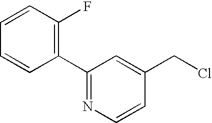 | 4.61 (s, 2H), 7.14-7.21 (m, 1H), 7.21-7.23 (m, 2H), 7.35-7.42 (m, 1H), 7.80 (s, 1H), 7.98 (1H, dt, J=8.0 Hz, 2.0 Hz), 8.73 (d, 1H, J=5.1 Hz) |
| 199 | 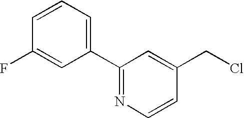 | 4.61 (s, 2H), 7.13 (1H, dt, J=8.4 Hz, 2.8 Hz), 7.28 (1H, d, J=5.0 Hz), 7.40-7.79 (m, 1H), 7.70-7.79 (m, 3H), 8.69 (d, 1H, J=5.0 Hz) |
| 200 | 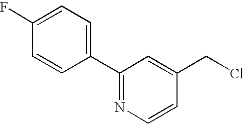 | 4.60 (s, 2H), 7.13-7.20 (m, 2H), 7.25 (1H, d, J=5.1 Hz), 7.70 (s, 1H), 7.95-8.03 (m, 2H), 8.66 (d, 1H, J=5.1 Hz) |
| 201 | 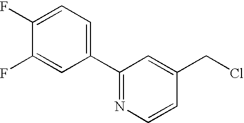 | 4.61 (s, 2H), 7.21-7.30 (m, 2H), 7.69 (s, 1H), 7.73-7.76 (m, 1H), 7.85-7.92 (m, 1H), 8.76 (d, 1H, J=4.9 Hz) |
| 202 | 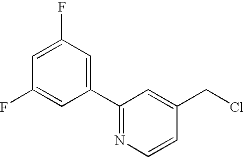 | 4.61 (s, 2H), 6.86-6.91 (m, 1H), 7.31 (1H, d, J=5.1 Hz), 7.51-7.59 (m, 2H), 7.71 (s, 1H), 8.69 (d, 1H, J=5.1 Hz) |
| 203 | 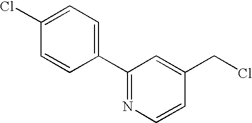 | 4.61 (s, 2H), 7.26 (d, 1H, J=4.9 Hz), 7.45 (d, 2H, J=8.4 Hz), 7.72 (s, 1H), 7.95 (d, 2H, J=8.4 Hz), 8.68 (s, 1H, J=4.9 Hz) |

### PREPARATION EXAMPLE 204

### Synthesis of 1-(tert-butoxycarbonyl)-4-[N-(4-methoxyphenyl)-N-[[(2-phenylpyridin-4-yl)methyl]amino]piperidine

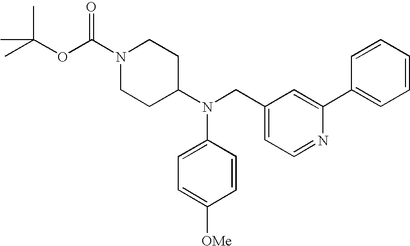

4-(p-Anisidino)-1-(tert-butoxycarbonyl)piperidine (612 mg) and 4-chloromethyl-2-phenylpyridine (204 mg) were condensed in the same manner as described in Example 9 to give the title compound.

Yield: 407 mg (43%).

### PREPARATION EXAMPLE 205

### Synthesis of 4-[N-(4-methoxyphenyl)-N-[(2-phenylpyridin-4-yl)methyl]amino]piperidine Dihydrochloride

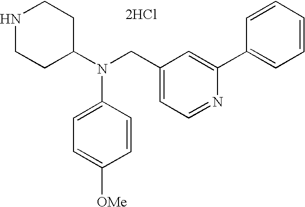

1-(tert-Butoxycarbonyl)-4-[N-(4-methoxyphenyl)-N-[(2-phenylpyridin-4-yl)-methyl]amino]piperidine (407 mg) was treated in the same manner as described in Preparation Example 94 to give the title compound.

Yield: 365 mg (95%).

### PREPARATION EXAMPLE 206

### Synthesis of 1-(tert-butoxycarbonyl)-4-[N-(4-methoxyphenyl)-N-[[2-(2-methoxyphenyl)pyridin-4-yl ]methyl]amino]piperidine

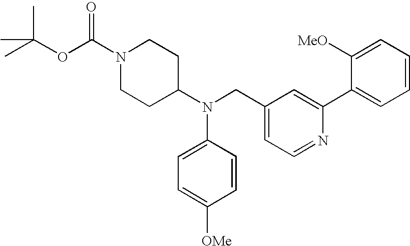

4-(p-Anisidino)-1-(tert-butoxycarbonyl)piperidine (306 mg) and 4-chloromethyl-2-(2-methoxyphenyl)pyridine (234 mg) were condensed in the same manner as described in Example 9 to give the title compound.

Yield: 237 mg (72%).

### PREPARATION EXAMPLE 207

### Synthesis of 4-[N-(4-methoxyphenyl)-N-[[2-(2-methoxyphenyl)pyridin-4-yl]methyl]amino]piperidine Dihydrochloride

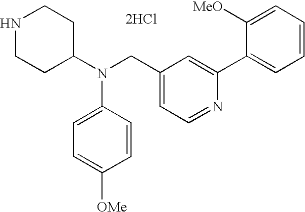

1-(tert-Butoxycarbonyl)-4-[N-(4-methoxyphenyl)-N-[[2-(2-methoxyphenyl)pyridin-4-yl]methyl]amino]piperidine (360 mg) was treated in the same manner as described in Preparation Example 94 to give the title compound.

Yield: 365 mg (65%).

### PREPARATION EXAMPLE 208

### Synthesis of 1-(tert-butoxycarbonyl)-4-[N-(4-methoxyphenyl)-N-[[2-(3-methoxyphenyl)pyridin-4-yl ]methyl]amino]piperidine:

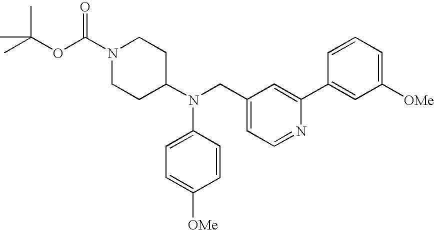

4-(p-Anisidino)-1-(tert-butoxycarbonyl)piperidine (306 mg) and 4-chloromethyl-2-(3-methoxyphenyl)pyridine (234 mg) were condensed in the same manner as described in Example 9 to give the title compound.

Yield: 550 mg (theoretical yield).

### PREPARATION EXAMPLE 209

### Synthesis of 4-[N-(4-methoxyphenyl)-N-[[2-(3-methoxyphenyl)pyridin-4-yl]methyl]amino]piperidine Dihydrochloride

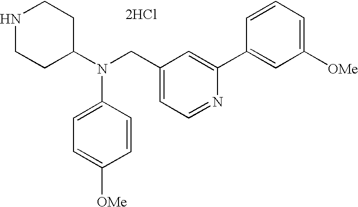

1-(tert-Butoxycarbonyl)-4-[N-(4-methoxyphenyl)-N-[[2-(3-methoxyphenyl)pyridin-4-yl]methyl]amino]piperidine (550 mg) was treated in the same manner as described in Preparation Example 94 to give the title compound.

Yield: 436 g (85%).

### PREPARATION EXAMPLE 210

### Synthesis of 1-(tert-butoxycarbonyl)-4-[N-[[2-(4-ethoxyphenyl)pyridin-4-yl]methyl]-N-(4-methoxyphenyl)]amino]piperidine

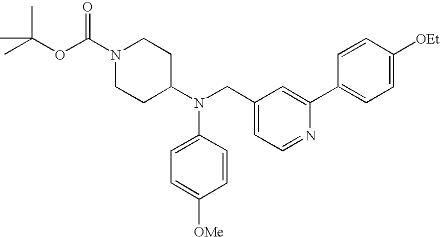

4-(p-Anisidino)-1-(tert-butoxycarbonyl)piperidine (306 mg) and 4-chloromethyl-2-(4-ethoxyphenyl)pyridine (248 mg) were condensed in the same manner as described in Example 9 to give the title compound.

Yield: 515 mg (99%).

### PREPARATION EXAMPLE 211

### Synthesis of 4-[N-[[2-(4-ethoxyphenyl)pyridin-4-yl]methyl-N-(4-methoxyphenyl)amino]piperidine Dihydrochloride

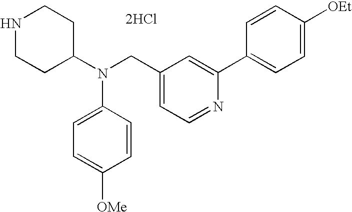

1-(tert-Butoxycarbonyl)-4-[N-[[2-(4-ethoxyphenyl)pyridin-4-yl]methyl-N-(4-methoxyphenyl)amino]piperidine (515 mg) was treated in the same manner as described in Preparation Example 94 to give the title compound.

Yield: 418 mg (80%).

### PREPARATION EXAMPLE 212

### Synthesis of 1-(tert-butoxycarbonyl)-4-[N-(4-methoxyphenyl)-N-[[2-(3,4-dimethoxyphenyl)pyridin-4-yl]methyl]amino]piperidine

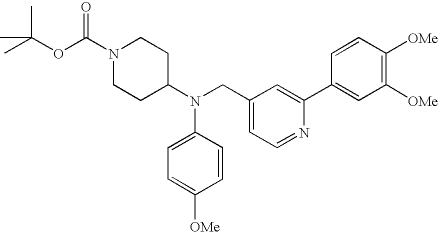

4-(p-Anisidino)-1-(tert-butoxycarbonyl)piperidine (306 mg) and 4-chloromethyl-2-(3,4-dimethoxyphenyl)pyridine (264 mg) were condensed in the same manner as described in Example 9 to give the title compound.

Yield: 600 mg (theoretical yield).

### PREPARATION EXAMPLE 213

### Synthesis of 4-[N-[[2-(3,4-dimethoxyphenyl)pyridin-4-yl]methyl]-N-(4-methoxyphenyl)amino]piperidine Dihydrochloride

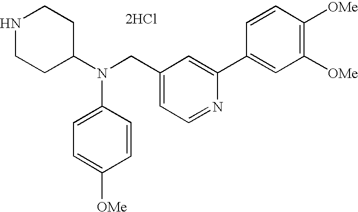

1-(tert-Butoxycarbonyl)-4-[N-[[2-(3,4-dimethoxyphenyl)pyridin-4-yl]methyl]-N-(4-methoxyphenyl)amino]piperidine (600 mg) was treated in the same manner as described in Preparation Example 94 to give the title compound.

Yield: 416 mg (77%).

### PREPARATION EXAMPLE 214

### Synthesis of 1-(tert-butoxycarbonyl)-4[N-[[2-(2-fluorophenyl)pyridin-4-yl]methyl]-N-(4-methoxyphenyl)amino]piperidine

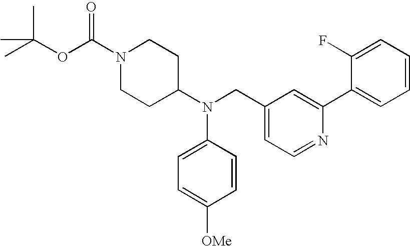

4-(p-Anisidino)-1-(tert-butoxycarbonyl)piperidine (306 mg) and 4-chloromethyl-2-(2-fluorophenyl)pyridine (222 mg) were condensed in the same manner as described in Example 9 to give the title compound.

Yield: 530 mg (theoretical yield).

### PREPARATION EXAMPLE 215

### Synthesis of 4[N-[[2-(2-fluorophenyl)pyridin-4-yl]methyl]-N-(4-methoxyphenyl)amino]piperidine Dihydrochloride

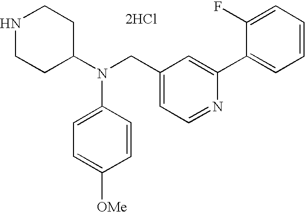

1-(tert-Butoxycarbonyl)-4-[N-[[2-(2-fluorophenyl)pyridin-4-yl]methyl]-N-(4-methoxyphenyl)amino]piperidine (530 mg) was treated in the same manner as described in Preparation Example 94 to give the title compound.

Yield: 423 mg (85%).

### PREPARATION EXAMPLE 216

### Synthesis of 1-(tert-butoxycarbonyl)-4[N-[[2-(3-fluorophenyl)pyridinyl]methyl]-N-(4-methoxyphenyl)amino]piperidine

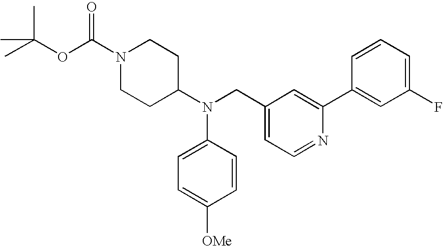

4-p-Anisidino)-1-(tert-butoxycarbonyl)piperidine (153 mg) and 4-chloromethyl-2-(3-fluorophenyl)pyridine (111 mg) were condensed in the same manner as described in Example 9 to give the title compound.

Yield: 270 mg (theoretical yield).

### PREPARATION EXAMPLE 217

### Synthesis of 4-[[[2-(3-fluorophenyl)pyridinyl]methyl]-N-(4-methoxyphenyl)amino]piperidine Dihydrochloride

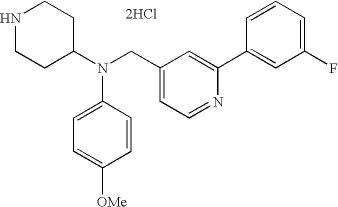

1-(tert-Butoxycarbonyl)-4[N-[[2-(3-fluorophenyl)pyridin-4-yl]methyl]-N-(4-methoxyphenyl)amino]piperidine (270 mg) was treated in the same manner as described in Preparation Example 94 to give the title compound.

Yield: 193 mg (70%).

### PREPARATION EXAMPLE 218

### Synthesis of

1-(tert-butoxycarbonyl)-4-[N-[[2-(4-fluorophenyl)pyridin-4-yl]methyl]-N-(4-methoxyphenyl)amino]piperidine:

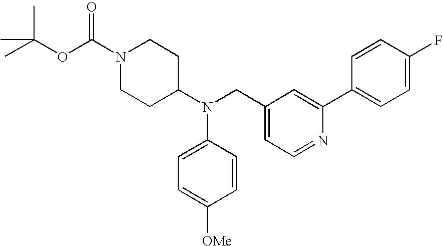

4-(p-Anisidino)-1-(tert-butoxycarbonyl)piperidine (306 mg) and 4-chloromethyl-2-(4-fluorophenyl)pyridine (222 mg) were condensed in the same manner as described in Example 9 to give the title compound.

Yield: 550 mg (theoretical yield).

### PREPARATION EXAMPLE 219

### Synthesis of 4-[N-[[2-(4-fluorophenyl)pyridin-4-yl]methyl-N-(4-methoxyphenyl)amino]piperidine Dihydrochloride

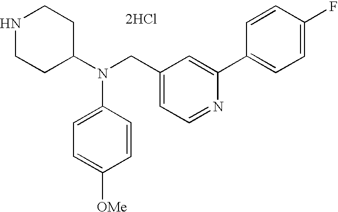

1-(tert-Butoxycarbonyl)-4-[N-[[2-(4-fluorophenyl)pyridin-4-yl]methy]-1-N-(4-methoxyphenyl)amino]piperidine (550 mg) was treated in the same manner as described in Preparation Example 94 to give the title compound.

Yield: 439 mg (880%).

### PREPARATION EXAMPLE 220

### Synthesis of 1-(tert-butoxycarbonyl)-4[N-[[2-(3,4-difluorophenyl)pyridinyl]methyl]-N-(4-methoxyphenyl)amino]piperidine

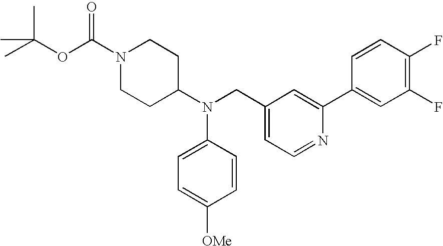

4-(p Anisidino)-1-(tert-butoxycarbonyl)piperidine (306 mg) and 4-chloromethyl-2-(3,4 difluorophenyl)pyridine (240 mg) were condensed in the same manner as described in Example 9 to give the title compound.

Yield: 590 mg (theoretical yield).

### PREPARATION EXAMPLE 221

### Synthesis of 4-[N-[[2-(3,4-difluorophenyl)pyridin-4-yl]methyl]-N-(4-methoxyphenyl)amino]piperidine dihydrochloride

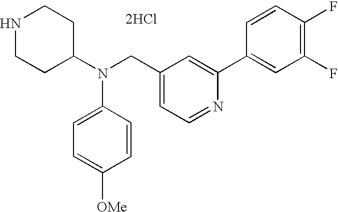

1-(tert-Butoxycarbonyl)-4-[-N-[[2-(3,4-difluorophenyl)pyridin-4-yl]methyl]-N -(4-methoxyphenyl)amino]piperidine (590 mg) was treated in the same manner as described in Preparation Example 94 to give the title compound.

Yield: 483 mg (93%).

### PREPARATION EXAMPLE 222

### Synthesis of 1-(tert-butoxycarbonyl)-4-[N-[[2-(3,5-difluorophenyl)pyridin-4-yl]methyl]-N-(4-methoxyphenyl)amino]piperidine

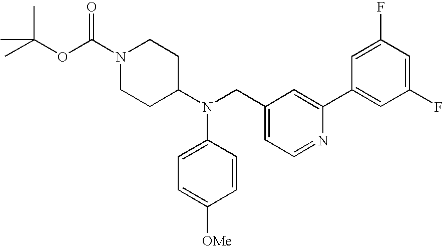

4-(p-Anisidino)-1-(tert-butoxycarbonyl)piperidine (306 mg) and 4-chloromethyl-2-(3,5-difluorophenyl)pyridine (240 mg) were condensed in the same manner as described in Example 9 to give the title compound.

Yield: 530 mg (theoretical yield).

### PREPARATION EXAMPLE 223

### Synthesis of 4-[N-[[2-(3,5-difluorophenyl)pyridin-4-yl]methyl]-N-(4-methoxyphenyl)amino]piperidine Dihydrochloride

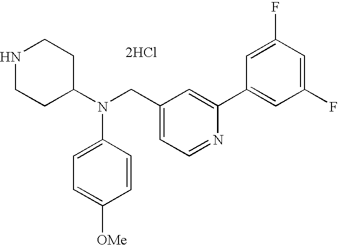

1 tert-Butoxycarbonyl)-4-[N-[[2-(3,5-difluorophenyl)pyridin-4-yl]methyl]-N-(4-methoxyphenyl)amino]piperidine: (530 mg) was treated in the same manner as described in Preparation Example 94 to give the title compound.

Yield: 418 mg (81%).

### PREPARATION EXAMPLE 224

### Synthesis of 1-(tert-butoxycarbonyl)-4-[N-[[2-(4-chlorophenyl)pyridin-4-yl]methyl]-N-(4-methoxyphenyl)amino]piperidine

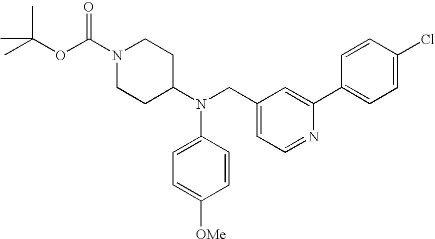

4-(p-Anisidino)-1-(tert-butoxycarbonyl)piperidine (306 mg) and 4-chloromethyl-2-(4-chlorophenyl)pyridine (238 mg) were condensed in the same manner as described in Example 9 to give the title compound.

Yield: 600 mg (theoretical yield).

### PREPARATION EXAMPLE 225

### Synthesis of 4-[N-[[2-(4-chlorophenyl)pyridin-4-yl]methyl]-N-(4-methoxyphenyl)amino]piperidine Dihydrochloride

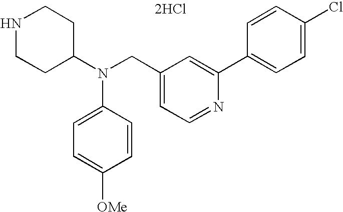

1-(tert-Butoxycarbonyl)-4-[N-[[2-(4-chlorophenyl)pyridin-4-yl]methyl]-N-(4-methoxyphenyl)amino]piperidine: (600 mg) was treated in the same manner as described in Preparation Example 94 to give the title compound.

Yield: 447 mg (86%).

### EXAMPLES 170 TO 202

These compounds were obtained by the condensation of amines obtained in Preparation Examples 96, 205, 207, 209, 211, 213, 215, 217, 219, 221, 223 and 225 with chloride derivatives obtained in Preparation Examples 3, 93, 94, 95, 96, 97, 98, 99, 100, 101, 102 and 103. Free bases obtained were then converted to the corresponding hydrochlorides. Yields and NMR data of their free bases are listed below.

| | | | NMR data (400 MHz, |
|---|---|---|---|
| | | | measured as free |
| Example | Structure | Yield | bases, CDCl₃) δ |
| 170 | 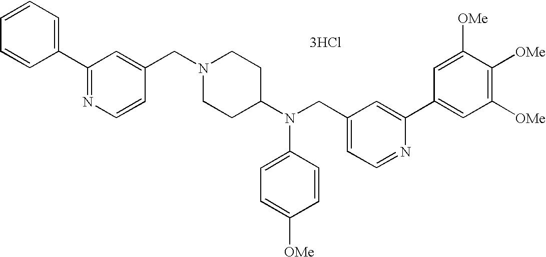 | 47% | 1.67-1.80 (m, 2H), 1.83-1.91 (m, 2H), 2.10-2.19 (m, 2H), 2.93-3.00 (m, 2H), 3.54-3.65 (m, 1H), 3.56 (s, 2H), 3.73 (s, 3H), 3.89 (s, 3H), 3.93 (s, 6H), 4.45 (s, 3H), 6.73 (d, 2H, J=9.4 Hz), 6.78 (d, 2H, J=9.4 Hz), 7.14-7.21 (m, 2H), 7.15 (s, 2H), 7.38-7.49 (m, 3H), 7.57 (s, 1H), 7.68 (s, 1H), 7.97 (d, 1H, J=1.0 Hz), 7.99 (d, 1H, J=1.6 Hz), 8.54 (d, 1H, J=5.1 Hz), 8.61 (d, |
| # 1H, J=5.1 Hz). | | | |
| 171 | 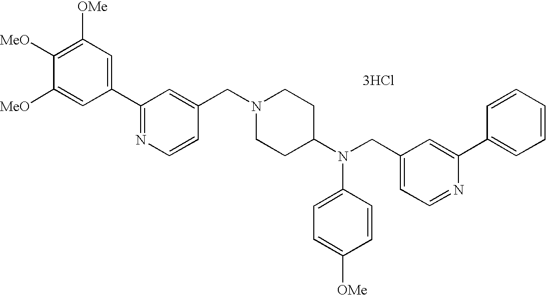 | 55% | 1.62-1.80 (m, 2H), 1.84-1.93 (m, 2H), 2.10-2.20 (m, 2H), 2.93-3.02 (m, 2H), 3.53-3.66 (m, 1H), 3.56 (s, 2H), 3.73 (s, 3H), 3.90 (s, 3H), 3.96 (s, 6H), 4.44 (s, 2H), 6.65-6.83 (m, 4H), 7.14-7.30 (m, 4H), 7.36-7.50 (m, 3H). 7.59 (s, 1H), 7.67 (s, 1H), 7.93 (d, 2H, J=7.0 Hz), 8.54-8.61 (m, 2H). |
| 172 | 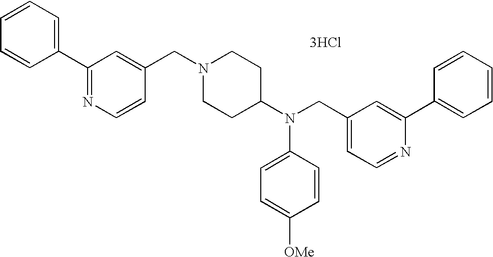 | 54% | 1.67-1.92 (m, 4H), 2.08-2.20 (m, 2H), 2.92-3.01 (m, 2H), 3.52-3.65 (m, 1H), 3.55 (s, 2H), 3.72 (s, 3H), 4.38 (s, 2H), 6.72 (d, 2H, J=9.2 Hz), 6.78 (d, 2H, J=9.0 Hz), 7.18 (dd, 2H, J=4.9 Hz, 4.9 Hz), 7.36-7.50 (m, 6H), 7.67 (s, 1H), 7.68 (s, 1H), 7.93 (dd, 2H, J=8.4 Hz, 1.2 Hz), 7.98 (dd, 2H, J=8.6 Hz, 1.4 Hz), 8.57 (d, 1H, J=5.1 Hz), 8.60 (d, 1H, J=5.1 Hz). |
| 173 | 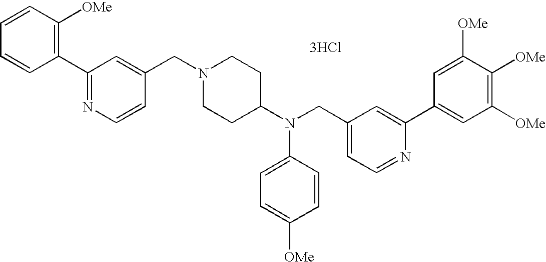 | 100% | 1.66-1.79 (m, 2H), 1.82-1.91 (m, 2H), 2.09-2.20 (m, 2H), 2.93-3.03 (m, 2H), 3.56 (s, 2H), 3.56-3.59 (m, 1H), 3.73 (s, 3H), 3.80 (s, 3H), 3.89 (s, 3H), 3.93 (s, 6H), 4.45 (s, 2H), 6.73 (d, 2H, J=9.3 Hz), 6.78 (d, 2H, J=9.3 Hz), 6.98 (d, 1H, J=8.5 Hz), 7.07 (t, 1H J=7.6 Hz), 7.15 (s, 2H), 7.15-7.19 (m, 2H), 7.33-7.38 (m, 1H), 7.57 (s, 1H), 7.66-7.74 (m, 2H), 8.53 (d, 1H, |
| # J=5.1 Hz, 8.61 (d, 1H, J=4.9 Hz. | | | |
| 174 | 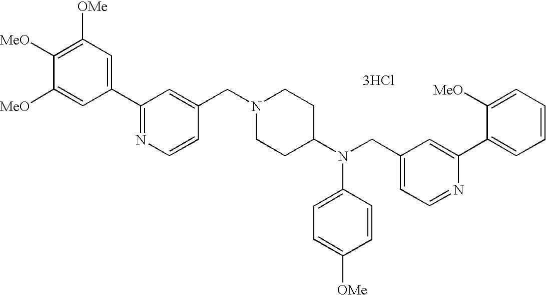 | 94% | 1.70-1.80 (m, 2H), 1.83-1.91 (m, 2H), 2.11-2.18 (m, 2H), 2.92-3.01 (m, 2H), 3.56 (s, 2H), 3.57-3.65 (m, 1H), 3.73 (s, 3H), 3.74 (s, 3H), 3.90 (s, 3H), 3.96 (s, 6H), 4.44 (s, 2H), 6.71 (d, 2H, J=9.0 Hz), 6.78 (d, 2H, J=9.0 Hz), 6.96 (d, 1H, J=8.3 Hz), 7.05 (dt, 1H, J=7.3 Hz, 1.0 Hz), 7.14 (d, 1H, J=5.2 Hz), 7.20 (d, 1H, J=5.2 Hz), 7.22 (2H, s), 7.32-7.37 (m, 1H), 7.59 (s, |
| # 1H), 7.71-7.75 (m, 2H), 8.56-8.60 (m, 2H). | | | |
| 175 | 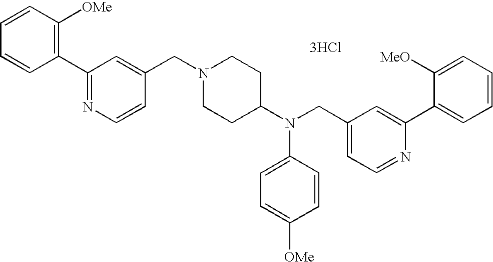 | 98% | 1.67-1.80 (m, 2H), 1.83-1.90 (m, 2H), 2.10-2.19 (m, 2H), 2.94-3.03 (m, 2H), 3.50-3.67 (m, 1H), 3.56 (s, 2H), 3.73 (s, 3H), 3.74 (s, 3H), 3.79 (s, 3H), 4.44 (s, 2H), 6.70 (d, 2H, J=9.3 Hz), 6.78 (d, 2H, J=9.3 Hz), 6.96 (d, 1H, J=8.3 Hz), 6.98 (d, 1H, J=8.8 Hz), 7.04 (dd, 1H, J=7.6 Hz, 1.0 Hz), 7.07 (dd, 1H, 7.6, J=1.0 Hz), 7.12-7.19 (m, 2H), 7.32-7.39 (m, 2H), 7.70-7.75 |
| # (m, 4H), 8.58 (d, 1H, J=5.1 Hz), 8.61 (d, 1H, J=4.9 Hz). | | | |
| 176 | 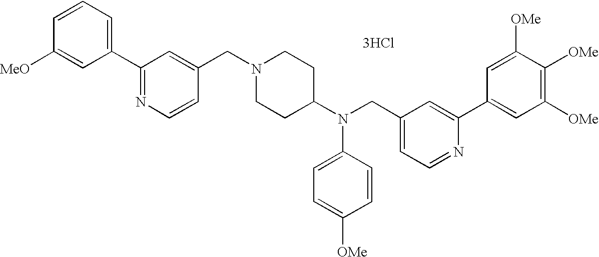 | 100% | 1.68-1.79 (m, 2H), 1.82-1.90 (m, 2H), 2.10-2.19 (m, 2H), 2.90-3.01 (m, 2H), 3.56 (s, 2H), 3.56-3.58 (m, 1H), 3.73 (s, 3H), 3.89 (s, 3H), 3.91 (s, 3H), 3.93 (s, 6H), 4.45 (s, 2H), 6.73 (d, 2H, J=9.3 Hz), 6.78 (d, 2H, J=9.3 Hz), 6.93-6.99 (m, 1H), 7.15 (s, 2H), 7.16-7.20 (m, 2H), 7.37 (t, 1H, J=7.8 Hz), 7.52-7.59 (m, 3H), 7.67 (s, 1H), 8.54 (d, 1H, J=5.1 Hz), 8.60 (d, 1H, |
| # J=5.1 Hz). | | | |
| 177 | 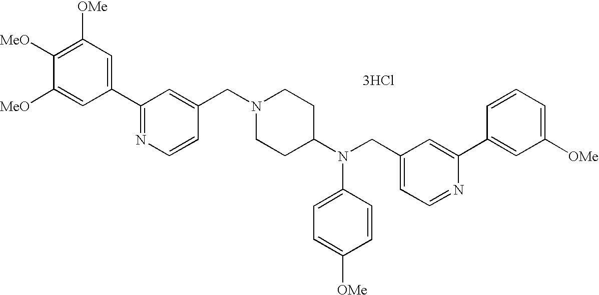 | 100% | 1.68-1.79 (m, 2H), 1.83-1.92 (m, 2H), 2.11-2.16 (m, 2H), 2.91-3.02 (m, 2H), 3.56 (s, 2H), 3.55-3.65 (m, 1H), 3.73 (s, 3H), 3.88 (s, 3H), 3.90 (s, 3H), 3.96 (s, 6H), 4.43 (s, 2H), 6.72 (d, 2H, J=9.3 Hz), 6.78 (d, 2H, J=9.3 Hz), 6.95 (dd, 1H. J=8.3 Hz, 2.7 Hz), 7.16-7.21 (m, 2H), 7.22 (s, 2H), 7.35 (t, 1H, J=7.8 Hz), 7.48 (d, 1H, J=7.8 Hz), 7.53 (t, 1H, J=2.7 Hz), 7.59 (s, |
| # 1H), 7.65 (s, 1H), 8.55-8.60 (m, 2H). | | | |
| 178 | 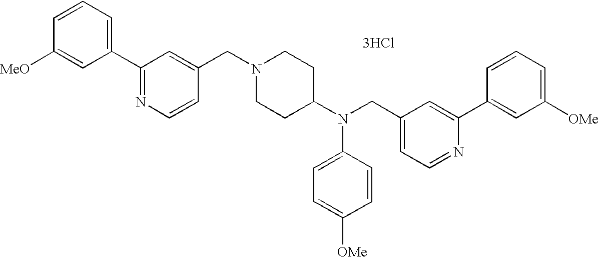 | 100% | 1.65-1.79 (m, 2H), 1.82-1.90 (m, 2H), 2.09-2.19 (m, 2H), 2.92-3.00 (m, 2H), 3.50-3.66 (m, 1H), 3.56 (s, 2H), 3.73 (s, 3H), 3.73 (s, 3H), 3.88 (s, 3H), 3.89 (s, 3H), 4.44 (s, 2H), 6.72 (d, 2H, J=9.3 Hz), 6.78 (d, 2H, J=9.3 Hz), 6.92-6.98 (m, 2H), 7.16-7.21 (m, 2H), 7.34 (d, 1H, J=7.8 Hz), 7.38 (d, 1H, J=8.5 Hz), 7.46-7.59 (m, 4H), 7.65 (s, 1H), 7.67 (s, 1H), 8.57 (dd, 1H, |
| # J=5.1 Hz, 0.7 Hz), 8.60 (d, 1H, J=5.1 Hz). | | | |
| 179 | 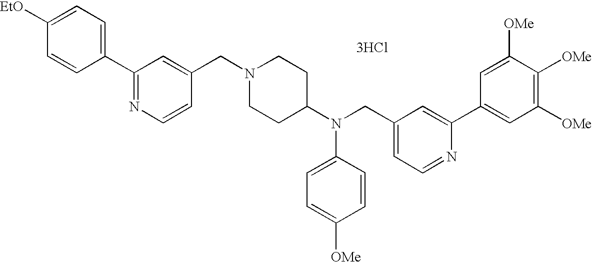 | 76% | 1.44 (t, 3H, J=7.1 Hz), 1.70-1.80 (m, 2H), 1.82-1.91 (m, 2H), 2.10-2.19 (m, 2H), 2.90-3.02 (m, 2H), 3.54 (s, 2H), 3.73-3.78 (m, 1H), 3.73 (s, 3H), 3.88 (s, 3H), 3.93 (s, 6H), 4.09 (q, 2H, J=7.1 Hz), 4.45 (s, 2H), 6.73 (d, 2H, J=9.2 Hz), 6.78 (d, 2H, J=9.2 Hz), 6.97 (d, 2H, J=8.8 Hz), 7.10-7.18 (m, 2H), 7.15 (s, 2H), 7.57 (s, 1H), 7.61 (s, 1H), 7.92 (d, 2H, J=8.8 Hz), 8.52- |
| # 8.58 (m, 2H). | | | |
| 180 | 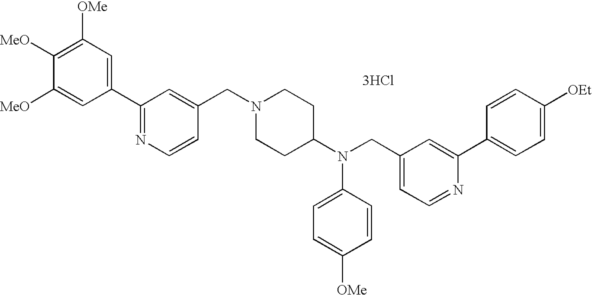 | 93% | 1.43 (t, 3H, J=6.8 Hz), 1.68-1.80 (m, 2H), 1.82-1.92 (m, 2H), 2.10-2.19 (m, 2H), 2.90-3.01 (m, 2H), 3.56 (s, 2H), 3.57-3.64 (m, 1H), 3.73 (s, 3H), 3.90 (s, 3H), 3.96 (s, 6H), 4.08 (q, 2H, J=6.8 Hz), 4.42 (s, 2H), 6.72 (d, 2H, J=9.0 Hz), 6.78 (d, 2H, J=9.3 Hz), 6.95 (d, 2H, J=8.8 Hz), 7.11 (d, 1H, J=5.1 Hz), 7.20 (d, 1H, J=5.1 Hz), 7.22 (s, 2H), 7.58-7.62 (m, 2H), 7.87 (d, |
| # 2H, J=8.8 Hz), 8.52 (d, 1H, J=5.1 Hz), 8.58 (d, 1H, J=5.1 Hz). | | | |
| 181 | 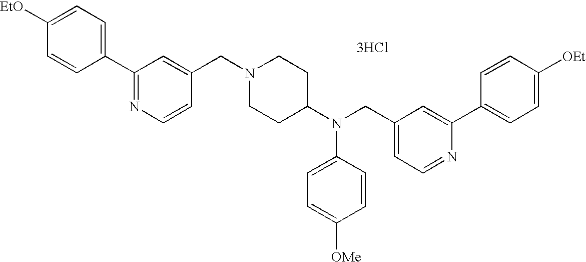 | 100% | 1.43 (t, 3H, J=7.1 Hz), 1.44 (t, 3H, J=7.1 Hz), 1.67-1.78 (m, 2H), 1.82-1.90 (m, 2H), 2.09-2.18 (m, 2H), 2.92-3.00 (m, 2H), 3.54 (s, 2H), 3.55-3.65 (m, 1H), 3.73 (s, 3H), 4.08 (q, 2H, J=7.1 Hz), 4.09 (q, 2H, J=6.8 Hz), 4.42 (s, 2H), 6.71 (d, 2H, J=9.0 Hz), 6.78 (d, 2H, J=9.0 Hz), 6.93-7.00 (m, 4H), 7.10-7.14 (m, 2H), 7.60 (s, 2H), 7.88 (s, 2H), 7.88 (d, 2H, J=8.8 Hz), |
| # 7.93 (d, 2H, J=8.8 Hz), 8.52 (d, 1H, J=5.1 Hz), 8.56 (d, 1H, J=4.9 Hz). | | | |
| 182 | 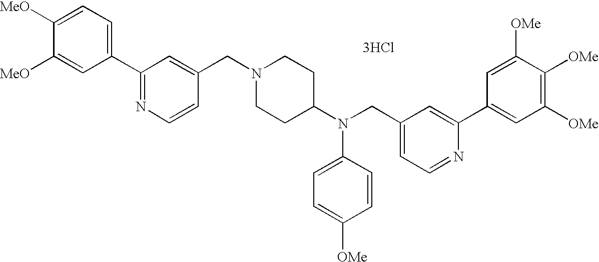 | 100% | 1.68-1.79 (m, 2H), 1.82-1.90 (m, 2H), 2.10-2.19 (m, 2H), 2.90-3.01 (m, 2H), 3.55 (s, 2H), 3.56-3.59 (m, 1H), 3.73 (s, 3H), 3.89 (s, 3H), 3.93 (s, 6H), 3.94 (s, 3H), 3.99 (s, 3H), 4.45 (s, 2H), 6.76 (d, 2H, J=9.5 Hz), 6.78 (d, 2H, J=9.5 Hz), 6.94 (d, 1H, J=8.3 Hz), 7.15 (s, 2H), 7.16-7.19 (m, 2H), 7.49-7.66 (m, 4H), 8.54 (d, 1H, J=4.9 Hz), 8.57 (d, 1H, J=5.1 Hz). |
| 183 | 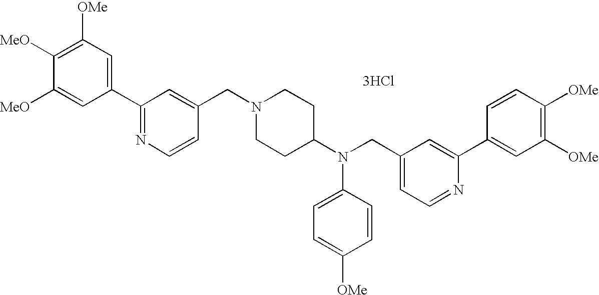 | 100% | 1.68-1.78 (m, 2H), 1.82-1.91 (m, 2H), 2.10-2.18 (m, 2H), 2.93-3.00 (m, 2H), 3.56 (s, 2H), 3.56-3.62 (m, 1H), 3.73 (s, 3H), 3.90 (s, 3H), 3.93 (s, 3H), 3.96 (s, 6H), 3.97 (S, 3H), 4.43 (s, 2H), 6.72 (d, 2H, J=9.3 Hz), 6.78 (d, 2H, J=9.3 Hz), 6.92 (d, 1H, J=8.3 Hz), 7.12 (d, 1H, J=5.1 Hz), 7.20 (d, 1H, J=5.1 Hz), 7.22 (s, 2H), 7.42 (d, 1H, J=8.5 Hz, 2.2 Hz), 7.58-7.63 (m, |
| # 3H), 8.53 (d, 1H, J=4.9 Hz), 8.58 (d, 1H, J=5.1 Hz). | | | |
| 184 | 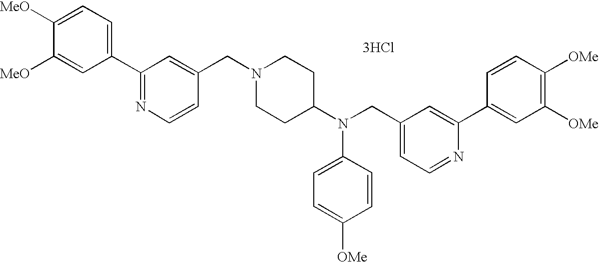 | 89% | 1.67-1.79 (m, 2H), 1.84-1.90 (m, 2H), 2.10-2.19 (m, 2H), 2.93-3.01 (m, 2H), 3.50-3.65 (m, 1H), 3.55 (s, 2H), 3.73 (s, 3H), 3.94 (s, 3H), 3.97 (s, 3H), 3.99 (s, 3H), 4.43 (s, 2H), 6.72 (d, 2H, J=9.3 Hz), 6.78 (d, 2H, J=9.3 Hz), 6.92 (d, 1H, J=8.6 Hz), 6.94 (d, 1H, J=8.3 Hz), 7.14 (d, 1H, J=5.6 Hz), 7.15 (d, 1H, J=6.4 Hz), 7.43 (dd, 1H, J=8.6 Hz, 2.0 Hz), 7.50 (dd, 1H, J=8.3 |
| # Hz, 1.9 Hz), 7.60-7.63 (m, 3H), 7.66 (d, 1H, J=2.2 Hz), 8.53 (d, 1H, J=5.1 Hz), 8.57 (d, 1H, J=4.9 Hz). | | | |
| 185 | 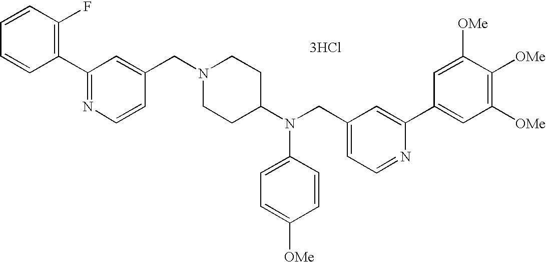 | 100% | 1.68-1.79 (m, 2H), 1.82-1.90 (m, 2H), 2.10-2.20 (m, 2H), 2.93-3.01 (m, 2H), 3.57 (s, 2H), 3.57-3.65 (m, 1H), 3.73 (s, 3H), 3.89 (s, 3H), 3.93 (s, 6H), 4.46 (s, 2H), 6.73 (d, 2H, J=7.3 Hz), 6.78 (d, 2H, J=7.3 Hz), 7.11-7.19 (m, 2H), 7.15 (s, 2H), 7.22-7.29 (m, 2H), 7.34-7.40 (m, 1H), 7.58 (s, 1H), 7.73 (s, 1H), 7.94 (t, 1H, J=8.3 Hz), 8.54 (d, 1H, J=5.1 Hz), 8.64 (d, 1H, J= |
| # 4.9 Hz). | | | |
| 186 | 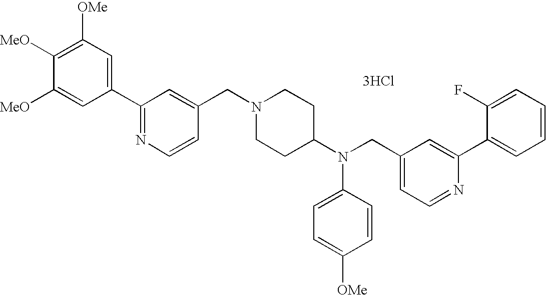 | 88% | 1.68-1.79 (m, 2H), 1.83-1.92 (m, 2H), 2.09-2.16 (m, 2H), 2.93-3.01 (m, 2H), 3.56 (s, 2H), 3.56-3.62 (m, 1H), 3.73 (s, 3H), 3.90 (s, 3H), 3.96 (s, 6H), 4.44 (s, 2H), 6.71 (d, 2H, J=9.3 Hz), 6.77 (d, 2H, J=9.3 Hz), 7.10-7.16 (m, 1H), 7.17-7.26 (m, 3H), 7.22 (s, 2H), 7.32-7.38 (m, 1H), 7.59 (s, 1H), 7.73 (s, 1H), 7.92 (dt, 1H, J=8.0 Hz, 2.0 Hz), 8.57-8.61 (m, 2H). |
| 187 | 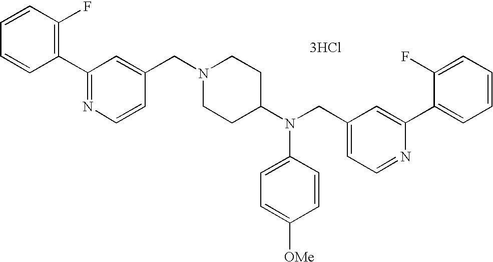 | 100% | 1.66-1.80 (m, 2H), 1.83-1.93 (m, 2H), 2.10-2.20 (m, 2H), 2.92-3.02 (m, 2H), 3.53-3.65 (m, 1H), 3.57 (s, 2H), 3.73 (s, 3H), 4.44 (s, 2H), 6.71 (d, 2H, J=9.0 Hz), 6.78 (d, 2H, J=9.3 Hz), 7.10-7.18 (m, 2H), 7.19-7.29 (m, 4H), 7.32-7.40 (m, 2H), 7.73 (s, 2H), 7.91 (dd, 1H, J=8.1 Hz, 1.4 Hz), 7.95 (dd, 1H, J=7.6 Hz, 1.5 Hz), 8.60 (d, 1H, J=4.9 Hz), 8.64 (d, 1H, J=5.1 Hz). |
| 188 | 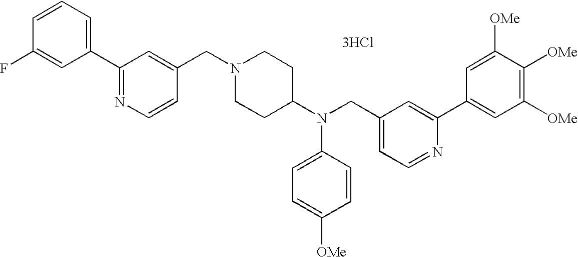 | 96% | 1.67-1.80 (m, 2H), 1.82-1.92 (m, 2H), 2.10-2.20 (m, 2H), 2.91-3.01 (m, 2H), 3.56 (s, 2H), 3.56-3.61 (m, 1H), 3.73 (s, 3H), 3.89 (s, 3H), 3.93 (s, 6H), 4.46 (s, 2H), 6.73 (d, 2H, J=9.3 Hz), 6.78 (d, 2H, J=9.3 Hz), 7.06-7.19 (m, 2H), 7.15 (s, 2H), 7.20-7.26 (m, 1H), 7.38-7.45 (m, 1H), 7.56 (s, 1H), 766-7.78 (m, 3H), 8.54 (d, 1H, J=5.1 Hz), 8.61 (d, 1H, J=4.9 Hz). |
| 189 | 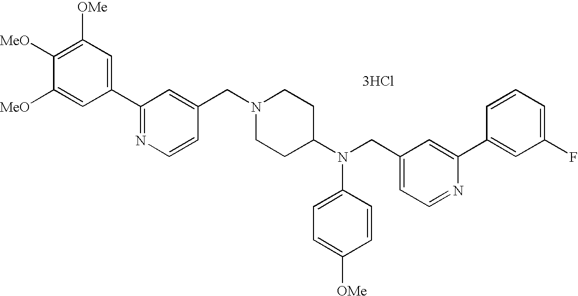 | 92% | 1.65-1.78 (m, 2H), 1.79-1.92 (m, 2H), 2.21-2.26 (m, 2H), 2.90-3.01 (m, 2H), 3.56 (s, 2H), 3.56-3.63 (m, 1H), 3.73 (s, 3H), 3.90 (s, 3H), 3.96 (s, 6H), 4.44 (s, 2H), 6.72 (d, 2H, J=9.3 Hz), 6.78 (d, 2H, J=9.3 Hz), 7.08 (dt, 1H, J=8.3 Hz, 1.7 Hz), 7.18-7.40 (m, 2H), 7.22 (s, 2H), 7.37-7.43 (m, 1H), 7.56-7.72 (m, 4H), 8.55-8.60 (m, 2H). |
| 190 | 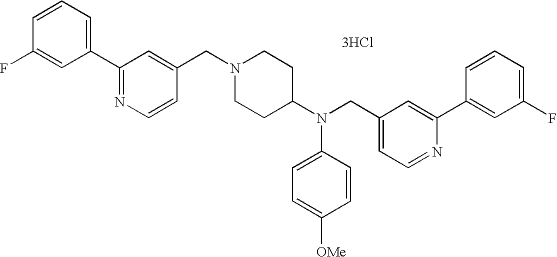 | 55% | 1.66-1.79 (m, 2H), 1.80-1.91 (m, 2H), 2.10-2.20 (m, 2H), 2.88-3.01 (m, 2H), 3.50-3.66 (m, 1H), 3.56 (s, 2H), 3.73 (s, 3H), 4.45 (s, 2H), 6.72 (d, 2H, J=8.5 Hz), 6.79 (d, 2H, J=9.0 Hz), 7.04-7.13 (m, 2H), 7.19-7.25 (m, 2H), 7.35-7.46 (m, 2H), 7.62-7.79 (m, 6H), 8.57 (d, 1H, J=5.1 Hz), 8.61 (d, 1H, J=4.9 Hz). |
| 191 | 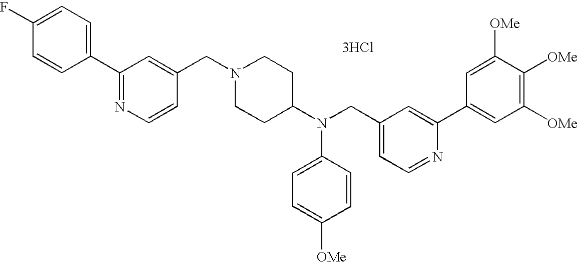 | 100% | 1.68-1.79 (m, 2H), 1.82-1.91 (m, 2H), 2.10-2.19 (m, 2H), 2.92-3.00 (m, 2H), 3.55 (s, 2H), 3.56-3.63 (m, 1H), 3.73 (s, 3H), 3.89 (s, 3H), 3.93 (s, 6H), 4.45 (s, 2H), 6.73 (d, 2H, J=9.3 Hz), 6.78 (d, 2H, J=9.3 Hz), 7.11-7.19 (m, 4H), 7.15 (s, 2H), 7.57 (s, 1H), 7.63 (s, 1H), 7.92-8.01 (m, 2H), 8.54 (d, 1H, J=5.1 Hz), 8.58 (d, 1H, J=5.1 Hz). |
| 192 | 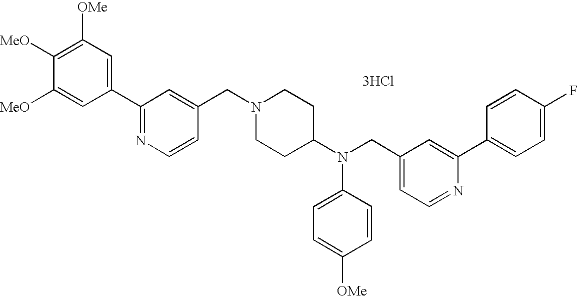 | 100% | 1.68-1.79 (m, 2H), 1.83-1.92 (m, 2H), 2.11-2.19 (m, 2H), 2.93-3.01 (m, 2H), 3.56 (s, 2H), 3.57-3.62 (m, 1H), 3.73 (s, 3H), 3.90 (s, 3H), 3.96 (s, 6H), 4.43 (s, 2H), 6.72 (d, 2H, J=9.3 Hz), 6.78 (d, 2H, J=9.3 Hz), 7.10-7.22 (m, 4H), 7.22 (s, 2H), 7.54-7.66 (m, 2H), 7.88-7.94 (m, 2H), 8.55 (d, 1H, J=4.9 Hz), 8.58 (d, 1H, J=4.9 Hz). |
| 193 | 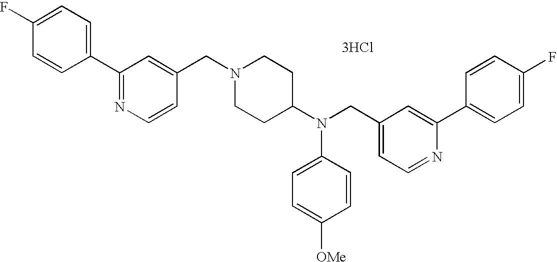 | 90% | 1.66-1.80 (m, 2H), 1.83-1.91 (m, 2H), 2.10-2.19 (m, 2H), 2.92-3.00 (m, 2H), 3.50-3.66 (m, 1H), 3.55 (s, 2H), 3.73 (s, 3H), 4.44 (s, 2H), 6.72 (d, 2H, J=9.3 Hz), 7.78 (d, 2H, J=9.3 Hz), 7.09-7.20 (m, 6H), 7.62 (s, 1H), 7.63 (s, 1H), 7.89-8.00 (m, 4H), 8.55 (d, 1H, J=5.1 Hz, 8.58 (d, 1H, J=4.9 Hz). |
| 194 | 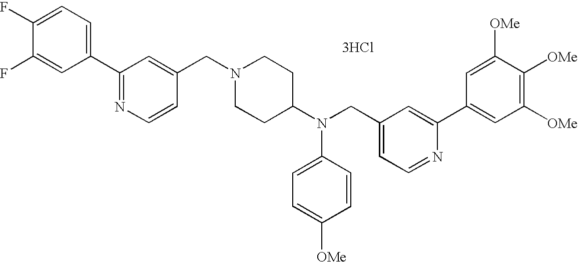 | 36% | 1.68-1.80 (m, 2H), 1.82-1.90 (m, 2H), 2.11-2.19 (m, 2H), 2.91-2.99 (m, 2H), 3.55 (s, 2H), 3.56-3.62 (m, 1H), 3.73 (s, 3H), 3.89 (s, 3H), 3.93 (s, 6H), 4.45 (s, 2H), 6.73 (d, 2H, J=9.3 Hz), 6.78 (d, 2H, J=9.3 Hz), 7.15 (s, 2H), 7.16-7.26 (m, 3H), 7.57 (s, 1H), 7.62 (s, 1H), 7.71 (br, 1H), 7.80-7.90 (m, 1H), 8.54 (d, 1H, J=5.1 Hz), 8.58 (d, 1H, J=4.9 Hz). |
| 195 | 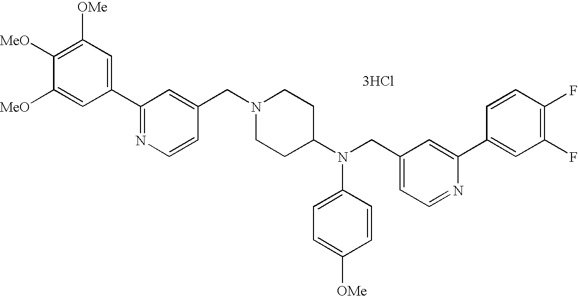 | 100% | 1.60-1.80 (m, 2H), 1.82-1.91 (m, 2H), 2.12-2.19 (m, 2H), 2.91-3.00 (m, 2H), 3.56 (s, 2H), 3.56-3.64 (m, 1H), 3.73 (s, 3H), 3.90 (s, 3H), 3.96 (s, 6H), 4.45 (s, 2H), 6.72 (d, 2H, J=9.0 Hz), 6.78 (d, 2H, J=9.0 Hz), 7.17-7.24 (m, 4H), 7.25-7.27 (m, 1H), 7.60 (s, 2H), 7.65 (br, 1H), 7.77-7.84 (m, 1H), 8.53-8.61 (m, 2H). |
| 196 | 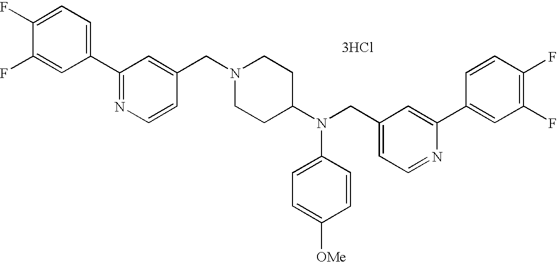 | 100% | 1.66-1.79 (m, 2H), 1.82-1.91 (m, 2H), 2.09-2.20 (m, 2H), 2.90-3.00 (m, 2H), 3.50-3.65 (m, 1H), 3.55 (s, 2H), 3.73 (s, 3H), 4.44 (s, 2H), 6.72 (d, 2H, J=9.3 Hz), 6.79 (d, 2H, J=9.3 Hz), 7.18-7.28 (m, 4H), 7.60 (s, 1H), 7.62 (s, 1H), 7.63-7.68 (m, 1H), 7.70-7.75 (m, 1H), 7.77-7.89 (m, 2H), 8.55 (d, 1H, J=4.9 Hz), 8.58 (d, 1H, J=5.1 Hz). |
| 197 | 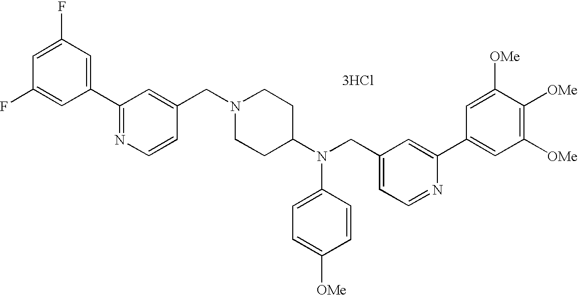 | 100% | 1.68-1.80 (m, 2H), 1.82-1.90 (m, 2H), 2.10-2.21 (m, 2H), 2.90-3.00 (m, 2H), 3.56 (s, 2H), 3.56-3.63 (m, 1H), 3.73 (s, 3H), 3.89 (s, 3H), 3.93 (s, 6H), 4.56 (s, 2H), 6.73 (d, 2H, J=9.3 Hz), 6.78 (d, 2H, J=9.3 Hz), 6.81-6.87 (m, 1H), 7.15 (s, 2H), 7.18 (d, 1H, J=4.2 Hz), 7.22-7.26 (m, 1H), 7.51-7.59 (m, 3H), 7.65 (s, 1H), 8.54 (d, 1H, J=4.9 Hz), 8.59 (d, 1H, J=5.1 Hz). |
| 198 | 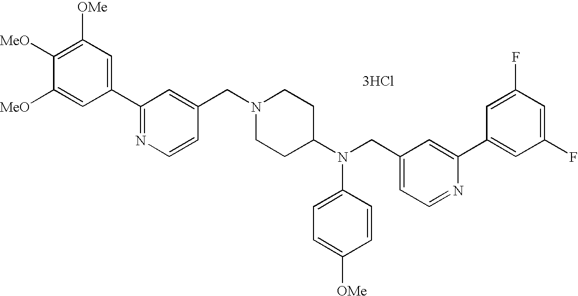 | 100% | 1.65-1.79 (m, 2H), 1.80-1.94 (m, 2H), 2.22-2.25 (m, 2H), 2.90-3.05 (m, 2H), 3.56 (s, 2H), 3.56-3.65 (m, 1H), 3.73 (s, 3H), 3.90 (s, 3H), 3.96 (s, 6H), 4.44 (s, 2H), 6.72 (d, 2H, J=9.2 Hz), 6.78 (d, 2H, J=9.2 Hz), 6.80-6.94 (m, 2H), 7.22 (s, 2H), 7.19-7.28 (m, 1H), 7.45-7.51 (m, 2H), 7.59 (s, 1H), 7.62 (s, 1H), 8.56 (d, 1H, J=4.9 Hz), 8.59 (d, 1H, J=5.1 Hz). |
| 199 | 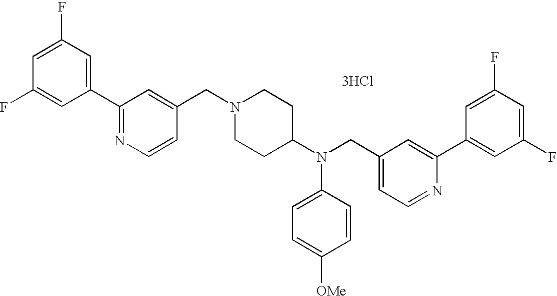 | 100% | 1.67-1.79 (m, 2H), 1.82-1.92 (m, 2H), 2.12-2.20 (m, 2H), 2.92-2.99 (m, 2H), 3.50-3.65 (m, 1H), 3.56 (s, 2H), 3.73 (s, 3H), 4.45 (s, 2H), 6.72 (d, 2H, J=9.0 Hz), 6.79 (d, 2H, J=9.3 Hz), 6.80-6.88 (m, 2H), 7.23-7.27 (m, 2H), 7.48 (dd, 2H, J=8.8 Hz, 2.2 Hz), 7.55 (dd, 2H, J=8.8 Hz, 2.2 Hz), 7.63 (s, 1H), 7.65 (s, 1H), 8.57 (d, 1H, J=4.9 Hz), 8.60 (d, 1H, J=4.9 Hz). |
| 200 | 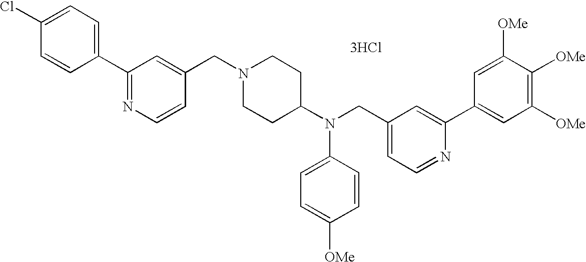 | 84% | 1.68-1.80 (m, 2H), 1.83-1.92 (m, 2H), 2.10-2.21 (m, 2H), 2.91-3.00 (m, 2H), 3.56 (s, 2H), 3.57-3.62 (m, 1H), 3.73 (s, 3H), 3.89 (s, 3H), 3.93 (s, 6H), 4.45 (s, 2H), 6.73 (d, 2H, J=9.3 Hz), 6.78 (d, 2H, J=9.3 Hz), 7.15 (s, 2H), 7.17 (d, 1H, J=4.9 Hz), 7.20 (d, 1H, J=5.1 Hz), 7.43 (d, 2H, J=8.3 Hz), 7.57 (s, 1H), 7.65 (s, 1H), 7.93 (d, 2H, J=8.3 Hz), 8.54 (d, 1H, J=4.9 Hz), |
| # 8.59 (d, 1H, J=5.1 Hz). | | | |
| 201 | 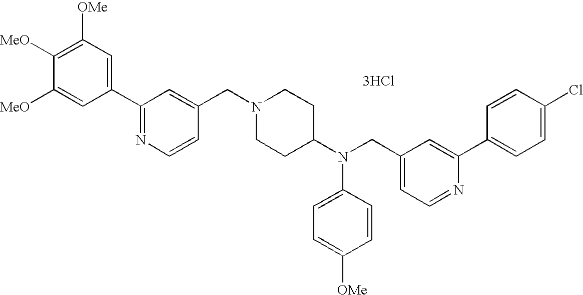 | 72% | 1.65-1.78 (m, 2H), 1.82-1.91 (m, 2H), 2.10-2.16 (m, 2H), 2.91-3.02 (m, 2H), 3.56 (s, 2H), 3.56-3.64 (m, 1H), 3.73 (s, 3H), 3.90 (s, 3H), 3.96 (s, 6H), 4.43 (s, 2H), 6.72 (d, 2H, J=9.3 Hz), 6.78 (d, 2H, J=9.3 Hz), 7.17-7.21 (m, 1H), 7.22 (2H, s), 7.41 (d, 2H, J=8.7 Hz), 7.48 (d, 1H, J=7.8 Hz), 7.59 (s, 1H), 7.63 (s, 1H) 7.87 (d, 2H, J=8.7 Hz), 8.56 (d, 1H, J=4.9 Hz), 8.58 |
| # (d, 1H, J=5.1 Hz). | | | |
| 202 | 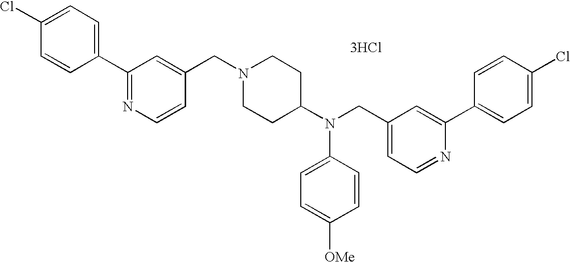 | 94% | 1.67-1.88 (m, 2H), 1.83-1.90 (m, 2H), 2.10-2.17 (m, 2H), 2.92-2.99 (m, 2H), 3.50-3.65 (m, 1H), 3.55 (s, 2H), 3.73 (s, 3H), 4.44 (s, 2H), 6.72 (d, 2H, J=9.0 Hz), 6.78 (d, 2H, J=9.3 Hz), 7.17-7.22 (m, 2H), 7.39-7.45 (m, 4H), 7.63 (s, 1H), 7.65 (s, 1H), 7.88 (d, 2H, J=8.6 Hz), 7.93 (d, 2H, J=8.5 Hz), 8.56 (d, 1H, J=4.9 Hz), 8.59 (d, 1H, J=4.9 Hz). |

### PREPARATION EXAMPLE 226

### Synthesis of 4-[N-[3-(3,4,5-trimethoxyphenyl)benzyl]-N-[4-(methylsulfonyl)phenyl]amino]piperidine

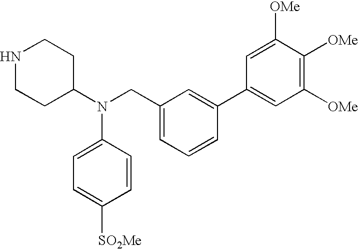

To a solution of 4-[N-[3-(3,4,5-trimethoxyphenyl)benzyl]-N-[4-(methylthio)phenylamino]piperidine hydrochloride (52 mg, obtained in the Preparation Example 145) in dichloromethane (1 mL) was added 3-chloroperbenzoic acid (69 mg) at 0° C. The mixture was stirred at room temperature for 3 hours and saturated aqueous sodium hydrogen carbonate was added. After separating the organic layer, the aqueous layer was extracted with chloroform. Organic layers were combined, washed with brine, dried over anhydrous sodium sulfate and evaporated to give pale yellow oil of the title compound which was used for the next step without further purification.

### EXAMPLE 203

### Synthesis of 4-[N-[4-(methylsulfonyl)phenyl]-N-[3-(3,4,5-trimethoxyphenyl)benzyl]amino]-1-[[2-(3,4,5-trimethoxyphenyl)pyridin-4-yl]methyl]piperidine Dihydrochloride

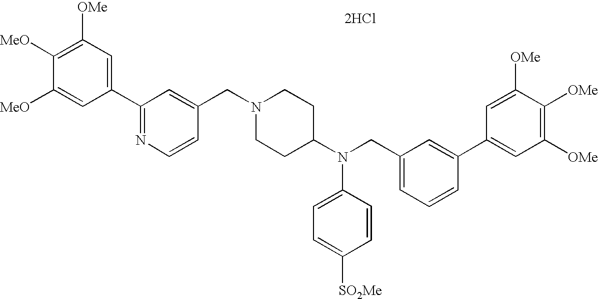

Crude 4-[N-[3-(3,4,5-trimethoxyphenyl)benzyl]-N-[4-(methylsulfonyl)phenyl]amino]piperidine and 4-chloromethyl-2-(3,4,5-trimethoxyphenyl)pylidine (29 mg) were condensed in the same manner as described in Example 9. The title compound was obtained as pale yellow powder after converting a free base to a dihydrochloride.

Yield: 23 mg (26% in 2 steps).

¹H-NMR (400 MHz, measured as a free base, CDCl₃) δ: 1.70-1.97 (m, 4H), 2.16-2.28 (m, 2H), 2.95-3.04 (m, 2H), 2.99 (s, 3H), 3.59 (s, 2H), 3.82 (s, 3H), 3.87-3.97 (m, 1H), 3.90 (s, 3H), 3.91 (s, 3H), 3.92 (s, 31), 3.96 (s, 9H), 4.65 (s, 2H), 6.59 (s, 1H), 6.75 (d, 2H, J=9.3 Hz), 7.19-7.30 (m, 7H), 7.39 (dd, 1H, J=7.6, 7.6 Hz), 7.60 (s, 1H), 7.68 (d, 2H, J=9.0 Hz), 8.60 (d, 1H, J=4.9 Hz).

### EXAMPLE 204

### Synthesis of 4-[N-(4-metoxyphenyl)-N-[[2-(3,4,5-trimethoxyphenyl)pyridin-4-yl]methyl]amino]-1-[[2-(3-methoxyphenyl)pyridin-4-yl]methyl]piperidine Trihydrochloride

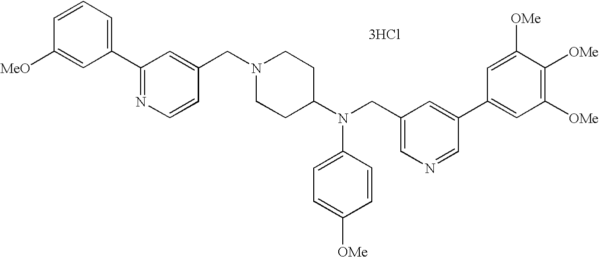

4-[N-(4-Methoxyphenyl)-N-[[3-(3,4,5-trimethoxyphenyl)pyridin-5-yl]methyl]amino]piperidine dihydrochloride (139 mg, obtained in the Preparation Example 98) and 4-chloromethyl-2-(3-methoxyphenyl)pyridine (70 mg, obtained in the Preparation Example 195) were condensed in the same manner described in the Example 9 to give the title compound as a trihydrochloride.

Yield: 131 mg (66%).

¹H-NMR (400 MHz, measured as a free base, CDCl₃) δ: 1.70-1.95 (m, 4H), 2.05-2.25 (m, 2H), 2.90-3.08 (m, 2H), 3.45-3.68 (m, 3H), 3.72 (s, 3H), 3.88 (s, 3H), 3.90 (s, 9H), 4.46 (s, 2H), 6.66 (s, 2H), 6.70-6.85 (m, 4H), 6.96 (d, 1H, J=8.3 Hz), 7.21 (br, 1H), 7.38 (t, 1H, J=7.8 Hz), 7.55 (t, 1H, J=7.8 Hz), 7.59 (s, 1H), 7.63-7.75 (m, 2H), 8.50 (s, 1H), 8.62 (m, 2H).

### EXAMPLE 205

### Synthesis of 4-[N-(4-metoxyphenyl)-N-[[2-(3,4,5-t ethoxyphenyl)pyridin-4-yl]methyl]amino]-1-[[2-(3,4-dimethoxyphenyl)pyridin-4-yl]methyl]piperidine Trihydrochloride

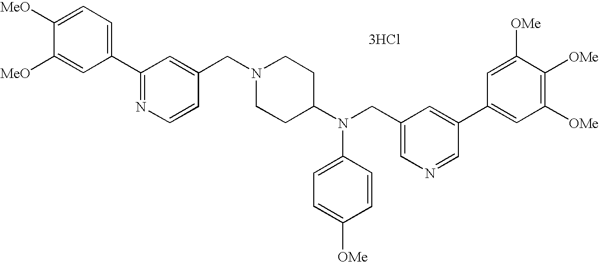

4-[N-(4-Methoxyphenyl)-N-[[3-(3,4,5-trimethoxyphenyl)pyridin-5-yl]methyl]amino]piperidine dihydrochloride (139 mg, obtained in the Preparation Example 98) and 4-chloromethyl-2-(3,4-dimethoxyphenyl)pyridine (80 mg, obtained in the Preparation Example 197) were condensed in the same manner described in the Example 9 to give the title compound as a trihydrochloride.

Yield: 139 mg (67%).

¹H-NMR (400 MHz, measured as a free base, CDCl₃) δ: 1.70-1.95 (m, 4H), 2.05-2.20 (m, 2H), 2.90-3.05 (m, 2H), 3.45-3.60 (m, 3H), 3.73 (s, 3H), 3.88 (s, 3H), 3.89 (s, 6H), 3.94 (s, 3H), 4.00 (s, 3H), 4.46 (s, 2H), 6.65 (s, 2H), 6.74-6.82 (m, 4H), 6.94 (d, 1H, J=8.3 Hz), 7.15 (br, 1H), 7.52 (br, 1H), 7.58-7.71 (m, 3H), 8.50 (s, 1H), 8.57 (d, 1H, J=5.2 Hz), 8.62 (br, 1H).

### EXAMPLE 206

### Synthesis of 4-[N-(4-fluorophenyl)-N-[[3-(3,4,5-trimethoxyphenyl)pyridin-5-yl]methyl]amino]-1-[[2-(3-methoxyphenyl)pyridin-4-yl]methyl]piperidine Trihydrochloride

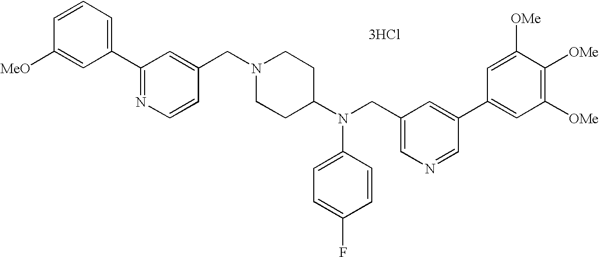

4-[N-(4-Fluorophenyl)-N-[[3-(3,4,5-trimethoxyphenyl)pyridin-5-yl]methyl]amino]piperidine dihydrochloride (135 mg, obtained in the Preparation Example 183) and 4-chloromethyl-2-(3-methoxyphenyl)pyridine (70 mg, obtained in the Preparation Example 195) were condensed in the same manner described in the Example 9 to give the title compound as a trihydrochloride.

Yield: 178 mg (92%).

¹H-NMR (400 MHz, measured as a free base, CDCl₃) δ: 1.73-1.95 (m, 4H), 2.10-2.25 (m, 2H), 2.93-3.05 (m, 2H), 3.57 (s, 2H), 3.64 (br, 1H), 3.88 (s, 3H), 3.89 (s, 9H), 4.51 (s, 2H), 6.66 (s, 2H), 6.70-6.76 (m, 2H), 6.90 (t, 2H, J=8.3 Hz), 6.96 (d, 1H, J=8.3 Hz), 7.21 (br, 1H), 7.38 (t, 1H, J=8.0 Hz), 7.54 (d, 1H, J=7.8 Hz), 7.58 (s, 1H), 7.65 (s, 1H), 7.74 (br, 1H), 8.50 (s, 1H), 8.61 (d, 1H, J=5.1 Hz), 8.65 (br, 1H).

### EXAMPLE 207

### Synthesis of 4-[N-(4-fluorophenyl)-N-[[3-(3,4,5-trimethoxyphenyl)pyridin-5-yl]methyl]amino]-1-[[2-(3,4-dimethoxyphenyl)pyridin-4-yl]methyl]piperidine Trihydrochloride

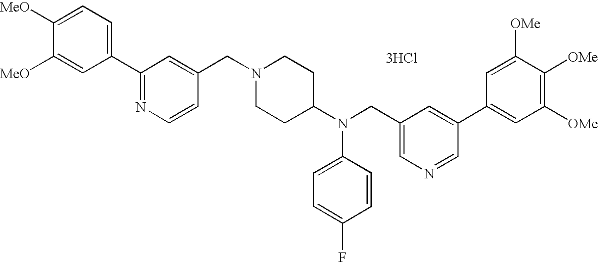

4-[N-(4-Fluorophenyl)-N-[[3-(3,4,5-trimethoxyphenyl)pyridin-5-yl]methyl]amino]piperidine dihydrochloride (135 mg, obtained in the Preparation Example 183) and 4-chloromethyl-2-(3,4-dimethoxyphenyl)pyridine (80 mg, obtained in the Preparation Example 197) were condensed in the same manner described in the Example 9 to give the title compound as a trihydrochloride.

Yield: 195 mg (96%).

¹H-NMR (400 MHz, measured as a free base, CDCl₃) δ: 1.70-1.95 (m, 4H), 2.10-2.24 (m, 2H), 2.94-3.09 (m, 2H), 3.57 (s, 2H), 3.64 (br, 1H), 3.88 (s, 3H), 3.89 (s, 6H), 3.94 (s, 3H), 4.00 (s, 3H), 4.51 (s, 2H), 6.65 (s, 2H), 6.69-6.78 (m, 2H), 6.86-6.97 (m, 3H), 7.16 (d, 1H, J=4.9 Hz), 7.51 (d, 1H, J=8.5 Hz), 7.60-7.70 (m, 3H), 8.50 (s, 1H), 8.58 (d, 1H, J=4.9 Hz), 8.65 (s, 1H).

### EXAMPLE 208

### Synthesis of 4-[N-(3,4,5-difluorophenyl)-N-[[3-(3,4,5-trimethoxyphenyl)pyridin-5-yl]methyl]amino]-1-[[2-(3-methoxyphenyl)pyridinyl]methyl]piperidine Trihydrochloride

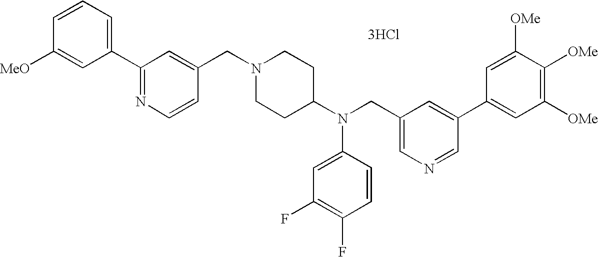

4N-(3,4-Difluorophenyl)-N-[[3-(3,4,5-trimethoxyphenyl)pyridin-5-yl]methyl]amino]piperidine dihydrochloride (160 mg, obtained in the Preparation Example 176) and 4-chloromethyl-2-(3-methoxyphenyl)pyridine (80 mg, obtained in the Preparation Example 195) were condensed in the same manner described in the Example 9 to give the title compound as a trihydrochloride.

Yield: 130 mg (57%).

¹H-NMR (400 MHz, measured as a free base, CDCl₃) δ: 1.73-1.90 (m, 4H), 2.01-2.24 (m, 2H), 2.92-3.05 (m, 2H), 3.57 (s, 2H), 3.67 (br, 1H), 3.88 (s, 3H), 3.89 (s, 3H), 3.90 (s, 6H), 4.52 (s, 2H), 6.36-6.42 (m, 1H), 6.50-6.58 (m, 1H), 6.67 (s, 2H), 6.93-7.01 (m, 2H), 7.20 (br, 1H), 7.38 (t, 1H, J=7.8 Hz), 7.52-7.62 (m, 2H), 7.62-7.72 (m, 2H), 8.48 (br, 1H), 8.61 (br, 1H), 8.66 (d, 1H, J=2.0 Hz).

### EXAMPLE 209

### Synthesis of 1-[[2-(3-methoxyphenyl)pyridin-4-yl]methyl]-4 [N-[[3-(3,4,5-trimethoxyphenyl)pyridin-5-yl]methyl]-N-(4-methylthiophenyl)amino]piperidine

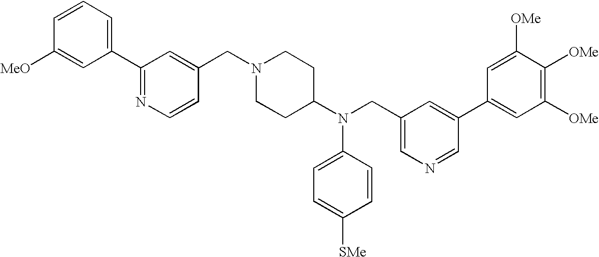

4-[N-(4-Metythiophenyl)-N-[[3-(3,4,5-trimethoxyphenyl)pyridin-5-yl]methyl]amino]piperidine dihydrochloride (121 mg, obtained in the Preparation Example 143) and 4-chloromethyl-2-(4-methoxyphenyl)pyridine (55 mg, obtained in the Preparation Example 195) were condensed in the same manner described in the Example 9 to give the title compound.

Yield: 71 mg (44%).

¹H-NMR (400 MHz, measured as a free base, CDCl₃) δ: 1.72-1.83 (m, 4H), 2.12-2.20 (m, 2H), 2.37 (s, 3H), 2.97 (d, 2H, J=10.8 Hz), 3.56 (s, 2H), 3.75-3.81 (m, 1H), 3.86 (s, 3H), 3.87 (s, 61), 4.54 (s, 2H), 6.64-6.69 (m, 3H), 6.94 (dd, 1H, J=7.8 Hz, 1.9 Hz), 7.17-7.26 (m, 4H), 7.35 (t, 1H, J=7.8 Hz), 7.51-7.66 (m, 4H), 8.47 (s, 1H), 8.59 (d, 1H, J=4.6 Hz), 8.63 (s, 1H).

### TEST EXAMPLE 1

Human umbilical venous endothelial cells (HUVECs) were placed in 10 cm dishes (3×10⁵ cells/dish). Two days thereafter, Trichostatin A (TSA, produced by Upstate) dissolved in dimethyl sulfoxide (DMSO) and the compound prepared in Example 10 dissolved in DMSO were individually added to a final concentrations of 10 μM and 1 μM, respectively. Each sample was stimulated with TNFα (final concentration: 10 ng/mL, Genzyme-Techne). Four hours later, total RNA was extracted with ISOGEN (Nippon Gene Co., Ltd.). The subsequent procedure was performed in accordance with the manufacturer's protocol (Affymetrix). From the thus obtained total RNA, mRNA was purified by a conventional method. cDNA was synthesized from the purified mRNA, and then biotin-labeled cRNA was synthesized by in vitro transcription. The cRNA was purified and subjected to heat treatment for fragmentation. The fragmented cRNA was used in gene expression analysis.

Method of gene expression analysis: The thus-prepared fragmented cRNA was injected to a HuGene human FL array (Affymetrix), and allowed to hybridize for 16 hours at 45° C. After washing, streptavidin labeled with phycoerythrin, and biotinylated anti-streptavidin antibody were added to each sample in order to cause reaction. Gene expression information was read by use of a dedicated scanner for GeneChip™ (Hewlett Packard). The thus-obtained information was analyzed with GeneChip Software (Affymetrix) for comparison in terms of level of expression.

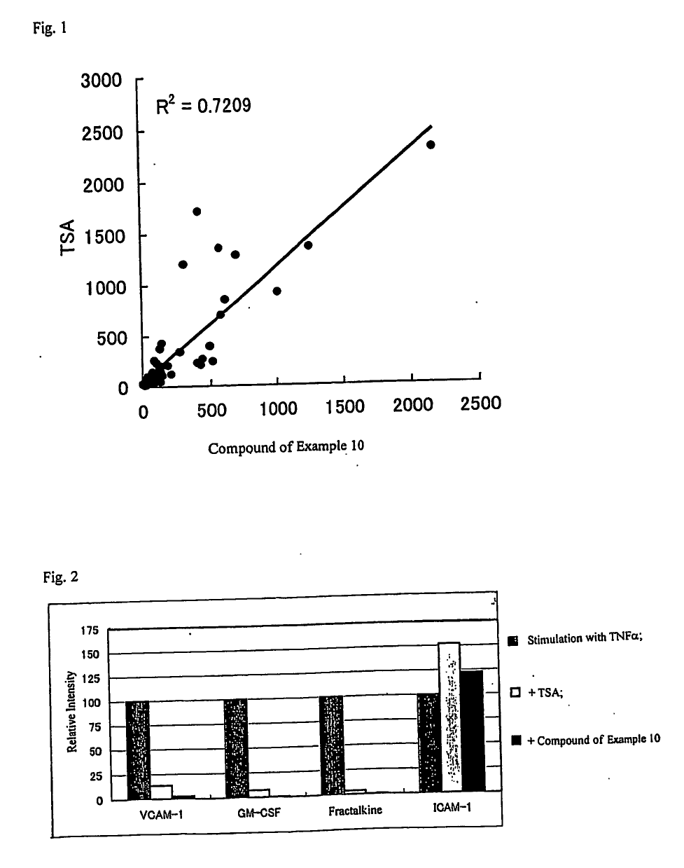

The mRNA expression levels of 52 genes were twice or more increased by stimulation with TNFα. As shown in FIG. 1, the mRNA expression levels of these genes under addition of TSA and those under addition of the compound prepared in Example 10 have a positive correlation. In 25 genes (including VCAM-1, fractalkine, lymphotoxin β, and RDC-1) out of these genes, expression was inhibited by TSA and also by the compound prepared in Example 10. Conversely, expression was enhanced in 6 genes (including ICAM-1). The above results demonstrate that TSA and the compound prepared in Example 10 have similar actions on TNFα-stimulated HUVECs.

TABLE 1
Genes with suppressed expression in the presence
of the two agents

| | No | TNFα | + Compound | |
|---|---|---|---|---|
| Genes | stimulation | stimulation | of Example 10 | + TSA |
| OB-cadherin-2 | 47 | 97 | 24 | 62 |
| caspase-like | 86 | 245 | 76 | 50 |
| apoptosis | | | | |
| regulatory | | | | |
| protein 2 (clarp) | | | | |
| Nef associated | 241 | 844 | 496 | 396 |
| factor 1 | | | | |
| M-Ras- | 46 | 119 | 37 | 39 |
| regulated GEF | | | | |
| Spliceosomal | 37 | 96 | 40 | 79 |
| Protein Sap 49 | | | | |
| ets-2 | 33 | 140 | 90 | 38 |
| cytoplasmic | 60 | 142 | 78 | 37 |
| antiproteinase 2 | | | | |
| (CAP2) | | | | |
| MCP-1 | 41 | 151 | 43 | 46 |
| IL-7R | 49 | 143 | 44 | 44 |
| VCAM-1 | 18 | 873 | 83 | 96 |
| EphrinA1 | 96 | 356 | 148 | 113 |
| p50-NF-kappa | 5 | 158 | 33 | 57 |
| B homolog | | | | |
| Cox-2 | 22 | 154 | 0 | 30 |
| BCL3 | 114 | 283 | 125 | 198 |
| IFNGR2 | 59 | 418 | 186 | 209 |
| Na/K-ATPase | 87 | 200 | 78 | 148 |
| beta-1 | | | | |
| TRAF1 | 46 | 600 | 88 | 262 |
| IAP homolog B | 68 | 177 | 42 | 99 |
| RDC1 | 8 | 293 | 27 | 21 |
| ninjurin1 | 104 | 182 | 135 | 150 |
| fractalkine | −15 | 433 | 7 | 38 |
| lymphotoxin beta | −78 | 258 | −56 | −40 |
| metalloproteinase | 45 | 98 | 54 | 69 |
| stromelysin-2 | | | | |
| ABC transporter | 37 | 185 | 69 | 66 |
| B2 | | | | |
| beta-galactoside | 27 | 96 | 14 | 19 |
| alpha-2; 6- | | | | |
| sialyltransferase | | | | |

Genes with enhanced expression in the presence
of the two agents

| | No | TNFα | + Compound | |
|---|---|---|---|---|
| Genes | stimulation | stimulation | of Ex. 10 | + TSA |
| ICAM-1 | −19 | 1601 | 2174 | 2303 |
| I kappa B alpha | 271 | 1174 | 1259 | 1363 |
| B94 | 5 | 610 | 1010 | 924 |
| junB | 5 | 99 | 210 | 123 |
| exodus-1 | −19 | 157 | 310 | 1206 |
| Gro1 | 131 | 466 | 614 | 855 |

### TEST EXAMPLE 2

TSA (final concentration: 10 μM) or the compound prepared in Example 10 (final concentration: 1 μM) was added to HUVECs. The samples were stimulated with TNFα (final concentration: 10 ng/mL) for five hours, and RNA was recovered by use of an RNeasy Mini Kit (QIAGEN) in accordance with the manufacturer's protocol. Subsequently, cDNA was synthesized from the recovered RNA through a conventional method. The cDNA was subjected to quantitative PCR by the TaqMan probe method with a real-time quantitative PCR apparatus (ABI PRISM 7900HT, Applied Biosystems). The assay was performed for VCAM-1, GM-CSF, fractalkine, and ICAM-1. The expression level without stimulation was subtracted from the expression level with stimulation of TNFα, and assuming the resulting value to be 100, relative expression level was calculated. The results are shown in FIG. 2. TSA and the compound prepared in Example 10 exhibited either inhibitory or enhancing action on gene expressions. The results support the analysis results obtained from the test using GeneChip (see Test Example 1).

### TEST EXAMPLE 3

Cultured human cancer cells were placed in a 96-well plate. On the following day, a solution of the compound prepared in Example 10 (in five concentrations resulting from 10-fold stepwise dilution: 10⁻⁴, 10⁻⁵, 10⁻⁶, 10⁻⁷, or 10⁻⁸ M) was added, followed by incubation for two days. Cell count after growth was determined in each plate through colorimetry using sulforhodamine B. The concentration at which the cell count after growth was inhibited to 50% of that of the cell count of the control (in the absence of the compound prepared in Example 10) was calculated (GI50). Simultaneously, on the basis of the cell count just before addition of the compound prepared in Example 10 (time zero), the following value (concentration) was calculated.
TGI: a concentration at which cell growth is inhibited to a cell count equal to that at time zero (concentration at which no change in cell count is observed)
LC50: a concentration at which cell count is reduced to 50% of the cell count at time zero (cell-killing effect).

Table 2 shows the growth inhibitory effect of the compound prepared in Example 10 on 9 typical cancer cells.

TABLE 2

| Cancer cell lines | GI50 (μM) | TGI (μM) | LC50 (μM) |
|---|---|---|---|
| MCF-7 (breast cancer) | 0.16 | >100 | >100 |
| SF-539 (brain tumor) | 0.83 | >100 | >100 |
| HCC2998 (colon cancer) | 0.33 | 10 | 40 |
| DMS114 (lung cancer) | 0.038 | 2.6 | >100 |
| LOX-IMVI (melanoma) | 0.18 | 1.2 | 41 |
| OVCAR-3 (ovarian cancer) | 0.35 | 39 | >100 |
| ACHN (renal cancer) | 1.9 | >100 | >100 |
| MKN74 (stomach cancer) | 0.026 | 0.56 | >100 |
| PC-3 (prostatic carcinoma) | 26.3 | >100 | >100 |

As is apparent from Table 2, the compound prepared in Example 10 exhibits strong growth inhibitory effect (GI50) on typical cultured human cancer cells. Moreover, LC50 values suggest that the compound produces reduced side effects.

### TEST EXAMPLE 4

Cultured human cancer cells were added to a 96-well plate. On the following day, a solution of each of the compounds prepared in Examples 13, 23, 29, 36, and 114 (in five concentrations resulting from 10-fold stepwise dilution: 10⁻⁴, 10^{-5, 10}⁻⁶, 10⁻⁷, or 10⁻⁸ M) was added, followed by incubation for 48 hours. Subsequently, % growth of the cells was measured through colorimetry by use of a WST-1 (Dojindo) reagent for measurement of cell count. From the measurement data, % growth was calculated by use of the following equation, and 50% growth inhibitory concentration (GI50) was calculated from the dose-response curve of each compound:
% growth={[(OD as measured after 48 hours from addition of compound)−(OD at time zero)]/[(OD of control as measured after 48 hours)−(OD at time zero)]}×100.

As is apparent from Table 3, the compounds prepared in Examples 13, 23, 29, 36, and 114 all exhibited strong growth inhibitory effect on cultured human cancer cells.

TABLE 3

| | MCF-7 | HCT-15 | MKN-45 | MKN-74 |
|---|---|---|---|---|
| Compound | (breast | (colon | (stomach | (stomach |
| (Example | cancer) | cancer) | cancer) | cancer) |
| No.) | [GI50 (μM)] | [GI50 (μM)] | [GI50 (μM)] | [GI50 (μM)] |
| 13 | 0.7 | 0.8 | 3 | 0.7 |
| 23 | 0.6 | 0.9 | 4 | 0.7 |
| 29 | 0.7 | 0.8 | 3 | 0.4 |
| 36 | 0.7 | 1 | 2 | 0.6 |
| 114 | 0.6 | 0.8 | 2 | 0.2 |

### Industrial Applicability

The present invention can provide a method for treating cancer with reduced side effects.

1. A histone deacetylase inhibitor comprising a cyclic amine compound represented by the following formula (1): 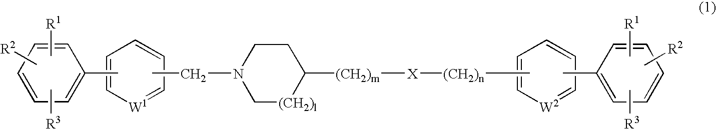
wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a solvate thereof.

2. The inhibitor according to claim 1, wherein R¹, R², and R³ are each independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxyl group, an alkoxycarbonyl group having a C1-C6 alkyl group, or an alkanoyl group having a C1-C6 allyl group.

3. The inhibitor according to claim 1, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

4. The inhibitor according to claim 3, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

5. The inhibitor according to claim 1, wherein the active ingredient is 4-[N -(4-methoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methoxyphenyl)-N-[[5-(3,4,5-trimethoxyphenyl)pyridin-3-yl]methyl]amino]-1-[[2-(3,4,5-trimethoxyphenyl)pyridin 4-yl]methyl]piperidine, 4-[N-(3,5-dimethoxyphenyl)-N-[[2-(3,4,5-trimethoxyphenyl)pyridin-4-yl]methyl]amino]-1-[[2-(3,4,5-trimethoxyphenyl)pyridin 4-yl]methyl]piperidine, 4-[N-(3,4-methylenedioxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, or a salt thereof.

6. A medicine for treating cancer comprising a cyclic amine compound represented by the following formula (1): 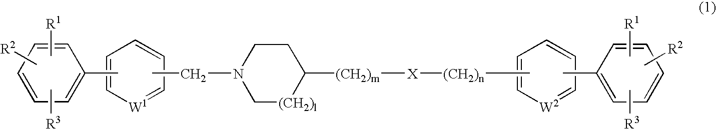
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a solvate thereof.

7. The medicine according to claim 6, wherein R¹, R², and R³ are each independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxy group, an alkoxycarbonyl group having a C1-C6 alkyl group, or an alkanoyl group having a C1-C6 alkyl group.

8. The medicine according to claim 6, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

9. The medicine according to claim 8, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

10. The medicine according to claim 6, wherein the active ingredient is 4-[N -(4-methoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methoxyphenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, 4-[N-(3,5-dimethoxyphenyl)-N-[[2-(3,4,5-trimethoxyphenyl)pyridin 4-yl]methyl]amino]-1-[[2-(3,4,5-trimethoxyphenyl)pyridin-4-yl]methyl]piperidine, 4-[N-(3,4-methylenedioxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin 4-yl]methyl]amino]-1-[[2-(3,4,5-trimethoxyphenyl)pyridin-4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, or a salt thereof.

11. A gene therapy facilitater comprising administering an effective amount of a cyclic amine compound represented by the following formula (1): 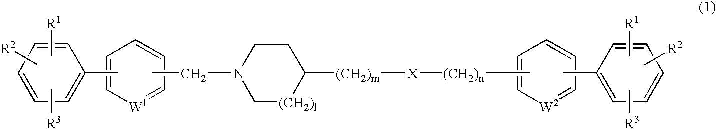
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a solvate thereof.

12. The facilitater according to claim 11, wherein R¹, R², and R³ each are independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxy group, an alkoxycarbonyl group having a C1-C6 alkyl group, or an alkanoyl group having a C1-C6 alkyl group.

13. The facilitater according to claim 11, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

14. The facilitater according to claim 13, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

15. The facilitater according to claim 11, wherein the active ingredient is 4-[N-(4-methoxyphenyl)-N-[[2-(3,4,5-trimethoxyphenyl)pyridin 4-yl]methyl]amino]-1-[[2-(3,4,5-trimethoxyphenyl)pyridin-4-yl]methyl]piperidine, 4-[N-(4-methoxyphenyl)-N-[[5-(3,4,5-trimethoxyphenyl)pyridin-3-yl]methyl]amino]-1-[[2-(3,4,5-trimethoxyphenyl)pyridin 4-yl]methyl]piperidine, 4-[N-(3,5-dimethoxyphenyl)-N-[[2-(3,4,5-trimethoxyphenyl)pyridin-4-yl]methyl]amino]-1-[[2-(3,4,5-trimethoxyphenyl)pyridin 4-yl]methyl]piperidine, 4-[N-(3,4-methylenedioxyphenyl)-N-[[2-(3,4,5-trimethoxyphenyl)pyridin 4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin 4-yl]methyl]piperidine, or a salt thereof.

16. A histone deacetylase inhibiting composition comprising a cyclic amine compound represented by the following formula (1): 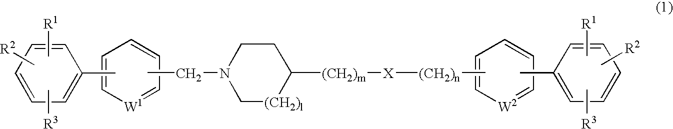
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a solvate thereof, and a pharmaceutically acceptable carrier.

17. The composition according to claim 16, wherein R¹, R², and R³ are each independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxyl group, an alkoxycarbonyl group having a C1-C6 alkyl group, or an alkanoyl group having a C1-C6 alkyl group.

18. The composition according to claim 16, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

19. The composition according to claim 18, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

20. The composition according to claim 16, wherein the active ingredient is 4-[N-(4-methoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4 -[N-(4-methoxyphenyl)-N-[[5-(3,4,5-trimethoxyphenyl)pyridin-3-yl]methylamino]-1-[[2-(3,4,5-trimethoxyphenyl)pyridin-4-yl]methyl]piperidine, 4-[N-(3,5-dimethoxyphenyl)-N-[[2-(3,4,5-trimethoxyphenyl)pyridin 4-yl]methyl]amino]-1-[[2-(3,4,5-trimethoxyphenyl)pyridin-4-yl]methyl]piperidine, 4-[N-(3,4-methylenedioxyphenyl)-N-[[2-(3,4,5-trimethoxyphenyl)pyridin 4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, or a salt thereof.

21. A medicinal composition for treating cancer comprising a cyclic amine compound represented by the following formula (1): 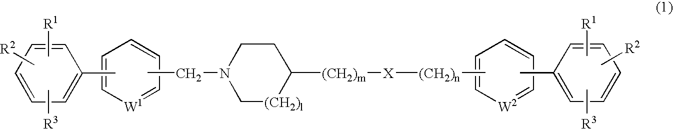
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a solvate thereof, and a pharmaceutically acceptable carrier.

22. The composition according to claim 21, wherein R¹, R², and R³ are each independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxy group, an alkoxycarbonyl group having a C1-C6 alkyl group, or an alkanoyl group having a C1-C6 alkyl group.

23. The composition according to claim 21, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

24. The composition according to claim 23, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

25. The composition according to claim 21, wherein the active ingredient is 4-[N-(4-methoxyphenyl)-N-[[2-(3,4,5-trimethoxyphenyl)pyridin 4-yl]methyl]amino]-1-[[2-(3,4,5-trimethoxyphenyl)pyridin-4-yl]methyl]piperidine, 4-[N-(4-methoxyphenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, 4-[N-(3,5-dimethoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(3,4-methylenedioxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, or a salt thereof.

26. A gene therapy facilitating composition comprising a cyclic amine compound represented by the following formula (1): 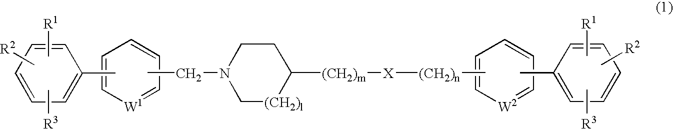
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a solvate thereof, and a pharmaceutically acceptable carrier.

27. The composition according to claim 26, wherein R¹, R², and R³ each are independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxy group, an alkoxycarbonyl group having a C1-C6 alkyl group, or an alkanoyl group having a C1-C6 alkyl group.

28. The composition according to claim 26, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

29. The composition according to claim 28, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

30. The composition according to claim 26, wherein the active ingredient is 4-[N-(4-methoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-N-(4-methoxyphenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, 4-[N-(3,5-dimethoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(3,4-methylenedioxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin 4-yl]methyl]piperidine, or a salt thereof.

31. Use, for producing histone deacetylase inhibitor of a cyclic amine compound represented by the following formula (1): 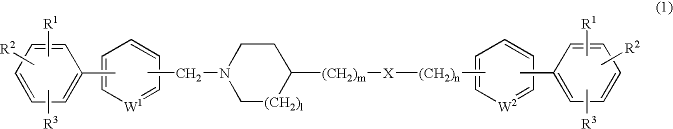
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and 1, m, and n each represent a number of 0 or 1), a salt thereof, or a solvate thereof.

32. The use according to claim 31, wherein R¹, R², and R³ are each independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxyl group, an alkoxycarbonyl group having a C1-C6 alkyl group, or an alkanoyl group having a C1-C6 alkyl group.

33. The use according to claim 31, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

34. The use according to claim 33, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

35. The use according to claim 31, wherein the active ingredient is 4-[N-(4-methoxyphenyl)-N-[[2-(3,4,5-trimethoxyphenyl)pyridin-4-yl]methyl]amino]-1-[[2-(3,4,5-trimethoxyphenyl)pyridin 4-yl]methyl]piperidine, 4-[N-(4-methoxyphenyl)-N-[[5-(3,4,5-trimethoxyphenyl)pyridin-3-yl]methyl]amino]-1-[[2-(3,4,5 -trimethoxyphenyl)pyridin 4-yl]methyl]piperidine, 4-[N-(3,5-dimethoxyphenyl)-N -[[2-(3,4,5-trimethoxyphenyl)pyridin-4-yl]methyl]amino]-1-[[2-(3,4,5-trimethoxyphenyl)pyridin 4-yl]methyl]piperidine, 4-[N-(3,4-methylenedioxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin-4-yl]methyl]amino]-1-[[2-(3,4,5-trimethoxyphenyl)pyridin 4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, or a salt thereof.

36. Use, for producing medicine for treating cancer of a cyclic amine compound represented by the following formula (1): 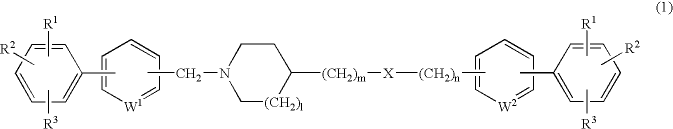
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a solvate thereof.

37. The use according to claim 36, wherein R¹, R², and R³ are each independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxy group, an alkoxycarbonyl group having a C1-C6 alkyl group, or an alkanoyl group having a C1-C6 alkyl group.

38. The use according to claim 36, wherein R⁴ is a hydrogen atom, a C1-C8 alkyl group, a C3-C8 alkenyl group, a C3-C8 allyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

39. The use according to claim 38, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

40. The use according to claim 36, wherein the active ingredient is 4-[N-(4-methoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methoxyphenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, 4-[N-(3,5-dimethoxyphenyl)-N -[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(3,4-methylenedioxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin 4-yl]methyl]piperidine, or a salt thereof.

41. Use, for producing gene therapy facilitator, of a cyclic amine compound represented by the following formula (1): 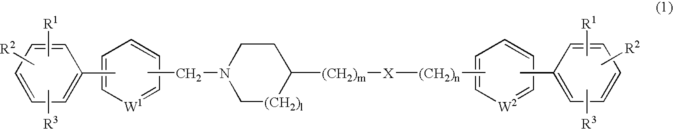
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a solvate thereof.

42. The use according to claim 41, wherein R¹, R², and R³ each are independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxy group, an alkoxycarbonyl group having a C1-C6 alkyl group, or an alkanoyl group having a C1-C6 alkyl group.

43. The use according to claim 41, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

44. The use according to claim 43, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

45. The use according to claim 41, wherein the active ingredient is 4-[N-(4-methoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methoxyphenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, 4-N-(3,5-dimethoxyphenyl)-N -[[2-(3,4,5-trimethoxyphenyl)pyridin-4-yl]methyl]amino]-1-[[2-(3,4,5-trimethoxyphenyl)pyridin 4-yl]methyl]piperidine, 4-[N-(3,4-methylenedioxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, or a salt thereof.

46. A method for inhibiting histone deacetylase, comprising administering an effective amount of a cyclic amine compound represented by the following formula (1): 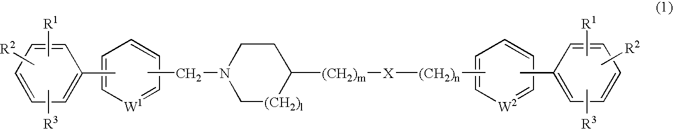
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a solvate thereof.

47. The method according to claim 46, wherein R¹, R², and R³ are each independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxyl group, an alkoxycarbonyl group having a C1-C6 alkyl group, or an alkanoyl group having a C1-C6 alkyl group.

48. The method according to claim 46, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

49. The method according to claim 48, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

50. The method according to claim 46, wherein the active ingredient is 4-[N -(4-methoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methoxyphenyl)-N-[[5-(3,4,5-trimethoxyphenyl)pyridin-3-yl]methyl]amino]-1-[[2 -(3,4,5-trimethoxyphenyl)pyridin-4-yl]methyl]piperidine, 4-[N-(3,5-dimethoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(3,4-methylenedioxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin-4-yl]methyl]amino]-1-[[2-(3,4,5-trimethoxyphenyl)pyridin 4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, or a salt thereof.

51. A method for treating cancer, comprising administering an effective amount of a cyclic amine compound represented by the following formula (1): 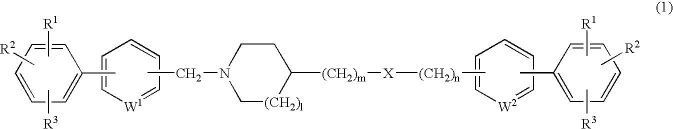
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NR⁴, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and 1, m, and n each represent a number of 0 or 1), a salt thereof, or a solvate thereof.

52. The method according to claim 51, wherein R¹, R², and R³ are each independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxy group, an alkoxycarbonyl group having a C1-C6 alkyl group, or an alkanoyl group having a C1-C6 alkyl group.

53. The method according to claim 51, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

54. The method according to claim 53, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

55. The method according to claim 51, wherein the active ingredient is 4-[N -(4-methoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-N-(4-methoxyphenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, 4-[N-(3,5-dimethoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-N-(3,4-methylenedioxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, or a salt thereof.

56. A method for facilitating gene therapy, comprising administering an effective amount of a cyclic amine compound represented by the following formula (1): 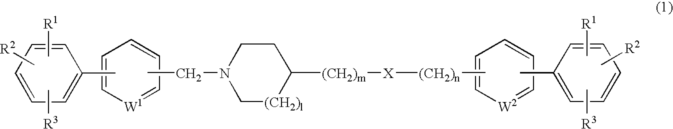
(wherein R¹, R², and R³ each independently represent a hydrogen atom, a halogen atom, a hydroxy group, an alkyl group, a halogen-substituted alkyl group, an alkoxy group, an alkylthio group, a carboxyl group, an alkoxycarbonyl group, or an alkanoyl group; W¹ and W² each independently represent N or CH; X represents O, NRW, CONR⁴, or NR⁴CO; R⁴ represents a hydrogen atom, an alkyl group, an alkenyl group, an alkynyl group, a substituted or unsubstituted aryl group, a substituted or unsubstituted heteroaryl group, a substituted or unsubstituted aralkyl group, or a substituted or unsubstituted heteroaralkyl group; and l, m, and n each represent a number of 0 or 1), a salt thereof, or a solvate thereof.

57. The method according to claim 56, wherein R¹, R², and R³ each are independently a hydrogen atom, a halogen atom, a hydroxy group, a C1-C8 alkyl group, a halogen-substituted C1-C8 alkyl group, an alkoxy group having a C1-C8 alkyl group, an alkylthio group having a C1-C8 alkyl group, a carboxy group, an alkoxycarbonyl group having a C1-C6 alkyl group, or an alkanoyl group having a C1-C6 alkyl group.

58. The method according to claim 56, wherein R⁴ is a hydrogen atom, a C1-C8 alkyl group, a C3-C8 alkenyl group, a C3-C8 alkynyl group, a substituted or unsubstituted C6-C14 aryl group, a substituted or unsubstituted heteroaryl group containing a 5- or 6-membered ring having one to four nitrogen atoms, a substituted or unsubstituted (C6-C14)-aryl —(C1-C6)-alkyl group, or a substituted or unsubstituted heteroaryl —(C1-C6)-alkyl group containing a 5- or 6-membered ring having one to four nitrogen atoms.

59. The method according to claim 58, wherein the substituent(s) of the aryl group, the aryl group of the aralkyl group, the heteroaryl group, or the heteroaryl group of the heteroaralkyl group represented by R⁴ is (are) one to three groups or atoms selected from an alkyl group, an alkoxy group, an alkylthio group, a halogen atom, a nitro group, an amino group, an acetylamino group, a trifluoromethyl group, and an alkylenedioxy group.

60. The method according to claim 56, wherein the active ingredient is 4-[N -(4-methoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(4-methoxyphenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, 4-[N-(3,5-dimethoxyphenyl)-N-[[2-(3,4,5-trimethoxyphenyl)pyridin-4-yl]methyl]amino]-1-[[2-(3,4,5-trimethoxyphenyl)pyridin-4-yl]methyl]piperidine, 4-[N-(3,4-methylenedioxyphenyl)-N-[[2-(3,4,5-trimethoxyphenyl)pyridin 4-yl]methyl]amino]-1-[[2-(3,4,5-trimethoxyphenyl)pyridin-4-yl]methyl]piperidine, 4-[N-methyl-N-[[2-(3,4,5-trimethoxyphenyl)pyridin-4-yl]methyl]amino]-1-[[2-(3,4,5-trimethoxyphenyl)pyridin 4-yl]methyl]piperidine, 4-[N-(4-methylthiophenyl)-N-[[5-(3,4,5-trimethoxyphenyl)pyridin-3-yl]methyl]amino]-1-[[2-(3,4,5-trimethoxyphenyl)pyridin-4-yl]methyl]piperidine, or a salt thereof.
